SEQUENCE LISTING (3 sequences)

SEQ ID NO: 1 - DNA, 36 bp, Artificial Sequence
  Oligonucleotide
tagtaccgccaccctcagaacctttttttttttttt

SEQ ID NO: 2 - DNA, 22 bp, Artificial Sequence
  Oligonucleotide
atcatggcggtgggagtcttgg

SEQ ID NO: 3 - DNA, 22 bp, Artificial Sequence
  Oligonucleotide
tagtaccgccaccctcagaacc

(19) United States
(12) Patent Application Publication
(10) Pub. No.: US 2009/0047246 A1
(43) Pub. Date: Feb. 19, 2009

(54) NOVEL INHIBITORS OF HEPATITIS C VIRUS REPLICATION

(75) Inventors: Leonid Beigelman, San Mateo, CA (US); Brad Buckman, Oakland, CA (US); Vladimir Serebryany, Burlingame, CA (US); Guangyi Wang, Carlsbad, CA (US); Jasenka Matulic-Adamic, Brisbane, CA (US); Antitsa Dimitrova Stoycheva, Half Moon Bay, CA (US); Steven W. Andrews, Longmont, CO (US); Shawn Maurice Misialek, Redwood City, CA (US); P.T. Ravi Rajagopalan, San Ramon, CA (US); Andrew M. Fryer, Erie, CO (US); Indrani Gunawardana, Longmont, CO (US); Julia Haas, Boulder, CO (US); Lily Huang, Longmont, CO (US); Machender R. Madduru, Erie, CO (US); Gan Zhang, Longmont, CO (US); Karl Kossen, Brisbane, CA (US); Scott D. Seiwert, Pacifica, CA (US); Lawrence M. Blatt, San Francisco, CA (US)

Correspondence Address: KNOBBE MARTENS OLSON & BEAR LLP, 2040 MAIN STREET, FOURTEENTH FLOOR, IRVINE, CA, 92614, US

(73) Assignee: InterMune, Inc., Brisbane, CA (US)

(21) Appl. No.: 12/029,399

(22) Filed: Feb. 11, 2008

Related U.S. Application Data
(60) Provisional application No. 60/889,433, filed on Feb. 12, 2007.

(51) Int. Cl. A61K 38/21; C07D 409/06; A61K 31/405; A61K 31/506; C07D 403/08; A61K 31/404; C07D 209/20; A61K 31/675; C07F 9/572; A61K 31/437; C07D 471/02; C12N 9/99; A61K 31/44; A61K 39/395; A61K 31/427; C12N 9/50; C12N 9/00; C12Q 1/68; G01N 33/53

(52) U.S. Cl. 424/85.5; 548/454; 514/414; 514/256; 544/333; 548/491; 514/419; 514/80; 548/414; 546/113; 514/300; 435/184; 514/350; 514/365; 435/219; 435/183; 435/6; 435/7.1; 424/85.7

(57) ABSTRACT

The embodiments provide compounds of the general Formula I, as well as compositions, including pharmaceutical compositions, comprising a subject compound. The embodiments further provide treatment methods, including methods of treating a hepatitis C virus infection, the methods generally involving administering to an individual in need thereof an effective amount of a subject compound or composition.

### RELATED APPLICATION

This application claims the benefit of U.S. Provisional Application No. 60/889,433, filed Feb. 12, 2007, which is incorporated herein by reference in its entirety.

### BACKGROUND OF THE INVENTION

1. Field of the Invention

The present invention relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for NS3-mediated processing events and enhances proteolytic efficiency at all sites recognized by NS3. The NS3 protease also exhibits nucleoside triphosphatase and RNA helicase activities (the region of the protein corresponding to the RNA helicase activity is herein referred to as “NS3 helicase”). The helicase activity unwinds viral RNA as a necessary step prior to replication. NS3 helicase is thought to be essential for viral replication to occur in cells and therefore inhibition of the domain of NS3 is an attractive method for treating HCV replication in man. NS5B is an RNA-dependent RNA polymerase involved in the replication of HCV RNA.

### Literature

Gallinari, P. (1998) Journal of Virology, p. 6758-6769; Kim, J. W. (2003) Journal of Virology, p. 571-582; Chang, S. C. (2000) Journal of Virology, p. 9732-9737; Phillip, S. P. (2002) The EMBO Journal 21 (5): 1168-1176; Sameer, S., and Velankar (1999) Cell 97:75-84; Serebrov, V. Nature 430:476-480.

### SUMMARY OF THE INVENTION

The present embodiments provide compounds of the general formula (I)

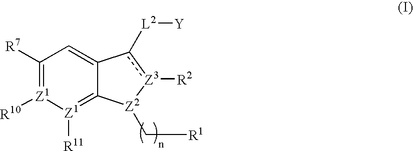

wherein:
n is an integer from 0 to 3;
R¹ is selected from the group consisting of H, -A¹-L¹-A², and an optionally substituted: alkyl, alkenyl, alkynyl, cycloalkyl, heterocyclyl, aryl, heteroaryl, —C(O)-aryl, —C(O)-aralkyl, or —C(O)-heterocyclyl-aralkyl; or R¹ is absent and n is 0 when Z² is O or S;
   wherein if R¹ is —C(O)-aryl, —C(O)-aralkyl, or —C(O)-heterocyclyl-aralkyl, then n is not 0;
A¹ and A² are independently selected from the group consisting of optionally substituted aryl and optionally substituted heteroaryl;
L¹ is oxy, C₁₋₆ alkoxy, —NR⁵C(O)-alkyl-, —NR⁵C(O)CH₂S—, —NR⁵CH₂—, or absent;
L² is —CR^{3a}R^{3b}—, —CR^{3a}R^{3b}CR^{3a}R^{3b}—, —CR^{3a}═CR^{3a}—, or absent;
each R^{3a} and each R^{3b} are independently selected from the group consisting of H, halo, hydroxy, NH₃⁺, —NHC(O)NH₂, —NHC(O)OR⁹, —NHC(O)R⁹, and an optionally substituted: C₁₋₆ alkyl, cycloalkyl-alkyl, heterocyclyl-alkyl, heteroaralkyl, aralkyl, or aryl, or an R^{3a} and R^{3b} together form an oxo;
an R^{3a} together with R² optionally form an optionally substituted cycloalkyl or optionally substituted heterocyclyl;
Y is selected from the group consisting of H, halo, ethynyl, —C(O)H, —CN, —C(O)OR⁴, —C(O)NR⁵R⁶, —C(O)NHSO₂R⁹, —PO₃H₂, 1H-tetrazol-5-yl, 1H-1,2,4-triazol-5-yl, 1H-pyrazol-5-yl, 1,2-dihydro-1,2,4-triazol-3-on-5-yl, and 1,2-dihydro-pyrazol-3-on-5-yl,
   wherein if Y is H, then:
      at least one R^{3a} or R^{3b} is an optionally substituted aryl, or
      R¹ is -A¹-L¹-A² or an optionally substituted: aryl, heteroaryl, —C(O)-aryl, —C(O)-aralkyl, or —C(O)-heterocyclyl-aralkyl;
R⁷ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl;
R¹⁰ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, heteroaralkyl, or is absent, or R⁷ and R¹⁰ together form an optionally substituted ring or ring system;
R¹¹ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl, or is absent;
each Z¹ are independently C or N;
Z² is CH, N, O, or S;
Z³ is C or N;
R² is selected from the group consisting of H, —C(O)OR⁴, —C(O)NR⁵R⁶, —C(O)-A¹-L¹-A², —CH₂-A¹-L¹-A², —C(O)CH₂-A¹-L¹-A², —C(O)NHCH₂-A¹-L¹-A², and an optionally substituted: alkyl, —C(O)-alkyl, aryl, —C(O)-aryl, aralkyl, —C(O)-aralkyl, or heteroaralkyl,
   wherein if
      R¹ is not -A¹-L¹-A² or an optionally substituted: aryl, heteroaryl, —C(O)-aryl, —C(O)-aralkyl, or —C(O)-heterocyclyl-aralkyl, then:
      R² is selected from the group consisting of —C(O)-A¹-L¹-A², —CH₂-A¹-L¹-A², —C(O)CH₂-A¹-L¹-A², —CH₂— (optionally substituted heteroaryl), and optionally substituted —C(O)-aralkyl,
   at least one R^{3a} or R^{3b} is an optionally substituted heteroaralkyl,
      Y is —C(O)OH or —C(O)H and at least one Z¹ is N.
      Y is —C(O)OH or —C(O)H and R¹⁰ is phenyl or —O-benzyl,
      Y is —C(O)OH or —C(O)H and R¹¹ is —O-(optionally substituted phenyl), or
      Y is —C(O)OH or —C(O)H, R⁷ is —O-benzyl, and R¹⁰ is —O-methyl;
R⁴ is H or optionally substituted: alkyl, alkenyl, alkynyl, aryl, aralkyl, heteroaryl, or heteroaralkyl;
R⁵ and R⁶ are each independently selected from the group consisting of H, CN, and an optionally substituted: C₁₋₆ alkyl, C₃₋₇ cycloalkyl, hetereocyclyl, -hetereocyclyl-C(O)OR⁴, aryl, heteroaryl, aralkyl, heteroaralkyl, or cycloalkyl-alkyl, or R⁵ and R⁶ together form an optionally substituted ring or ring system; and
R⁹ is selected from the group consisting of alkyl, cycloalkyl, and aryl;
with the proviso that:
   if R¹ is a pyridine, pyrimidine, or quinoline, or if R¹ is naphthalene and n is not 0, then Y is not CO₂H;
   if R¹ is an unsubstituted phenyl, then Y is not —C(O)OMe, —C(O)OEt, —C(O)O-t-Bu, —C(O)OBn, —C(O)NMe₂, —C(O)NEt₂, or —C(O)N(i-Pr)₂;
   if n is less than 3 and R¹ is an unsubstituted phenyl or unsubstituted biphenyl and Y is —C(O)OH, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn or Br;
   if Y is —C(O)OH and R¹ is phenyl substituted with a single halogen, —SO₂Me, —OCF₃, —OCF₂CF₃, —OCF₂CF₂H, —NC(O)CH₂Br, -Me, —SCH₃, or -t-Bu or R¹ is phenyl fused with a dioxolane ring, then R⁷ is —OBn or Br;
   if Y is —C(O)OMe and R¹ is phenyl substituted with a single Cl, then R⁷ is —OBn;
   if Y is —C(O)OEt and R¹ is phenyl substituted with a single halogen, —SO₂Me, —NH₂, —OH, —OCH₃, or —NO₂, or two Cl, then R⁷ is —OBn;
   if Y is —C(O)O-(substituted phenyl) and R¹ is phenyl substituted with two Cl, then R⁷ is —OBn;
   if Y is —C(O)O-alkyl-phenyl and R¹ is unsubstituted phenyl or phenyl substituted with a single Br, then R⁷ is —OBn;
   if n is 0 and R¹ is unsubstituted phenyl or phenyl substituted by a single methyl, then R² is selected from the group consisting of —C(O)-A¹-L¹-A2, —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn;
   if R¹ is -A¹-L¹-A², L¹ is methoxy, A¹ is unsubstituted phenyl, A² is phenyl substituted with a single CF₃, and Y is —C(O)OH, then R⁷ is —OBn;
   if R¹ is -A¹-L¹-A², L¹ is absent, A¹ is benzofuran, A² is thiazole, and Y is —C(O)OH, then R⁷ is —OBn; and
   if R¹ is -A¹-L¹-A², L¹ is methoxy or absent, A¹ is unsubstituted phenyl, A² is unsubstituted phenyl, R² is alkyl, and Y is —C(O)O-alkyl, then R⁷ is —OBn.

Other embodiments provide compounds of formula I having the following definitions:
n is an integer from 0 to 3;
R¹ is selected from the group consisting of H, -A¹-L¹-A², and an optionally substituted: alkyl, alkenyl, alkynyl, cycloalkyl, heterocyclyl, aryl, heteroaryl, —C(O)-aryl, —C(O)-aralkyl, —C(O)-heteroaryl, or —C(O)-heterocyclyl-aralkyl; or R¹ is absent and n is 0 when Z² is O or S;
   wherein if R¹ is —C(O)-aryl, —C(O)-aralkyl, or —C(O)-heterocyclyl-aralkyl, then n is not 0;
A¹ and A² are independently selected from the group consisting of optionally substituted aryl and optionally substituted heteroaryl;
L¹ is oxy, C₁₋₆ alkoxy, —NR⁵C(O)-alkyl-, —NR⁵C(O)CH₂S—, —NR⁵CH₂—, —NR⁵ or absent;
L² is —CR^{3a}R^{3b}—, —CR^{3a}R^{3b}CR^{3a}R^{3b}—, —CR^{3a}═CR^{3a}—, or absent;
each R^{3a} and each R^{3b} are independently selected from the group consisting of H, halo, hydroxy, NH₃⁺, —NHC(O)NH₂, —NHC(O)OR⁹, —NHC(O)R⁹, —C(O)R⁴ and an optionally substituted: C₁₋₆ alkyl, cycloalkyl-alkyl, heterocyclyl-alkyl, heteroaralkyl, aralkyl, or aryl, or an R^{3a} and R^{3b} together form an oxo;
an R^{3a} together with R² optionally form an optionally substituted cycloalkyl or optionally substituted heterocyclyl;
Y is selected from the group consisting of H, halo, ethynyl, —C(O)H, —CN, —C(O)OR⁴, —C(O)NR⁵R⁶, —C(O)NHSO₂R⁹, —C(O)NHOR⁴, —C(O)OCH₃OC(O)R⁴, —NHC(O)R⁴, —C(O)NHOR⁴, —C(O)OCH₃OR⁴, —PO₃H₂, 1H-tetrazol-5-yl, 1H-1,2,4-triazol-5-yl, 1H-pyrazol-5-yl, 1,2-dihydro-1,2,4-triazol-3-on-5-yl, and 1,2-dihydro-pyrazol-3-on-5-yl,
   wherein if Y is H, then:
      at least one R^{3a} or R^{3b} is an optionally substituted aryl, or
      R¹ is -A¹-L¹-A² or an optionally substituted: aryl, heteroaryl, —C(O)-aryl, —C(O)-aralkyl, or —C(O)-heterocyclyl-aralkyl;
R⁷ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl;
R¹⁰ is selected from the group consisting of H, halo, —CN, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, heterocyclyl, cycloalkylalkoxy, aryl, aralkyl, heteroaryl, heteroaralkyl, or is absent, or R⁷ and R¹⁰ together form an optionally substituted ring or ring system;
R¹¹ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl, or is absent;
each Z¹ are independently C or N;
Z² is CH, N, O, or S;
Z³ is C or N;
R² is selected from the group consisting of H, —C(O)OR⁴, —C(O)NR⁵R⁶, —C(O)-A¹-L¹-A², —CH₂-A¹-L¹-A², —C(O)CH₂-A¹-L¹-A², —C(O)NHCH₂-A¹-L¹-A², and an optionally substituted: alkyl, —C(O)-alkyl, aryl, —C(O)-aryl, aralkyl, —C(O)-aralkyl, or heteroaralkyl,
   wherein if
      R¹ is not -A¹-L¹-A² or an optionally substituted: aryl, heteroaryl, —C(O)-aryl, —C(O)-aralkyl, or —C(O)-heterocyclyl-aralkyl, then:
      R² is selected from the group consisting of —C(O)-A¹-L¹-A², —CH₂-A¹-L¹-A², —C(O)CH₂-A¹-L¹-A², —CH₂— (optionally substituted heteroaryl), and optionally substituted —C(O)-aralkyl,
      at least one R^{3a} or R^{3b} is an optionally substituted heteroaralkyl,
      Y is —C(O)OH or —C(O)H and at least one Z¹ is N.
      Y is —C(O)OH or —C(O)H and R¹⁰ is phenyl, phenyl substituted with one or more amino, or —O-benzyl,
      Y is —C(O)OH or —C(O)H and R¹¹ is —O-(optionally substituted phenyl), or
      Y is —C(O)OH or —C(O)H, R⁷ is —O-benzyl, and R¹⁰ is —O-methyl;
R⁴ is H or optionally substituted: alkyl, alkenyl, alkynyl, aryl, aralkyl, heteroaryl, heterocyclyl, or heteroaralkyl;
R⁵ and R⁶ are each independently selected from the group consisting of H, CN, and an optionally substituted: C₁₋₆ alkyl, C₃₋₇ cycloalkyl, hetereocyclyl, -hetereocyclyl-C(O)OR⁴, aryl, heteroaryl, aralkyl, heteroaralkyl, or cycloalkyl-alkyl, or R⁵ and R⁶ together form an optionally substituted ring or ring system; and
R⁹ is selected from the group consisting of alkyl, cycloalkyl, and aryl;
with the proviso that:
   if R¹ is a pyridine, pyrimidine, or quinoline, or if R¹ is naphthalene and n is not 0, then Y is not CO₂H;
   if R¹ is an unsubstituted phenyl, then Y is not —C(O)OMe, —C(O)OEt, —C(O)O-t-Bu, —C(O)OBn, —C(O)NMe₂, —C(O)NEt₂, or —C(O)N(i-Pr)₂;
   if n is less than 3 and R¹ is an unsubstituted phenyl or unsubstituted biphenyl and Y is —C(O)OH, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn, Br, or phenyl substituted with one or more amino;
   if Y is —C(O)OH and R¹ is phenyl substituted with a single halogen, —SO₂Me, —OCF₃, —OCF₂CF₃, —OCF₂CF₂H, —NC(O)CH₂Br, -Me, —SCH₃, or -t-Bu or R¹ is phenyl fused with a dioxolane ring, then R⁷ is —OBn or Br;
   if Y is —C(O)OMe and R¹ is phenyl substituted with a single Cl, then R⁷ is —OBn;
   if Y is —C(O)OEt and R¹ is phenyl substituted with a single halogen, —SO₂Me, —NH₂, —OH, —OCH₃, or —NO₂, or two Cl, then R⁷ is —OBn or R¹⁰ is phenyl substituted with one or more nitro;
   if Y is —C(O)O-(substituted phenyl) and R¹ is phenyl substituted with two Cl, then R⁷ is —OBn;
   if Y is —C(O)O-alkyl-phenyl and R¹ is unsubstituted phenyl or phenyl substituted with a single Br, then R⁷ is —OBn;
   if n is 0 and R¹ is unsubstituted phenyl or phenyl substituted by a single methyl, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn;
   if R¹ is -A¹-L¹-A², L¹ is methoxy, A¹ is unsubstituted phenyl, A² is phenyl substituted with a single CF₃, and Y is —C(O)OH, then R⁷ is —OBn;
   if R¹ is -A¹-L¹-A², L¹ is absent, A¹ is benzofuran, A² is thiazole, and Y is —C(O)OH, then R⁷ is —OBn; and
   if R¹ is -A¹-L¹-A², L¹ is methoxy or absent, A¹ is unsubstituted phenyl, A² is unsubstituted phenyl, R² is alkyl, and Y is —C(O)O-alkyl, then R⁷ is —OBn.

The present embodiments provide for a method of inhibiting NS3/NS4 helicase activity comprising contacting a NS3/NS4 helicase with a compound disclosed herein.

The present embodiments provide for a method of treating hepatitis by modulating NS3/NS4 helicase comprising contacting a NS3/NS4 helicase with a compound disclosed herein.

Preferred embodiments provide a pharmaceutical composition comprising a preferred compound; and a pharmaceutically acceptable carrier.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

Preferred embodiments provide for a method of modulating NS3 activity comprising contacting an NS3 protein with a compound disclosed herein.

Preferred embodiments provide for a method of treating hepatitis by modulating NS3 helicase comprising contacting an NS3 helicase with the compound disclosed herein.

### BRIEF DESCRIPTION OF THE DRAWINGS

FIG. 1 depicts a graph charting the helicase activity of NS3 in the presence of various buffers.

FIG. 2A depicts a graph charting NS3 helicase activity as a function of time for varying concentrations of NS3 enzyme.

FIG. 2B depicts a graph charting the initial rate of the unwinding reaction (RFU/second) as a function of NS3 enzyme concentration.

FIG. 2C depicts a graph charting the initial rate (RFU (average)) of the unwinding reaction as a function of time for varying concentrations of NS3 enzyme.

FIG. 2D depicts a graph charting the amplitude (final RFU) of the unwinding reaction as a function of enzyme concentration.

FIG. 3 depicts a graph charting NS3 helicase activity in solutions comprising varying amounts of MgCl₂.

FIGS. 4A and 4B depict graphs charting NS3 helicase activity in assays comprising varying amounts of ATP.

FIGS. 5A and 5B depicts graphs charting NS3 helicase activity in assays comprising varying amounts of oligonucleotide substrate.

FIG. 5C depicts a graph charting the slope of plots depicting NS3 helicase activity in assays comprising varying amounts of oligonucleotide substrate versus the oligonucleotide substrate concentration.

### DETAILED DESCRIPTION OF THE EMBODIMENTS

### Definitions

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “Type II interferon receptor agonist” refers to any naturally occurring or non-naturally occurring ligand of human Type II interferon receptor that binds to and causes signal transduction via the receptor. Type II interferon receptor agonists include native human interferon-γ, recombinant IFN-γ species, glycosylated IFN-γ species, pegylated IFN-γ species, modified or variant IFN-γ species, IFN-γ fusion proteins, antibody agonists specific for the receptor, non-peptide agonists, and the like.

As used herein, the term “a Type III interferon receptor agonist” refers to any naturally occurring or non-naturally occurring ligand of humanIL-28 receptor α (“IL-28R”), the amino acid sequence of which is described by Sheppard, et al., infra., that binds to and causes signal transduction via the receptor.

As used herein, the term “interferon receptor agonist” refers to any Type I interferon receptor agonist, Type II interferon receptor agonist, or Type III interferon receptor agonist.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., a Type I or Type III interferon receptor agonist, e.g., IFN-α) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC8 hr) is no more than about 20% above or about 20% below, and preferably no more than about 15% above or about 15% below, and more preferably no more than about 10% above or about 10% below, the average area under the curve of the biological parameter over an 8 hour period during the time course (AUC8 hr average). The AUC8 hr average is defined as the quotient (q) of the area under the curve of the biological parameter over the entirety of the time course (AUCtotal) divided by the number of 8 hour intervals in the time course (total/3 days), i.e., q=(AUCtotal)/(total/3 days). For example, in the context of a serum concentration of a drug, the serum concentration of the drug is maintained at a substantially steady state during a time course when the area under the curve of serum concentration of the drug over time for any 8 hour period during the time course (AUC8 hr) is no more than about 20% above or about 20% below the average area under the curve of serum concentration of the drug over an 8 hour period in the time course (AUC8 hr average), i.e., the AUC8 hr is no more than 20% above or 20% below the AUC8 hr average for the serum concentration of the drug over the time course.

The term “alkyl” used herein refers to a monovalent straight or branched chain radical of from one to twenty carbon atoms, including, but not limited to, methyl, ethyl, n-propyl, isopropyl, n-butyl, isobutyl, tert-butyl, n-hexyl, and the like.

The term “halo” used herein refers to fluoro, chloro, bromo, or iodo.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “aryl” used herein refers to homocyclic aromatic radical whether fused or not fused. Examples of aryl groups include, but are not limited to, phenyl, naphthyl, phenanthrenyl, naphthacenyl, and the like. The aryl may be fused to other aryl rings, heteroaryl rings, cycloalkyl rings, cycloalkenyl rings, or heterocyclyl rings.

The term “cycloalkyl” used herein refers to saturated aliphatic ring system radical having three to twenty carbon atoms including, but not limited to, cyclopropyl, cyclopentyl, cyclohexyl, cycloheptyl, and the like. The cycloalkyl may be fused to other cycloalkyl rings, aryl rings, heteroaryl rings, cycloalkenyl rings, or heterocyclyl rings.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, and the like. The cycloalkenyl may be fused to other cycloalkenyl rings, aryl rings, heteroaryl rings, cycloalkyl rings, or heterocyclyl rings

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantyl, norbornyl, and the like.

The term “polycycloalkenyl” used herein refers to aliphatic ring system radical having at least two rings that are fused with or without bridgehead carbons in which at least one of the rings has a carbon-carbon double bond. Examples of polycycloalkenyl groups include, but are not limited to, norbornylenyl, 1,1′-bicyclopentenyl, and the like.

The term “polycyclic hydrocarbon” used herein refers to a ring system radical in which all of the ring members are carbon atoms. Polycyclic hydrocarbons can be aromatic or can contain less than the maximum number of non-cumulative double bonds. Examples of polycyclic hydrocarbon include, but are not limited to, naphthyl, dihydronaphthyl, indenyl, fluorenyl, and the like.

The term “heterocyclic” or “heterocyclyl” used herein refers to non-aromatic cyclic ring system radical having at least one ring system in which one or more ring atoms are not carbon, namely heteroatom. Examples of heterocyclic groups include, but are not limited to, morpholinyl, tetrahydrofuranyl, dioxolanyl, pyrrolidinyl, pyranyl, pyridyl, pyrimidinyl, and the like. The heterocyclyl may be fused to other heterocyclyl rings, aryl rings, heteroaryl rings, cycloalkyl rings, or cycloalkenyl rings

The term “heteroaryl” used herein refers to heterocyclic group, whether one or more rings, formally derived from an arene by replacement of one or more methine and/or vinylene groups by trivalent or divalent heteroatoms, respectively, in such a way as to maintain the aromatic system in one or more rings. Examples of heteroaryl groups include, but are not limited to, pyridyl, pyrrolyl, oxazolyl, indolyl, and the like. The heteroaryl may be fused to other heteroaryl rings, aryl rings, cycloalkyl rings, cycloalkenyl rings, or heterocyclyl rings

The phrase “ring or ring system” used herein refers to a cycloalkyl, cycloalkenyl, polycycloalkyl, polycycloalkenyl, heterocyclyl, or heteroaryl radical.

The term “arylalkyl” or “aralkyl” used herein refers to one or more aryl groups appended to an alkyl radical. Examples of arylalkyl groups include, but are not limited to, benzyl, phenethyl, phenpropyl, phenbutyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like.

The term “heteroarylalkyl” or “heteroaralkyl” used herein refers to one or more heteroaryl groups appended to an alkyl radical. Examples of heteroarylalkyl include, but are not limited to, pyridylmethyl, furanylmethyl, thiophenylethyl, and the like.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like.

The term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage. Examples of alkoxy groups include, but are not limited to, methoxy, ethoxy, propoxy, isopropoxy, butoxy, n-butoxy, sec-butoxy, t-butoxy and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “carbamyl” used herein refers to RNHCOO—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO—.

The term “thiocarbonyl” used herein refers to C═S.

The term “thiocarboxy” used herein refers to CSOH.

The term “C-amido” used herein refers to —C(O)NR₂, where each R is independently H or C₁-C₆ alkyl.

The term “N-amido” used herein refers to —NR₂C(O)R, where each R is independently H or C₁-C₆ alkyl.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, a substituted group is derived from the unsubstituted parent structure in which there has been an exchange of one or more hydrogen atoms for another atom or group. When substituted, the substituent group(s) is (are) one or more group(s) individually and independently selected from C₁-C₆ alkyl, C₁-C₆ alkenyl, C₁-C₆ alkynyl, C₃-C₆ cycloalkyl, C₃-C₆ heterocycloalkyl (e.g., tetrahydrofuryl), aryl, aralkyl, heteroaryl, halo (e.g., chloro, bromo, iodo and fluoro), cyano, hydroxy, hydroxy-C₁-C₆ alkyl, halogenated C₁-C₆ alkyl, C₁-C₆ alkoxy, halogenated C₁-C₆ alkoxy (e.g., perhalogenated C₁-C₆ alkoxy), aryloxy, sulfhydryl (mercapto), C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, C₁-C₆ alkylamino-C₁-C₆ alkylamino, acyanoamino, nitro, N-carbamyl (e.g., —NHC(O)O-t-butyl, —N(cyclopropyl)C(O)O-t-butyl, etc.), C-carbamate, keto (oxy), carbonyl, O-carboxy (e.g., —OC(O)CH₃, etc.), urea, C-carboxy (e.g., —C(O)OCH₃, —C(O)O-alkyl, etc.), C₁-C₆-alkylcarboxy, C-amido (e.g., —C(O)N(CH₃)₂, —C(O)NH₂, etc.), N-amido (e.g., —N(CH₃)C(O)CH₃, —NHC(O)CH₃, —N(CH₃)C(O)H, —N(CH₂CH₃C(O)H, etc.), C₁-C₆-alkyl-OC(O)NH—C₁-C₆-alkyl, glycolyl, glycyl, hydrazino, guanyl, sulfamyl, sulfonyl (e.g., C₁-C₆-alkylsulfonyl, hydroxy-C₁-C₆-alkylsulfonyl), sulfonylamino (e.g., C₁-C₆-alkylsulfonylamino (e.g., —N(CH₃)SO₂CH₃)), sulfinyl, thiocarbonyl, thiocarboxy, and combinations thereof. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The present embodiments provide compounds of Formula I, as well as pharmaceutical compositions and formulations comprising any compound of Formula I. A subject compound is useful for treating HCV infection and other disorders, as discussed below.

### Compositions

The present embodiments provide compounds of the general formula (I)

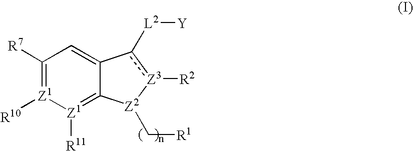

wherein:
n is an integer from 0 to 3;
R¹ is selected from the group consisting of H, -A¹-L¹-A², and an optionally substituted: alkyl, alkenyl, alkynyl, cycloalkyl, heterocyclyl, aryl, heteroaryl, —C(O)-aryl, —C(O)-aralkyl, or —C(O)-heterocyclyl-aralkyl; or R¹ is absent and n is 0 when Z² is O or S;
   wherein if R¹ is —C(O)-aryl, —C(O)-aralkyl, or —C(O)-heterocyclyl-aralkyl, then n is not 0;
A¹ and A² are independently selected from the group consisting of optionally substituted aryl and optionally substituted heteroaryl;
L¹ is oxy, C₁₋₆ alkoxy, —NR⁵C(O)-alkyl-, —NR⁵C(O)CH₂S—, —NR⁵CH₂—, or absent;
L² is —CR^{3a}R^{3b}—, —CR^{3a}R^{3b}CR^{3a}R^{3b}—, —CR^{3a}═CR^{3a}—, or absent;
each R^{3a} and each R^{3b} are independently selected from the group consisting of H, halo, hydroxy, NH₃⁺, —NHC(O)NH₂, —NHC(O)OR⁹, —NHC(O)R⁹, and an optionally substituted: C₁₋₆ alkyl, cycloalkyl-alkyl, heterocyclyl-alkyl, heteroaralkyl, aralkyl, or aryl, or an R^{3a} and R^{3b} together form an oxo;
an R^{3a} together with R² optionally form an optionally substituted cycloalkyl or optionally substituted heterocyclyl;
Y is selected from the group consisting of H, halo, ethynyl, —C(O)H, —CN, —C(O)OR⁴, —C(O)NR⁵R⁶, —C(O)NHSO₂R⁹, —PO₃H₂, 1H-tetrazol-5-yl, 1H-1,2,4-triazol-5-yl, 1H-pyrazol-5-yl, 1,2-dihydro-1,2,4-triazol-3-on-5-yl, and 1,2-dihydro-pyrazol-3-on-5-yl,
   wherein if Y is H, then:
      at least one R^{3a} or R^{3b} is an optionally substituted aryl, or
      R¹ is -A¹-L¹-A² or an optionally substituted: aryl, heteroaryl, —C(O)-aryl, —C(O)-aralkyl, or —C(O)-heterocyclyl-aralkyl;
R⁷ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl;
R¹⁰ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, heteroaralkyl, or is absent, or R⁷ and R¹⁰ together form an optionally substituted ring or ring system;
R¹¹ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl, or is absent;
each Z¹ are independently C or N;
Z² is CH, N, O, or S;
Z³ is C or N;
R² is selected from the group consisting of H, —C(O)OR⁴, —C(O)NR⁵R⁶, —C(O)-A¹-L¹-A², —CH₂-A¹-L¹-A², —C(O)CH₂-A¹-L¹-A², —C(O)NHCH₂-A¹-L¹-A², and an optionally substituted: alkyl, —C(O)-alkyl, aryl, —C(O)-aryl, aralkyl, —C(O)-aralkyl, or heteroaralkyl,
   wherein if
      R¹ is not -A¹-L¹-A² or an optionally substituted: aryl, heteroaryl, —C(O)-aryl, —C(O)-aralkyl, or —C(O)-heterocyclyl-aralkyl, then:
      R² is selected from the group consisting of —C(O)-A¹-L¹-A², —CH₂-A¹-L¹-A², —C(O)CH₂-A¹-L¹-A², —CH₂— (optionally substituted heteroaryl), and optionally substituted —C(O)-aralkyl, at least one R^{3a} or R^{3b} is an optionally substituted heteroaralkyl,
      Y is —C(O)OH or —C(O)H and at least one Z¹ is N.
      Y is —C(O)OH or —C(O)H and R¹⁰ is phenyl or —O-benzyl,
      Y is —C(O)OH or —C(O)H and R¹¹ is —O-(optionally substituted phenyl), or
      Y is —C(O)OH or —C(O)H, R⁷ is —O-benzyl, and R¹⁰ is —O-methyl;
R⁴ is H or optionally substituted: alkyl, alkenyl, alkynyl, aryl, aralkyl, heteroaryl, or heteroaralkyl;
R⁵ and R⁶ are each independently selected from the group consisting of H, CN, and an optionally substituted: C₁₋₆ alkyl, C₃₋₇ cycloalkyl, hetereocyclyl, -hetereocyclyl-C(O)OR⁴, aryl, heteroaryl, aralkyl, heteroaralkyl, or cycloalkyl-alkyl, or R⁵ and R⁶ together form an optionally substituted ring or ring system; and
R⁹ is selected from the group consisting of alkyl, cycloalkyl, and aryl;
with the proviso that:
   if R¹ is a pyridine, pyrimidine, or quinoline, or if R¹ is naphthalene and n is not 0, then Y is not CO₂H;
   if R¹ is an unsubstituted phenyl, then Y is not —C(O)OMe, —C(O)OEt, —C(O)O-t-Bu, —C(O)OBn, —C(O)NMe₂, —C(O)NEt₂, or —C(O)N(i-Pr)₂;
   if n is less than 3 and R¹ is an unsubstituted phenyl or unsubstituted biphenyl and Y is —C(O)OH, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn or Br;
   if Y is —C(O)OH and R¹ is phenyl substituted with a single halogen, —SO₂Me, —OCF₃, —OCF₂CF₃, —OCF₂CF₂H, —NC(O)CH₂Br, -Me, —SCH₃, or -t-Bu or R¹ is phenyl fused with a dioxolane ring, then R⁷ is —OBn or Br;
   if Y is —C(O)OMe and R¹ is phenyl substituted with a single Cl, then R⁷ is —OBn;
   if Y is —C(O)OEt and R¹ is phenyl substituted with a single halogen, —SO₂Me, —NH₂, —OH, —OCH₃, or —NO₂, or two Cl, then R⁷ is —OBn;
   if Y is —C(O)O-(substituted phenyl) and R¹ is phenyl substituted with two Cl, then R⁷ is —OBn;
   if Y is —C(O)O-alkyl-phenyl and R¹ is unsubstituted phenyl or phenyl substituted with a single Br, then R⁷ is —OBn;
   if n is 0 and R¹ is unsubstituted phenyl or phenyl substituted by a single methyl, then R² is selected from the group consisting of —C(O)-A¹-L¹-A2, —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn;
   if R¹ is -A¹-L¹-A², L¹ is methoxy, A¹ is unsubstituted phenyl, A² is phenyl substituted with a single CF₃, and Y is —C(O)OH, then R⁷ is —OBn;
   if R¹ is -A¹-L¹-A², L¹ is absent, A¹ is benzofuran, A² is thiazole, and Y is —C(O)OH, then R⁷ is —OBn; and
   if R¹ is -A¹-L¹-A², L¹ is methoxy or absent, A¹ is unsubstituted phenyl, A² is unsubstituted phenyl, R² is alkyl, and Y is —C(O)O-alkyl, then R⁷ is —OBn.

In some alternative embodiments, compounds of formula I have the following definitions:
n is an integer from 0 to 3;
R¹ is selected from the group consisting of H, -A¹-L¹-A², and an optionally substituted: alkyl, alkenyl, alkynyl, cycloalkyl, heterocyclyl, aryl, heteroaryl, —C(O)-aryl, —C(O)-aralkyl, —C(O)-heteroaryl, or —C(O)-heterocyclyl-aralkyl; or R¹ is absent and n is 0 when Z² is O or S;
   wherein if R¹ is —C(O)-aryl, —C(O)-aralkyl, or —C(O)-heterocyclyl-aralkyl, then n is not 0;
A¹ and A² are independently selected from the group consisting of optionally substituted aryl and optionally substituted heteroaryl;
L¹ is oxy, C₁₋₆ alkoxy, —NR⁵C(O)-alkyl-, —NR⁵C(O)CH₂S—, —NR⁵CH₂—, —NR⁵ or absent;
L² is —CR^{3a}R^{3b}—, —CR^{3a}R^{3b}CR^{3a}R^{3b}—, —CR^{3a}═CR^{3a}—, or absent;
each R^{3a} and each R^{3b} are independently selected from the group consisting of H, halo, hydroxy, NH₃⁺, —NHC(O)NH₂, —NHC(O)OR⁹, —NHC(O)R⁹, —C(O)R⁴ and an optionally substituted: C₁₋₆ alkyl, cycloalkyl-alkyl, heterocyclyl-alkyl, heteroaralkyl, aralkyl, or aryl, or an R^{3a} and R^{3b} together form an oxo;
an R^{3a} together with R² optionally form an optionally substituted cycloalkyl or optionally substituted heterocyclyl;
Y is selected from the group consisting of H, halo, ethynyl, —C(O)H, —CN, —C(O)OR⁴, —C(O)NR⁵R⁶, —C(O)NHSO₂R⁹, —C(O)NHOR⁴, —C(O)OCH₃OC(O)R⁴, —NHC(O)R⁴, —C(O)NHOR⁴, —C(O)OCH₃OR⁴, —PO₃H₂, 1H-tetrazol-5-yl, 1H-1,2,4-triazol-5-yl, 1H-pyrazol-5-yl, 1,2-dihydro-1,2,4-triazol-3-on-5-yl, and 1,2-dihydro-pyrazol-3-on-5-yl,
   wherein if Y is H, then:
      at least one R^{3a} or R^{3b} is an optionally substituted aryl, or
      R¹ is -A¹-L¹-A² or an optionally substituted: aryl, heteroaryl, —C(O)-aryl, —C(O)-aralkyl, or —C(O)-heterocyclyl-aralkyl;
R⁷ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl;
R¹⁰ is selected from the group consisting of H, halo, —CN, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, heterocyclyl, cycloalkylalkoxy, aryl, aralkyl, heteroaryl, heteroaralkyl, or is absent, or R⁷ and R¹⁰ together form an optionally substituted ring or ring system;
R¹¹ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl, or is absent;
each Z¹ are independently C or N;
Z² is CH, N, O, or S;
Z³ is C or N;
R² is selected from the group consisting of H, —C(O)OR⁴, —C(O)NR⁵R⁶, —C(O)-A¹-L¹-A², —CH₂-A¹-L¹-A², —C(O)CH₂-A¹-L¹-A², —C(O)NHCH₂-A¹-L¹-A², and an optionally substituted: alkyl, —C(O)-alkyl, aryl, —C(O)-aryl, aralkyl, —C(O)-aralkyl, or heteroaralkyl,
   wherein if
      R¹ is not -A¹-L¹-A² or an optionally substituted: aryl, heteroaryl, —C(O)-aryl, —C(O)-aralkyl, or —C(O)-heterocyclyl-aralkyl, then:
      R² is selected from the group consisting of —C(O)-A¹-L¹-A², —CH₂-A¹-L¹-A², —C(O)CH₂-A¹-L¹-A², —CH₂-(optionally substituted heteroaryl), and optionally substituted —C(O)-aralkyl,
      at least one R^{3a} or R^{3b} is an optionally substituted heteroaralkyl,
      Y is —C(O)OH or —C(O)H and at least one Z¹ is N.
      Y is —C(O)OH or —C(O)H and R¹⁰ is phenyl, phenyl substituted with one or more amino, or —O-benzyl,
      Y is —C(O)OH or —C(O)H and R¹¹ is —O-(optionally substituted phenyl), or
      Y is —C(O)OH or —C(O)H, R⁷ is —O-benzyl, and R¹⁰ is —O-methyl;
R⁴ is H or optionally substituted: alkyl, alkenyl, alkynyl, aryl, aralkyl, heteroaryl, heterocyclyl, or heteroaralkyl;
R⁵ and R⁶ are each independently selected from the group consisting of H, CN, and an optionally substituted: C₁₋₆ alkyl, C₃₋₇ cycloalkyl, hetereocyclyl, -hetereocyclyl-C(O)OR⁴, aryl, heteroaryl, aralkyl, heteroaralkyl, or cycloalkyl-alkyl, or R⁵ and R⁶ together form an optionally substituted ring or ring system; and
R⁹ is selected from the group consisting of alkyl, cycloalkyl, and aryl;
with the proviso that:
   if R¹ is a pyridine, pyrimidine, or quinoline, or if R¹ is naphthalene and n is not 0, then Y is not CO₂H;
   if R¹ is an unsubstituted phenyl, then Y is not —C(O)OMe, —C(O)OEt, —C(O)O-t-Bu, —C(O)OBn, —C(O)NMe₂, —C(O)NEt₂, or —C(O)N(i-Pr)₂;
   if n is less than 3 and R¹ is an unsubstituted phenyl or unsubstituted biphenyl and Y is —C(O)OH, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn, Br, or phenyl substituted with one or more amino;
   if Y is —C(O)OH and R¹ is phenyl substituted with a single halogen, —SO₂Me, —OCF₃, —OCF₂CF₃, —OCF₂CF₂H, —NC(O)CH₂Br, -Me, —SCH₃, or -t-Bu or R¹ is phenyl fused with a dioxolane ring, then R⁷ is —OBn or Br;
   if Y is —C(O)OMe and R¹ is phenyl substituted with a single Cl, then R⁷ is —OBn;
   if Y is —C(O)OEt and R¹ is phenyl substituted with a single halogen, —SO₂Me, —NH₂, —OH, —OCH₃, or —NO₂, or two Cl, then R⁷ is —OBn or R¹⁰ is phenyl substituted with one or more nitro;
   if Y is —C(O)O-(substituted phenyl) and R¹ is phenyl substituted with two Cl, then R⁷ is —OBn;
   if Y is —C(O)O-alkyl-phenyl and R¹ is unsubstituted phenyl or phenyl substituted with a single Br, then R⁷ is —OBn;
   if n is 0 and R¹ is unsubstituted phenyl or phenyl substituted by a single methyl, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn;
   if R¹ is -A¹-L¹-A², L¹ is methoxy, A¹ is unsubstituted phenyl, A² is phenyl substituted with a single CF₃, and Y is —C(O)OH, then R⁷ is —OBn;
   if R¹ is -A¹-L¹-A², L¹ is absent, A¹ is benzofuran, A² is thiazole, and Y is —C(O)OH, then R⁷ is —OBn; and
   if R¹ is -A¹-L¹-A², L¹ is methoxy or absent, A¹ is unsubstituted phenyl, A² is unsubstituted phenyl, R² is alkyl, and Y is —C(O)O-alkyl, then R⁷ is —OBn.

The present embodiments provide for a method of inhibiting NS3 helicase activity comprising contacting a NS3 helicase with a compound disclosed herein.

The present embodiments provide for a method of treating hepatitis by modulating NS3 helicase comprising contacting a NS3 helicase with a compound disclosed herein.

Exemplary compounds of Formula I are set forth in Tables 1 through 39 and compounds disclosed therein below.

Preferred compounds include Compounds 1-795 described below.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

The present embodiments further provide compositions, including pharmaceutical compositions, comprising compounds of the general Formula I, including salts, esters, or other derivatives thereof A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In many embodiments, a subject compound inhibits the enzymatic activity of a hepatitis virus C(HCV) NS3 helicase. Whether a subject compound inhibits HCV NS3 helicase can be readily determined using any known method. Typical methods involve determination of whether NS3 helicase-mediated unwinding is inhibited in the presence of the agent. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits enzymatic activity of an HCV NS3 helicase with an IC₅₀ of less than about 50 μM, e.g., a subject compound inhibits an HCV NS3 helicase with an IC₅₀ of less than about 40 μM, less than about 25 μM, less than about 10 μM, less than about 1 μM, less than about 500 nM, less than about 250 nM, less than about 125 nM, or less.

In many embodiments, a subject compound inhibits the enzymatic activity of a hepatitis virus C(HCV) NS3 helicase. Whether a subject compound inhibits HCV NS3 helicase can be readily determined using any known method. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Treating a Hepatitis Virus Infection

The methods and compositions described herein are generally useful in treatment of an of HCV infection.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formula I, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound of Formula I, optionally in combination with an effective amount of one or more additional antiviral agents. In some embodiments, an effective amount of a compound of Formula I, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formula I, and optionally one or more additional antiviral agents, is an amount that is effective to reduce viral load to lower than 100 genome copies/mL serum.

In some embodiments, an effective amount of a compound of Formula I, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formula I, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of formula I, and optionally one or more additional antiviral agents, is an amount effective to reduce ALT levels to less than about 45 IU/ml serum.

A therapeutically effective amount of a compound of Formula I, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formula I, and an additional antiviral agent is a synergistic amount. As used herein, a “synergistic combination” or a “synergistic amount” of a compound of Formula I, and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formula I, when administered at that same dosage as a monotherapy and (ii) the therapeutic or prophylactic benefit of the additional antiviral agent when administered at the same dosage as a monotherapy.

In some embodiments, a selected amount of a compound of Formula I, and a selected amount of an additional antiviral agent are effective when used in combination therapy for a disease, but the selected amount of the compound of Formula I, and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of the compound of Formula I, when used in combination therapy for a disease, where the selected amount of the additional antiviral agent provides no therapeutic benefit when used in monotherapy for the disease (2) regimens in which a selected amount of the compound of Formula I, enhances the therapeutic benefit of a selected amount of the additional antiviral agent when used in combination therapy for a disease, where the selected amount of the compound of Formula I, provides no therapeutic benefit when used in monotherapy for the disease and (3) regimens in which a selected amount of the compound of Formula I, and a selected amount of the additional antiviral agent provide a therapeutic benefit when used in combination therapy for a disease, where each of the selected amounts of the compound of Formula I, and the additional antiviral agent, respectively, provides no therapeutic benefit when used in monotherapy for the disease. As used herein, a “synergistically effective amount” of a compound of Formula I, and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formula I, and optionally one or more additional antiviral agents. Effective amounts of compounds of Formula I, with and without one or more additional antiviral agents, as well as dosing regimens, are as discussed below.

Whether treatment with a compound of Formula I, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of formula I, and optionally one or more additional antiviral agents, is an amount that effects a change of one unit or more in the fibrosis stage based on pre- and post-therapy liver biopsies. In particular embodiments, a therapeutically effective amount of a compound of formula I, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formula I. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formula I, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formula I, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formula I, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formula I, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound of Formula I, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formula I, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formula I, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formula I, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations and Routes of Administration

In the subject methods, the active agent(s) (e.g., compound of Formula I, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) can be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Other Antiviral or Antifibrotic Agents

As discussed above, a subject method will in some embodiments be carried out by administering an NS3 inhibitor that is a compound of Formula I, and optionally one or more additional antiviral agent(s).

In some embodiments, the method further includes administration of one or more interferon receptor agonist(s). Interferon receptor agonists are described above.

In other embodiments, the method further includes administration of pirfenidone or a pirfenidone analog. Pirfenidone and pirfenidone analogs are described above.

Additional antiviral agents that are suitable for use in combination therapy include, but are not limited to, nucleotide and nucleoside analogs. Non-limiting examples include azidothymidine (AZT) (zidovudine), and analogs and derivatives thereof, 2′,3′-dideoxyinosine (DDI) (didanosine), and analogs and derivatives thereof, 2′,3′-dideoxycytidine (DDC) (dideoxycytidine), and analogs and derivatives thereof, 2′3,′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β3-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the interferon receptor agonist. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester [5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man. Because of its strong inhibitory effect on cytochrome P450 3A and the inhibitory effect on cytochrome P450 2D6, ritonavir at doses below the normal therapeutic dosage may be combined with other viral enzyme inhibitors to achieve therapeutic levels of the second viral enzyme inhibitor while reducing the number of dosage units required, the dosing frequency, or both. An NS-3 helicase inhibitor is a viral enzyme inhibitor.

Coadministration of low-dose ritonavir may also be used to compensate for drug interactions that tend to decrease levels of a viral enzyme inhibitor metabolized by CYP3A. Its structure, synthesis, manufacture and formulation are described in U.S. Pat. No. 5,541,206 U.S. Pat. No. 5,635,523 U.S. Pat. No. 5,648,497 U.S. Pat. No. 5,846,987 and U.S. Pat. No. 6,232,333. The ritonavir may be administered orally in capsule or tablet or oral solution form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, an additional antiviral agent is administered during the entire course of NS3 inhibitor compound treatment. In other embodiments, an additional antiviral agent is administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The NS3 inhibitor compounds described herein may be used in acute or chronic therapy for HCV disease. In many embodiments, the NS3 inhibitor compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound is administered as a continuous infusion.

In many embodiments, an NS3 inhibitor compound of the embodiments is administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound are readily determinable by those of skill in the art by a variety of means. A preferred means is to measure the physiological potency of a given interferon receptor agonist.

In many embodiments, multiple doses of NS3 inhibitor compound are administered. For example, an NS3 inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Ribavirin

In some embodiments, the methods provide for combination therapy comprising administering an NS3 inhibitor compound as described above, and an effective amount of ribavirin. Ribavirin can be administered in dosages of about 400 mg, about 800 mg, about 1000 mg, or about 1200 mg per day.

One embodiment provides any of the above-described methods modified to include co-administering to the patient a therapeutically effective amount of ribavirin for the duration of the desired course of NS3 inhibitor compound treatment.

Another embodiment provides any of the above-described methods modified to include co-administering to the patient about 800 mg to about 1200 mg ribavirin orally per day for the duration of the desired course of NS3 inhibitor compound treatment. In another embodiment, any of the above-described methods may be modified to include co-administering to the patient (a) 1000 mg ribavirin orally per day if the patient has a body weight less than 75 kg or (b) 1200 mg ribavirin orally per day if the patient has a body weight greater than or equal to 75 kg, where the daily dosage of ribavirin is optionally divided into to 2 doses for the duration of the desired course of NS3 inhibitor compound treatment.

Combination Therapies with Levovirin

In some embodiments, the methods provide for combination therapy comprising administering an NS3 inhibitor compound as described above, and an effective amount of levovirin. Levovirin is generally administered in an amount ranging from about 30 mg to about 60 mg, from about 60 mg to about 125 mg, from about 125 mg to about 200 mg, from about 200 mg to about 300 mg, from about 300 mg to about 400 mg, from about 400 mg to about 1200 mg, from about 600 mg to about 1000 mg, or from about 700 to about 900 mg per day, or about 10 mg/kg body weight per day. In some embodiments, levovirin is administered orally in dosages of about 400, about 800, about 1000, or about 1200 mg per day for the desired course of NS3 inhibitor compound treatment.

Combination Therapies with Viramidine

In some embodiments, the methods provide for combination therapy comprising administering an NS3 inhibitor compound as described above, and an effective amount of viramidine. Viramidine is generally administered in an amount ranging from about 30 mg to about 60 mg, from about 60 mg to about 125 mg, from about 125 mg to about 200 mg, from about 200 mg to about 300 mg, from about 300 mg to about 400 mg, from about 400 mg to about 1200 mg, from about 600 mg to about 1000 mg, or from about 700 to about 900 mg per day, or about 10 mg/kg body weight per day. In some embodiments, viramidine is administered orally in dosages of about 800, or about 1600 mg per day for the desired course of NS3 inhibitor compound treatment.

Combination Therapies with Ritonavir

In some embodiments, the methods provide for combination therapy comprising administering an NS3 inhibitor compound as described above, and an effective amount of ritonavir. Ritonavir is generally administered in an amount ranging from about 50 mg to about 100 mg, from about 100 mg to about 200 mg, from about 200 mg to about 300 mg, from about 300 mg to about 400 mg, from about 400 mg to about 500 mg, or from about 500 mg to about 600 mg, twice per day. In some embodiments, ritonavir is administered orally in dosages of about 300 mg, or about 400 mg, or about 600 mg twice per day for the desired course of NS3 inhibitor compound treatment.

Combination Therapies with Alpha-Glucosidase Inhibitors

Suitable α-glucosidase inhibitors include any of the above-described imino-sugars, including long-alkyl chain derivatives of imino sugars as disclosed in U.S. Patent Publication No. 2004/0110795; inhibitors of endoplasmic reticulum-associated α-glucosidases; inhibitors of membrane bound α-glucosidase; miglitol (Glyset®), and active derivatives, and analogs thereof, and acarbose (Precose®), and active derivatives, and analogs thereof.

In many embodiments, the methods provide for combination therapy comprising administering an NS3 inhibitor compound as described above, and an effective amount of an α-glucosidase inhibitor administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

An α-glucosidase inhibitor can be administered 5 times per day, 4 times per day, tid (three times daily), bid, qd, qod, biw, tiw, qw, qow, three times per month, or once monthly. In other embodiments, an α-glucosidase inhibitor is administered as a continuous infusion.

In many embodiments, an α-glucosidase inhibitor is administered orally.

In connection with the above-described methods for the treatment of a flavivirus infection, treatment of HCV infection, and treatment of liver fibrosis that occurs as a result of an HCV infection, the methods provide for combination therapy comprising administering an NS3 inhibitor compound as described above, and an effective amount of α-glucosidase inhibitor administered to the patient at a dosage of from about 10 mg per day to about 600 mg per day in divided doses, e.g., from about 10 mg per day to about 30 mg per day, from about 30 mg per day to about 60 mg per day, from about 60 mg per day to about 75 mg per day, from about 75 mg per day to about 90 mg per day, from about 90 mg per day to about 120 mg per day, from about 120 mg per day to about 150 mg per day, from about 150 mg per day to about 180 mg per day, from about 180 mg per day to about 210 mg per day, from about 210 mg per day to about 240 mg per day, from about 240 mg per day to about 270 mg per day, from about 270 mg per day to about 300 mg per day, from about 300 mg per day to about 360 mg per day, from about 360 mg per day to about 420 mg per day, from about 420 mg per day to about 480 mg per day, or from about 480 mg to about 600 mg per day.

In some embodiments, the methods provide for combination therapy comprising administering an NS3 inhibitor compound as described above, and an effective amount of α-glucosidase inhibitor administered in a dosage of about 10 mg three times daily. In some embodiments, an α-glucosidase inhibitor is administered in a dosage of about 15 mg three times daily. In some embodiments, an α-glucosidase inhibitor is administered in a dosage of about 20 mg three times daily. In some embodiments, an α-glucosidase inhibitor is administered in a dosage of about 25 mg three times daily. In some embodiments, an α-glucosidase inhibitor is administered in a dosage of about 30 mg three times daily. In some embodiments, an α-glucosidase inhibitor is administered in a dosage of about 40 mg three times daily. In some embodiments, an α-glucosidase inhibitor is administered in a dosage of about 50 mg three times daily. In some embodiments, an α-glucosidase inhibitor is administered in a dosage of about 100 mg three times daily. In some embodiments, an α-glucosidase inhibitor is administered in a dosage of about 75 mg per day to about 150 mg per day in two or three divided doses, where the individual weighs 60 kg or less. In some embodiments, an α-glucosidase inhibitor is administered in a dosage of about 75 mg per day to about 300 mg per day in two or three divided doses, where the individual weighs 60 kg or more.

The amount of active ingredient (e.g., α-glucosidase inhibitor) that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific α-glucosidase inhibitor, the severity of the symptoms and the susceptibility of the subject to side effects. Preferred dosages for a given α-glucosidase inhibitor are readily determinable by those of skill in the art by a variety of means. A typical means is to measure the physiological potency of a given active agent.

In many embodiments, multiple doses of an α-glucosidase inhibitor are administered. For example, the methods provide for combination therapy comprising administering an NS3 inhibitor compound as described above, and an effective amount of α-glucosidase inhibitor administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Thymosin-α

In some embodiments, the methods provide for combination therapy comprising administering an NS3 inhibitor compound as described above, and an effective amount of thymosin-α. Thymosin-α (Zadaxin™) is generally administered by subcutaneous injection. Thymosin-α can be administered tid, bid, qd, qod, biw, tiw, qw, qow, three times per month, once monthly, substantially continuously, or continuously for the desired course of NS3 inhibitor compound treatment. In many embodiments, thymosin-α is administered twice per week for the desired course of NS3 inhibitor compound treatment. Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

Thymosin-α can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In one embodiment, thymosin-α is administered for the desired course of NS3 inhibitor compound treatment.

Combination Therapies with Interferon(s)

In many embodiments, the methods provide for combination therapy comprising administering an NS3 inhibitor compound as described above, and an effective amount of an interferon receptor agonist. In some embodiments, a compound of Formula I, and a Type I or III interferon receptor agonist are co-administered in the treatment methods described herein. Type I interferon receptor agonists suitable for use herein include any interferon-α (IFN-α). In certain embodiments, the interferon-α is a PEGylated interferon-α. In certain other embodiments, the interferon-α is a consensus interferon, such as INFERGEN® interferon alfacon-1. In still other embodiments, the interferon-α is a monoPEG (30 kD, linear)-ylated consensus interferon.

Effective dosages of an IFN-α range from about 3 μg to about 27 μg, from about 3 MU to about 10 MU, from about 90 μg to about 180 μg, or from about 18 μg to about 90 μg. Effective dosages of Infergen® consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS®PEGylated IFN-α2a contain an amount of about 90 μg to 270 μg, or about 180 μg, of drug per dose. Effective dosages of PEG-INTRON®PEGylated IFN-α2b contain an amount of about 0.5 μg to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 μg, or about 60 μg to about 180 μg, or about 90 μg to about 120 μg, of drug per dose. IFN-α can be administered daily, every other day, once a week, three times a week, every other week, three times per month, once monthly, substantially continuously or continuously.

In many embodiments, the Type I or Type III interferon receptor agonist and/or the Type II interferon receptor agonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations. Some embodiments provide any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration. In other embodiments, any of the above-described methods may be practiced in which the desired dosage of PEGylated IFN-α (PEG-IFN-α) is administered subcutaneously to the patient by bolus delivery qw, qow, three times per month, or monthly for the desired treatment duration.

In other embodiments, an NS3 inhibitor compound and a Type II interferon receptor agonist are co-administered in the treatment methods of the embodiments. Type II interferon receptor agonists suitable for use herein include any interferon-γ (IFN-γ).

Effective dosages of IFN-γ can range from about 0.5 μg/m² to about 500 μg/m², usually from about 1.5 μg/m² to 200 μg/m², depending on the size of the patient. This activity is based on 106 international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In some embodiments, a Type I or a Type III interferon receptor agonist is administered in a first dosing regimen, followed by a second dosing regimen. The first dosing regimen of Type I or a Type III interferon receptor agonist (also referred to as “the induction regimen”) generally involves administration of a higher dosage of the Type I or Type III interferon receptor agonist. For example, in the case of Infergen® consensus IFN-α (CIFN), the first dosing regimen comprises administering CIFN at about 9 μg, about 15 μg, about 18 μg, or about 27 μg. The first dosing regimen can encompass a single dosing event, or at least two or more dosing events. The first dosing regimen of the Type I or Type III interferon receptor agonist can be administered daily, every other day, three times a week, every other week, three times per month, once monthly, substantially continuously or continuously.

The first dosing regimen of the Type I or Type III interferon receptor agonist is administered for a first period of time, which time period can be at least about 4 weeks, at least about 8 weeks, or at least about 12 weeks.

The second dosing regimen of the Type I or Type III interferon receptor agonist (also referred to as “the maintenance dose”) generally involves administration of a lower amount of the Type I or Type III interferon receptor agonist. For example, in the case of CIFN, the second dosing regimen comprises administering CIFN at a dose of at least about 3 μg, at least about 9 μg, at least about 15 μg, or at least about 18 μg. The second dosing regimen can encompass a single dosing event, or at least two or more dosing events.

The second dosing regimen of the Type I or Type III interferon receptor agonist can be administered daily, every other day, three times a week, every other week, three times per month, once monthly, substantially continuously or continuously.

In some embodiments, where an “induction”/“maintenance” dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase.

In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, the Type II interferon receptor agonist treatment is discontinued once Type I or a Type III interferon receptor agonist treatment begins.

In other embodiments, the Type I or Type III interferon receptor agonist is administered in single dosing regimen. For example, in the case of CIFN, the dose of CIFN is generally in a range of from about 3 μg to about 15 μg, or from about 9 μg to about 15 μg. The dose of Type I or a Type III interferon receptor agonist is generally administered daily, every other day, three times a week, every other week, three times per month, once monthly, or substantially continuously. The dose of the Type I or Type III interferon receptor agonist is administered for a period of time, which period can be, for example, from at least about 24 weeks to at least about 48 weeks, or longer.

In some embodiments, where a single dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase. In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, Type II interferon receptor agonist treatment is discontinued once Type I or a Type III interferon receptor agonist treatment begins.

In additional embodiments, an NS3 inhibitor compound, a Type I or III interferon receptor agonist, and a Type II interferon receptor agonist are co-administered for the desired duration of treatment in the methods described herein. In some embodiments, an NS3 inhibitor compound, an interferon-α, and an interferon-γ are co-administered for the desired duration of treatment in the methods described herein.

In some embodiments, the invention provides methods using an amount of a Type I or Type III interferon receptor agonist, a Type II interferon receptor agonist, and an NS3 inhibitor compound, effective for the treatment of HCV infection in a patient. Some embodiments provide methods using an effective amount of an IFN-α, IFN-γ, and an NS3 inhibitor compound in the treatment of HCV infection in a patient. One embodiment provides a method using an effective amount of a consensus IFN-α, IFN-γ and an NS3 inhibitor compound in the treatment of HCV infection in a patient.

In general, an effective amount of a consensus interferon (CIFN) and IFN-γ suitable for use in the methods of the embodiments is provided by a dosage ratio of 1 μg CIFN: 10 μg IFN-γ, where both CIFN and IFN-γ are unPEGylated and unglycosylated species.

In one embodiment, the invention provides any of the above-described methods modified to use an effective amount of INFERGEN®consensus IFN-α and IFN-γ in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 50 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 9 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 90 μg to about 100 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 30 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 200 μg to about 300 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a total weekly dosage of IFN-γ containing an amount of about 30 μg to about 1,000 μg of drug per week in divided doses administered subcutaneously qd, qod, tiw, biw, or administered substantially continuously or continuously, for the desired duration of treatment with an NS3 inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a total weekly dosage of IFN-γ containing an amount of about 100 μg to about 300 μg of drug per week in divided doses administered subcutaneously qd, qod, tiw, biw, or substantially continuously or continuously, for the desired duration of treatment with an NS3 inhibitor compound.

In general, an effective amount of IFN-α 2a or 2b or 2c and IFN-γ suitable for use in the methods of the embodiments is provided by a dosage ratio of 1 million Units (MU) IFN-α 2a or 2b or 2c: 30 μg IFN-γ, where both IFN-α2a or 2b or 2c and IFN-γ are unPEGylated and unglycosylated species.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 1 MU to about 20 MU of drug per dose of IFN-α 2a, 2b or 2c subcutaneously qd, qod, tiw, biw, or per day substantially continuously or continuously, in combination with a dosage of IFN-γ containing an amount of about 30 μg to about 600 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of IFN-γ containing an amount of about 100 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 10 MU of drug per dose of IFN-α 2a, 2b or 2c subcutaneously qd, qod, tiw, biw, or per day substantially continuously or continuously, in combination with a dosage of IFN-γ containing an amount of about 300 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of PEGASYS®PEGylated IFN-α 2a and IFN-γ in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a total weekly dosage of IFN-γ containing an amount of about 30 μg to about 1,000 μg, of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 180 μg of drug per dose of PEGASYS®, subcutaneously qw, qow, three times per month, or monthly, in combination with a total weekly dosage of IFN-γ containing an amount of about 100 μg to about 300 μg, of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a total weekly dosage of IFN-γ containing an amount of about 30 μg to about 1,000 μg of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a total weekly dosage of IFN-γ containing an amount of about 100 μg to about 300 μg of drug per week administered in divided doses subcutaneously qd, qod, tiw, or biw, or administered substantially continuously or continuously, for the desired duration of treatment with an NS3 inhibitor compound.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 25 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 200 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 25 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 200 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

Any of the above-described methods involving administering an NS3 inhibitor, a Type I interferon receptor agonist (e.g., an IFN-α), and a Type II interferon receptor agonist (e.g., an IFN-γ), can be augmented by administration of an effective amount of a TNF-α antagonist (e.g., a TNF-α antagonist other than pirfenidone or a pirfenidone analog). Exemplary, non-limiting TNF-α antagonists that are suitable for use in such combination therapies include ENBREL®, REMICADE®, and HUMIRA™.

One embodiment provides a method using an effective amount of ENBREL®; an effective amount of IFN-α; an effective amount of IFN-γ; and an effective amount of an NS3 inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment.

One embodiment provides a method using an effective amount of REMICADE®, an effective amount of IFN-α; an effective amount of IFN-γ; and an effective amount of an NS3 inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment.

One embodiment provides a method using an effective amount of HUMIRA™, an effective amount of IFN-α; an effective amount of IFN-γ; and an effective amount of an NS3 inhibitor in the treatment of an HCV infection in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment.

Combination Therapies with Pirfenidone

In many embodiments, the methods provide for combination therapy comprising administering an NS3 inhibitor compound as described above, and an effective amount of pirfenidone or a pirfenidone analog. In some embodiments, an NS3 inhibitor compound, one or more interferon receptor agonist(s), and pirfenidone or pirfenidone analog are co-administered in the treatment methods of the embodiments. In certain embodiments, an NS3 inhibitor compound, a Type I interferon receptor agonist, and pirfenidone (or a pirfenidone analog) are co-administered. In other embodiments, an NS3 inhibitor compound, a Type I interferon receptor agonist, a Type II interferon receptor agonist, and pirfenidone (or a pirfenidone analog) are co-administered. Type I interferon receptor agonists suitable for use herein include any IFN-α, such as interferon alfa-2a, interferon alfa-2b, interferon alfacon-1, and PEGylated IFN-α's, such as peginterferon alfa-2a, peginterferon alfa-2b, and PEGylated consensus interferons, such as monoPEG (30 kD, linear)-ylated consensus interferon. Type II interferon receptor agonists suitable for use herein include any interferon-γ.

Pirfenidone or a pirfenidone analog can be administered once per month, twice per month, three times per month, once per week, twice per week, three times per week, four times per week, five times per week, six times per week, daily, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or a specific pirfenidone analog include a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally in one to five divided doses per day. Other doses and formulations of pirfenidone and specific pirfenidone analogs suitable for use in the treatment of fibrotic diseases are described in U.S. Pat. Nos. 5,310,562; 5,518,729; 5,716,632; and 6,090,822.

One embodiment provides any of the above-described methods modified to include co-administering to the patient a therapeutically effective amount of pirfenidone or a pirfenidone analog for the duration of the desired course of NS3 inhibitor compound treatment.

Combination Therapies with TNF-α Antagonists

In many embodiments, the methods provide for combination therapy comprising administering an effective amount of an NS3 inhibitor compound as described above, and an effective amount of TNF-α antagonist, in combination therapy for treatment of an HCV infection.

Effective dosages of a TNF-α antagonist range from 0.1 μg to 40 mg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, effective dosages of a TNF-α antagonist are expressed as mg/kg body weight. In these embodiments, effective dosages of a TNF-α antagonist are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, a TNF-α antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The TNF-α antagonist can be administered tid, bid, qd, qod, biw, tiw, qw, qow, three times per month, once monthly, substantially continuously, or continuously.

In many embodiments, multiple doses of a TNF-α antagonist are administered. For example, a TNF-α antagonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A TNF-α antagonist and an NS3 inhibitor are generally administered in separate formulations. A TNF-α antagonist and an NS3 inhibitor may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

One embodiment provides a method using an effective amount of a TNF-α antagonist and an effective amount of an NS3 inhibitor in the treatment of an HCV infection in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides a method using an effective amount of ENBREL® and an effective amount of an NS3 inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment with an NS3 inhibitor compound.

One embodiment provides a method using an effective amount of REMICADE® and an effective amount of an NS3 inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment with an NS3 inhibitor compound.

One embodiment provides a method using an effective amount of HUMIRA™ and an effective amount of an NS3 inhibitor in the treatment of an HCV infection in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment with an NS3 inhibitor compound.

Combination Therapies with Thymosin-α

In many embodiments, the methods provide for combination therapy comprising administering an effective amount of an NS3 inhibitor compound as described above, and an effective amount of thymosin-α, in combination therapy for treatment of an HCV infection.

Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

One embodiment provides a method using an effective amount of ZADAXIN™ thymosin-α and an effective amount of an NS3 inhibitor in the treatment of an HCV infection in a patient, comprising administering to the patient a dosage of ZADAXIN™ containing an amount of from about 1.0 mg to about 1.6 mg per dose, subcutaneously twice per week for the desired duration of treatment with the NS3 inhibitor compound.

Combination Therapies with a TNF-α Antagonist and an Interferon

Some embodiments provide a method of treating an HCV infection in an individual having an HCV infection, the method comprising administering an effective amount of an NS3 inhibitor, and effective amount of a TNF-α antagonist, and an effective amount of one or more interferons.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 30 μg to about 1,000 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 100 μg to about 300 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 1 MU to about 20 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 10 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 180 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Combination Therapies with Other Antiviral Agents

Other agents such as inhibitors of HCV NS3 helicase are also attractive drugs for combinational therapy, and are contemplated for use in combination therapies described herein. Ribozymes such as Heptazyme and phosphorothioate oligonucleotides which are complementary to HCV protein sequences and which inhibit the expression of viral core proteins are also suitable for use in combination therapies described herein.

In some embodiments, the additional antiviral agent(s) is administered during the entire course of treatment with the NS3 inhibitor compound described herein, and the beginning and end of the treatment periods coincide. In other embodiments, the additional antiviral agent(s) is administered for a period of time that is overlapping with that of the NS3 inhibitor compound treatment, e.g., treatment with the additional antiviral agent(s) begins before the NS3 inhibitor compound treatment begins and ends before the NS3 inhibitor compound treatment ends; treatment with the additional antiviral agent(s) begins after the NS3 inhibitor compound treatment begins and ends after the NS3 inhibitor compound treatment ends; treatment with the additional antiviral agent(s) begins after the NS3 inhibitor compound treatment begins and ends before the NS3 inhibitor compound treatment ends; or treatment with the additional antiviral agent(s) begins before the NS3 inhibitor compound treatment begins and ends after the NS3 inhibitor compound treatment ends.

The NS3 inhibitor compound can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional antiviral agents.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 100 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 150 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 200 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 9 μg of drug per dose, subcutaneously once daily or three times per week for the desired treatment duration with an NS3 inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 15 μg of drug per dose, subcutaneously once daily or three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 25 μg of drug per dose, subcutaneously three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 50 μg of drug per dose, subcutaneously three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 100 μg of drug per dose, subcutaneously three times per week for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring a TNF antagonist regimen can be modified to replace the subject TNF antagonist regimen with a TNF antagonist regimen comprising administering a dosage of a TNF antagonist selected from the group of: (a) etanercept in an amount of 25 mg of drug per dose subcutaneously twice per week, (b) infliximab in an amount of 3 mg of drug per kilogram of body weight per dose intravenously at weeks 0, 2 and 6, and every 8 weeks thereafter, or (c) adalimumab in an amount of 40 mg of drug per dose subcutaneously once weekly or once every 2 weeks; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 9 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 15 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 25 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 50 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 100 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2a comprising administering a dosage of peginterferon alfa-2a containing an amount of 180 μg of drug per dose, subcutaneously once weekly for the desired treatment duration with an NS3 inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2b comprising administering a dosage of peginterferon alfa-2b containing an amount of 1.0 μg to 1.5 μg of drug per kilogram of body weight per dose, subcutaneously once or twice weekly for the desired treatment duration with an NS3 inhibitor compound.

As non-limiting examples, any of the above-described methods can be modified to include administering a dosage of ribavirin containing an amount of 400 mg, 800 mg, 1000 mg or 1200 mg of drug orally per day, optionally in two or more divided doses per day, for the desired treatment duration with an NS3 inhibitor compound.

As non-limiting examples, any of the above-described methods can be modified to include administering a dosage of ribavirin containing (i) an amount of 1000 mg of drug orally per day for patients having a body weight of less than 75 kg or (ii) an amount of 1200 mg of drug orally per day for patients having a body weight of greater than or equal to 75 kg, optionally in two or more divided doses per day, for the desired treatment duration with an NS3 inhibitor compound.

As non-limiting examples, any of the above-described methods can be modified to replace the subject NS3 inhibitor regimen with an NS3 inhibitor regimen comprising administering a dosage of 0.01 mg to 0.1 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 0.1 mg to 1 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 1 mg to 10 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 10 mg to 100 mg of drug per kilogram of body weight orally daily, optionally in two or more divided doses per day, for the desired treatment duration with the NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 0.01 mg to 0.1 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 0.1 mg to 1 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 1 mg to 10 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 10 mg to 100 mg of drug per kilogram of body weight orally daily, optionally in two or more divided doses per day, for the desired treatment duration with an NS3 inhibitor compound.

### Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, some embodiments provide any of the above-described methods for the treatment of HCV infection in which the subject method is modified to treat a treatment failure patient for a duration of 48 weeks.

Other embodiments provide any of the above-described methods for HCV in which the subject method is modified to treat a non-responder patient, where the patient receives a 48 week course of therapy.

Other embodiments provide any of the above-described methods for the treatment of HCV infection in which the subject method is modified to treat a relapser patient, where the patient receives a 48 week course of therapy.

Other embodiments provide any of the above-described methods for the treatment of HCV infection in which the subject method is modified to treat a naïve patient infected with HCV genotype 1, where the patient receives a 48 week course of therapy.

Other embodiments provide any of the above-described methods for the treatment of HCV infection in which the subject method is modified to treat a naïve patient infected with HCV genotype 4, where the patient receives a 48 week course of therapy.

Other embodiments provide any of the above-described methods for the treatment of HCV infection in which the subject method is modified to treat a naïve patient infected with HCV genotype 1, where the patient has a high viral load (HVL), where “HVL” refers to an HCV viral load of greater than 2×10⁶ HCV genome copies per mL serum, and where the patient receives a 48 week course of therapy.

One embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 24 weeks to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

Another embodiment provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

### Subjects Suitable for Treatment

Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### Assays

Although assays currently exist to measure the protease, helicase and ATPase activity of NS3, the low activity of NS3 in solution require greater concentrations of enzyme than substrate to detect any enzyme activity. Assays incorporating such high enzyme concentration are prone to promiscuous inhibition, resulting in an excessive number of false positive results. There is currently a need in the art for assays with sufficient sensitivity and specificity to detect the activity of NS3 protease, helicase, and ATPase activity. In some embodiments, these assays can be utilized to detect inhibition of the protease, helicase, and ATPase activity of NS3 by inhibitive compounds, including the compounds disclosed herein.

In some embodiments, an NS3 enzyme with increased helicase activity is incorporated into a standard helicase assay to measure the helicase activity. The incorporation of an NS3 enzyme with increased helicase activity into a standard helicase assay can result in increased sensitivity and/or specificity of assays measuring the helicase activity of the NS3 enzyme.

In some embodiments, an NS3 enzyme with increased protease activity is incorporated into a standard protease assay to measure the protease activity. The incorporation of an NS3 enzyme with increased protease activity into a standard protease assay can result in increased sensitivity and/or specificity of assays measuring the protease activity of the NS3 enzyme.

In some embodiments, an NS3 enzyme with increased ATPase activity is incorporated into a standard ATPase assay to measure the ATPase activity. The incorporation of an NS3 enzyme with increased ATPase activity into a standard ATPase assay can result in increased sensitivity and/or specificity of assays measuring the ATPase activity of the NS3 enzyme.

In one embodiment, an amine oxide is added to the NS3 to improve the helicase activity. In some embodiments, the amine oxide is selected from the group consisting of lauryl (dimethyl)-amine oxide (LDAO), N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide. In a preferred embodiment, LDAO is used. In some embodiments, LDAO is added to a solution containing NS3 wherein the final concentration of LDAO in solution is about, at least, at least about, more than, more than about, between, between about 0.01 mM, 0.02 mM, 0.03 mM, 0.04 mM, 0.05 mM, 0.06 mM, 0.07 mM, 0.08 mM, 0.09 mM, 0.10 mM, 0.12 mM, 0.14 mM, 0.16 mM, 0.18 mM, 0.20 mM, 0.3 mM, 0.4 mM, 0.5 mM, 0.6 mM, 0.7 mM, 0.8 mM, 0.9 mM, 1.0 mM, 1.5 mM, and/or 20. mM.

In another embodiment, at least one detergent is added to a solution containing NS3 to improve the helicase activity. In some embodiments, the detergent is selected from the group consisting of LDAO, Tween 20, Triton X100, Pluronic F127, CHAPS, β-octyl glucoside, laurylmaltoside, N-lauroylsarcosine, and hexadecyltrimethylammonium bromide. In a preferred embodiment, LDAO is added. In a more preferred embodiment, at least one additional detergent in added to the solution containing NS3 and LDAO. In some embodiments, the additional detergent(s) is/are selected from the group consisting of Tween 20, Triton X100, Pluronic F127, CHAPS, β-octyl glucoside, laurylmaltoside, N-lauroylsarcosine, and hexadecyltrimethylammonium bromide. Table A represents the helicase activity of NS3 in the presence of LDAO and at least one additional detergent; and, in the absence of LDAO and the presence of at least one other detergent.

TABLE A
Activity of NS3 Helicase in Presence
of Detergent with or without LDAO

| | | Concen- | Slope | Activity |
|---|---|---|---|---|
| | Detergents | tration | [mP/min] | [%] |
| plus LDAO | Control | Control | −0.38 | 100 |
| 0.6 mM | Tween 20 | 0.020% | −1.36 | 358 |
| | Tween 20 | 0.075% | −1.13 | 297 |
| | Triton X100 | 0.020% | −1.51 | 397 |
| | Triton X100 | 0.075% | −1.19 | 313 |
| | Pluronic F127 | 0.020% | −1.14 | 300 |
| | Pluronic F127 | 0.075% | −1.29 | 339 |
| | Control | Control | −0.61 | 100 |
| | CHAPS | 0.020% | −0.77 | 126 |
| | CHAPS | 0.075% | −1.16 | 190 |
| | β-octyl glucoside | 0.020% | −0.45 | 74 |
| | β-octyl glucoside | 0.075% | −0.38 | 62 |
| | laurylmaltoside | 0.05 mM | −0.8 | 131 |
| | laurylmaltoside | 0.2 mM | −1.23 | 202 |
| | N-lauroylsarcosine | 0.05 mM | −0.53 | 87 |
| | N-lauroylsarcosine | 0.2 mM | −0.28 | 46 |
| | hexadecyltrimethyl- | 0.05 mM | 0.24 | −39 |
| | ammonium bromide | | | |
| | hexadecyltrimethyl- | 0.2 mM | 0.74 | −121 |
| | ammonium bromide | | | |
| no LDAO | Tween 20 | 0.020% | −0.39 | 103 |
| | Tween 20 | 0.075% | −0.28 | 74 |
| | Triton X100 | 0.020% | −0.25 | 66 |
| | Triton X100 | 0.075% | −0.2 | 53 |
| | Pluronic F127 | 0.020% | −0.48 | 126 |
| | Pluronic F127 | 0.075% | −0.59 | 155 |

In one embodiment, an amine oxide is added to the NS3 to improve the protease activity. In some embodiments, the amine oxide is selected from the group consisting of lauryl (dimethyl)-amine oxide (LDAO), N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide. In a preferred embodiment, LDAO is used. In some embodiments, LDAO is added to a solution containing NS3 wherein the final concentration of LDAO in solution is about, at least, at least about, more than, more than about, between, between about 0.01 mM, 0.02 mM, 0.03 mM, 0.04 mM, 0.05 mM, 0.06 mM, 0.07 mM, 0.08 mM, 0.09 mM, 0.10 mM, 0.12 mM, 0.14 mM, 0.16 mM, 0.18 mM, 0.20 mM, 0.3 mM, 0.4 mM, 0.5 mM, 0.6 mM, 0.7 mM, 0.8 mM, 0.9 mM, 1.0 mM, 1.5 mM, and/or 2.0 mM.

In another embodiment, at least one detergent is added to a solution containing NS3 to improve the protease activity. In some embodiments, the detergent is selected from the group consisting of LDAO, Tween 20, Triton X100, Pluronic F127, CHAPS, β-octyl glucoside, laurylmaltoside, N-lauroylsarcosine, and hexadecyltrimethylammonium bromide. In a preferred embodiment, LDAO is added. In a more preferred embodiment, at least one additional detergent in added to the solution containing NS3 and LDAO. In some embodiments, the additional detergent(s) is/are selected from the group consisting of Tween 20, Triton X100, Pluronic F127, CHAPS, β-octyl glucoside, laurylmaltoside, N-lauroylsarcosine, and hexadecyltrimethylammonium bromide. In another embodiment, salts, solvents and stabilizers can be added to a solution containing NS3 to improve its protease activity.

In one embodiment, the NS3 helicase assay is conducted with a helicase concentration about, at least, at least about, more than, more than about, between, between about 0.001 nM, 0.01 nM, 0.1 nM, 0.2 nM, 0.3 nM, 0.4 nM, 0.5 nM, 0.6 nM, 0.7 nM, 0.8 nM, 0.9 nM, 1.0 nM, 1.1 nM, 1.2 nM, 1.3 nM, 1.4 nM, 1.5 nM, 1.6 nM, 1.7 nM, 1.8 nM, 1.9 nM, 2.0 nM, 2.1 nM, 2.2 nM, 2.3 nM, 2.4 nM, 2.5 nM, 2.6 nM, 2.7 nM, 2.8 nM, 2.9 nM, 3.0 nM, 3.1 nM, 3.2 nM, 3.3 nM, 3.4 nM, 3.5 nM, 3.6 nM, 3.7 nM, 3.8 nM, 3.9 nM, 4.0 nM, 4.1 nM, 4.2 nM, 4.3 nM, 4.4 nM, 4.5 nM, 4.6 nM, 4.7 nM, 4.8 nM, 4.9 nM, 5.0 nM, 5.2 nM, 5.4 nM, 5.6 nM, 5.8 nM, 6.0 nM, 6.2 nM, 6.4 nM, 6.6 nM, 6.8 nM, 7.0 nM, 7.2 nM, 7.4 nM, 7.6 nM, 7.8 nM, 8.0 nM, 8.2 nM, 8.4 nM, 8.6 nM, 8.8 nM, 9.0 nM, 9.2 nM, 9.4 nM, 9.6 nM, 9.8 nM, 10.0 nM, 20 nM, 30 nM, 40 nM, 50 nM, 60 nM, 70 nM, 80 nM, 90 nM, and 100 nM. In a preferred embodiment, the helicase assay is conducted with a helicase concentration of 5 nM.

In one embodiment, the NS3 helicase assay is conducted with the addition of Tris to the assay buffer at a final concentration about, at least, at least about, more than, more than about, between, between about 0.001 mM, 0.01 mM, 0.1 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 52 mM, 54 mM, 56 mM, 58 mM, 60 mM, 62 mM, 64 mM, 66 mM, 68 mM, 70 mM, 72 mM, 74 mM, 76 mM, 78 mM, 80 mM, 82 mM, 84 mM, 86 mM, 88 mM, 90 mM, 92 mM, 94 mM, 96 mM, 98 mM, 100 mM, 200 mM, 300 mM, 400 mM, and 500 mM in the assay buffer. In a preferred embodiment, the NS3 helicase assay is conducted with the addition of Tris to the assay buffer at a final concentration of 50 mM in the assay buffer.

In one embodiment, the NS3 helicase assay is conducted with the addition of MgCl₂ to the assay buffer at a final concentration about, at least, at least about, more than, more than about, between, between about 0.001 mM, 0.01 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.2 mM, 5.4 mM, 5.6 mM, 5.8 mM, 6.0 mM, 6.2 mM, 6.4 mM, 6.6 mM, 6.8 mM, 7.0 mM, 7.2 mM, 7.4 mM, 7.6 mM, 7.8 mM, 8.0 mM, 8.2 mM, 8.4 mM, 8.6 mM, 8.8 mM, 9.0 mM, 9.2 mM, 9.4 mM, 9.6 mM, 9.8 mM, 10 mM, 20 mM, 30 mM, 40 mM, and 50 mM in the assay buffer. In a preferred embodiment, the NS3 helicase assays is conducted with the addition of MgCl₂ to the assay buffer at a final concentration of 5 mM in the assay buffer.

In one embodiment, the NS3 helicase assays is conducted with an ATP substrate concentration about, at least, at least about, more than, more than about, between, between about 0.001 mM, 0.01 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 4.0 mM, 5.0 mM, 10 mM, 25 mM, 50 mM, and 100 mM. In a preferred embodiment, the NS3 helicase assays is conducted with an ATP substrate concentration of 1.5 mM.

In one embodiment, the NS3 helicase assay is conducted with a duplex oligonucleotide concentration about, at least, at least about, more than, more than about, between, between about 0.001 nM, 0.01 nM, 0.1 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 M, 50 nM, 52 nM, 54 nM, 56 nM, 58 nM, 60 nM, 62 nM, 64 nM, 66 nM, 68 nM, 70 nM, 72 nM, 74 nM, 76 nM, 78 nM, 80 nM, 82 nM, 84 nM, 86 nM, 88 nM, 90 nM, 92 nM, 94 nM, 96 nM, 98 nM, 100 nM, 200 nM, 300 nM, 400 nM, and 500 nM. In a preferred embodiment, the NS3 helicase assay is conducted with a duplex oligonucleotide concentration of 50 nM.

In one embodiment, the NS3 helicase assay is conducted with a capture strand concentration about, at least, at least about, more than, more than about, between, between about 0.001 mM, 0.01 mM, 0.1 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, 500 mM, 520 mM, 540 mM, 560 mM, 580 mM, 600 mM, 620 mM, 640 mM, 660 mM, 680 mM, 700 mM, 720 mM, 740 mM, 760 mM, 780 mM, 800 mM, 820 mM, 840 mM, 860 mM, 880 mM, 900 mM, 920 mM, 940 mM, 960 mM, 980 mM, 1 M, 2 M, 3 M, 4 M, and 5 M. In a preferred embodiment, the NS3 helicase assay is conducted with a capture strand concentration of 250 nM.

In one embodiment, the NS3 helicase assay is conducted with the addition of DTT to the assay buffer at a final concentration about, at least, at least about, more than, more than about, between, between about 0.001 mM, 0.01 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.2 mM, 5.4 mM, 5.6 mM, 5.8 mM, 6.0 mM, 6.2 mM, 6.4 mM, 6.6 mM, 6.8 mM, 7.0 mM, 7.2 mM, 7.4 mM, 7.6 mM, 7.8 mM, 8.0 mM, 8.2 mM, 8.4 mM, 8.6 mM, 8.8 mM, 9.0 mM, 9.2 mM, 9.4 mM, 9.6 mM, 9.8 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 40 mM, 50 mM, 60 mM, 70 mM, 80 mM, 90 mM, and 100 mM in the assay buffer. In a preferred embodiment, the NS3 helicase assay is conducted with the addition of DTT to the assay buffer at a concentration of 10 mM in the assay buffer.

In one embodiment, the NS3 helicase assay is conducted with the addition of glycerol to the assay buffer at a final concentration about, at least, at least about, more than, more than about, between, between about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, and 40% in the assay buffer. In a preferred embodiment, the NS3 helicase assay is conducted with the addition of glycerol to the assay buffer at a final concentration of 15% in the assay buffer.

In some embodiments, an ATPase assay is employed to analyze the ATPase activity of NS3. This assay for HCV NS3 ATPase activity is an indirect detectable marker assay principle. In some embodiments, the assay for HCV NS3 ATPase activity is an indirect fluorescence polarization assay principle applied on the basis of a commercially available assay kit (Transcreener™ Kinase Plus, Bellbrook Labs, U.S.A.). In some embodiments, ATP substrate is de-phosphorylated at the y-position and converted to ADP as a result of the activity of NS3 ATPase. As a result, increasing concentrations of the generated product ADP compete with an ADP tracer molecule, labeled with a detectable marker, for binding to an ADP-specific antibody. In a preferred embodiment, the ADP tracer molecule is labeled with a fluorescent marker.

In some embodiments, after a defined incubation period NS3 ATPase is inactivated by addition of a stop solution. Potential inhibitors of NS3 ATPase can then be identified as binding of the ADP tracer, linked to a detectable marker, to the antibody results in capture of a signal from the detectable marker. Binding of the fluorescence coupled ADP tracer to the antibody results in a fluorescence polarized signal. In contrast, the presence of active NS3 ATPase leads to displacement of fluorescent ADP tracer from the antibody leading to low fluorescence polarization signals.

In a preferred embodiment, NS3 with increased helicase activity, as described in the embodiments presented herein, is used in conjunction with the indirect fluorescence assay to deliver more specific and/or sensitive results for the ATPase activity of NS3.

In one embodiment, a helicase assay is employed to analyze the helicase activity of NS3. In embodiments of this assay a double stranded DNA oligonucleotide is used as the substrate for the helicase unwinding reaction. In some embodiments, one strand of the duplex comprises a detectable marker wherein the opposite strand contains a quenching moiety that is able to quench signal from the detectable marker. In a preferred embodiment, the (+)-DNA strand is labeled with redshifted dyes, including, but not limited to, MR121 and Att0647 at its 5′-end. In a preferred embodiment, the 3′-end of the (−)-DNA strand is composed of a stretch of three guanosine (“G”) nucleotides which come into close proximity with the dye of the complementary (+)-DNA strand. Interaction of fluorescence dyes with guanosine bases lead to energy transfer and effective quenching of the emitted signals.

In some embodiments, NS3 helicase is incubated with an oligonucleotide substrate described in the embodiments presented herein. In some embodiments, the NS3 helicase facilitates ATP-dependent unwinding of the DNA duplex and separation of both single strands. In a preferred embodiment, a “capture” DNA single strand is added to prevent re-annealing of the dissociated DNA strands. In some embodiments, the “capture” oligonucleotide is complementary to the (−) DNA strand. In other embodiments, the “capture” oligonucleotide is complementary to the (+) DNA strand.

In some embodiments, the described “G”-quench effect of the guanosine residues is further amplified by additional labeling of the 3′-end of the DNA strand containing the guanosine residues with biotin. This modification allows for tight binding of the intact duplex to streptavidin which in some embodiments is included in the stop solution. As a consequence, the dye comes into close proximity to streptavidin leading to further quenching of the signal from the red-shifted dyes.

### Assay Example 1

In order to identify reaction conditions that give high levels of HCV NS3/4a protease activity, several additives were analyzed to determine their effect on reaction rate. The base buffer used was 50 mM Tris-HCl, pH 7.5 containing 15% glycerol. The FRET-based assay substrate used (sequence: Ac-DE-Dap(QXL520)-EE-Abu-ψ-[COO]-AS-Cys(5-FAMsp)-NH₂) was obtained from Anaspec, Inc. (San Jose, Calif.). The NS4a surrogate peptide used (KGSVVIVGRIILSGRK) was obtained from Midwest Biotech (Fishers, Ind.). The NS3 enzyme used was the benchmark wild-type full length enzyme derived from HCV genotype 1b-K2040. The reaction rate for the NS3 catalyzed hydrolysis of 0.5 μM substrate in base buffer was used as a reference. The effect of additives at varying concentrations on the reaction rate was studied and the data are summarized in Table B below.

TABLE B

| Additive tested | Concentrations of additive tested | | Conclusion |
|---|---|---|---|
| DTT | 0, 1, 10 and 30 | mM | 10 and 30 mM DTT improve activity. |
| β-ME | 0, 1, 10 and 100 | mM | Little improvement in activity |
| TCEP | 0, 0.5 and 1 | mM | detrimental to activity |
| LDAO | 0, 0.06 and 0.6 | mM | LDAO at 0.6 mM raised activity |
| | | | significantly (~10-fold) |
| CHAPS | 0, 0.2 and 2 | mM | CHAPS at 2 mM increased activity |
| β-OG | 0, 0.5 and 5 | mM | No improvement in activity |
| Tween-20 | 0, 1.2 and 12 | μM | Improvement in activity at 12 μM |
| Triton X-100 | 0, 6 and 60 | μM | Improvement in activity at 12 μM |
| NaCl | 0, 100 and 500 | mM | NaCl detrimental to activity |
| NS4a peptide | 0, 2.5, 25, 250 and 2500 | μM | Best activity at 25 and 2500 μM |

The composition that generated maximal protease activity was found to be

50 mM Tris-HCl, pH 7.5

15% glycerol

0.6 mM LDAO

10 mM DTT

25 μM NS4a peptide

### Assay Example 2

Various assay conditions were analyzed to determine the effect on the helicase activity of NS3. Helicase activity was measured using a double stranded DNA oligonucleotide as the substrate for the helicase unwinding reaction. The (+) strand of the duplex comprised the fluorophonre FAM and (−) strand contained the quenching moiety black hole quenching (BHQ-1) which was able to quench signal from the FAM when the duplex was in tact. The NS3 helicase, under various assay conditions described below, was incubated with the oligonucleotide substrate, facilitating ATP-dependent unwinding of the DNA duplex and separation of both single strands. A “capture” DNA single strand was added to prevent re-annealing of the dissociated DNA strands. The fluorescent signal from the FAM was measured to determine the level of NS3 activity.

### Various Buffer Conditions

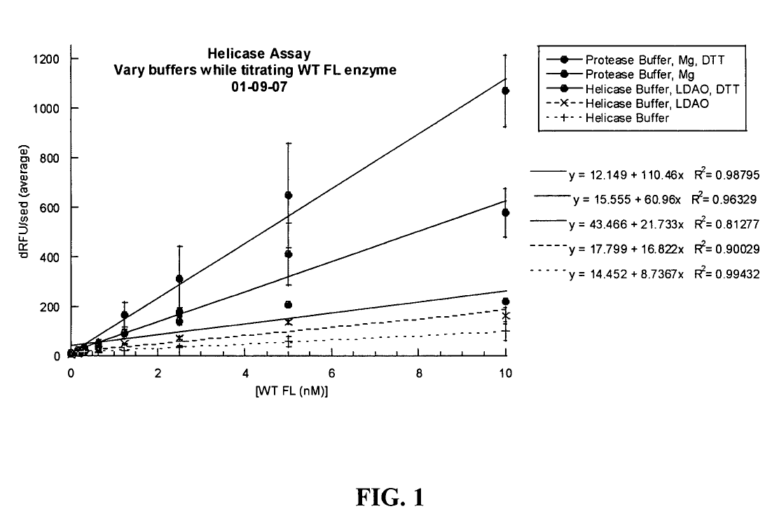

Helicase activity was analyzed using various buffer conditions while varying enzyme concentration. As a starting point, standard stocks of helicase or protease buffers were used. The stock helicase buffer contained 25 mM MOPS, ph 7.0, 1.5 mM MgCl₂, 0.005% Triton X-100. The stock protease buffer contained 50 mM Tris, ph 7.5, 0.6 mM LDAO, and 15% glycerol. Helicase assay was analyzed using various concentrations of enzyme with the addition of stock buffers supplemented with Mg, DTT, and/or LDAO. FIG. 1 depicts the helicase activity of the enzyme in the presence of various buffers.

The results of the optimization analysis revealed that stock protease buffer supplemented with 1.5 mM MgCl₂, and 10 mM DTT produced the best results for helicase activity, followed by stock protease buffer supplemented with MgCl₂. The next best results were achieved with helicase buffer supplemented with LDAO and DTT, followed by helicase buffer supplemented with LDAO. Helicase buffer alone provided a control and displayed the lowest NS3 helicase activity.

### Varying Amounts of Enzyme

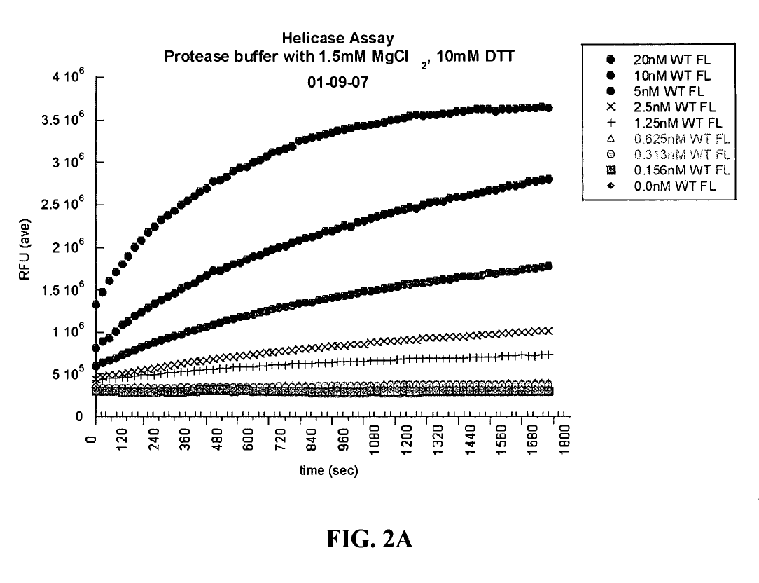

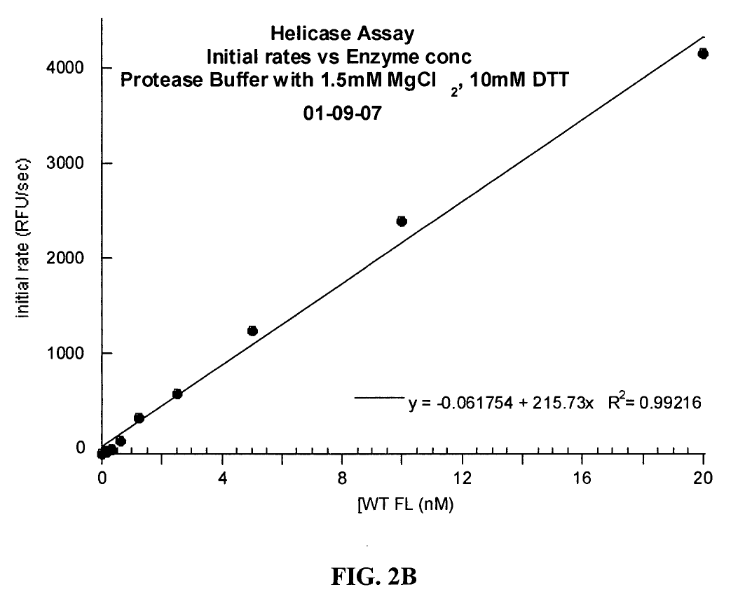

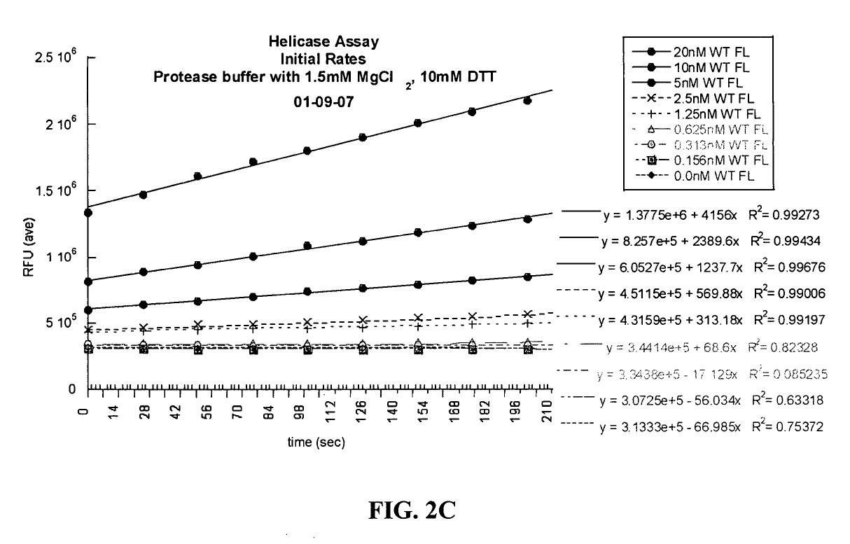

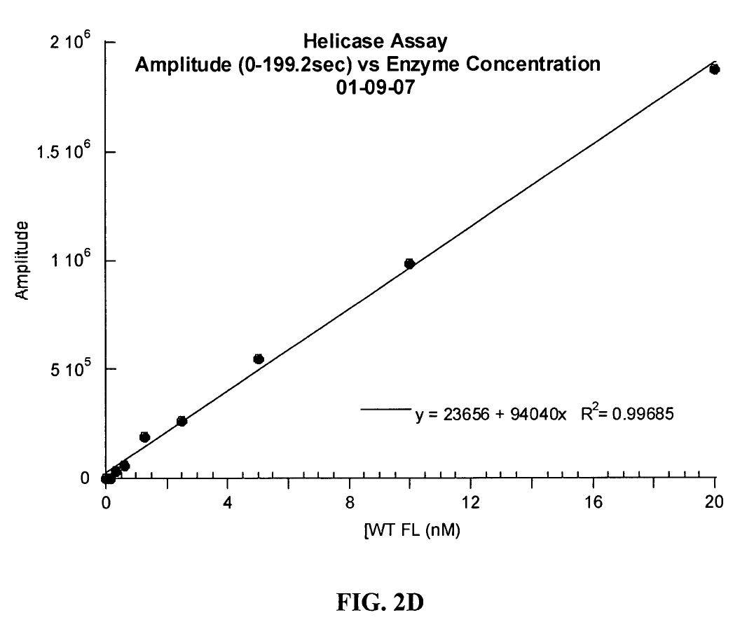

Helicase activity was analyzed using various concentrations of full length wild type (WT FL) NS3 enzyme. The helicase assay was performed as recited above in the presence of protease buffer supplemented with 1.5 mM MgCl₂ and 10 mM DTT. FIGS. 2A-2D depict the results of NS3 helicase assay in the presence of varying concentrations of NS3 enzyme. FIG. 2A depicts the relative fluorescence units (RFU) as a function of time. FIG. 2B depicts the initial rate of the unwinding reaction (RFU/second) as a function of enzyme concentration. FIG. 2C depicts the initial rate (RFU (average)) of the unwinding reaction as a function of time. FIG. 2D measures the amplitude (measured by the final RFU) of the unwinding reaction as a function of enzyme concentration.

### Varying Amounts of MgCl₂

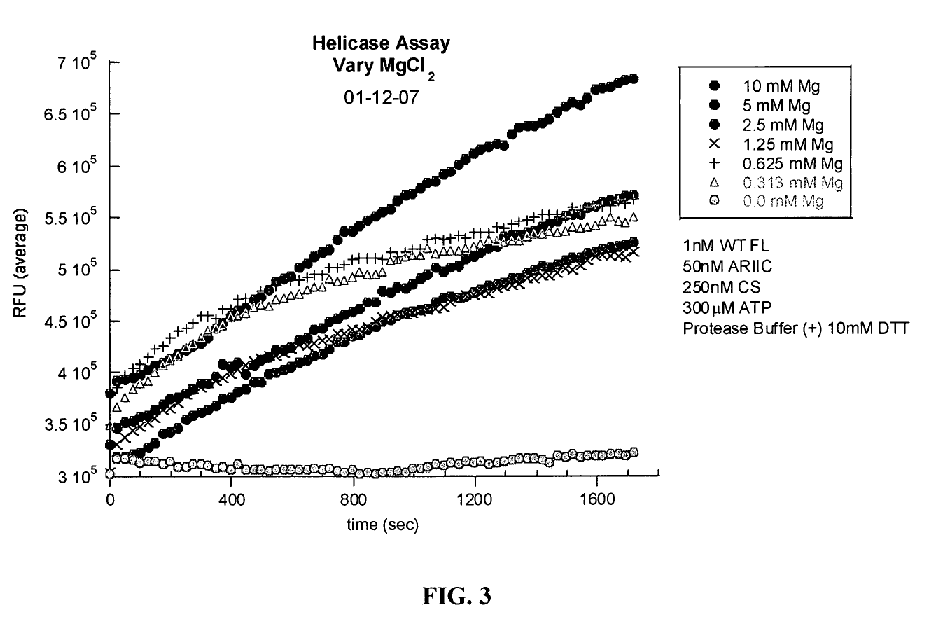

The helicase activity of NS3 was analyzed using various concentrations of MgCl₂ in the assay buffer. Helicase activity was measured as described below wherein the reaction consisted of 1 nM WT FL NS3 enzyme, 50 nM oligonucleotide substrate, 250 nM capture oligonucleotide, 300 μM ATP in an assay buffer containing protease buffer supplemented with 10 mM DTT. Various amounts of MgCl₂ were added to the assay buffer to evaluate optimal MgCl₂ concentrations. 10 mM, 5 mM, 2.5 mM, 1.25, 0.625 mM, 0.313 mM, and 0 mM concentrations of MgCl₂ were analyzed. FIG. 3 depicts the results of the MgCl₂ optimization evaluation.

### Varying Amounts of ATP

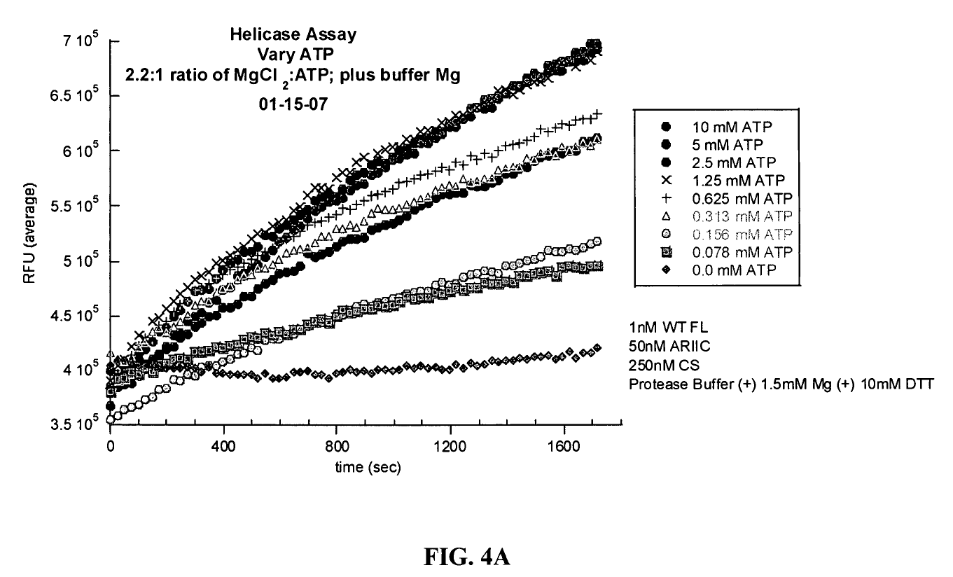

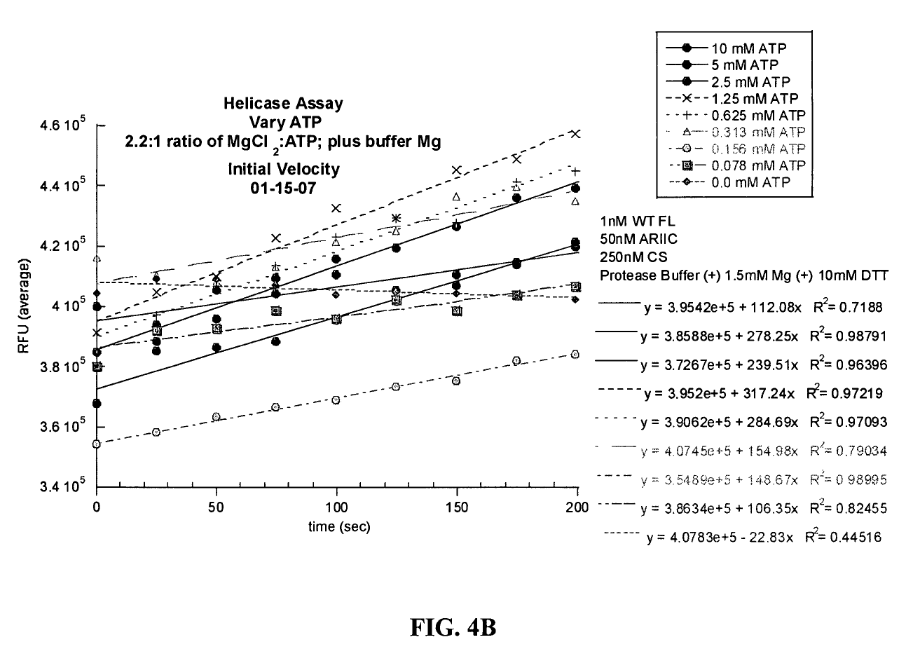

The helicase activity of NS3 was analyzed using various concentrations of ATP in the assay buffer. Helicase activity was measured as described below wherein the reaction consisted of 1 nM WT FL NS3 enzyme, 50 nM oligonucleotide substrate, 250 nM capture oligonucleotide, 300 μM ATP in an assay buffer containing protease buffer supplemented with 1.5 mM MgCl₂ and 10 mM DTT. Various amounts of ATP were added to the assay buffer to evaluate optimal ATP concentrations. 10 mM, 5 mM, 2.5 mM, 1.25, 0.625 mM, 0.313 mM, 0.156 mM, 0.078 mM, and 0 mM concentrations of ATP were analyzed. FIGS. 4A and 4B depicts the results of the ATP optimization evaluation.

### Varying Amounts of Duplex Oligonucleotide Substrate

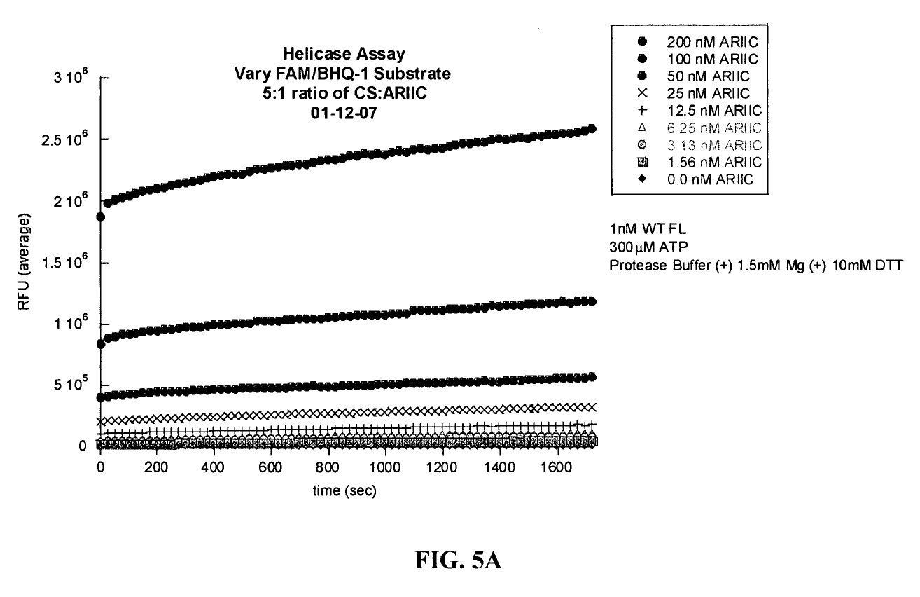

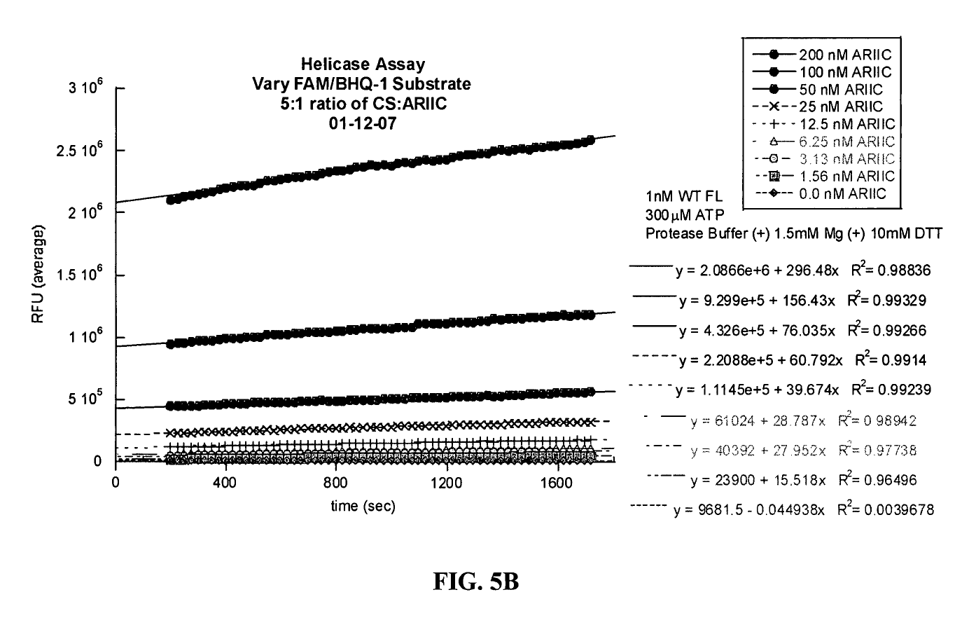

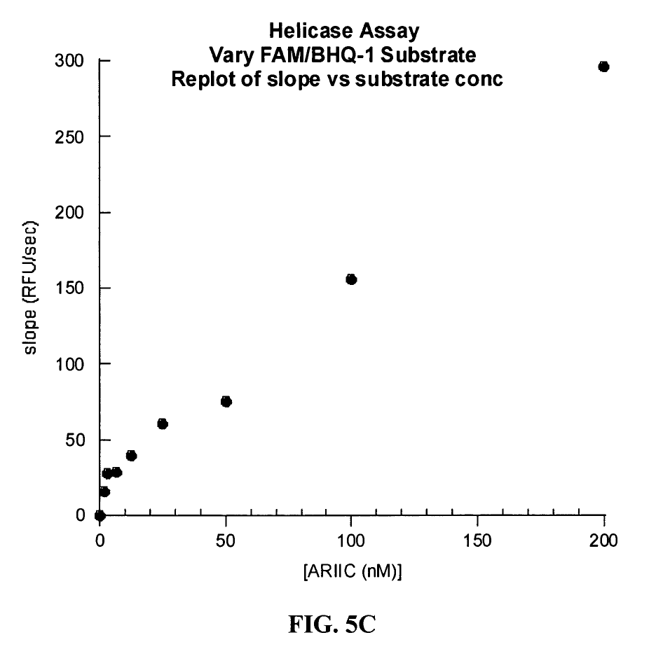

The helicase activity of NS3 was analyzed using various concentrations of duplex oligonucleotide substrate in the assay buffer. Helicase activity was measured as described below wherein the reaction consisted of 1 nM WT FL NS3 enzyme, 300 μM ATP, in an assay buffer containing protease buffer supplemented with 1.5 mM MgCl₂ and 10 mM DTT. Various amounts of duplex oligonucleotide substrate were added to the assay buffer to evaluate optimal oligonucleotide concentrations. 200 nM, 100 nM, 50 nM, 25 nM, 12.5 nM, 6.25 nM, 3.13 nM, 1.56 nM, and 0 mM concentrations of duplex oligonucleotide substrate were analyzed. FIGS. 5A to C depicts the results of the ATP optimization evaluation.

As a result of the optimizations, optimal assay conditions were develop which include 5 nM enzyme, 50 mM Tris pH 7.5, 0.6 mM LDAO, 5 mM MgCl₂, 1.5 mM ATP, 50 nM duplex oligonucleotide substrate, 250 nM capture strand, 10 mM DTT, and 15% glycerol. The observed rate of unwinding (k_{obs}) using the optimal assay conditions was 0.02 min⁻¹.

### Preparation of NS3 Inhibitors

Methodology

The HCV helicase inhibitors in the following sections can be prepared according to the procedures and schemes shown in each section. Certain compounds and intermediates used in the syntheses have been described elsewhere. The numberings in each of the following Preparation of NS3 Inhibitor sections are meant for that specific section only, and should not be construed or confused with the same numberings in other sections.

Preparation of NS3 Inhibitors: Section I

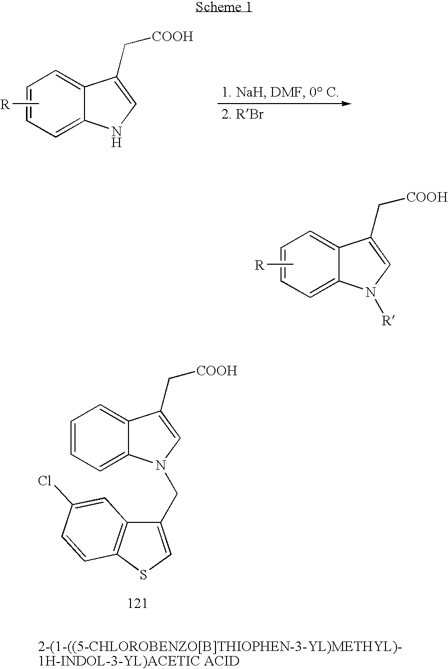

To a mixture of indole-3-acetic acid (100 mg, 0.57 mmol) in 1 ml DMF at 0° C. was added sodium hydride (60% dispersion in mineral oil, 54.8 mg 1.37 mmol). The mixture was stirred at 0° C. for 30 min. 3-(Bromomethyl)-5-chlorobenzo[b]thiophene (179 mg, 0.79 mmol) was added and the stirring continued for 1 hour. The reaction was quenched with ice water. The resultant mixture was washed three times with Et₂O (Et=ethyl) and the aqueous solution was then acidified with 1N HCl. The cloudy mixture was extracted with EtOAc (3×) and the organic extracts were washed with 1N HCl (3×) and dried with Na₂SO₄ and concentrated. The desired product, 2-(1-((5-chlorobenzo[b]thiophen-3-yl)methyl)-1H-indol-3-yl)acetic acid, was obtained as a white powder by tritriration of the crude product with Et₂O, yield 64 mg. APCI(neg) 354.0 (M-1).

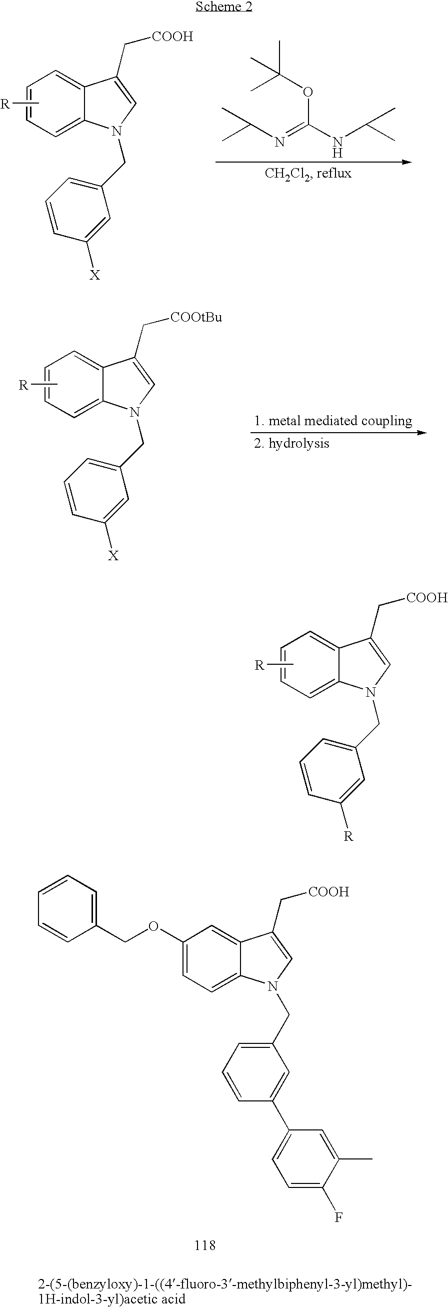

### Step 1: Preparation of tert-butyl 2-(5-(benzyloxy)-1-(3-bromobenzyl)-1H-indol-3-yl)acetate

To a 100 mL round-bottomed flask was placed 2-(5-(benzyloxy)-1-(3-bromobenzyl)-1H-indol-3-yl)acetic acid (0.69 g, 1.5 mmol) in 10 mL DCM. tert-Butyl N,N′-diisopropylcarbamimidate (0.46 g, 2.3 mmol) was then added. The mixture was heated to reflux. The solution turned from clear pale yellow to cloudy white. After 2 hours, additional tert-Butyl N,N′-diisopropylcarbamimidate (0.46 g, 2.3 mmol) was added and refluxed for 1 hours and stayed at room temperature overnight. Additional tert-Butyl N,N′-diisopropylcarbamimidate (0.46 g, 2.3 mmol) was added the next day and refluxed for 6 hours. The reaction was cooled to room temperature. A white solid was filtered and washed with dichloromethane. The mixture was concentrated to a brown oil and purified on column, eluted with Hexane to 5:1 Hexane/EtOAc to get the desired product tert-Butyl 2-(5-(benzyloxy)-1-(3-bromobenzyl)-1H-indol-3-yl)acetate as a clear oil (0.68 g, 88% yield).

### Step 2: Preparation of tert-Butyl 2-(5-(benzyloxy)-1-((4′-fluoro-3′-methylbiphenyl-3-yl)methyl)-1H-indol-3-yl)acetate

To a degassed solution of tert-Butyl 2-(5-(benzyloxy)-1-(3-bromobenzyl)-1H-indol-3-yl)acetate (100 mg, 0.20 mmol) and 4-fluoro-3-methylphenylboronic acid (30 mg, 0.20 mmol) in a mixture of DME (4 mL) and 2M aqueous Na₂CO₃ (2 mL) in a sealed tube was added tetrakis (triphenylphosphine) palladium (11.4 mg. 0.010 mmol). The reaction mixture was stirred at reflux overnight. The reaction mixture was diluted with ethyl acetate and water. The organic layer was separated, the aqueous layer was extracted with EtOAc (2×) and dried over Na₂SO₄ and concentrated. The residue was purified by column chromatography, eluted with Hex to 10% EtOAc/Hex to get the product tert-Butyl 2-(5-(benzyloxy)-1-((4′-fluoro-3′-methylbiphenyl-3-yl)methyl)-1H-indol-3-yl)acetate (80 mg, 76% yield).

### Step 3: Preparation of 2-(5-(benzyloxy)-1-((4′-fluoro-3′-methylbiphenyl-3-yl)methyl)-1H-indol-3-yl)acetic acid

To tert-butyl 2-(5-(benzyloxy)-1-((4′-fluoro-3′-methylbiphenyl-3-yl)methyl)-1H-indol-3-yl)acetate (40 mg, 0.0075 mmol) in a flask was added 2 mL 10% TFA/CH₂Cl₂ solution (TFA=trifluoroacetic acid). The mixture was stirred at room temperature overnight. The solvent was removed. The residue was then purified by reverse phase column on Horizon, eluted with 10%˜85% of CH₃CN/water to give the desired product 2-(5-(benzyloxy)-1-((4′-fluoro-3′-methylbiphenyl-3-yl)methyl)-1H-indol-3-yl)acetic acid (26 mg, 73% yield). APCI(neg) 478.1 (M-1).

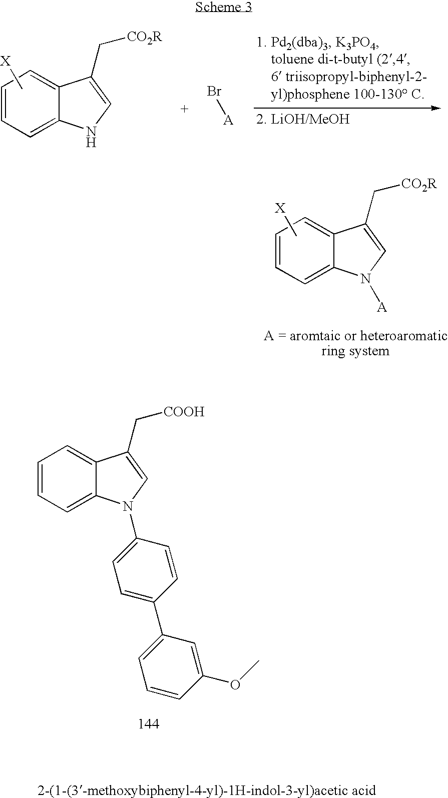

### Step 1: Preparation of ethyl 2-(1-(3′-methoxybiphenyl-4-yl)-1H-indol-3-yl)acetate

In a sealed tube was charged with ethyl 2-(1H-indol-3-yl)acetate (210 mg, 10 mmol), 4′-bromo-3-methoxybiphenyl (326 mg, 1.24 mmol), di-t-butyl (2′,4′,6′ triisopropyl-biphenyl-2-yl)phosphene 33 mg, 0.078 mmol), potassium phosphate (307 mg, 1.45 mmol) and Tris(dibenzylideneacetone) dipalladium (0) (24 mg, 0.026 mmol) in 2 mL toluene. The system was purged with N₂ for 10 min then heated to 108° C. for 16 hours. The reaction was cooled down to room temperature and diluted with EtOAc. The mixture was filtered through celite and concentrated. The crude product was purified on Biotage, eluted with 20:1 to 10:1 Hex/EtOAc to get the desired product ethyl 2-(1-(3′-methoxybiphenyl-4-yl)-1H-indol-3-yl)acetate (300 mg, 75% yield). APCI(neg) 356.1 (M-1).

### Step 2: Preparation of 2-(1-(3′-methoxybiphenyl-4-yl)-1H-indol-3-yl)acetic acid

Ethyl 2-(1-(3′-methoxybiphenyl-4-yl)-1H-indol-3-yl)acetate (100 mg, 0.26 mmol) was dissolved in 2 mL 1:1 THF/MeOH (THF=tetrahydrofuran, Me=methyl). Lithium hydroxide hydrate (44 mg, 1.1 mmol) was added and the reaction was stirred at the room temperature for three hours. The solvent was evaporated. Water was added and a few drops of 1N NaOH was added. The mixture was then extracted with Et₂O (3×) and the Et₂O extract was discarded. The aqueous layer was then acidified with 1N HCl and extracted with EtOAc (3×). The organic layer was dried with Na₂SO₄. The product was obtained by crystallized from EtOAc/Hex to get a yellow solid (65 mg, 70% yield).

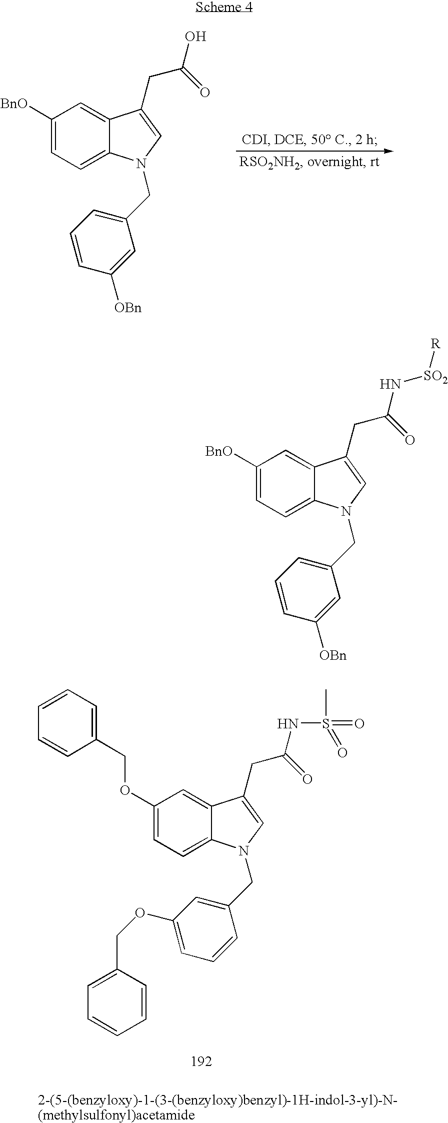

A solution of acid (10 mg, 0.021 mmol) in 1,2-dichloroethane (0.5 mL) was added 1,1′-carbonyldiimidazole (4.5 mg, 0.027 mmol) and stirred at 50° C. After 2 h, it was added methane sulfonamide (2.4 mg, 0.025 mmol), DBU (6.4 mg, 0.042 mmol) and stirred for overnight at room temperature. Portion of the reaction mixture was purified by preparative TLC. LCMS (APCI)⁻ at m/z 553.3 (M-H), Rt=3.03 min.

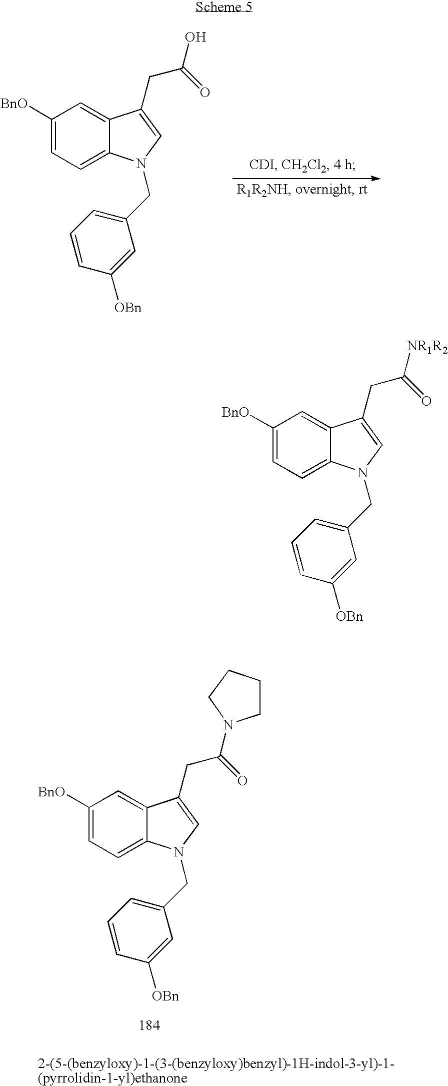

A solution of acid (50 mg, 0.105 mmol) in dichloromethane (1.0 mL) was added 1,1′-carbonyldiimidazole (23.75 mg, 0.146 mmol) and stirred at room temperature. After h, it was added cyclopentylamine (8.2 mg, 0.12 mmol) and stirred for overnight at room temperature. Portion of the reaction mixture was purified by preparative TLC. LCMS (APCI)⁻ at m/z 529.2 (M-H).

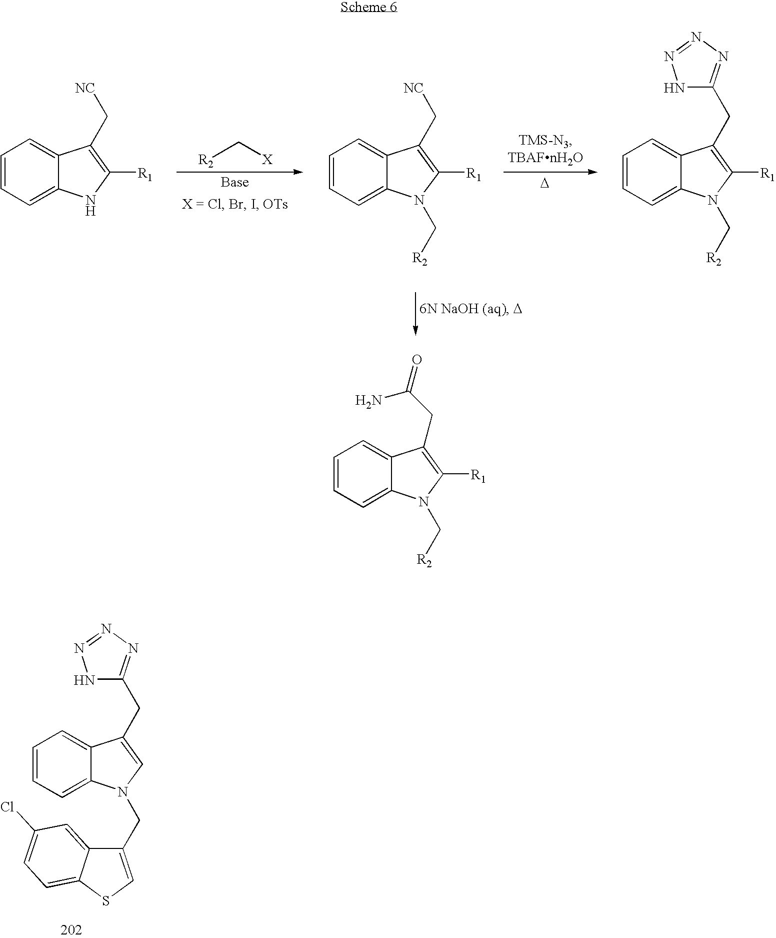

### Step 1: Preparation of 2-(1-((5-chlorobenzo[b]thiophen-3-yl)methyl)-1H-indol-3-yl)acetonitrile

To a solution of 2-(1H-indol-3-yl)acetonitrile (300 mg, 1.92 mmol) in THF (6 mL) at 0° C. was added a 1.0 M solution of NaHMDS in THF (1.92 mL, 1.92 mmol) and the reaction was stirred at 0° C. for 5 minutes and then 3-(bromomethyl)-5-chlorobenzo[b]thiophene (502 mg, 1.92 mmol) was added. The reaction was allowed to warm to room temperature overnight. The reaction was diluted with EtOAc (20 mL) and the organics were washed with 1N HCl (aq). The aqueous phase was washed with brine, dried (MgSO₄) and the solvents were removed in vacuo. The residue was purified by silica gel column eluting with 5:1 hexanes/EtOAc. This material was then further purified by PLC eluting with dichloromethane (DCM) and the band at R_{f} 0.45 provided 2-(1-((5-chlorobenzo[b]thiophen-3-yl)methyl)-1H-indol-3-yl)acetonitrile (180 mg, 28% yield) as a yellow oil. ¹H NMR (400 mHz. CDCl₃) δ 7.78 (d, J=8.6 Hz, 1H), 7.65-7.03 (m, 8H), 5.46 (s, 2H).

### Step 2: Preparation of 3-((1H-tetrazol-5-yl)methyl)-1-((5-chlorobenzo[b]thiophen-3-yl)methyl)-1H-indole

To a solution of 2-(1-((5-chlorobenzo[b]thiophen-3-yl)methyl)-1H-indol-3-yl)acetonitrile (100 mg, 0.297 mmol) in DCM (2 mL) was added TMSN₃ (0.119 mL, 0.891 mmol) and TBAF-3H₂O (46.8 mg, 0.148 mmol). The reaction was stirred at 120 C (external temp) in a sealed tube for 15 h. The reaction mixture was concentrated in vacuo and the residue was purified by PLC eluting with 2:1 DCM/EtOAc. 3-((1H-tetrazol-5-yl)methyl)-1-((5-chlorobenzo[b]thiophen-3-yl)methyl)-1H-indole (14 mg, 12% yield) was isolated as an off-white solid. MS APCI (−) m/z 380 detected. ¹H NMR (400 mHz. CD₃OD) δ 7.85 (d, J=8.6 Hz, 1H), 7.75 (s, 1H), 7.45-7.03 (m, 7H), 5.56 (s, 2H), 4.42 (s, 2H).

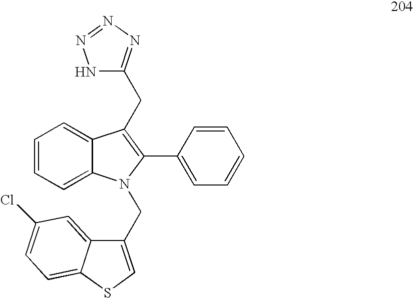

### Step 1: Preparation of 2-(1-((5-chlorobenzo[b]thiophen-3-yl)methyl)-2-phenyl-1H-indol-3-yl)acetonitrile

To a solution of 2-(2-phenyl-1H-indol-3-yl)acetonitrile (500 mg, 2.15 mmol) in THF (20 mL) at 0° C. was added NaHMDS (2.15 mL, 2.15 mmol) slowly over 2 mins. The 3-(bromomethyl)-5-chlorobenzo[b]thiophene (563 mg, 2.15 mmol) was then added in one portion. The reaction was warmed to room temperature over 4 h. Water (20 mL) was added and the beige solids were filtered off and dried. The solids were triturated with DCM with heating and sonication, filtered and dried. 2-(1-((5-chlorobenzo[b]thiophen-3-yl)methyl)-2-phenyl-1H-indol-3-yl)acetonitrile was isolated as a beige solid (404 mg, 45%). MS APCI (+) m/z 413 detected.

### Step 2: Preparation of 3-((1H-tetrazol-5-yl)methyl)-1-((5-chlorobenzo[b]thiophen-3-yl)methyl)-2-phenyl-1H-indole

To 2-(1-((5-chlorobenzo[b]thiophen-3-yl)methyl)-2-phenyl-1H-indol-3-yl)acetonitrile (150 mg, 0.363 mmol) was added TBAF-3H₂O (57.3 mg, 0.182 mmol) and TMSN₃ (0.146 mL, 1.09 mmol). Toluene (0.1 ml) was added and the reaction was heated at 120 C for 15 h. After cooling, 1N HCl (aq) was added. The solids were filtered off and triturated with DCM. Desired 3-((1H-tetrazol-5-yl)methyl)-1-((5-chlorobenzo[b]thiophen-3-yl)methyl)-2-phenyl-1H-indole (124 mg, 74.9% yield) product was obtained as a beige solid. MS APCI (−) m/z 454 detected. ¹H NMR (400 mHz. d₆-DMSO) δ 7.99-6.97 (m, 13H), 5.55 (s, 2H), 4.28 (s, 2H).

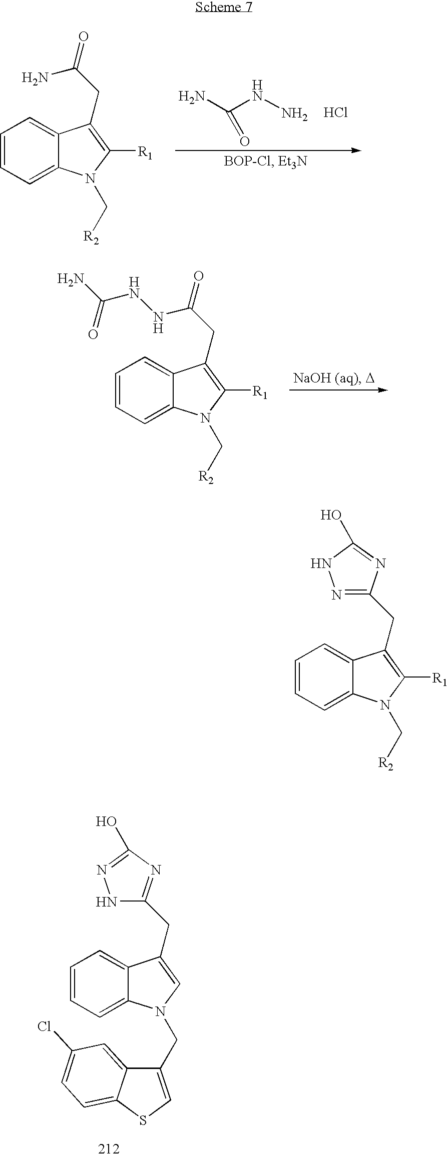

### Step 1: Preparation of 1-(2-(1-((5-chlorobenzo[b]thiophen-3-yl)methyl)-1H-indol-3-yl)acetyl)semicarbazide

To a solution of 2-(1-((5-chlorobenzo[b]thiophen-3-yl)methyl)-1H-indol-3-yl)acetic acid (165 mg, 0.464 mmol) in DCM (5 mL) was added BOP-Cl (118 mg, 0.464 mmol) and the reaction was stirred at room temperature for 5 mins. triethylamine (0.0776 mL, 0.556 mmol) was then added followed by semicarbazide hydrochloride (51.7 mg, 0.464 mmol). The reaction was stirred at room temperature overnight. The reaction was filtered and the filtrate was concentrated in vacuo. The residue was purified by PLC eluting with 9:1 DCM/MeOH. 1-(2-(1-((5-chlorobenzo[b]thiophen-3-yl)methyl)-1H-indol-3-yl)acetyl)semicarbazide (130 mg, 67.9% yield) was isolated as a pale beige solid. MS APCI (−) m/z 411 detected. ¹H NMR (400 mHz. CD₃OD) δ 7.86-7.08 (m, 9H), 5.57 (s, 2H), 3.71 (s, 2H).

### Step 2: Preparation of 5-((1-((5-chlorobenzo[b]thiophen-3-yl)methyl)-1H-indol-3-yl)methyl)-4H-1,2,4-triazol-3-ol

A solution of 1-(2-(1-((5-chlorobenzo[b]thiophen-3-yl)methyl)-1H-indol-3-yl)acetyl)semicarbazide (125 mg, 0.303 mmol) in 1N NaOH (aq) (10 mL) was heated under reflux for 3 h. 1N HCl (aq) was added to acidify the reaction mixture (pH 1) and the crude product was extracted with DCM (2×40 mL). The combined organics were washed with brine, dried (MgSO4) and the solvent was removed in vacuo. The beige solid was triturated with ether and the solids were filtered off to give 5-((1-((5-chlorobenzo[b]thiophen-3-yl)methyl)-1H-indol-3-yl)methyl)-4H-1,2,4-triazol-3-ol (23 mg, 19.2% yield) as a pale beige solid. MS APCI (−) m/z 393 detected. ¹H NMR (400 mHz. CD₃OD) δ 11.2 (s, 1H), 11.12 (s, 1H), 8.04-7.03 (m, 9H), 5.64 (s, 2H), 3.83 (s, 2H).

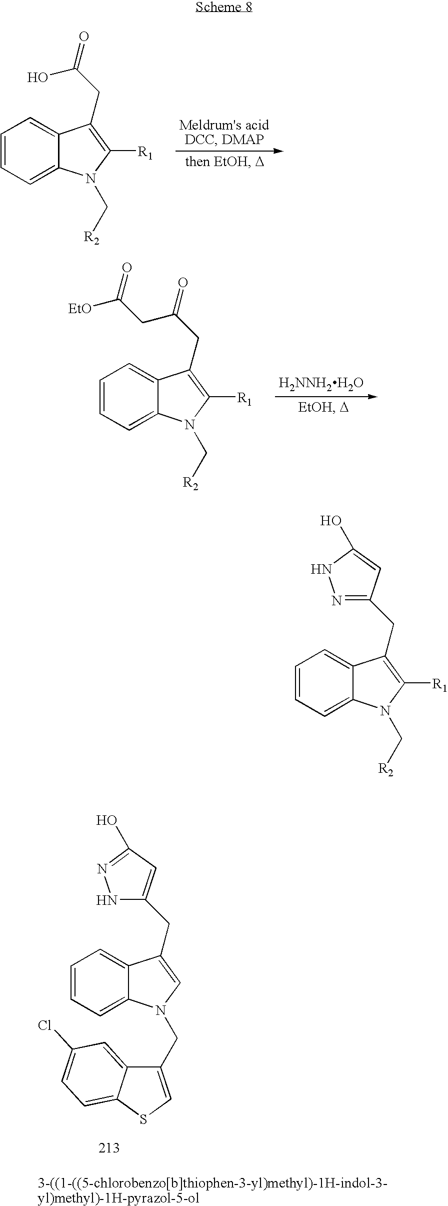

To a solution of 2-(1-((5-chlorobenzo[b]thiophen-3-yl)methyl)-1H-indol-3-yl)acetic acid (329 mg, 0.925 mmol) in DCM (10 mL) was added DCC (191 mg, 0.925 mmol) and DMAP (5.65 mg, 0.0462 mmol) and the reaction was stirred at room temperature for 5 mins. 2,2-dimethyl-1,3-dioxane-4,6-dione (133 mg, 0.925 mmol) was added and the reaction was stirred at room temperature for 15 h. The white precipitate (DCU) was filtered off and the filtrate was concentrated in vacuo. To this crude ethyl 4-(1-((5-chlorobenzo[b]thiophen-3-yl)methyl)-1H-indol-3-yl)-3-oxobutanoate (200 mg, 0.4696 mmol) in EtOH (10 mL) was added hydrazine monohydrate (0.02278 ml, 0.4696 mmol) and the reaction was heated at reflux for 6 h. The reaction was concentrated in vacuo. The residue was purified by PLC eluting with 9:1 DCM/MeOH. The band at R_{f} 0.3 gave 3-((1-((5-chlorobenzo[b]thiophen-3-yl)methyl)-1H-indol-3-yl)methyl)-1H-pyrazol-5-ol (4 mg, 2.163% yield) as a beige solid. MS APCI (−) m/z 392 detected. ¹H NMR (400 mHz. CD₃OD) δ 7.86-7.02 (m, 10H), 5.55 (s, 2H), 4.00 (s, 2H).

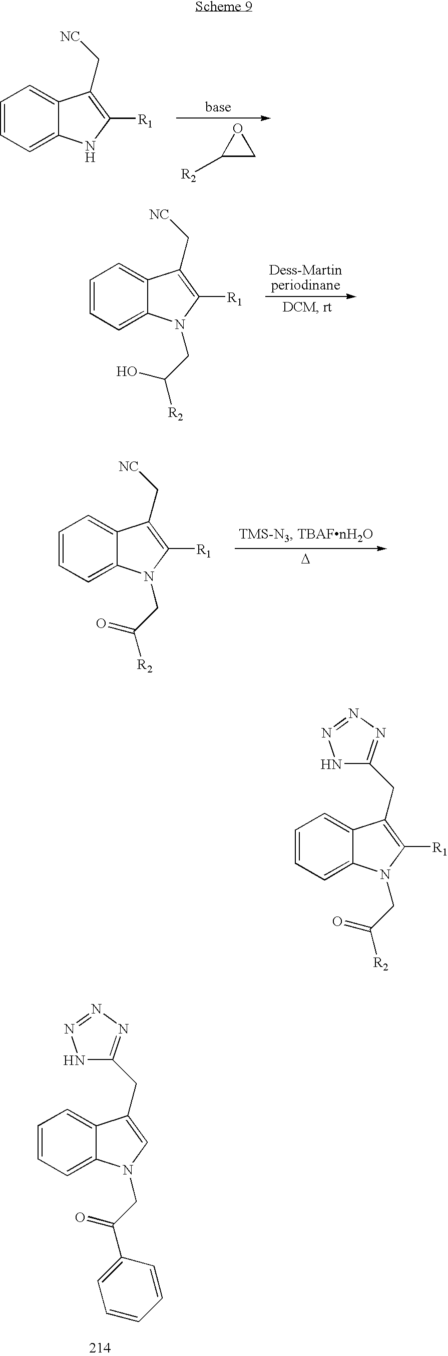

### Step 1: Preparation of 2-(1-(2-hydroxy-2-phenylethyl)-1H-indol-3-yl)acetonitrile

To a solution of 2-(1H-indol-3-yl)acetonitrile (2.00 g, 12.81 mmol) in DMF (20 mL) at 0° C. was added NaHMDS (12.81 ml, 12.81 mmol). The reaction was stirred at 0° C. for 5 mins and then 2-phenyloxirane (1.460 ml, 12.81 mmol) was added in one portion. The reaction continued to stir for 15 h at rt. Saturated NaHCO₃ (aq) was added and the organics were extracted with EtOAc (50 mL). The organic layer was washed with brine, dried (MgSO₄) and the solvents were removed in vacuo. The residue was purified by silica gel plug eluting with DCM (Rf 0.18) to give product as a yellow oil which crystallized on standing. 2-(1-(2-hydroxy-2-phenylethyl)-1H-indol-3-yl)acetonitrile (1.46 g, 41.26% yield) was isolated as a yellow crystalline solid. MS APCI (+) m/z 259 (—H₂O) detected. ¹H NMR (400 mHz. CD₃OD) δ 7.56-7.06 (m, 10H), 4.98 (m, 1H), 4.29 (m, 2H), 3.87 (s, 2H).

### Step 2: Preparation of 2-(3-((1H-tetrazol-5-yl)methyl)-1H-indol-1-yl)-1-phenylethanol

To 2-(1-(2-hydroxy-2-phenylethyl)-1H-indol-3-yl)acetonitrile (200 mg, 0.7238 mmol) was added azidotrimethylsilane (0.2909 mL, 2.171 mmol) and N,N,N-tributyl-N-fluorobutan-1-amine trihydrate (114.2 mg, 0.3619 mmol). The reaction was stirred at 120 C for 15 h in a sealed tube. Reaction mixture was concentrated in vacuo and the residue was purified by silica gel plug eluting with 9:1 DCM/MeOH Rf 0.3. (Product contained TBAF). The residue was dissolved in 1N NaOH (aq), washed with ether and the aqueous phase was neutralized with 1N HCl (aq). The product was extracted with ether (20 mL). The organic layer was washed with brine, dried (MgSO₄) and the solvent was removed in vacuo. The product was then crystallized from MeOH. 2-(3-((1H-tetrazol-5-yl)methyl)-1H-indol-1-yl)-1-phenylethanol (61 mg, 26.39% yield) was isolated as a pale yellow crystalline solid. MS APCI (−) m/z 318 detected. ¹H NMR (400 mHz. CD₃OD) δ 7.39-6.99 (m, 10H), 5.01 (m, 1H), 4.39 (s, 2H), 4.31 (m, 2H).

### Step 3: Preparation of 2-(3-((1H-tetrazol-5-yl)methyl)-1H-indol-1-yl)-1-phenylethanone

To a solution of 2-(3-((1H-tetrazol-5-yl)methyl)-1H-indol-1-yl)-1-phenylethanol (20 mg, 0.063 mmol) in DCM (5 mL) was added Dess-Martin periodinane (29 mg, 0.069 mmol) and the reaction was stirred at room temperature for 2 h. The reaction was diluted with DCM (20 mL) and washed with satd. NaHCO₃ (aq). The organic layer was washed with brine, dried (MgSO₄) and the solvent was removed in vacuo. The residue was purified by PLC eluting with 9:1 DCM/MeOH (Rf 0.4) to give 2-(3-((1H-tetrazol-5-yl)methyl)-1H-indol-1-yl)-1-phenylethanone (12 mg, 60% yield) as a white solid. MS APCI (−) m/z 316 detected. ¹H NMR (400 mHz. CD₃OD) δ 8.13-7.03 (m, 10H), 5.79 (s, 2H), 4.46 (s, 2H).

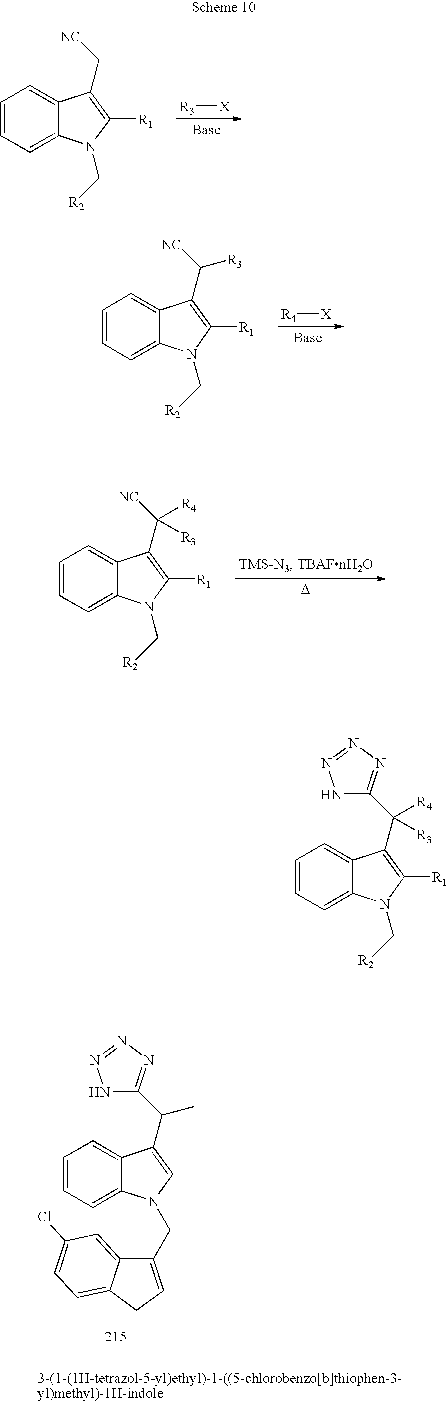

To a solution of 2-(1-((5-chlorobenzo[b]thiophen-3-yl)methyl)-1H-indol-3-yl)acetonitrile (125 mg, 0.371 mmol) in THF (3 mL) at −78° C. was added a 1.0 M solution of NaHMDS (0.371 mL, 0.371 mmol) in THF and the reaction was stirred at −78° C. for 10 mins. Iodomethane (0.0231 mLl, 0.371 mmol) was then added in one portion and the reaction was stirred at −78° C. for 10 mins then warmed to room temperature over 15 h. EtOAc (30 mL) was added and the reaction was quenched with 1N HCl (aq). The organic layer was washed with brine, dried (MgSO₄) and the solvent was removed in vacuo. The brown residue was purified by PLC eluting with 1:1 hexanes/DCM. The band at Rf 0.6 contained a mixture of 2-(1-((5-chlorobenzo[b]thiophen-3-yl)methyl)-1H-indol-3-yl)propanenitrile and 2-(1-((5-chlorobenzo[b]thiophen-3-yl)methyl)-1H-indol-3-yl)-2-methylpropanenitrile. To this mixture containing 2-(1-((5-chlorobenzo[b]thiophen-3-yl)methyl)-1H-indol-3-yl)propanenitrile (90 mg, 0.257 mmol) was added TMSN₃ (0.206 ml, 1.54 mmol) and TBAF.3H₂O (40.5 mg, 0.128 mmol). The reaction was stirred at 120 C for 15 h. Reaction mixture was concentrated in vacuo and the residue was purified by silica gel plug eluting with 2:1 DCM/EtOAc Rf 0.15. The residue was then purified by PLC eluting with 9:1 DCM/MeOH. 3-(1-(1H-tetrazol-5-yl)ethyl)-1-((5-chlorobenzo[b]thiophen-3-yl)methyl)-1H-indole (4 mg, 3.96% yield) was isolated as an off-white solid from the band at Rf 0.5 after crystallization of the residue from MeOH. MS APCI (−) m/z 392 detected. ¹H NMR (400 mHz. CD₃OD) δ 7.87-6.99 (m, 9H), 5.59 (s, 2H), 4.81 (m, 1H), 1.84 (d, J=7.0 Hz, 2H).

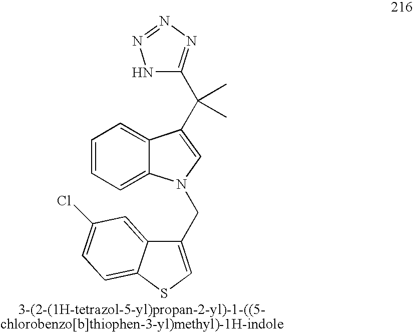

To a solution of 2-(1-((5-chlorobenzo[b]thiophen-3-yl)methyl)-1H-indol-3-yl)acetonitrile (125 mg, 0.371 mmol) in THF (3 mL) at −78 C was added a 1.0 M solution of NaHMDS (0.371 mL, 0.371 mmol) in THF and the reaction was stirred at −78° C. for 10 mins. Iodomethane (0.0231 mL, 0.371 mmol) was then added in one portion and the reaction was stirred at −78° C. for 10 mins then warmed to room temperature over 15 h. EtOAc (30 mL) was added and the reaction was quenched with 1N HCl (aq). The organic layer was washed with brine, dried (MgSO₄) and the solvent was removed in vacuo. The brown residue was purified by PLC eluting with 1:1 hexanes/DCM. The band at Rf 0.6 contained a mixture of 2-(1-((5-chlorobenzo[b]thiophen-3-yl)methyl)-1H-indol-3-yl)propanenitrile and 2-(1-((5-chlorobenzo[b]thiophen-3-yl)methyl)-1H-indol-3-yl)-2-methylpropanenitrile. To this mixture containing 2-(1-((5-chlorobenzo[b]thiophen-3-yl)methyl)-1H-indol-3-yl)-2-methylpropanenitrile (90 mg, 0.257 mmol) was added TMSN₃ (0.206 ml, 1.54 mmol) and TBAF-3H₂O (40.5 mg, 0.128 mmol). The reaction was stirred at 120° C. for 15 h. Reaction mixture was concentrated in vacuo and the residue was purified by silica gel plug eluting with 2:1 DCM/EtOAc Rf 0.15. The residue was then purified by PLC eluting with 9:1 DCM/MeOH. 3-(2-(1H-tetrazol-5-yl)propan-2-yl)-1-((5-chlorobenzo[b]thiophen-3-yl)methyl)-1H-indole (3 mg, 2.87% yield) was also isolated by PLC as a white solid from band at Rf 0.4 (NMR-3, MS-3). MS APCI (−) m/z 406 detected. ¹H NMR (400 mHz. CD₃OD) δ 7.88-6.91 (m, 9H), 5.62 (s, 2H), 1.91 (s, 6H).

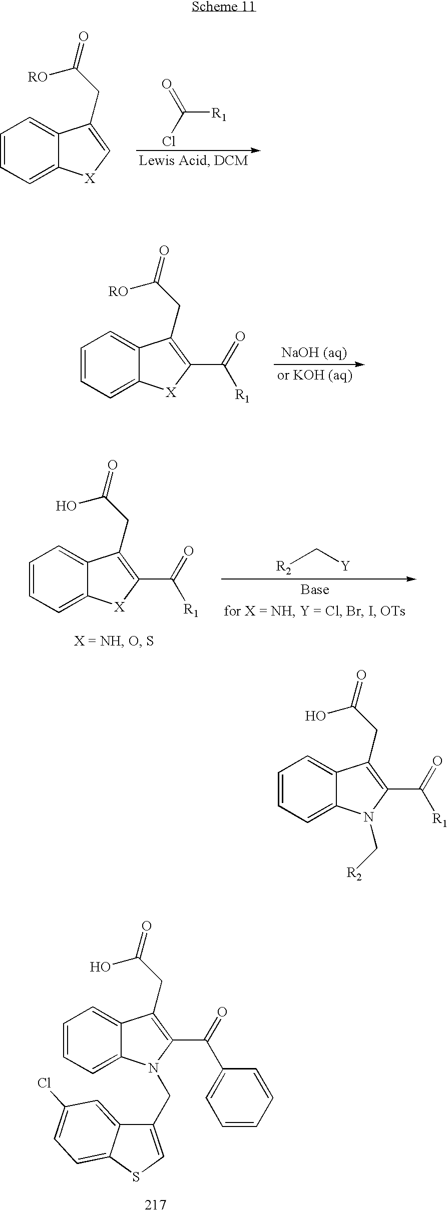

### Step 1: Preparation of ethyl 2-(2-benzoyl-1H-indol-3-yl)acetate

To a solution of ethyl 2-(1H-indol-3-yl)acetate (1.00 g, 4.920 mmol) in 1:1 v/v THF/DMF (6 mL) at 0° C. was added ZnCl₂ (2.012 g, 14.76 mmol) and benzoyl chloride (0.5711 ml, 4.920 mmol). The reaction was stirred at room temperature for 14 h. The reaction was diluted with DCM (20 mL) and the mixture was washed with 1N HCl (aq). The organic layer was washed with satd. NaHCO₃ (aq), brine, dried (MgSO₄) and the solvent was removed in vacuo. The product crystallized on concentration. The crystalline solid was triturated with ether (20 mL) and the beige solid was filtered off ethyl 2-(2-benzoyl-1H-indol-3-yl)acetate (840 mg, 55.55% yield) was obtained as a beige solid. MS APCI (+) m/z 308 detected. ¹H NMR (400 mHz. CDCl₃) δ 8.86 (brs, 1H), 7.82-7.16 (m, 9H), 4.12 (m, 2H), 3.83 (s, 2H), 1.22 (m, 3H).

### Step 2: Preparation of ethyl 2-(2-benzoyl-1H-indol-3-yl)acetic acid

To a solution of ethyl 2-(2-benzoyl-1H-indol-3-yl)acetate (797 mg, 2.59 mmol) in THF/MeOH (1:1 v/v) (10 mL) was added 2N KOH (8 mL) and the reaction was stirred at room temperature for 4 h. Ether (20 mL) was added and the layers were separated. The aqueous phase was acidified to pH1 with c. HCl. The crude product was extracted with ether (20 mL) and the extraction was washed with brine, dried (MgSO₄) and the solvents were removed in vacuo. The residue was triturated with ether and the pale yellow solids were filtered off and dried. 2-(2-benzoyl-1H-indol-3-yl)acetic acid (590 mg, 81.5% yield) was obtained as a pale yellow solid. MS APCI (−) m/z 278 detected. ¹H NMR (400 mHz. d₆-DMSO) δ 12.13 (brs, 1H), 11.56 (brs, 1H), 7.71-7.05 (m, 9H), 3.75 (s, 2H).

### Step 3: Preparation of 2-(2-benzoyl-1-((5-chlorobenzo[b]thiophen-3-yl)methyl)-1H-indol-3-yl)acetic acid

To a solution of 2-(2-benzoyl-1H-indol-3-yl)acetic acid (100 mg, 0.3581 mmol) in DMF (4 mL) at −78° C. was added a 1.0M solution of NaHMDS in THF (0.7161 ml, 0.7161 mmol). The reaction was stirred at −78° C. for 4 mins then 3-(bromomethyl)-5-chlorobenzo[b]thiophene (93.65 mg, 0.3581 mmol) was added. The reaction was warmed to room temperature over 15 h. 1N HCl (aq) (10 mL) was added and the crude product was extracted with ether (20 ml). The ether layer was washed with brine and dried (MgSO₄). The solvent was removed in vacuo. The residue was purified by PLC eluting with 9:1 DCM/MeOH. Rf 0.45. The band at Rf 0.45 gave 2-(2-benzoyl-1-((5-chlorobenzo[b]thiophen-3-yl)methyl)-1H-indol-3-yl)acetic acid (6 mg, 3.643% yield) as a pale yellow/green solid. MS APCI (−) m/z 459 detected. ¹H NMR (400 mHz. CDCl₃) δ 7.80-6.68 (m, 13H), 5.53 (s, 2H), 3.77 (s, 2H).

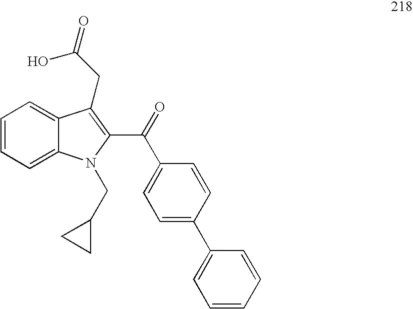

### Step 1: Preparation of ethyl 2-(2-(biphenylcarbonyl)-1H-indol-3-yl)acetate

To a solution of ethyl 2-(1H-indol-3-yl)acetate (2.00 g, 9.841 mmol) in 1:1 V/V THF/DMF (6 mL) at 0 C was added ZnCl₂ (4.024 g, 29.52 mmol) and biphenyl-4-carbonyl chloride (2.132 g, 9.841 mmol). The reaction was stirred at room temperature for 14 h. The reaction was diluted with DCM (20 mL) and the mixture was filtered through a glass sinter funnel. The residue was washed with DCM (50 mL). The solid residue was washed with 1N HCl (aq) with sonication to break up lumps. The beige solid was triturated from cold MeCN. The filtrate containing product began to solidify. This solid was crystallized from hot acetonitrile (15 mL). Two crops were obtained to give ethyl 2-(2-(biphenylcarbonyl)-1H-indol-3-yl)acetate (1.57 g, 41.61% yield) as a pale yellow solid. MS APCI (+) m/z 384 detected. ¹H NMR (400 mHz. CDCl₃) δ 8.91 (brs, 1H), 7.92-7.19 (m, 13H), 4.12 (m, 2H), 3.90 (s, 2H), 1.21 (m, 3H).

### Step 2: Preparation of 2-(2-(biphenylcarbonyl)-1H-indol-3-yl)acetic acid

To a solution of ethyl 2-(2-(biphenylcarbonyl)-1H-indol-3-yl)acetate (1.50 g, 3.912 mmol) in THF/MeOH (1:1 v/v) (10 mL) was added 2N KOH (10 mL) and the reaction was stirred at room temperature for 4 h. Ether (20 mL) was added and the layers were separated. The aqueous phase was acidifed to pH 1 with conc. HCl. The crude product was extracted with ether (20 mL) and the extraction was washed with brine, dried (MgSO₄) and the solvents were removed in vacuo. The residue was triturated with ether and the pale yellow solids were filtered off and dried. 2-(2-(biphenylcarbonyl)-1H-indol-3-yl)acetic acid (1.105 g, 79.48% yield) was obtained as a pale yellow solid. MS APCI (−) m/z 354 detected. ¹H NMR (400 mHz. d₆-DMSO) δ 12.21 (brs, 1H), 11.66 (s, 1H), 7.88-7.12 (m, 13H), 3.88 (s, 2H).

### Step 3: Preparation of 2-(2-(biphenylcarbonyl)-1-(cyclopropylmethyl)-1H-indol-3-yl)acetic acid

To a solution of 2-(2-(biphenylcarbonyl)-1H-indol-3-yl)acetic acid (150 mg, 0.4221 mmol) in DMF (4 ml) at −78° C. was added a 1.0M solution of NaHMDS in THF (0.8442 mLl, 0.8442 mmol). The reaction was stirred at −78° C. for 4 mins then (bromomethyl)cyclopropane (0.04093 ml, 0.4221 mmol) was added. The reaction was warmed to room temperature over 15 h. 1N HCl (aq) (10 mL) was added and the crude product was extracted with ether (20 ml). The ether layer was washed with brine and dried (MgSO₄). The solvent was removed in vacuo and the residue was purified by PLC eluting with 9:1 DCM/MeOH. The band at Rf 0.5 contained product plus an impurity. This yellow gum residue was purified by crystallization from acetonitrile (3 mL). 2-(2-(biphenylcarbonyl)-1-(cyclopropylmethyl)-1H-indol-3-yl)acetic acid (35 mg, 20.25% yield) was isolated as a pale yellow crystalline solid. MS APCI (−) m/z 409 detected. ¹H NMR (400 mHz. d₆-DMSO) δ 12.2 (brs, 1H), 7.87-7.15 (m, 13H), 4.25 (d, J=6.6 Hz, 2H), 3.52 (s, 2H), 1.09 (m, 1H), 0.38 (m, 2H), 0.20 (m, 2H).

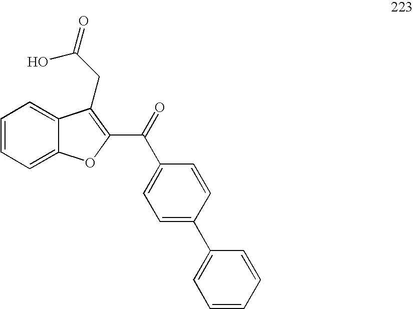

### Step 1: Preparation of ethyl 2-(2-(biphenylcarbonyl)benzofuran-3-yl)acetate

To a solution of ethyl 2-(benzofuran-3-yl)acetate (200 mg, 0.9793 mmol) in DCM (6 mL) at room temperature was added biphenyl-4-carbonyl chloride (212.2 mg, 0.9793 mmol) and SnCl₄ (0.3438 ml, 2.938 mmol). The reaction was stirred at room temperature for 15 h. The reaction was diluted with DCM (20 mL) and washed with 1N HCl (aq) (20 ml), 1N NaOH (aq) and brine. After drying (MgSO₄), the solvent was removed in vacuo. Material was crystallized from ether to give ethyl 2-(2-(biphenylcarbonyl)benzofuran-3-yl)acetate (186 mg, 49.41% yield) as a white solid.

### Step 2: Preparation of 2-(2-(biphenylcarbonyl)benzofuran-3-yl)acetic acid

To a solution of ethyl 2-(2-(biphenylcarbonyl)benzofuran-3-yl)acetate (150 mg, 0.390 mmol) in 1:1 v/v THF/MeOH (8 mL) was added 15% aq KOH solution (0.5 mL) and water (0.5 mL) and the reaction was stirred at room temperature for 1 h. Ether (20 mL) and water (20 mL) were added and the layers were separated. The aqueous phase was acidified to pH1 with conc. HCl and the white precipitate that formed was filtered off and dried. 2-(2-(biphenylcarbonyl)benzofuran-3-yl)acetic acid (127 mg, 91.3% yield) was obtained as a white solid. MS APCI (−) m/z 356 detected. ¹H NMR (400 mHz. d₆-DMSO) δ 12.55 (brs, 1H), 8.17-7.43 (m, 13H), 4.17 (s, 2H).

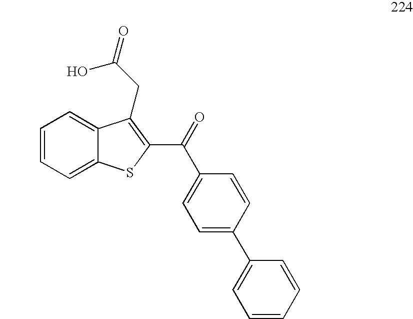

### Step 1: Preparation of methyl 2-(2-(biphenylcarbonyl)benzo[b]thiophen-3-yl)acetate

To a solution of methyl 2-(benzo[b]thiophen-3-yl)acetate (1.000 g, 4.848 mmol) in DCM (6 mL) at room temperature was added biphenyl-4-carbonyl chloride (1.050 g, 4.848 mmol) and SnCl₄ (1.702 ml, 14.54 mmol). The reaction was stirred at room temperature for 15 h. The reaction was diluted with DCM (20 mL) and washed with 1N HCl (aq) (20 mL), 1N NaOH (aq) and brine. After drying (MgSO₄), the solvent was removed in vacuo. The material was purified by silica gel plug eluting with DCM, Rf 0.2. Material was crystallized from MeOH to give methyl 2-(2-(biphenylcarbonyl)benzo[b]thiophen-3-yl)acetate (395 mg, 21.08% yield) as a pale beige crystalline solid.

### Step 2: Preparation of 2-(2-(biphenylcarbonyl)benzo[b]thiophen-3-yl)acetic acid

To a solution of methyl 2-(2-(biphenylcarbonyl)benzo[b]thiophen-3-yl)acetate (380 mg, 0.983 mmol) in THF/MeOH (1:1 v/v) (10 mL) was added 15% w/v KOH (0.5 mL) and the reaction was stirred at room temperature for 4 h. Water (15 mL) was added and ether (20 mL) and the layers were separated. The aqueous phase was acidifed to pH1 with c. HCl. The product precipitated out and was filtered off, rinsed with water and dried. 2-(2-(biphenylcarbonyl)benzo[b]thiophen-3-yl)acetic acid (260 mg, 71.0% yield) was obtained as a white solid. MS APCI (−) m/z 372 detected. ¹H NMR (400 mHz. d₆-DMSO) δ 12.47 (brs, 1H), 8.09-7.46 (m, 13H), 4.13 (s, 2H).

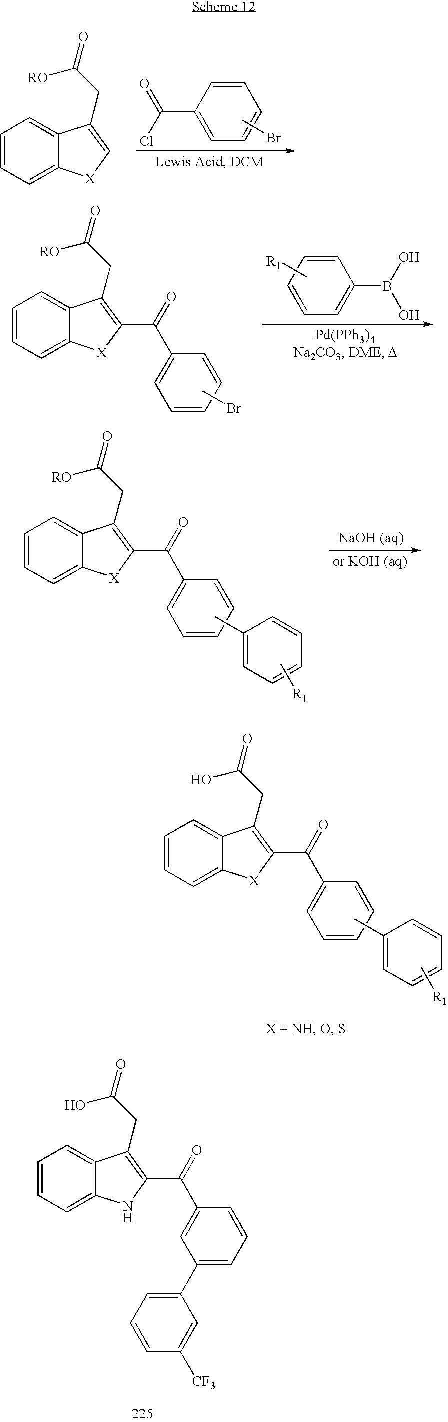

### Step 1: Preparation of ethyl 2-(2-(3-bromobenzoyl)-1H-indol-3-yl)acetate

To a solution of ethyl 2-(1H-indol-3-yl)acetate (18.600 g, 91.519 mmol) in DCM (180 mL) at room temperature was added 3 A Mol sieves, 3-bromobenzoyl chloride (20.085 g, 91.519 mmol) and ZnCl₂ (37.421 g, 274.56 mmol). The reaction was stirred at room temperature for 15 h. The reaction was diluted with DCM (150 mL) and washed with 1N HCl (aq) (200 mL), and brine. After drying (MgSO₄), the solvent was removed in vacuo. The brown oil was purified by silica gel plug eluting with DCM to give ethyl 2-(2-(3-bromobenzoyl)-1H-indol-3-yl)acetate (21.15 g, 59.833% yield) as a yellow oil which crystallized to a beige solid on standing for several days. MS APCI (+) m/z 386/388 detected. ¹H NMR (400 mHz. d₆-DMSO) δ 11.74 (s, 1H), 7.87-7.12 (m, 8H), 4.03 (m, 2H), 3.89 (s, 2H), 1.13 (m, 3H).

### Step 2: Preparation of ethyl 2-(2-(3′-(trifluoromethyl)biphenylcarbonyl)-1H-indol-3-yl)acetate

To ethyl 2-(2-(3-bromobenzoyl)-1H-indol-3-yl)acetate (200 mg, 0.518 mmol), Na₂CO₃ (165 mg, 1.55 mmol), and 3-(trifluoromethyl)phenylboronic acid (148 mg, 0.777 mmol) was added DME (6 mL), water (1 mL) and the mixture was degassed by bubbling nitrogen through for 5 mins. Pd(PPh₃)₄ (29.9 mg, 0.0259 mmol) was then added and the sealed tube was heated at 90° C. (external temp) for 14 h. DCM (20 mL) was added and the mixture was washed with satd. NaHCO₃ (aq). The layers were separated and the organic phase was dried (MgSO₄). The solvents were removed in vacuo. The crude residue was purified by PLC eluting with DCM. The band at Rf 0.4 gave ethyl 2-(2-(3′-(trifluoromethyl)biphenylcarbonyl)-1H-indol-3-yl)acetate (201 mg, 86.0% yield) as a yellow oil. MS APCI (−) m/z 450 detected.

### Step 3: Preparation of 2-(2-(3′-(trifluoromethyl)biphenylcarbonyl)-1H-indol-3-yl)acetic acid

To a solution of ethyl 2-(2-(3′-(trifluoromethyl)biphenylcarbonyl)-1H-indol-3-yl)acetate (201 mg, 0.445 mmol) in THF/MeOH (1:1 v/v) (10 mL) was added 20% w/v KOH (0.5 mL) and water (1 mL) and the reaction was stirred at room temperature for 4 h. Ether (20 mL) was added and the layers were separated. The aqueous phase was acidifed to pH 1 with conc. HCl. The product crystallized out and was filtered off and dried. 2-(2-(3′-(trifluoromethyl)biphenylcarbonyl)-1H-indol-3-yl)acetic acid (160 mg, 84.9% yield) was obtained as a yellow crystalline solid. MS APCI (−) m/z 422 detected. ¹H NMR (400 mHz. d₆-DMSO) δ 12.18 (s, 1H), 11.7 (s, 1H), 8.06-7.11 (m, 12H), 3.85 (s, 2H).

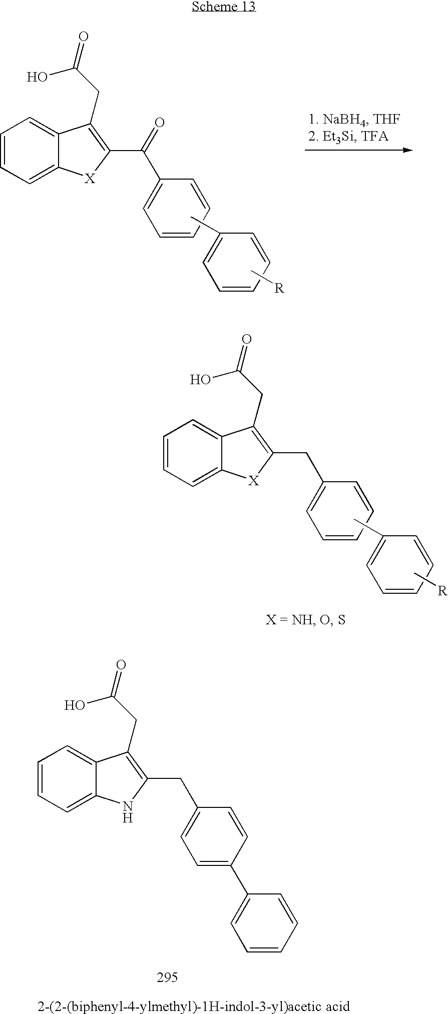

To a solution of 2-(2-(biphenylcarbonyl)-1H-indol-3-yl)acetic acid (100 mg, 0.281 mmol) in THF (4 mL) was added NaBH₄ (10.6 mg, 0.281 mmol). The reaction was stirred at room temperature for 3 h. The reaction was diluted with ether (20 mL) and 1N HCl (aq) was added. The layers were separated and the organic phase was washed with brine. After drying (MgSO₄), the solvents were removed in vacuo. To a suspension of this material in triethylsilane ml, 2.7992 mmol) was added TFA (3 mL). The reaction was stirred at room temperature for 15 h. The reaction mixture was concentrated in vacuo and the excess TFA was removed by azeotropic distillation with toluene (twice). The residue was triturated with ether and the solids were filtered off. The filtrate was concentrated in vacuo and purified by PLC eluting with 9:1 DCM/MeOH to give 2-(2-(biphenyl-4-ylmethyl)-1H-indol-3-yl)acetic acid (6 mg, 6.2785% yield) as a yellow gum. MS APCI (−) m/z 340 detected. ¹H NMR (400 mHz. CDCl₃) δ 7.73-7.12 (m, 13H), 4.16 (s, 2H), 3.79 (s, 2H).

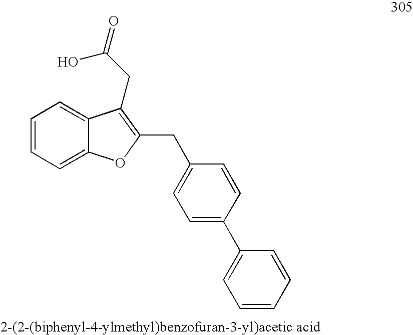

To a solution of 2-(2-(biphenylcarbonyl)benzofuran-3-yl)acetic acid (75 mg, 0.21 mmol) in THF (4 mL) was added NaBH₄ (8.0 mg, 0.21 mmol). The reaction was stirred at room temperature for 2 h. The reaction was diluted with DCM (20 mL) and 1N HCl (aq) was added. The layers were separated and the organic phase was washed with brine. After drying (MgSO₄), the solvents were removed in vacuo. To a solution of this material (72 mg, 0.2115 mmol) was added triethylsilane (0.3379 mL, 2.115 mmol) and TFA (1 mL). The reaction was stirred at room temperature for 3 h. The reaction mixture was concentrated in vacuo and the excess TFA was removed by azeotropic distillation with toluene (twice). The residue was purified by PLC eluting with 9:1 DCM/MeOH to give 2-(2-(biphenyl-4-ylmethyl)benzofuran-3-yl)acetic acid (48 mg, 66.27% yield) as a white crystalline solid. MS APCI (−) m/z 341 detected. ¹H NMR (400 mHz. CDCl₃) δ 7.56-7.19 (m, 13H), 4.16 (s, 2H), 3.69 (s, 2H).

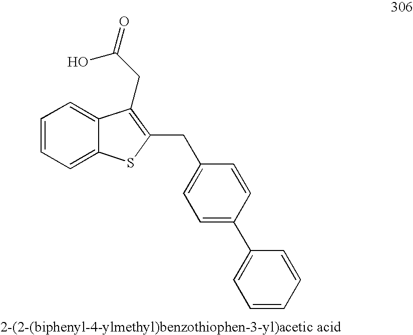

To a solution of 2-(2-(biphenylcarbonyl)benzo[b]thiophen-3-yl)acetic acid (75 mg, 0.20 mmol) in THF (4 mL) was added NaBH₄ (7.6 mg, 0.20 mmol). The reaction was stirred at room temperature for 2 h. The reaction was diluted with DCM (20 mL) and 1N HCl (aq) was added. The layers were separated and the organic phase was washed with brine. After drying (MgSO₄), the solvents were removed in vacuo. To a solution of this material (72 mg, 0.2020 mmol) was added triethylsilane (0.3226 mL, 2.020 mmol) and TFA (1 mL). The reaction was stirred at room temperature for 3 h. The reaction mixture was concentrated in vacuo and the excess TFA was removed by azeotropic distillation with toluene (twice). The residue was crystallized from toluene to give 2-(2-(biphenyl-4-ylmethyl)benzothiophen-3-yl)acetic acid (27 mg, 37.29% yield) as a white crystalline solid. MS APCI (−) m/z 357 detected. ¹H NMR (400 mHz. d₆-DMSO) δ 12.42 (brs, 1H), 7.78-7.27 (m, 13H), 4.25 (s, 2H), 3.88 (s, 2H).

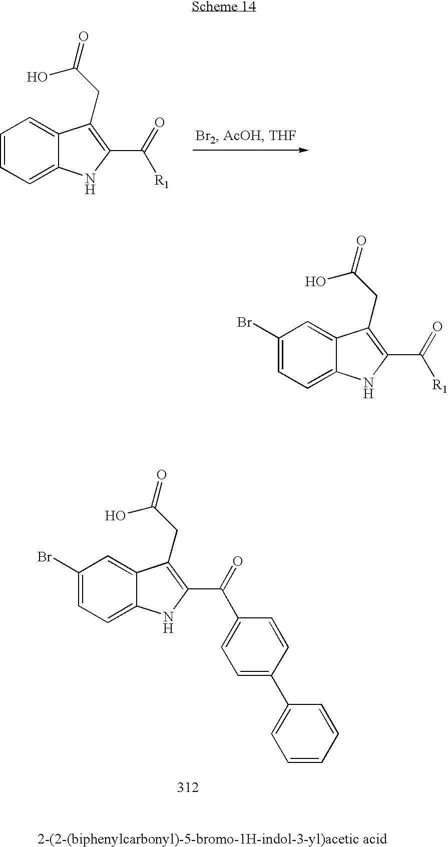

To a solution of 2-(2-(biphenylcarbonyl)-1H-indol-3-yl)acetic acid (50 mg, 0.141 mmol) in AcOH (4 mL) was added bromine (0.00721 mL, 0.141 mmol). The reaction was capped and stirred at room temperature for 14 h. Water (15 mL) was added and the crude product was extracted with ether (20 mL). The ether layer was washed with brine, dried (MgSO₄) and the solvents were removed in vacuo. The crude product was triturated with MeOH to remove minor impurities. 2-(2-(biphenylcarbonyl)-5-bromo-1H-indol-3-yl)acetic acid (45 mg, 73.6% yield) was obtained as a beige solid. MS APCI (−) m/z 432/434 detected. ¹H NMR (400 mHz. CD₃OD) δ 7.91-7.40 (m, 12H), 3.93 (s, 2H).

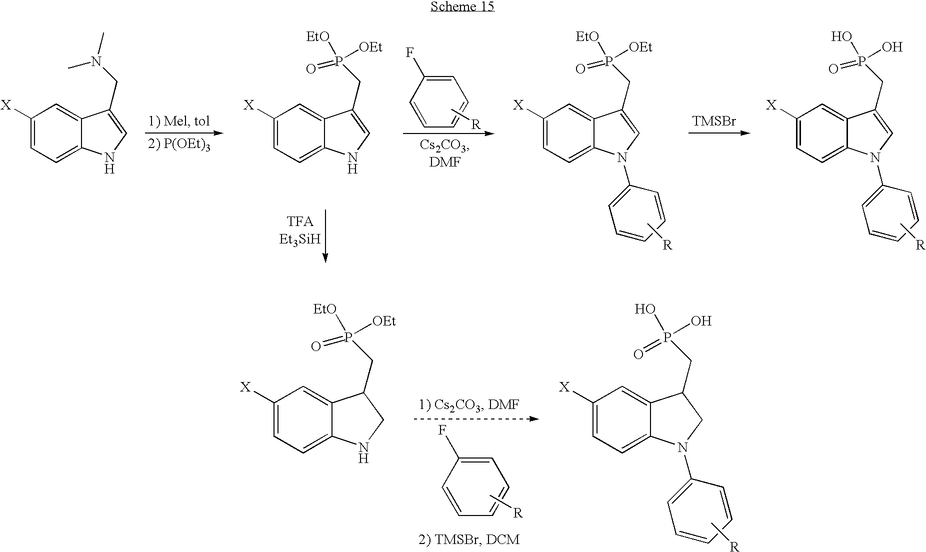

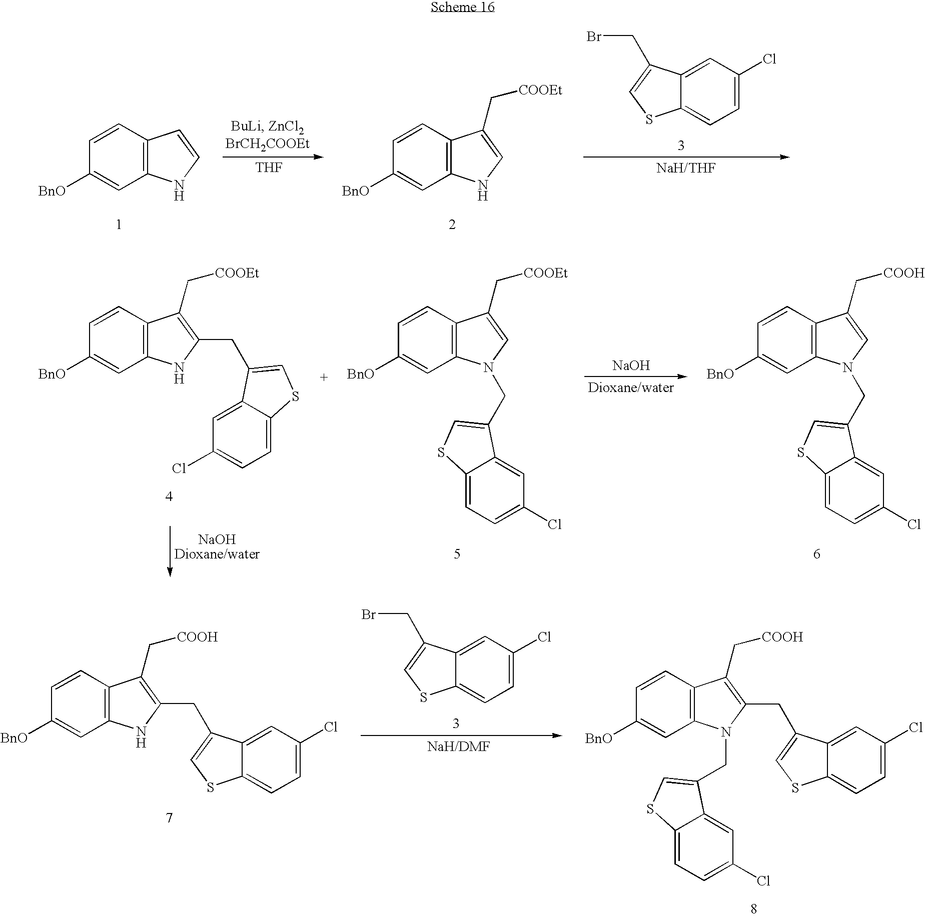

### Synthesis of Compound 2

Butyllithium (1.6 M in hexane, 15.4 mL, 24.63 mmol) was added to a stirred solution of 6-benzyloxyindole (1, 5.0 g, 22.39 mmol) in THF (40 mL) at 0° C. under argon. The resulting mixture was stirred at 0° C. for 30 min. Zinc chloride (1.0 M in ether, 24.6 mL) was added. The resulting mixture was warmed up to room temperature and stirred for 2 h. Ethyl bromoacetate (3.0 mL, 26.87 mmol) was added dropwise and the resulting reaction mixture was stirred at room temperature for 2 days. The mixture was concentrated, diluted with ethyl acetate, washed with brine, dried (Na₂SO₄), and concentrated to dryness. Chromatography on silica gel with 3-4% ethyl acetate in hexanes gave 3.1 g of compound 2 as slightly brown solid.

### Synthesis of Compounds 4 and 5

A solution of compound 2 (309 mg, 1.0 mmol) in THF (2 mL) was added to a stirred mixture of sodium hydride (60% in mineral oil, 44 mg, 1.1 mmol) in THF (4 mL) at 0° C. under argon. The resulting mixture was stirred at 0° C. for 15 min. Compound 3 (288 mg, 1.0 mmol) in THF (1 mL) was added. The resulting mixture was stirred at 0° C. for 2 h and at room temperature overnight. The mixture was diluted with ethyl acetate, washed with brine two times, dried (Na₂SO₄), and concentrated to dryness. Chromatography on silica gel with 1-2% ethyl acetate in DCM-hexanes (1:1) gave 69 mg of compound 4 and 56 mg of compound 5, both as a white solid.

### Synthesis of Compound 6

A solution of compound 5 (50 mg) was dissolved in dioxane (3 mL) and water (1 mL). Sodium hydroxide (2.0 M, 1 mL) was added. The resulting mixture was stirred at room temperature for 2 h, acidified with 2N HCl to pH 3, and concentrated. The residue was partitioned in DCM and brine. The organic phase was dried (Na₂SO₄) and concentrated. Crystallization from DCM-hexanes gave 44 mg of compound 6 as a white solid.

### Synthesis of Compound 7

By a similar procedure as described for compound 6, compound 4 (62 mg) was hydrolyzed to give compound 7 (56 mg) as a gray solid.

### Synthesis of Compound 8

Sodium hydride (60%, 10 mg, 0.25 mmol) was added to a stirred solution of DMF-coevaporated compound 7 (48 mg, 1.0 mmol) in DMF (1 mL) at 0° C. under argon. The resulting mixture was stirred at room temperature for 10 min. Compound 3 (34 mg, 0.13 mmol) in DMF (0.3 mL) was added. The resulting mixture was stirred at 0° C. for 2 h. After usual work-up, the residue was chromatographed on silica gel with 2% triethylamine and 2-2.5% methanol in DCM to give 18 mg of compound 8 as an off-white solid.

### Synthesis of Compounds 9-15

By a similar procedure as described for compound 8, compounds 9-15 as shown below were prepared.

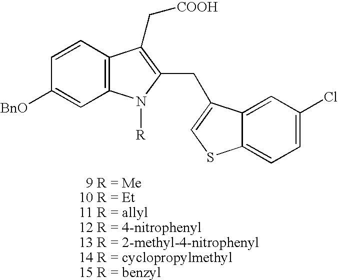

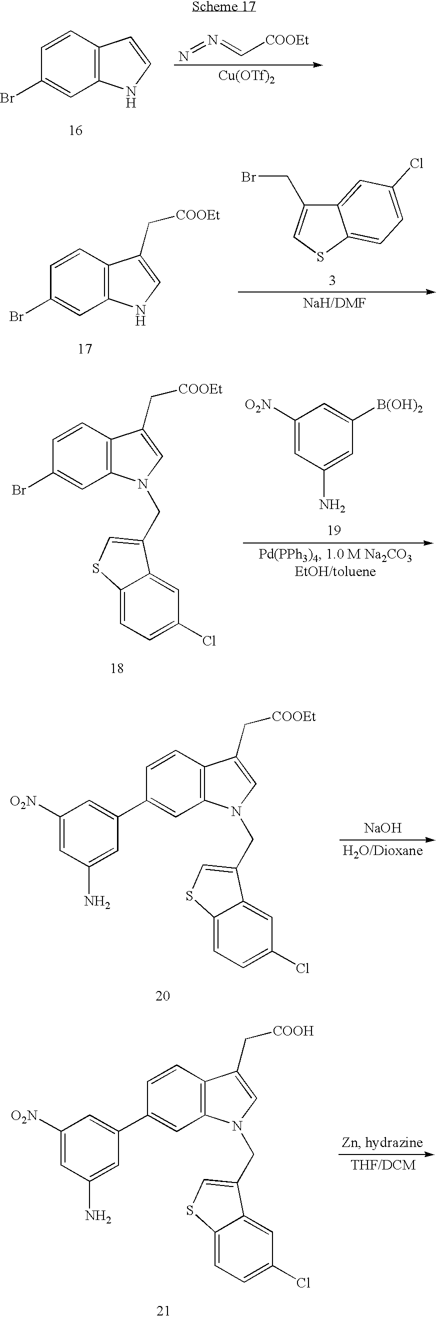

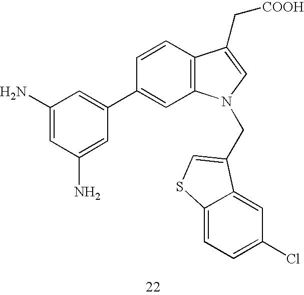

### Synthesis of Compound 17

Copper triflate (362 mg, 1.0 mmol) was added to a solution of 6-bromoindole (16, 3.92 g, 20 mmol) in DCM (100 mL) under argon, and the resulting suspension was cooled to 0° C. Ethyl diazoacetate (26 mmol, 2.7 mL) was added slowly and in portions during 30 min. The mixture was stirred at 0° C. to room temperature for 1 h, then at room temperature overnight, washed once with water, dried (Na₂SO₄), and concentrated. Flash chromatography on silica gel with DCM-hexane (4:1 to 5:1) gave a brownish residue. A second chromatography under the same condition gave 2.11 g of compound 17 as yellowish oil.

### Synthesis of Compound 18

Compound 17 (430 mg, 1.52 mmol) in DMF (2.5 mL) was added to a suspension of sodium hydride (60%, 1.60 mmol, 64 mg) in DMF (1 mL) at 0° C. under argon. The mixture was stirred at 0° C. for 15 min. 3-Bromomethyl-5-chlorobenzothiophene (3, 437 mg, 1.67 mmol) in DMF (1.5 mL) was added. The reaction mixture was stirred at 0° C. for 1.5 h. After usual work-up, the crude was chromatographed on silica gel with DCM/hexanes (1:2 to 1:1) to give 294 mg of desired compound 18 as a colorless foam.

### Synthesis of Compound 20

A mixture of compound 18 (185 mg, 0.4 mmol), compound 19 (146 mg, 0.8 mmol), Pd(PPh₃)₄ (18.6 mg, 0.016 mmol) and sodium carbonate (1.0 M, 0.8 mL) was stirred at 82-84° C. under argon overnight. The mixture was diluted with ethyl acetate, washed with brine four times, dried (Na₂SO₄) and concentrated. Chromatography on silica gel with DCM gave 121 mg of compound 20 as a yellowish solid.

### Synthesis of Compound 21

A solution of compound 20 (119 mg) was dissolved in dioxane (5 mL) and THF (5 mL). Sodium hydroxide (1.0 M, 0.6 mL) and water (1.9 mL) were added. The resulting mixture was stirred at room temperature overnight and then concentrated. Water was added and the mixture was acidified with 2 N HCl to pH 3. Precipitate was filtered and washed with water three times and dried under vacuum to give 96 mg of compound 21 as a yellow solid.

### Synthesis of Compound 22

Hydrazine (65%, 3 mL) was added to a stirred mixture of compound 21 (182 mg) and zinc dust (800 mg) in methanol (9 mL) and THF (12 mL). The resulting mixture was stirred at room temperature under argon for 4 h. Solid was filtered and washed with a mixture of THE and methanol (1:1). The filtrate was evaporated. The residue was purified two times on silica gel with 2% triethylamine and 5-6% methanol in DCM. The product was purified one more time on silica gel with 5-15% methanol in DCM to give 49 mg of compound 22 as pale-yellow solid.

### Synthesis of Compounds 23-26

By a similar procedure as described for compound 22, compounds 23-26 as shown below were prepared.

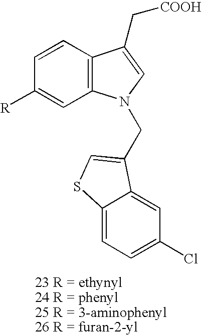

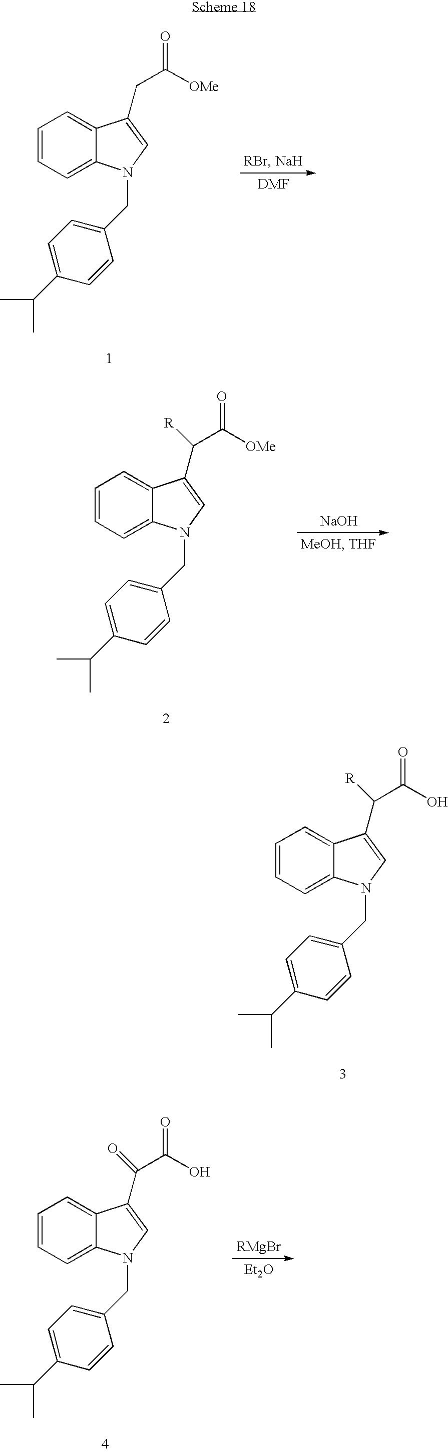

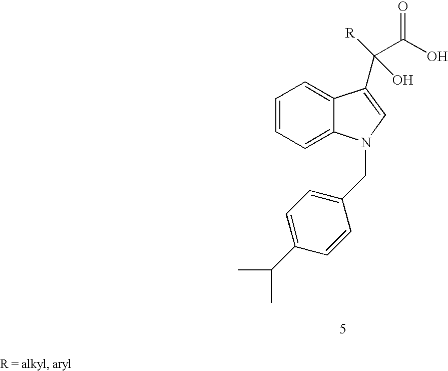

A general synthetic scheme for the preparation of α-alkyl(aryl) indole-3-acetic acid derivatives is illustrated in Scheme 18 below and exemplified by the following description of the synthesis of compound 3 (R=benzyl) and compound 5 (R=benzyl), respectively.

### Synthesis of α-Benzyl-N-1-(p-isopropylbenzyl)indole-3-acetic acid (3)

N-1-(p-isopropylbenzyl)indole-3-acetic acid 1 (0.2 g, 0.5 mmol) was dissolved in DMF (2 mL) and the solution was cooled in ice-bath. NaH (22 mg of 60% dispersion in oil, 0.55 mmol) was added and the reaction mixture was stirred at 0° C. for 10 min. Benzyl bromide (65 μL, 0.55 mmol) in DMF (1 mL) was added dropwise and the reaction mixture was stirred at room temperature for 30 min. Aqueous NH₄Cl was added, followed by extraction with EtOAc. Organic layer was washed with brine, dried (MgSO₄) and evaporated to dryness. Column chromatography on silica gel using 10% EtOAc in hexanes afforded 2 (R=benzyl) (80 mg, 33%).

To the solution of the above material (70 mg, 0.14 mmol)) in MeOH (2 ml) and THF (1 ml) 1N NaOH (2 ml) was added and the reaction mixture was stirred at RT for 3 hours. Dowex 50WX8 (pyr⁺ form) was added to neutralize the reaction mixture. The ion exchanger was then filtered off and the filtrate was evaporated to dryness. The residue was chromatographed on the column of silica gel using 2-4% gradient of MeOH in DCM to afford 3 (25 mg, 37%).

### Synthesis of α-Benzyl, α-hydroxy-N-1-(p-isopropylbenzyl)indole-3-acetic acid (5)

N-1-(p-isopropybenzyl)indole-3-glyoxylic acid 4 (0.16 g, 0.5 mmol was dissolved in THF (2.5 ml) and the solution was cooled in dry ice-acetone bath. Benzylmagnesium bromide (1.74 ml of 19% solution in THF, 1.5 mmol) was added drop wise and the reaction mixture was stirred at room temperature for 2 hours. The reaction was quenched with 2N HCl to pH 2-3 and extracted with EtOAc. Organic layer was dried (MgSO₄) and evaporated to dryness. Column chromatographic purification using 1-2% gradient of MeOH in DCM in the presence of 0.5% TEA afforded 5 (86 mg, 42%).

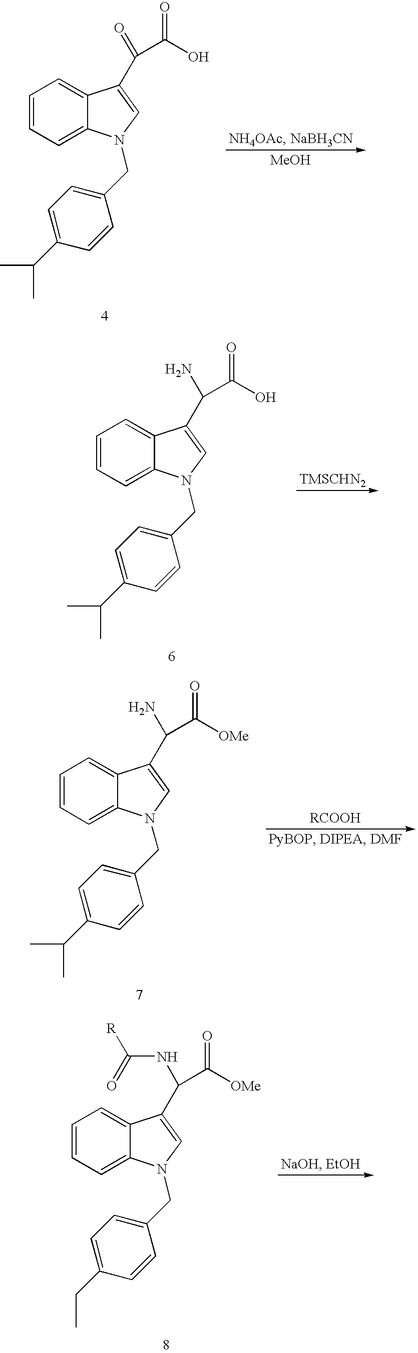

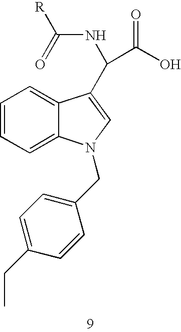

A general synthetic scheme for the preparation of α-amino indole-3-acetic acid derivatives is illustrated in Scheme 19 below and exemplified by the following description of the synthesis of compound 9 (R=Imidazole-4-yl).

### Synthesis of α-(Imidazole-4-carboxyl)amino-N-1-(p-isopropylbenzyl)indole-3-acetic acid (9)

To the solution of N-1-(p-isopropybenzyl)indole-3-glyoxylic acid 4 (1.05 g, 3.27 mmol) in MeOH, NH₄OAc (2.53 g, 32 mmol), NaBH₃CN (0.6 g, 9.6 mmol) and powdered molecular sieves 4 Å (1 g) were added. The reaction mixture was heated under reflux for 16 hours, filtered and concentrated in vacuo. The residue was partitioned between 2N HCl and EtOAc, organic layer was washed with brine, dried (MgSO₄) and concentrated in vacuo. Column chromatography on silica gel using 20% methanol in DCM (4% NH₄OH) afforded 6 (0.3 g, 19%).

α-Amino acid 6 was dissolved in MeOH (2 ml) and DCM (4 ml) and TMSCHN₂ (0.63 ml of 2N solution in hexane, 1.25 mmol) was added drop wise. The reaction mixture was stirred at RT for 30 min and evaporated to dryness to afford 7. This material was used in the next step without purification.

Ester 7 was dissolved in DMF (2 ml), PyBOP (198 mg, 0.38 mmol) and DIPEA (109 μM, 0.63 mmol) were added, followed by imidazole-4-carboxylic acid (42 mg, 0.38 mmol). The reaction mixture was stirred at room temperature for 5 hours, then partitioned between H₂O and EtOAc. Organic layer was washed with H₂O, dried (MgSO₄) and evaporated to dryness. Silica gel column chromatography using 2-4% gradient of MeOH in DCM (1% TEA) afforded 8 (60 mg, 56%).

Compound 8 (50 mg, 0.12 mmol) was dissolved in EtOH (2 mL) and 1N NaOH (2 mL) was added. The reaction mixture was stirred at room temperature for 30 min and then neutralized with Dowex 50×8 (pyr⁺ form). Ion exchanger was filtered off and the filtrate evaporated to dryness to afford the title compound 9 (30 mg, 63%).

A general synthetic scheme for the preparation of indoline-3-acetic acid derivatives is illustrated in Scheme 20 below and exemplified by the following description of the synthesis of compound 12 (R=Br, R₁=p-nitrotoluoyl)

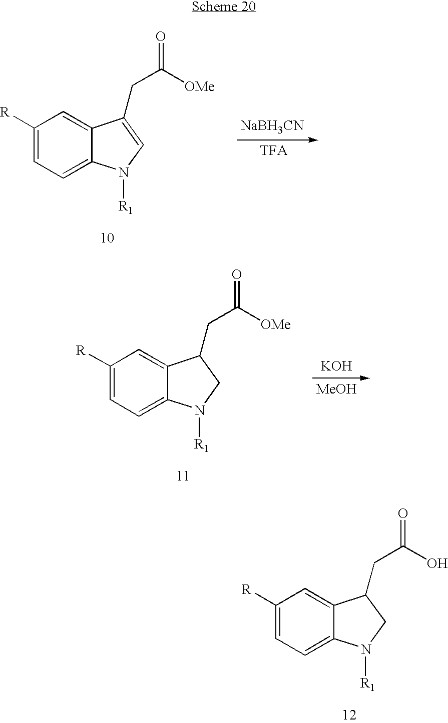

### Synthesis of 5-bromo-N-1-(p-nitrotoluoyl)indoline-3-acetic acid (12)

The mixture of 5-Bromo-N-1-p-nitrotoluoyl-indole-3-acetic acid methyl ester 10 (0.2 g, 0.5 mmol) and TFA (3 mL) was cooled to 0° C. and NaBH₃CN (314 mg, 5 mmol) was added portion wise. The reaction mixture was stirred at room temperature for 3 hours, then poured into aqueous NaHCO₃ solution and extracted with EtOAc. The organic layer was washed with brine (2×), H₂O (2×), dried (MgSO₄) and evaporated to a syrup. Silica gel column chromatography using 10% EtOAc in hexanes yielded 11 (140 mg, 70%).

Indoline 11 (130 mg, 0.32 mmol) was dissolved in the mixture of EtOH (1 ml), THF (1 mL) and 1N NaOH (1 mL). The reaction mixture was stirred at room temperature for 2 hours, then neutralized with Dowex 50×8 (pyr⁺ form). The ion-exchanger was filtered off and the filtrate evaporated to dryness to afford 12 (90 mg, 72%).

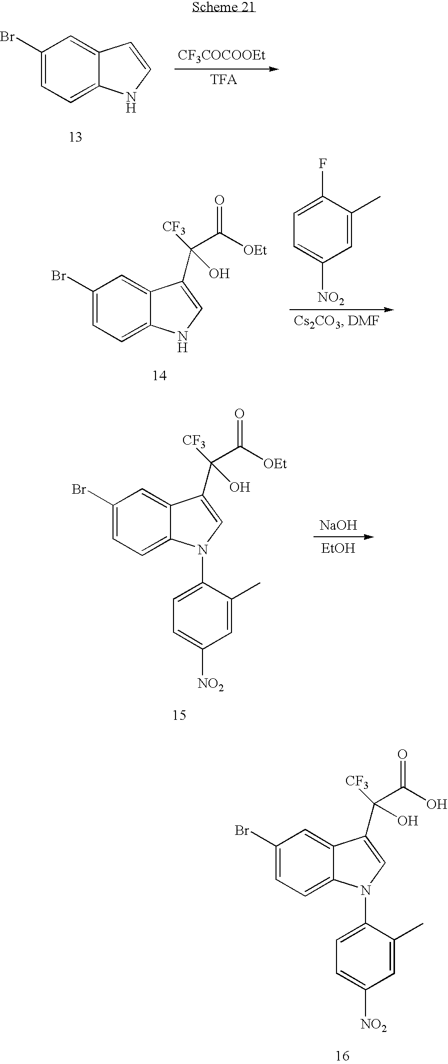

A general synthetic scheme for the preparation of 2-hydroxy-2-(indol-3-yl)-3,3,3-trifluoropropionic acid derivatives is illustrated in Scheme 21 below and exemplified by the following description of the synthesis of compound 16.

### 2-Hydroxy-2-[5-bromo-(N-1-p-nitrotoluoyl)indol-3-yl]-3,3,3-trifluoropropionic acid (16)

Indole 14 (0.73 g, 2 mmol, synthesized from 13 according to Abid, M. and Török, B. Adv. Synth. Catal. 2005, 347, 1797) was dissolved in DMF (15 mL) and Cs₂CO₃ (1.49 g, 4.57 mmol) and 2-fluoro-5-nitrotoluene (0.34 g, 2.2 mmol) were added. The reaction mixture was stirred at 60° C. for 2 hours, then kept at room temperature overnight. H₂O was added, the mixture extracted with EtOAc and organic layer dried (MgSO₄). After removal of solvents the residue was chromatographed on the column of silica gel using 30-60% gradient of DCM in EtOAc to afford 15 (0.65 g, 65%).

To the solution of 15 (100 mg, 0.2 mmol) in dioxane (1 mL) 1N NaOH (1 mL) was added. The reaction mixture was stirred at room temperature for 1 hour and then neutralized with Dowex 50×8 (pyr⁺ form). Ion exchanger was filtered off, filtrate evaporated to dryness in vacuo and the residue purified on the silica gel column. Elution using 1% MeOH in DCM (1% TEA) afforded the title compound 16 (65 mg, 69%).

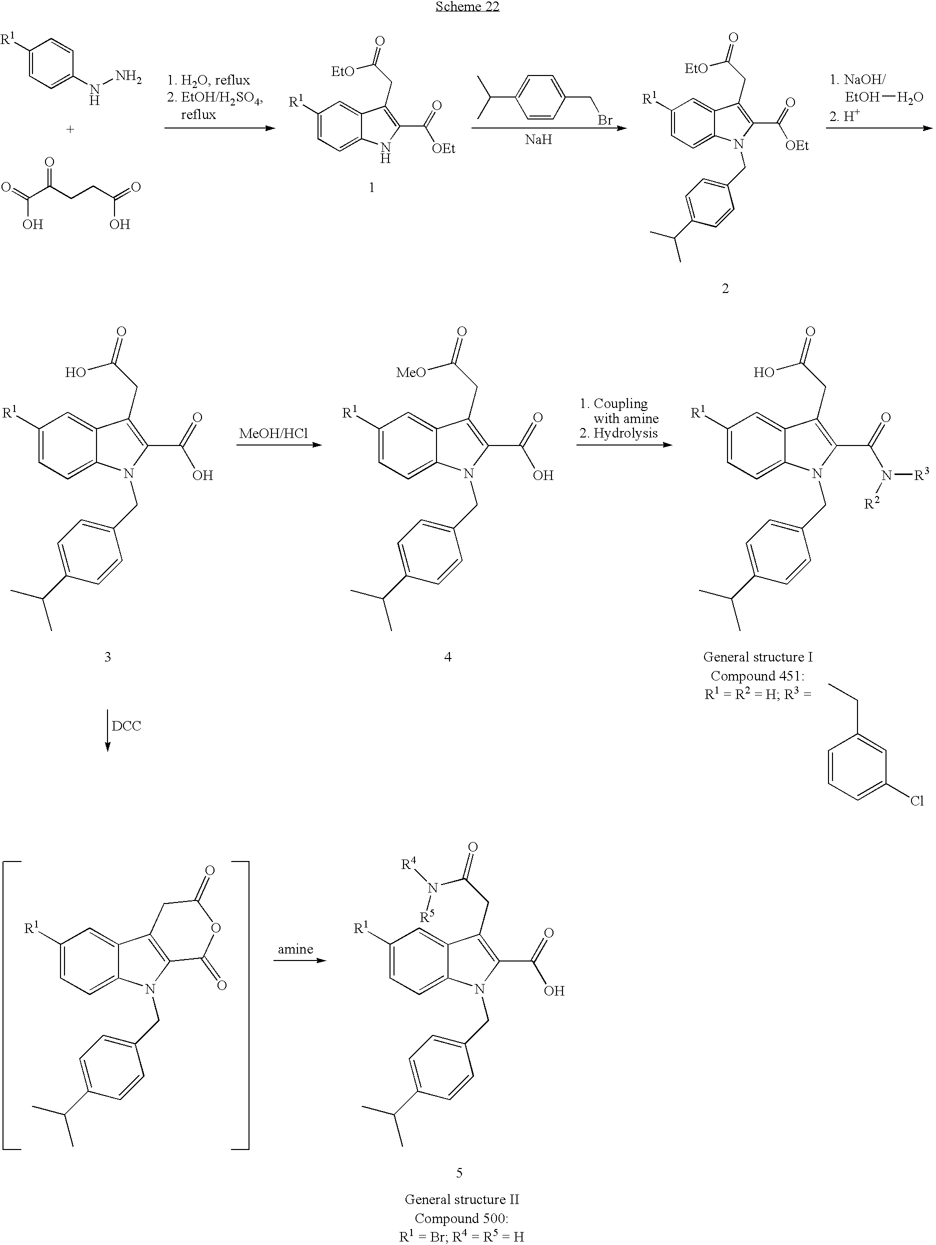

A synthetic scheme for the preparation of 2-carbamoylindole-3-acetic acid (general structure I below) and 2-carboxyindole-3-acetamides (general structure II below) helicase inhibitors is illustrated in Scheme 22 below. The synthesis is based on the publication in J. Med. Chem. 1991, 34, 1283-1292 and others and exemplified by the following description of the synthesis of compounds 451 and 500.

### Ethyl 2-ethoxycarbonyl-1H-indole-3-acetate (1, R¹=H)

Mixture of phenylhydrazine hydrochloride (14.5 g, 100 mmol), alpha-oxoglutaric acid (22 g, 150 mmol) and water (300 mL) was refluxed for 2 hours. The reaction mixture was cooled down and extracted with ethyl acetate. Organic phase was washed with water, dried over MgSO₄ and evaporated. The residue was dissolved in ethanol (500 mL), concentrated sulfuric acid (75 mL) was added carefully, and resulted mixture was refluxed for 20 hours. Ethanol was removed under reduced pressure and the residue was partitioned between ethyl acetate and water. Organic layer was washed with water followed by sodium bicarbonate and then dried over MgSO₄. The target bis-ester was isolated by chromatography (15-20% ethyl acetate-hexanes). Yield 13.6 g (63%)

### Ethyl 2-ethoxycarbonyl-1-(4-isopropylbenzyl)-1H-indole-3-acetate (2, R¹=H)

To a solution of compound 1, R¹=H (13.6 g, 49.5 mmol) in DMF was added sodium hydride (2.18 g, 60% suspension in mineral oil, 54.4 mmol) at 0° C. The mixture was stirred for 15 min. and 4-isopropylbenzyl bromide (10.2 mLl, 59.4 mmol) was added at −20° C. The reaction mixture was stirred at −20-−10° C. for one hour, when it was quenched with solid ammonium chloride and partitioned between water and ethyl acetate. Organic layer was dried over magnesium sulfate and evaporated. The title product was isolated by column chromatography (5-10% ethyl acetate-hexane). Yield 16.8 g (83%).

### 2-Carboxy-1-(4-isopropylbenzyl)-1H-indole-3-acetic acid (3, R¹=H)

To a solution of bis-ester 2, R¹=H (16.8 g, 41 mmol) in ethanol (200 mL) was added sodium hydroxide (2N, 100 mL) and the reaction was stirred at 60° C. for 1 hour. After cooling to room temperature, the reaction mixture was evaporated to solid, which was dissolved in water. The solution was acidified to pH 3 with hydrochloric acid and solid formed was filtered, washed with water, and re-crystallized from methanol. Yield 13.7 g (95%).

### Methyl 2-carboxy-1-(4-isopropylbenzyl)-1H-indole-3-acetate (4, R¹=H)

To a suspension of the bis-acid 3, R¹=H (13.7 g, 39 mmol) in methanol (200 mL) was added dioxane solution of HCl (4M, 20 mL). The suspension was stirred for 2 hours at room temperature, evaporated and dried under vacuum. Yield: 14.1 g, 100%.

### 2-(3-Chlorobenzylcarbamoyl)-1-(4-isopropylbenzyl)-1H-indol-3-acetic acid (representative compound 451)

To a solution of above the acid 4, R¹=H (50 mg, 0.14 mmol) in DCM (2 mL) were added HOBt (19 mg, 0.14 mmol) followed by DCC (0.14 mL, 1M solution in DCM). Reaction mixture was stirred for 15 min when 3-chlorobenzylamine (0.038 mL, 0.3 mmol) was added and the reaction mixture was stirred for one hour. Precipitate was filtered off, washed with ethyl acetate and the filtrate was washed successively with 0.1 N HCl, water and saturated sodium bicarbonate. The organic phase was dried over magnesium sulfate and evaporated. The title compound was isolated by column chromatography in 2% ethyl acetate in DCM. Yield 70 mg (100%).

To the solution of this ester (70 mg, 0.14 mmol) in dioxane (2 mL) was added 2N hydrochloric acid and the reaction was heated for two hours at 90° C. The colored reaction mixture was evaporated under vacuum and co-evaporated twice with toluene-ethanol mixture (3:1). The title compound was isolated by column chromatography in 5-10% MeOH-DCM. Yield: 20 mg (29%).

### 5-Bromo-2-carboxy-(4-isopropylbenzyl)-1H-indole-3-acetamide (Representative Compound 500)

To a solution of bis-acid 3, R¹=Br in DCM (2 mL) was added DCC and the mixture was stirred at room temperature for 3 hours, and then treated with ammonia (0.3 mL of 0.5M DCM solution, 1.5 mmol). After stirring for another hour, the reaction mixture was diluted with ethyl acetate, precipitate was filtered off and the filtrate was washed with 0.1 N hydrochloric acid followed by water. Organic solution was dried over magnesium sulfate and evaporated. The target compound was isolated by column chromatography in 10-15% methanol DCM. Yield: 25 mg (58%).

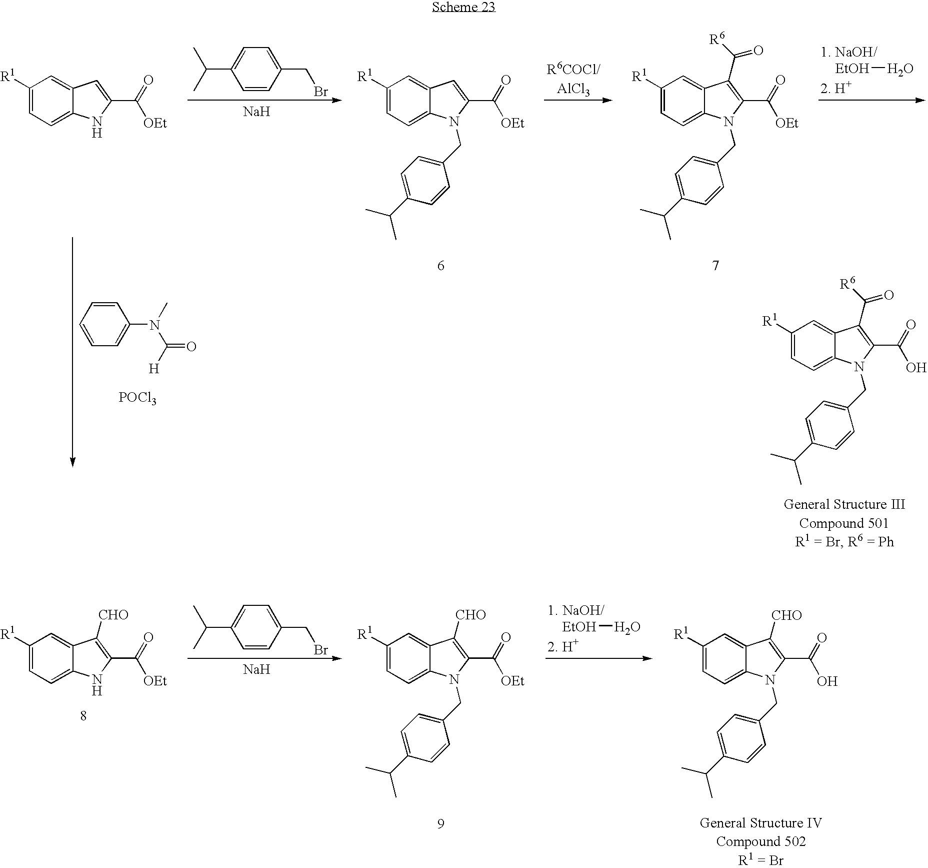

A synthetic scheme for the preparation of compounds general structures III and IV illustrated in Scheme 23 below. The synthesis is exemplified by the following description of the synthesis of compounds 501 and 502:

### Ethyl 5-bromo-1-(4-isopropylbenzyl)-1H-indole-2-carboxylate (6, R¹=Br)

To a solution of commercially available ethyl 5-bromo-1H-indole-2-carboxylate (804 mg, 3 mmol) in DMF (10 mL) was added sodium hydride (144 mg as 60% mineral oil suspension, 3.6 mmol) at 0° C. After 15 min of stirring 4-isopropylbenzyl bromide (0.68 ml, 4 mmol) was added and the reaction was left with stirring for 1 hour at room temperature. After quenching with solid ammonium chloride, the reaction was partitioned between water and ethyl acetate, organic phase was separated, dried over magnesium sulfate and evaporated. The title compound was isolated by column chromatography in 5-10% ethyl acetate-hexane. Yield 1.21 g (100%).

### Ethyl 3-benzoyl-5-bromo-1-(4-isopropylbenzyl)-1H-indole-2-carboxylate (7, R¹=Br)

The solution of indole 6 (R¹=Br, 128 mg, 0.32 mmol) in dichloroethane was treated with anhydrous aluminum chloride (85 mg, 0.64 mmol) and benzoic anhydride (108 mg, 0.48 mmol). After stirring overnight at room temperature, the mixture was diluted with DCM, washed with water followed by saturated sodium bicarbonate. This organic solution was dried over sodium sulfate and evaporated. The target compound was isolated by column chromatography in 10-15% ether-hexane. Yield: 30 mg (19%).

### 3-benzoyl-5-bromo-1-(4-isopropylbenzyl)-1H-indole-2-carboxylic acid (compound 501)

The solution of compound 7 (R¹=Br) (30 mg, 0.06 mmol) in ethanol (2 mL) was treated with lithium hydroxide (2 N, 0.2 mL). After 30 min of stirring the reaction mixture was acidified to pH 3 with 2N hydrochloric acid and evaporated. The target compound was isolated by column chromatography in 5-10% MeOH-DCM. Yield 26 mg (92%).

### Ethyl 5-bromo-3-formyl-1-1H-indole-2-carboxylate (8, R¹=Br)

Phosphorous oxochloride (0.73 mL, 8 mmol) and N-methylformanilide (0.98 mL, 8 mmol) were mixed together and stirred for 15 min at room temperature. To this mixture a solution of ethyl 5-bromo-1H-indole-2-carboxylate (1.072 g, 4 mmol) in dichloroethane (15 mL) was added and the reaction was allowed to proceed for two hours at 80° C. After cooling to room temperature, this reaction mixture was added drop wise to a solution of sodium acetate (5 g) in water (10 mL) to afford precipitation of the target compound. This solid was filtered off, washed with water followed by cold methanol and dried under vacuum. Yield 1.06 h (89%).

### Ethyl 5-bromo-3-formyl-(4-isopropylbenzyl)-1H-indole-2-carboxylate (9, R¹=Br)

Compound 9 was prepared from indole 8 under standard benzylation conditions using sodium hydride and 4-isopropylbenzyl bromide in DMF. The target compound was isolated by column chromatography in 5-10% ethyl acetate-hexane. Yield: 51%.

### 5-Bromo-3-formyl-(4-isopropylbenzyl)-1H-indole-2-carboxylic acid (compound 502)

The ester 9 (R¹=Br) (50 mg) was hydrolyzed using 2 N lithium hydroxide in ethanol-water solution at room temperature. After standard extractive work-up the target compound was isolated by column chromatography in 5-10% MeOH-DCM. Yield: 35 mg (76%).

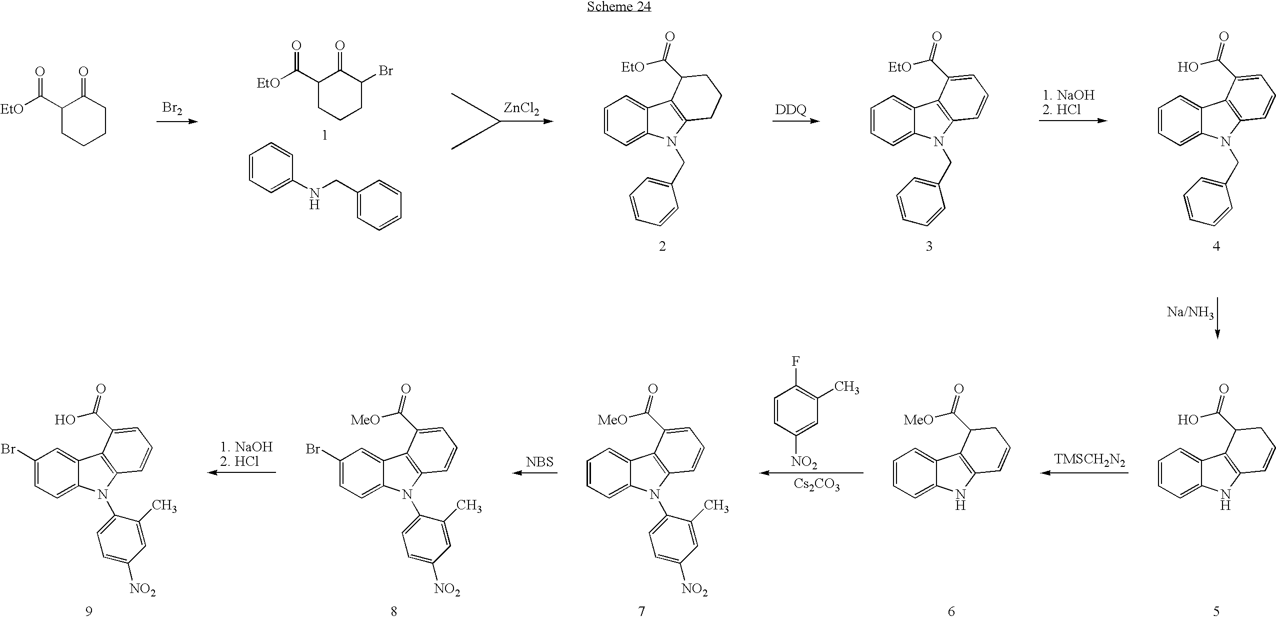

### Preparation of Carbazole Derivatives.

### Ethyl 3-Bromo-2-oxocyclohexanecarboxylate (1) (J. Med. Chem. 2005, 48, p. 8045-8054)

To a stirred at 0° C. solution of ethyl 2-oxocyclohexanecarboxylate (25 g, 0.15 mol) in diethyl ether (50 mL) bromine (7.7 mL, 0.15 mol) was added dropwise. After being stirred for 15 min at 0° C. and then 1.5 h at room temperature, the reaction mixture was carefully poured into stirred saturated aqueous sodium carbonate solution and then extracted with ethyl acetate. The combined organic layer was dried over magnesium sulfate end evaporated to give bromo ketone 1 as yellowish oil. Yield 37 g (99%).

### Ethyl 9-benzyl-1,2,3,4-tetrahydrocarbazole-4-carboxylate (2) (DE 2 127 352)

A mixture of phenylbenzylamine (4.42 g, 24 mmol) and keto ester 1 was stirred for 3 days at 35° C. To this brown mixture anhydrous zinc chloride (4 g) was added and the reaction mixture was stirred at 125-130° C. for 1 hour. After cooling down to room temperature the dark reaction mixture was partitioned between water and ethyl acetate. Organic layer was separated; water phase was additionally extracted with ethyl acetate. The combined organic extract was washed with 2 N HCl and water, dried over magnesium sulfate and evaporated. The tetrahydrocarbazole 2 was isolated by column chromatography using 30 to 40% ethyl acetate-hexane. Yield: 2.21 g (66%).

### Ethyl 9-benzylrocarbazole-4-carboxylate (3)

A solution of tetrahydrocarbazole 2 (500 mg, 1.50 mmol) and DDQ (1.14 g, 5.02 mmol) in o-xylene (15 mL) was stirred for 1 h at 120° C. The cooled reaction mixture was filtered through Celite and evaporated. The carbazole 3 was isolated as off-white crystalline material after column chromatography in 40% DCM-hexane. Yield: 420 mg (84%).

### 9-benzylrocarbazole-4-carboxylic acid (4)

Solution of the ester 3 (420 mg, 1.28 mmol) in ethanol (5 mL) was treated with sodium hydroxide (2 M, 2 mL). After stirring for 1 h at 80° C. the solvent was removed under vacuum and the residue was taken into water. After addition of 2 N HCl to pH 3 the acid 4 was isolated by standard extractive work up followed by crystallization from ethyl acetate-hexane mixture (2:1). Yield 300 mg (78%).

### 3,4-Dihydroarbazole-4-carboxylic acid (5)

Liquid ammonia (approximately 10 mL) was added to the acid 4 (250 mg, 0.83 mmol) followed by addition of sodium (53 mg, 2.3 mmol) at −50° C. The dark solution was stirred for 45 min. at the same temperature when the reaction was quenched with solid ammonium chloride and ammonia was allowed to distilled off. The dark residue was dissolved in water and the solution was acidified with 1 N HCl to pH 3. After usual extractive work up the acid 5 was isolated by column chromatography (3% MeOH-DCM) followed by crystallization from DCM-hexane (1:1). Yield 70 mg (40%).

### Methyl 3,4-Dihydroarbazole-4-carboxylate (6)

To a solution of the acid 5 (50 mg, 0.25 mmol) in methanol (1 mL) was added trimethylsilyldiazomethane as 2M solution in THF (1.25 ml, 2.5 mmol) at 0° C. The reaction mixture was stirred for 30 min at 0° C. and evaporated to leave crude ester 6 which was used on the next step without any additional purification.

### Methyl 9-(2-methyl-4-nitrophenyl)carbazole-4-carboxylate (7)

To a solution of the ester from the previous step in DMF (5 mL) 3-nitro-5-fluorotoluene (98 mg, 0.5 mmol) and anhydrous cesium carbonate (244 mg, 0.75 mmol) were added and the reaction mixture was stirred at 35° C. overnight. The resulted dark mixture was partitioned between water and ethyl acetate, and organic layer was washed with brine and dried over magnium sulfate. Column chromatography purification (5% ethyl acetate-hexane) furnished the title product as a yellow oil. Yield 80 mg (89%, two steps).

### Methyl 6-bromo-9-(2-methyl-4-nitrophenyl)carbazole-4-carboxylate (8)

To a solution of carbazole 7 (80 mg, 0.22 mmol) in DCM (2 mL) and acetic acid (2 mL) was added NBS (46 mg, 0.26 mmol) at 0° C. The reaction was stirred for 1 hour at the same temperature and poured into a stirred saturated sodium bicarbonate solution. The product was extracted with DCM. The organic solution dried over magnesium sulfate and evaporated to give bromocarbazole 8 which was used on the next without any additional purification. Yield 80 mg (83%).

### 6-Bromo-9-(2-methyl-4-nitrophenyl)carbazole-4-carboxylic acid (9)

The ester 8 (80 mg, 0.18 mmol) was hydrolized with sodium hydroxide (2 M, 1 mL) in ethanol (2 mL) at 80° C. for 1 h. The solvent was evaporated and the residue was taken into water. 2M HCl was added to pH 3 and the product was extracted with ethyl acetate. Organic solution was dried over magnesium sulfate and evaporated. Crystallization from DCM gave the acid 9 as a white solid. Yield 60 mg (78%).

Preparation of Indole derivatives is described in Scheme 25 to Scheme 35.

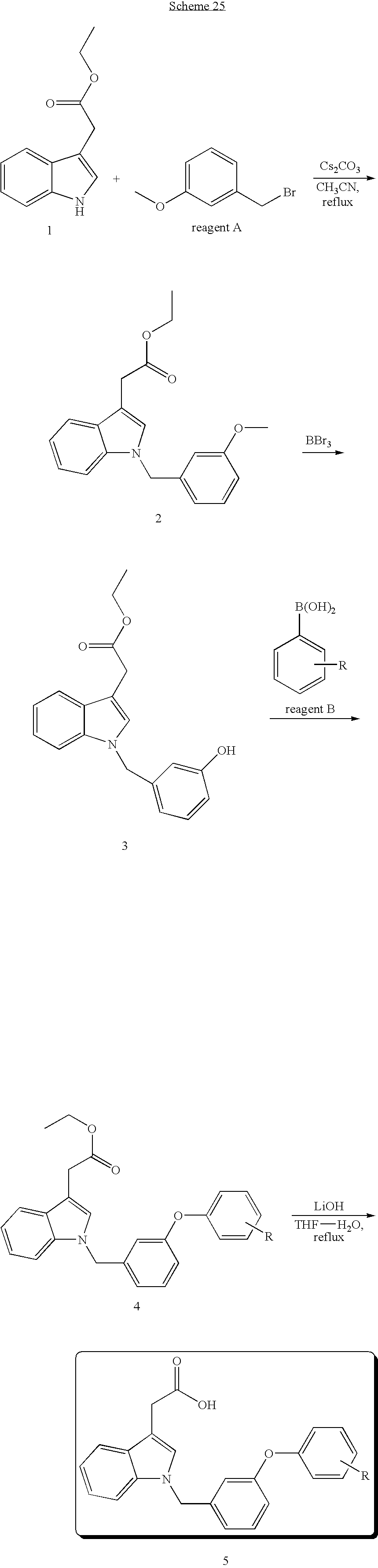

### Preparation of Indole Derivatives.

### Preparation of Compound 2

A mixture of compound 1 (2.03 g, 1 eq.), 1-(bromomethyl)-3-methoxybenzene (2.00 g, 1 eq.) and Cs₂CO₃ (4.88 g, 1.5 eq.) in CH₃CN (20 mL) was heated to reflux for about 8 h. The reaction was traced by TLC, After the completion of the reaction, the reaction mixture was cooled to room temperature, the solid was filtered off, the solvent was removed under reduced pressure, and the crude product was purified by chromatography column to give 2.20 g of compound 2 (68% yield).

### Preparation of Compound 3

Compound 2 (2.20 g, 1 eq.) was dissolved in anhydrous dichloromethane (10 mL). Boron tribromide (3 eq.) was added to this mixture under ice-cooling condition and the reaction mixture was stirred at room temperature for 14 hours. Then 1N aqueous sodium hydroxide was added to the reaction mixture, the reaction mixture was extracted with ethyl acetate (2×300 mL). The organic layer was washed with brine and then dried over anhydrous Na₂SO₄. The solvent was removed under reduced pressure, and the crude was purified by chromatography column to give 1.68 g of compound 3 (80% yield).

### Preparation of Compound 4

A mixture of compound 3 (1 eq.), reagent B (2-3 eq.), Cu(OAc)₂ (1.3 eq.), pyridine (5 eq.), pyridine N-Oxide (1.0 eq.) and molecular sieves 4 A in dichloromethane (5 ml/1 mmol compound 3) was stirred overnight at the room temperature opened to the air. The reactions were monitored by TLC, and when found to be completed, the reactions were washed with aqueous sodium bicarbonate, extracted by CH₂Cl₂ and the crude was isolated by pre-TLC to get compound 4.

### Preparation of Compound 5

Compound 4 (1 eq.) was dissolved in THF-H₂O (3:1, 4 ml) and LiOH (5 eq.) was added to the mixture. It was heated to 70-80° C. with stirring for over night. The reaction was monitored by TLC. After completion of the reaction, the mixture was acidified by 2M HCl to pH 2-3 then extracted by ethyl acetate (EA) for 3 times of the reaction volume, washed with brine. The organic layers were combined, then the solvent was removed, and the target products were purified by pre-TLC.

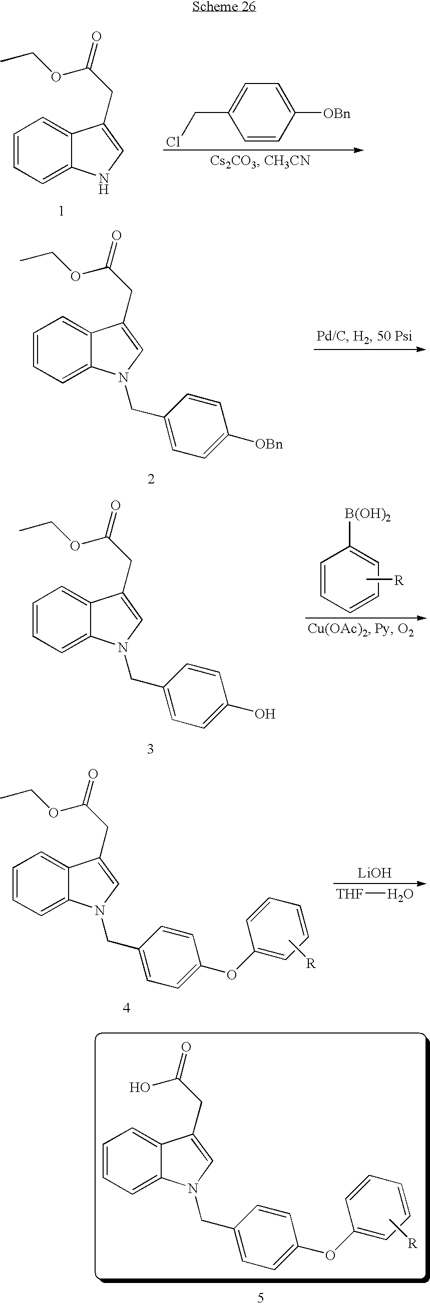

### Preparation of Compound 2

A mixture of compound 1 (2.68 g, 1 eq.), 1-benzyloxy-4-chloromethyl-benzene (4.0 g, 1 eq.) and Cs₂CO₃ (6.43 g, 1.5 eq.) in CH₃CN (30 mL) was heated to reflux for about 12 h. After cooling the solid was filtered off, the solvent was removed under reduced pressure. The resulting crude was purified by chromatography column to give 3.0 g of compound 2 (57% yield).

### Preparation of Compound 3

Compound 2 (1.4 g) was hydrogenated in MeOH/EtOAc (8:1) in the presence of Pd/C (280 mg) in an initial H₂ of 50 Psi at 40-50° C. for about 4 h. Then the catalyst was filtered off, the solvent was removed in vacuum to afford 0.86 g of compound 3 (79% yield) which was used in the next step without further purification.

### Preparation of Compound 4

A mixture of compound 3 (1 eq.), boronic acid (2-3 eq.), Cu(OAc)₂ (1.3 eq.), pyridine (5 eq.), pyridine N-Oxide (1.0 eq.) and molecular sieves 4 A in dichloromethane (5 mL/1 mmol compound 3) was stirred for 14-36 h at room temperature opened to the air. The reaction was monitored by TLC and LC-MS. After completion of the reaction, saturated, sodium bicarbonate was added to the reaction mixture. The organic layer was separated and the aqueous layer was extracted by CH₂Cl₂. The combined organic layer was washed with brine, dried over Na₂SO₄ and concentrated. The crude was purified by pre-TLC to give the pure compound 4.

### Preparation of Compound 5

To a solution of compound 4 (1 eq.) in THF-H₂O (3:1, 4 ml) was added LiOH (5 eq.) at room temperature. The mixture was heated to 70-80° C. overnight. After cooling the reaction mixture was acidified with 2M HCl to pH 2-3. The aqueous was extracted with EtOAc. The combined organic layer was washed with brine, dried and concentrated. The resulting crude was purified by pre-TLC to give the pure compound 5.

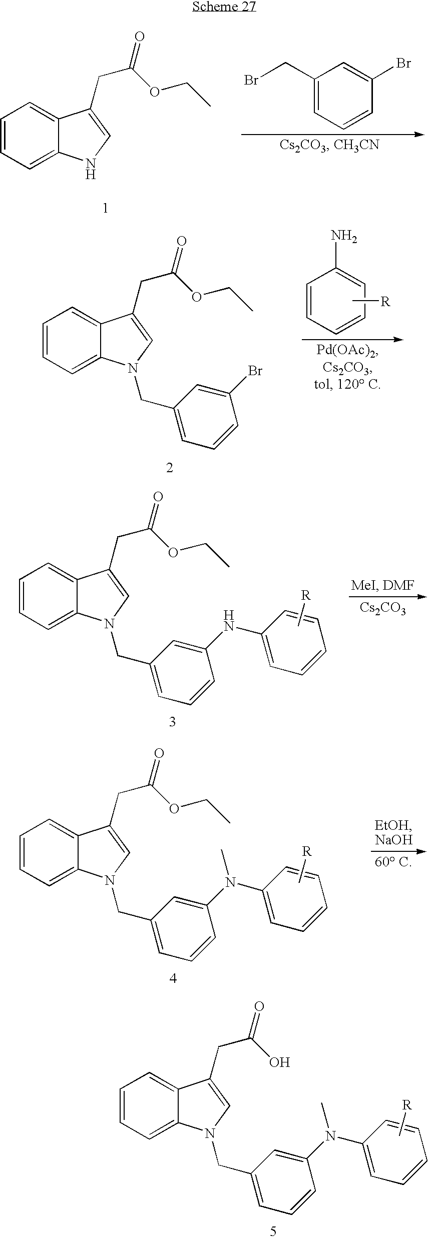

### Preparation of Compound 2

A mixture of compound 1 (1 eq.), 1-bromomethyl-4-bromobenzene (1 eq.) and Cs₂CO₃ (1.5 eq.) in CH₃CN (5 mL/l) was heated to reflux for about 12 h. After cooling the solid was filtered off, the solvent was removed under reduced pressure. The resulting crude was purified by chromatography column to give compound 2.

### Preparation of Compound 3

A mixture of compound 2 (1 eq.), phenyl-amine (1.5 eq.) and cesium carbonate (2 eq.) in toluene (5 mL/1 mmol reagent 2) was stirred at 110° C. in nitrogen atmosphere, then palladium acetate and xanphos were added. The mixture was stirred at the temperature overnight and monitored by TLC. Compound 3 was isolated by pre-TLC. (PE/EA=5:1)

### Preparation of Compound 4

A mixture of compound 3 (1 eq.), Cs₂CO₃ and Iodomethane (3 eq.) in DMF was stirred at r.t over night. The reaction was monitored by TLC. When the starting material was consumed, the mixture was extracted with EtOAc, washed with saturated brine, dried over anhydrous sodium sulfate, concentrated in vacuum and the target product was isolated by pre-TLC. (PE/EA=5:1)

### Preparation of Compound 5

A mixture of compound 4 (1 eq.), and NaOH (3 eq.) in methanol was stirred at 50° C. opened to the air. The reaction was monitored by TLC. When the starting material was consumed, the cooled mixture was extracted with EtOAc, washed with saturated brine, dried over anhydrous sodium sulfate, concentrated in vacuum and the target product was isolated by pre-TLC. (PE/EtOAc=1:1)

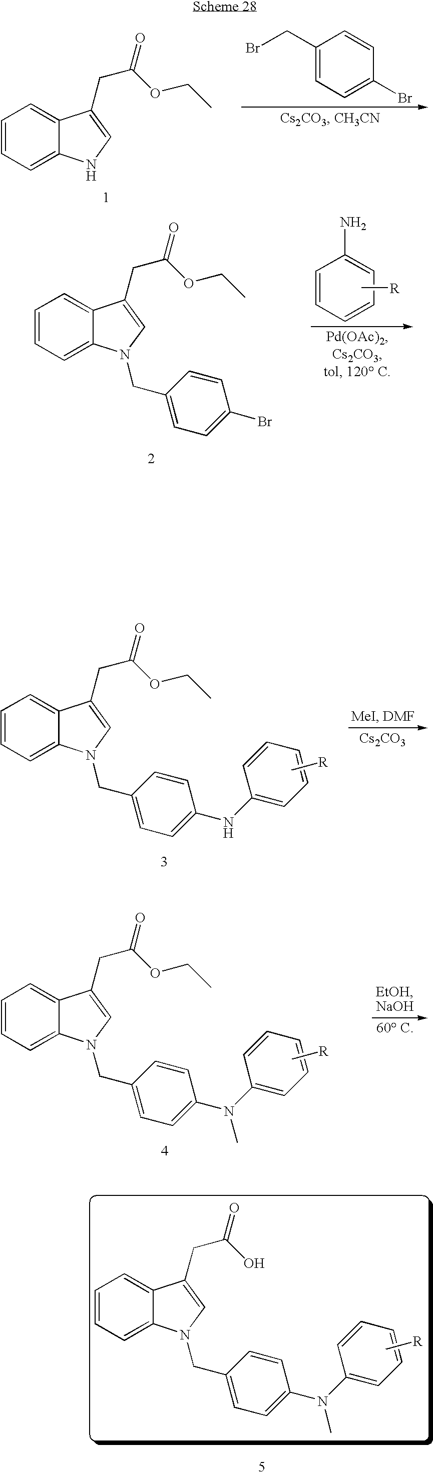

### Preparation of Compound 2

A mixture of compound 1 (1 eq.), 1-bromomethyl-4-bromobenzene (1 eq.) and Cs₂CO₃ (1.5 eq.) in CH₃CN (5 mL/l) was heated to reflux for about 12 h. After cooling the solid was filtered off, the solvent was removed under reduced pressure. The resulting crude was purified by chromatography column to give compound 2.

### Preparation of Compound 3

A mixture of compound 2 (1 eq.), phenyl-amine (1.5 eq.) and cesium carbonate (2 eq.) in toluene (5 mL/1 mmol reagent 2) was stirred at 110° C. in nitrogen atmosphere, then palladium acetate and xanphos were added. The mixture was stirred at the temperature overnight and monitored by TLC. Compound 3 was isolated by pre-TLC. (PE/EtOAc=5:1).

### Preparation of Compound 4

A mixture of compound 3 (1 eq.), Cs₂CO₃ and Iodomethane (3 eq.) in DMF was stirred at room temperature over night. The reaction was monitored by TLC. When the starting material was consumed, the mixture was extracted with EtOAc, washed with brine, dried over anhydrous sodium sulfate, concentrated in vacuum and the target product was isolated by pre-TLC. (PE/EA=5:1)

### Preparation of Compound 5

A mixture of compound 4 (1 eq.), and NaOH (3 eq.) in methanol was stirred at 50° C. opened to the air. The reaction was monitored by TLC. When the starting material was consumed, the cooled mixture was extracted with EtOAc, washed with saturated brine, dried over anhydrous sodium sulfate, concentrated in vacuum and the target product was isolated by pre-TLC. (PE/EtOAc=1:1)

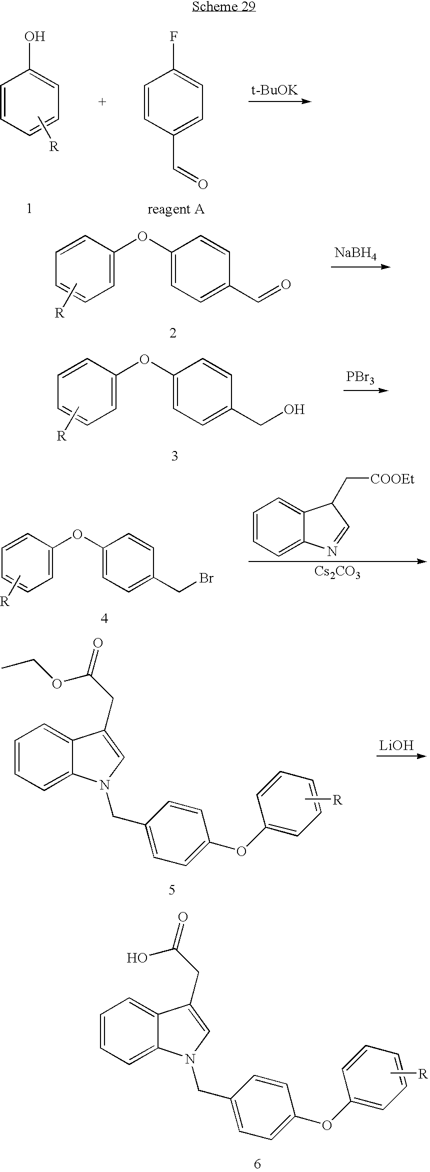

### Preparation of Compound 2

A mixture of compound 1 (100 mg, 1 eq.), t-BuOK (114.8 mg, 1.5 eq.) in DMA (10 mL) was stirred at 150° C. for 0.5 hour, then reagent A (124.03 mg, 1.5 eq.) was added. The resulting mixture was stirred for another 2 hours at 150° C. The reaction was detected by TLC, After completion of the reaction, mixture was diluted with water and EtOAc, and extracted with EtOAc. The organic layers were combined, solvent was removed under reduced pressure, the crude was purified by pre-TLC give 140 mg of compound 2 (about 50% yield).

### Preparation of Compound 3

Compound 2 (100 mg, 1 eq.) was dissolved in THF-EtOH (1:2.5) 3.5 mL, and the solution was cooled to 0° C., NaBH₄ (14.4 mg, 1 eq.) was added. The mixture was warmed to room temperature and stirred for about 2 hours. The reaction was monitored by TLC. After completion of the reaction, solution was poured into cold 25% aq. NH₄OAc. Then the mixture was extracted with EtOAc. Organic layers were combined, and dried by anhydrous Na₂SO₄. The solvent was removed under a reduced pressure, and the crude was purified by pre-TLC to give 100 mg of compound 3 (about 90% yield).

### Preparation of Compound 4

Compound 3 (100 mg, 1 eq.) was dissolved in dry THF (2 mL). The solution was cooled to 0° C. then PBr₃ was added drop wise. After PBr₃ was added, the mixture was warmed to room temperature and stirred for about 4 hours. The reaction was monitored by TLC. After completion of the reaction, solution was poured into cold saturated aq. NaHCO₃. Then the mixture was extracted with EtOAc. Organic layers were combined, and dried by anhydrous Na₂SO₄. The solvent was removed under a reduced pressure, and the product was purified by pre-TLC. 50 mg compound 4 was obtained (about 40% yield).

### Preparation of Compound 5

A mixture of compound 4 (50 mg, 1 eq.), (1H-indol-3-yl)acetic acid ethyl ester (33.8 mg, 1.1 eq.) and Cs₂CO₃ (73.1 mg, 1.5 eq.) in CH₃CN (5 mL) was heated to reflux for about 12 h. After cooling the solid was filtered off, the solvent was removed under a reduced pressure, and the crude was purified by pre-TLC to give 60 mg of compound 6 (about 70% yield).

### Preparation of Compound 6

Then compound 5 (60 mg, 1 eq.) was dissolved in THF-H₂O (3:1.4 ml), LiOH (5 eq.) was added, the mixture was heated to 70-80° C. and stirred over night. The reactions were monitored by TLC. When the reactions were completed, the mixture was acidified by 2M HCl to pH 2-3, extracted by EtOAc for 3 times, washed with brine. The organic layers were combined, and dried by anhydrous Na₂SO₄, the solvent was removed, and the target products were purified by pre-TLC.

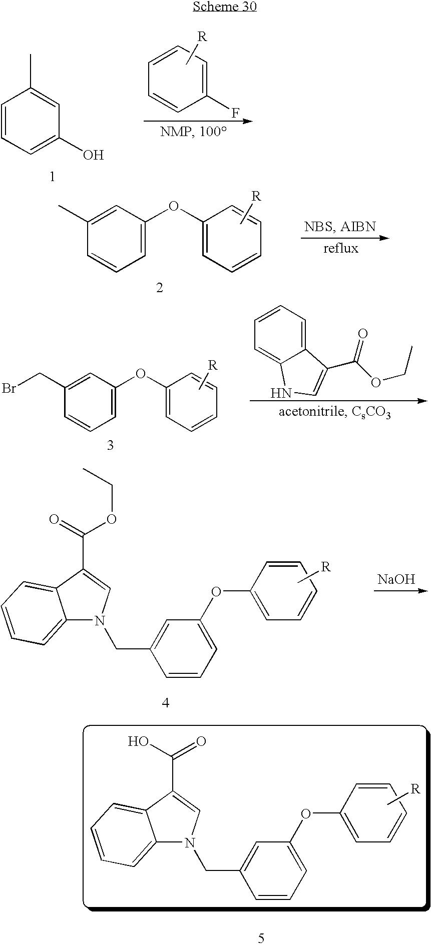

### Preparation of Compound 2

A mixture of compound 1 (1.08 g, 1 eq.), 1-fluoro-2,4-bis(trifluoromethyl)benzene (2.32 g, 1 eq.) and K₂CO₃ (1.5 g, 1.1 eq.)in NMP (20 mL) was heated to 100° C. for about 8 h. After cooling the solid was filtered off, the filtrate was extracted by EtOAc/H₂O, the organic layers were concentrated, the product was purified by chromatography column to give 2.56 g of compound 2 (80% yield).

### Preparation of Compound 3

Compound 2 (2.56 g, 1 eq.) was dissolved in CCl₄, then NBS (1.70 g, 1.2 eq.) and AINB (64 mg. 0.05 eq.) was added. After the completion of reaction, the reaction mixture was concentrated, the product was purified by chromatography column to give 2.71 g of compound 3 (85% yield).

### Preparation of Compound 4

A mixture of compound 3 (2.71 g, 1 eq.), ethyl 1H-indole-3-carboxylate (1.28 g, 1 eq.) and Cs₂CO₃ (2.64 g, 1.2 eq.) in CH₃CN (20 mL) was heated to reflux for about 8 h. After cooling to the room temperature, the solid was filtered off, the solvent was removed under reduced pressure, and the crude was purified by chromatography column to give 2.41 g of compound 4 (70% yield).

### Preparation of Compound 5

Compound 4 (1 eq.) was dissolved in THF-H₂O (3:1, 4 mL), and NaOH (3 eq.) was added to the mixture. The mixture was then heated to 70-80° C. and stirred over night. The reactions were monitored by TLC. When the reactions were completed, the mixture was acidified by 2M HCl to pH 3-4, then extracted by EtOAc for 3 times, washed with brine. The organic layers were combined, then the solvent was removed, and the target products were purified by pre-TLC (90% yield).

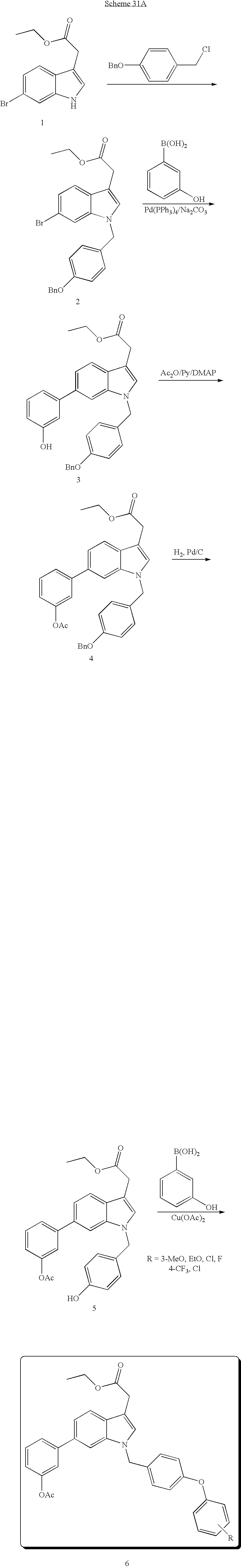

### Preparation of Compound 2

A mixture of compound 1 (5 g, 1 eq.), 1-Benzyloxy-4-chloromethyl-benzene (4.54 g, 1.2 eq.) and Cs₂CO₃ (8.67 g, 1.5 eq.) in CH₃CN (50 mL) was heated to reflux for about 12 h. After cooling the solid was filtered off, the solvent was removed under reduced pressure. The resulting crude was purified by chromatography column to give 6.0 g of compound 2 (70% yield).

### Preparation of Compound 3

A mixture of compound 2 (3.0 g, 1 eq.), 3-hydroxyphenylboronic acid (2.2 g, 2 eq.), Pd(PPh₃)₄ (363.2 mg, 0.1 eq.), aqueous Na₂CO₃ (1.67 g, 2.5 eq.) in 1:1 toluene-EtOH was heated to reflux under nitrogen protection, the reaction was detected by TLC, After completion of the reaction, the solid was filtered off, the solvent was removed under reduced pressure. The product was purified by chromatography column to give 1.95 g of compound 3 (63% yield).

### Preparation of Compound 4

Compound 3 (1.95 g) was dissolved in pyridine (10 mL), then acetic anhydride (1.62 g, 5 eq.), dimethyl-pyridin-4-yl-amine (532 mg, 1.1 eq.) was added, the resulting mixture was stirred at room temperature, the reaction was detected by TLC, After completion of the reaction, the pyridine was removed under reduced pressure. The product was purified by chromatography column to give 1.4 g of compound 4 (66% yield).

### Preparation of Compound 5

Compound 4 (1.6 g) was hydrogenated in MeOH/EtOAc (8:1) in the presence of Pd/C (320 mg) in an initial H₂ of 50 Psi at 40-50° C. for about 4 h. Then the catalyst was filtered off, the solvent was removed in vacuum to afford 0.89 g of compound 5 (79% yield) which was used in the next step without further purification.

### Preparation of Compound 6

A mixture of compound 5 (1 eq.), boronic acid (2-3 eq.), Cu(OAc)₂ (1.3 eq.), pyridine (5 eq.), pyridine N-Oxide (1.0 eq.) and molecular sieves 4 A in dichloromethane (5 ml/1 mmol compound 3) was stirred for 14-36 h at room temperature opened to the air. The reaction was monitored by TLC and LC-MS. After completion of the reaction, aqueous sodium bicarbonate was added to the reaction mixture. The organic layer was separated and the aqueous layer was extracted by CH₂Cl₂. The combined organic layer was washed with brine, dried over Na₂SO₄ and concentrated. The crude was purified by pre-TLC to give the pure compound 6.

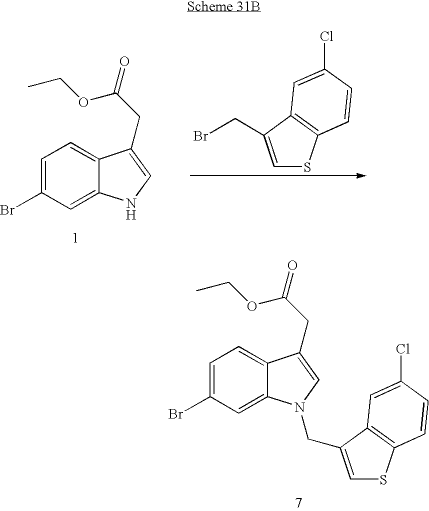

### Preparation of Compound 7

A mixture of compound 1 (5 g, 1 eq.), 3-(bromomethyl)-5-chlorobenzo[b]thiophene (5.5 g, 1.2 eq.) and Cs₂CO₃ (8.67 g, 1.5 eq.) in CH₃CN (50 mL) was heated to reflux for about 2-3 h. After cooling the solid was filtered off, the solvent was removed under reduced pressure. The resulting crude was purified by chromatography column to give 4.0 g of compound 7 (50% yield).

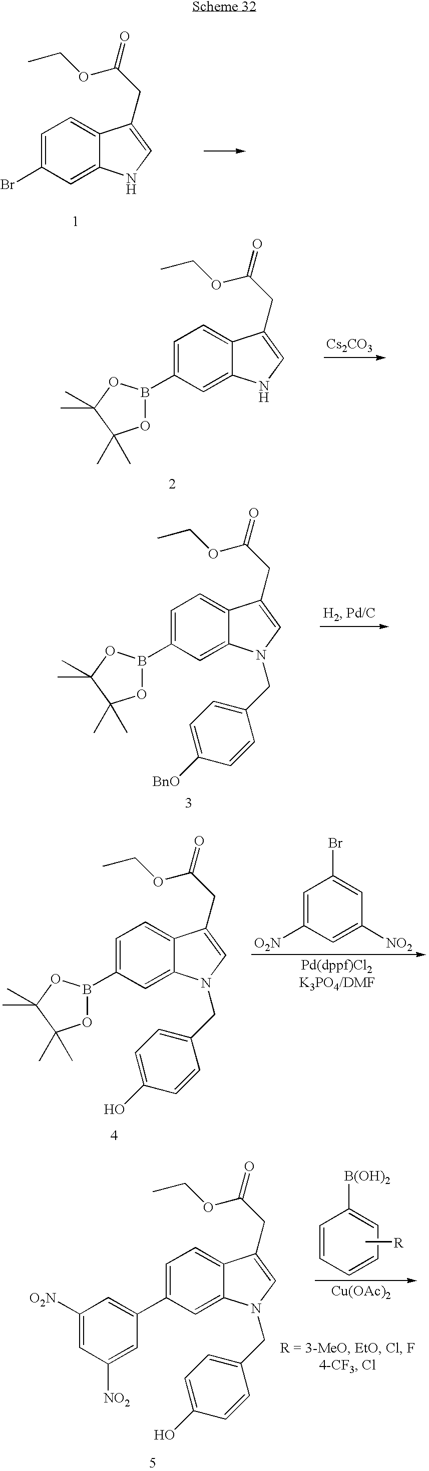

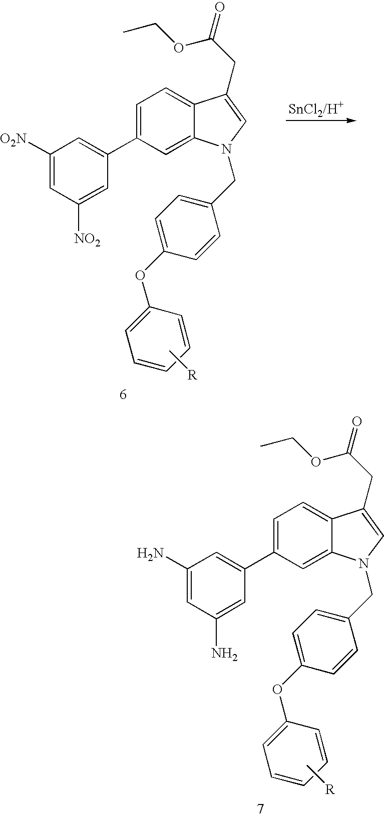

### Preparation of Compound 2

A mixture of compound 1 (15 g, 1 eq.), Bis(pinacolato)diboron (29.9 g, 2 eq.), KOAc (18 g, 3.4 eq., Ac=acetyl), Pd(dppf)Cl₂ (3.81 g, 0.1 eq., dppf=1,1′-bis(diphenylphosphino)ferrocene) in N,N-Dimethylformamide was heated to 80° C. under nitrogen protection, the reaction was detected by TLC. After completion of the reaction, of the reaction the solid was filtered off, the solvent was removed under reduced pressure. The product was purified by chromatography column. 16.1 g compound 2 was obtained (92% yield).

### Preparation of Compound 3

A mixture of compound 2 (22 g, 1 eq.), 1-Benzyloxy-4-chloromethyl-benzene (17.05 g, 1.2 eq.) and Cs₂CO₃ (32.6 g, 1.5 eq.) in CH₃CN (500 mL) was heated to reflux for about 12 h. After cooling the solid was filtered off, the solvent was removed under reduced pressure. The resulting crude was purified by chromatography column to give 14.7 g of compound 3 (43% yield).

### Preparation of Compound 4

Compound 3 (25 g) was hydrogenated in MeOH/EtOAc (8:1) in the presence of Pd/C (5.0 g) in an initial H₂ of 50 Psi at 40-50° C. for about 4 h. Then the catalyst was filtered off, the solvent was removed in vacuum. The resulting crude was purified by chromatography column to give 14 g of compound 4 (67.1% yield).

### Preparation of Compound 5

A mixture of compound 4 (3.0 g, 1 eq.), 1-bromo-3,5-dinitro-benzene (4.2 g, 2.5 eq.), Pd(dppf)Cl₂ (984 mg, 0.2 eq.), anhydrous K₃PO₄ (3.65 g, 2.5 eq.) in N,N-dimethylformamide was heated to 80° C. under nitrogen protection, the reaction was detected by TLC, After completion of the reaction, the solid was filtered off, the solvent was removed under reduced pressure. The product was purified by pre-HPLC to give 1.7 g of compound 5 (53% yield).

### Preparation of Compound 6

A mixture of compound 5 (1 eq.), boronic acid (2-3 eq.), Cu(OAc)₂ (1.3 eq.), pyridine (5 eq.), pyridine N-Oxide and molecular sieves 4 A in dichloromethane (5 mL/1 mmol compound 5) was stirred for 14-36 h at room temperature opened to the air. The reaction was monitored by TLC and LC-MS. After completion of the reaction, aqueous sodium bicarbonate was added to the reaction mixture. The organic layer was separated and the aqueous layer was extracted by CH₂Cl₂. The combined organic layer was washed with brine, dried over Na₂SO₄ and concentrated. The crude was purified by chromatography column to give the pure compound 6.

### Preparation of Compound 7

Compound 6 was dissolved in ethanol and dichloromethane (1:1), then concentrated hydrochloric acid (3 mL/1 mmol compound 6) and SnCl₂ (5.6 eq.) was added, the resulting mixture was heated to 50° C. for 2 h. The reaction was monitored by TLC. After completion of the reaction, aqueous sodium bicarbonate was added to the reaction mixture. The organic layer was separated and the aqueous layer was extracted by EtOAc. The combined organic layer was washed with brine, dried over Na₂SO₄ and concentrated. The crude was purified by pre-HPLC to give final product (basic condition).

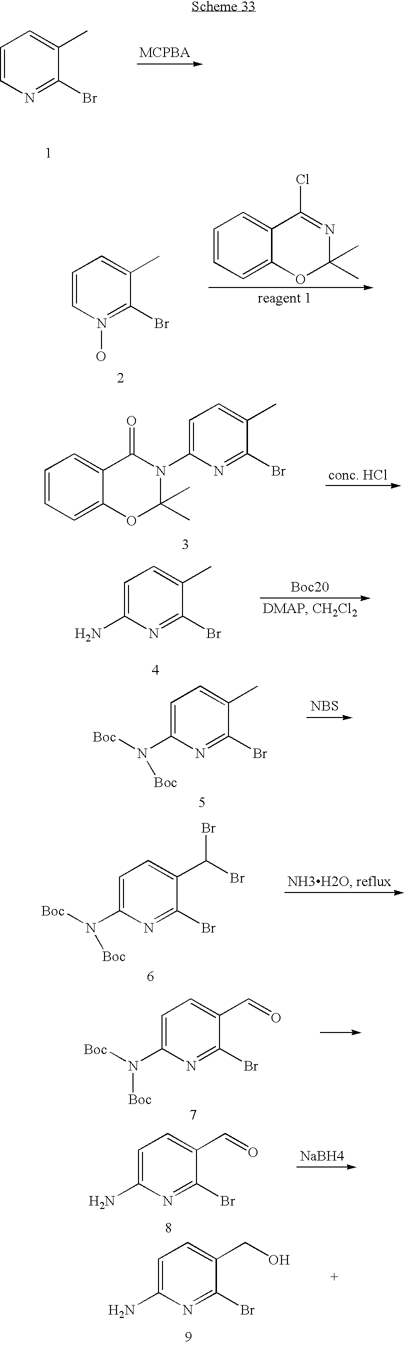

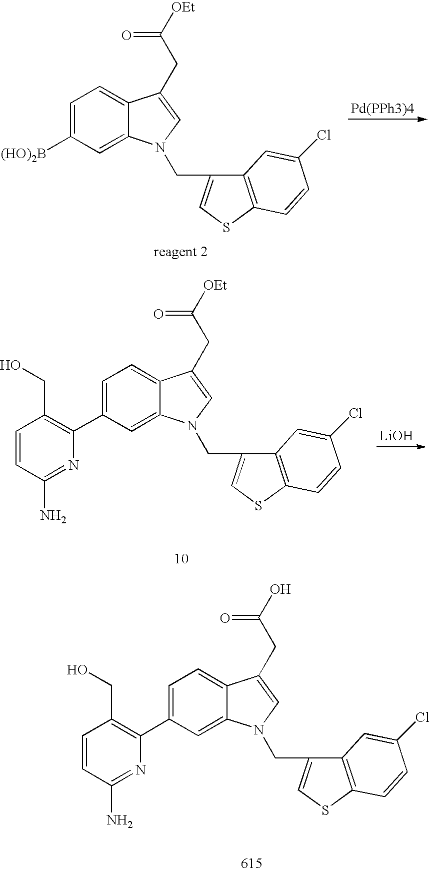

### Preparation of Compound 2

A mixture of compound 1 (20 g, 1 eq.), meta-chloroperoybenzoic acid (40.2 g, 2 eq.) in dichloromethane was stirred at room temperature, the reaction was monitored by TLC. After completion of the reaction, the mixture was quenched by aqueous sodium hyposulfite. then the solvent was removed under reduced pressure. The resulting crude was purified by chromatography column to give 19.3 g of compound 2 (88% yield)

### Preparation of Compound 3

A mixture of compound 2 (33.6 g, 2 eq.), reagent 1 (17.7 g, 1 eq.) in dichloromethane was heated to reflux. the reaction was monitored by TLC. After completion of the reaction, the solvent was removed under reduced pressure. The resulting crude was purified by chromatography column to give 15.7 g of compound 3 (50.6% yield)

### Preparation of Compound 4

The compound 3 (5.0 g) was dissolved in concentrated 50 mL HCl, the resulting mixture was heated to reflux for 12 h, the reaction was monitored by TLC and LCMS. After completion of the reaction, the mixture was concentrated, neutralized by aqueous NaHCO₃, extracted by CHCl₃ for 3 times. The combined organic layer was washed with brine, dried by anhydrous Na₂SO₄ and concentrated to give 2.1 g of compound 4. The product was used in next step without further purification (50.6% yield).

### Preparation of Compound 5

Compound 4 (2.0 g), di-tert-butyl dicarbonate (9.34 g, 4 eq.) and dimethyl-pyridin-4-yl-amine (1.31 g, 1 eq.) were dissolved in dichloromethane, the resulting mixture was stirred at room temperature. the reaction was monitored by TLC. After completion of the reaction, the solvent was removed under reduced pressure. The resulting crude was purified by chromatography column to give 2.4 g of compound 5 (58.5% yield).

### Preparation of Compound 6

Compound 5 (100 mg, 1 eq.) was dissolved in CCl₄, then N-bromosuccinimide (NBS, 48.1 g, 1.1 eq.) and AIBN (4.58 mg, 0.1 eq.) was added. the resulting mixture was heated to reflux for 1 h, then another 4.0 eq. NBS and 0.4 eq. AIBN was added, the reaction mixture was refluxed overnight. the reaction was monitored by TLC. After completion of the reaction, the solvent was removed under reduced pressure to give compound 6 (110 mg). The final product was used in next step without further purification (78.6% yield).

### Preparation of Compound 7

Compound 6 (2.5 g) was dissolved in 25 mL ethanol and 10 mL NH₃—H₂O, then the resulting mixture was heated to reflux. The reaction was monitored by TLC. After completion of the reaction, the mixture was poured into 1N HCl with stirring, aqueous NaHCO₃ was added to adjusted the pH=7, then the solution was extracted by ethyl acetate. The combined organic layer was washed with brine, dried by anhydrous Na₂SO₄ and concentrated to give compound 7. The final product was used in next step without further purification.

### Preparation of Compound 8

Compound 7 was dissolved in hydrochloric gas (methanol), and stirred at room temperature, the reaction was monitored by TLC, After completion of the reaction, aqueous NaHCO₃ was added to the reaction mixture adjusted the pH>7, then solution was extracted by ethyl acetate, The combined organic layer was washed with brine, dried over Na₂SO₄ and concentrated to give compound 8, The final product was used in next step without further purification.

### Preparation of Compound 9

Compound 8 (400 mg, 1 eq.) was dissolved in THF-MeOH (4:1) 10 mL. The mixture was cooled to 0° C., and NaBH₄ (76 mg, 1 eq.) was added. Then the reaction mixture was warmed to room temperature and stirred for another 2 h. the reaction was monitored by TLC, After completion of the reaction, the mixture was poured into aqueous NH₄OAc, extracted by ethyl acetate, The combined organic layer was washed with brine, dried over Na₂SO₄ and concentrated. The resulting crude was purified by chromatography column to give 320 mg of compound 9 (79% yield).

### Preparation of Compound 10

A mixture of compound 9 (200 mg, 1 eq.), reagent 2 (845.8 g, 2 eq.), Pd(PPh₃)₄ (115.3 mg, 0.1 eq.), aqueous Na₂CO₃ (264.9 g, 2.5 eq.) in 1:1 toluene-EtOH was heated to reflux under nitrogen protection, the reaction was detected by TLC, After completion of the reaction, the solid was filtered off, the solvent was removed under reduced pressure. The product was purified by chromatography column to give 500 mg of compound 10 (containing PPh₃).

### Preparation of Compound 615

To a solution of compound 10 (500 mg, 1 eq.) in THF-H₂O (3:1, 4 ml) was added LiOH (5 eq.) at room temperature. The mixture was heated to 70-80° C. overnight. After cooling to the room temperature, the reaction mixture was acidified with 2M HCl to pH 5-6. The aqueous layer was extracted with ethyl acetate. The combined organic layer was washed with brine, dried and concentrated. The resulting crude was purified by chromatography column to give 200 mg of pure compound 615.

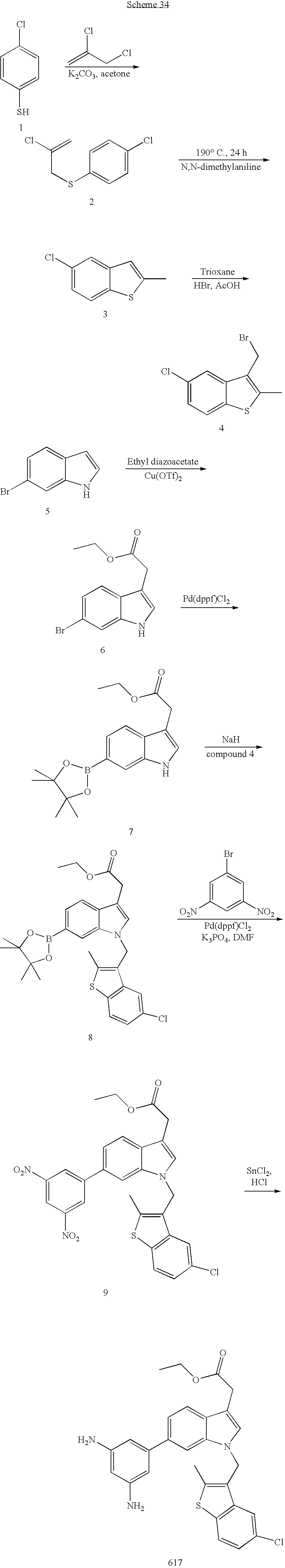

### Preparation of Compound 2

To a solution of 4-chlorothiophenol (10.0 g, 69.4 mmol) in acetone (200 mL) was added K₂CO₃ (19.2 g, 138.8 mmol) followed by 2,3-dichloropropene (7.6 g, 69.4 mmol). The resulting solution was heated to 60° C. for 1 h, then allowed to cool to room temperature. The acetone was removed under reduced pressure to give the crude residue, which was dissolved in ethyl acetate (100 mL) and washed with water (100 mL). The aqueous layer was then extracted with ethyl acetate (6×20 mL). The combined organic layers were dried, filtered and evaporated to dryness to give the compound 2 (14.0 g, 92%).

### Preparation of Compound 3

The compound 2 (6.0 g, 27.3 mmol) was dissolved in N,N-dimethylaniline (60 mL) and heated to 190° C. for 24 h, then allowed to cool to room temperature. 300 mL of t-butyl methyl ether (TBME) was added to the reaction mixture, which was then washed with 2M HCl (300 mL). The organic layer was dried (Na₂SO₄), filtered and evaporated to give the crude residue. The crude was purified by chromatography column (hexane) to give compound 3 (3.5 g, 70%).

### Preparation of Compound 4

To a solution of AcOH (60 drops) in HBr (48% in H₂O) (40 mL) was added 2-methyl-5-chloro benzothiophene (4 g, 21.8 mmol), followed by trioxane (3.5 g, 39 mmol) and cetyl trimethylammonium chloride (160 mg, 0.4 mmol). The resulting suspension was stirred for 12 h at room temperature, then the reaction mixture was diluted with water (50 mL) and filtered. The residue was washed with water (2×50 mL), air dried to give the title compound 4 as a white solid (5.8 g, 96%).

### Preparation of Compound 6

Cu(OTf)₂ (923 mg, 2.55 mmol) was added to a solution of 6-bromoindole (5.0 g, 25.5 mmol) in dichloromethane (50 mL) under nitrogen, and the resulting suspension was cooled to 0° C., ethyl diazoacetate in dichloromethane (20 mL) was added slowly. After that the reaction was stirred at room temperature for 12 h, the mixture was washed by 60 mL water, the organic layer was separated, dried, filtered and evaporated to give the crude residue, the crude was purified by HPLC-chromatography column to give compound 6 (1.9 g., 26%).

### Preparation of Compound 7

A mixture of compound 1 (15 g, 1 eq.), Bis(pinacolato)diboron (29.9 g, 2 eq.), KOAc (18 g, 3.4 eq.), Pd(dppf)Cl₂ (3.81 g, 0.1 eq.) in N,N-dimethylformamide was heated to 80° C. under nitrogen protection, the reaction was detected by TLC, After completion of the reaction, the solid was filtered off, the solvent was removed under reduced pressure. The crude was purified by chromatography column to give compound 7 (16.1 g, 92. %).

### Preparation of Compound 8

To a suspension of NaH (60% in mineral oil) (800 mg, 20.0 mmol) in DMF (40 mL) at 0° C. was added a solution of compound 7 (6.0 g, 18.2 mmol) in DMF (40 mL). The resulting solution was stirred for 10 min at 0° C., then a solution of the benzothiophene (5.8 g, 21.1 mmol) in DMF (40 mL) was added. The resulting solution was stirred at 0° C. for 3 h, The reaction was traced by TLC, After completion of the reaction, the reaction was diluted with EtOAc (400 mL) and 2M HCl (100 mL). The organic layer was separated and washed with brine, then dried (Na₂SO₄), filtered and evaporated to give the crude residue. The crude was purified by HPLC-chromatography column to give compound 8 (4.0 g, 42%).

### Preparation of Compound 9

To a solution of the compound 8 (2.0 g, 3.8 mmol) in DMF (20 mL) was added palladium (II) dichloridediphenylphosphinoferrocene (1.0 g, 50% by weight) followed by potassium phosphate (2.5 g, 11.8 mmol) and the 1-bromo-3,5-dinitro-benzene (1.9 g, 7.76 mmol). The resulting solution was heated to 75° C. for 2 h, then allowed to cool to room temperature, and diluted with EtOAc (200 mL). This solution was washed with 1M HCl (100 mL), then the aqueous layer was extracted into EtOAc (400 mL). The combined organic layers were washed by brine, dried (Na₂SO₄), filtered and evaporated to give the crude residue. The crude was purified by chromatography column (10% dichloromethane/petroleum) to give the title compound 9 as a bright yellow solid (0.9 g, 43%).

### Preparation of Compound 617

To a solution of the compound 9 (2.0 g, 3.55 mmol) in EtOH (100 mL) and EtOAc (50 mL) was added conc HCl (10.5 mL) and SnCl₂ (6.7 g, 35.5 mmol). The resulting suspension was stirred at 50° C. for 3 hours, After completion of the reaction, the reaction mixture was cooled to room temperature, Na₂CO₃ was added to neutralize the acid, the solid was filtered through celite and the filtrate was evaporated. The crude residue was purified by HPLC-chromatography column (basic condition) to give compound 617 (1.0 g, 56%).

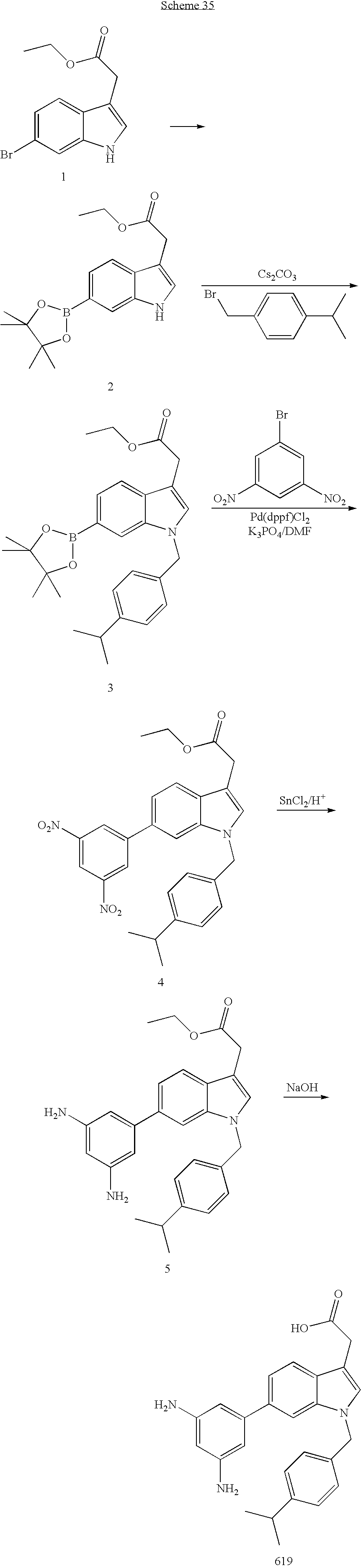

### Preparation of Compound 2

A mixture of compound 1 (15 g, 1 eq.), Bis(pinacolato)diboron (29.9 g, 2 eq.), KOAc (18 g, 3.4 eq.), Pd(dppf)Cl₂ (3.81 g, 0.1 eq.) in N,N-Dimethylformamide was heated to 80° C. under nitrogen protection, the reaction was detected by TLC, After completion of the reaction, the solid was filtered off, the solvent was removed under reduced pressure. The product was purified by chromatography column to give 16.1 g of compound 2 (92% yield).

### Preparation of Compound 3

A mixture of compound 2 (2.0 g, 1 eq.), 1-Bromomethyl-4-isopropyl-benzen (1.3 g, 1.2 eq.) and Cs₂CO₃ (3.0 g, 1.5 eq.) in CH₃CN (30 mL) was heated to reflux for about 12 h. After cooling the solid was filtered off, the solvent was removed under reduced pressure. The resulting crude was purified by chromatography column to give 1.0 g of compound 3 (35% yield).

### Preparation of Compound 4

A mixture of compound 3 (1.0 g, 1 eq.), 1-Bromo-3,5-dinitro-benzene (1.3 g, 2.5 eq.), Pd(dppf)Cl₂ (500 mg), anhydrous K₃PO₄ (1.1 g, 2.5 eq.) in N,N-Dimethylformamide was heated to 80° C. under nitrogen protection, the reaction was detected by TLC, After completion of the reaction, the solid was filtered off, the solvent was removed under reduced pressure. The product was purified by pre-HPLC to give 0.5 g of compound 4 (53% yield).

### Preparation of Compound 5

Compound 5 was dissolved in ethanol and dichloromethane (1:1, 5 mL/mmol compound 5), then concentrated hydrochloric acid (3 mL/1 mmol compound 5) and SnCl₂ (5.6 eq.) was added, the resulting mixture was heated to 50° C. for 2 h. The reaction was monitored by TLC. After completion of the reaction, sodium bicarbonate was added to the reaction mixture to neutralize the acid. The solid was filtered off, the solvent was concentrated. The crude was purified by pre-HPLC (basic condition).

### Preparation of Compound 619

To a solution of compound 5 (1 eq.) in EtOH—H₂O (3:1, 4 ml) was added NaOH (5 eq.) at room temperature. The mixture was heated to 70-80° C. overnight. After cooling the reaction mixture was acidified with 2M HCl to pH 2-3. The aqueous was extracted with EtOAc. The combined organic layer was washed with brine, dried and concentrated. The resulting crude was purified by pre-TLC to give the pure compound 619.

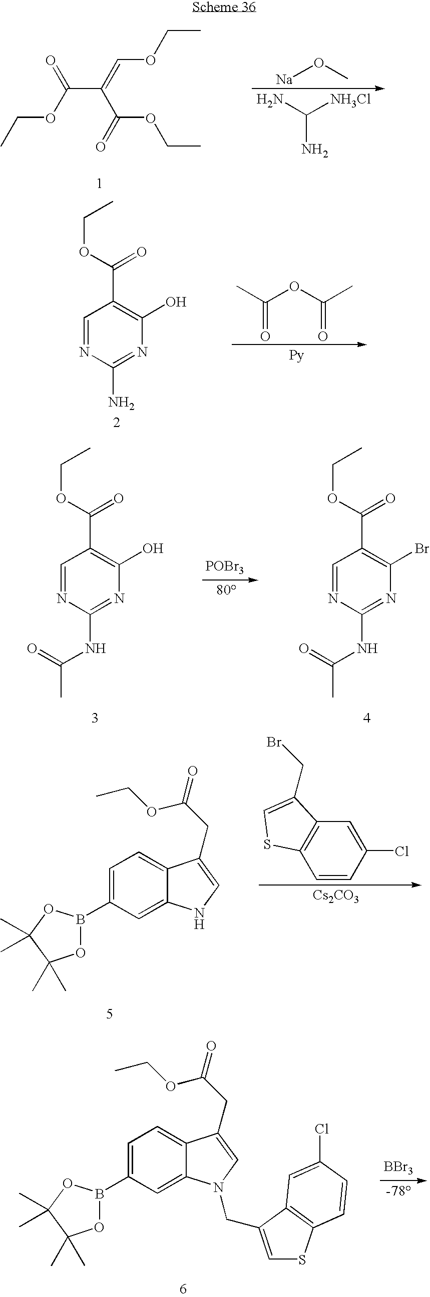

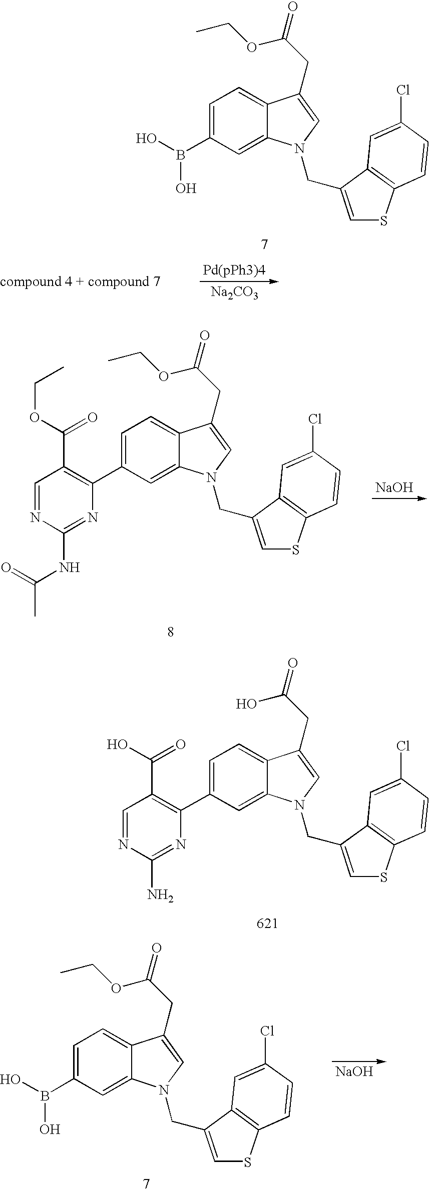

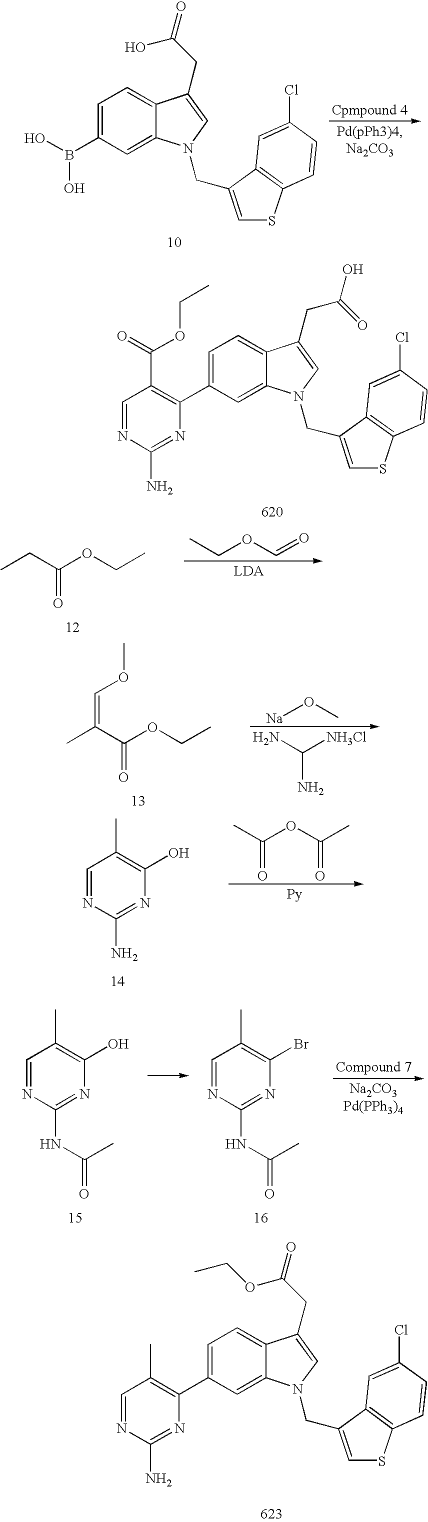

### Preparation of Compound 2

A mixture of compound 1 (21 g, 1 eq.), guanidine (9.7 g, 1 eq.), Sodium methanolate (5.4 g, 1 eq.), in methanol was heated to 60° C., the reaction was detected by TLC, after completion of the reaction, the solvent was neutralized by HCl. The precipitation was found, then product was filtrated, dried to give 16 g of compound 2 (87% yield).

### Preparation of Compound 3

A mixture of compound 2 (18 g, 1 eq.), acetic anhydride (20 g, 2 eq.) and Py (10 g) was heated to reflux for about 3 h. After cooling the solid was filtered to give 20 g of crude compound 3 (90% yield).

### Preparation of Compound 4

A mixture of compound 3 (5 g), in POBr₃ (20 g) was heated to 80° C. under nitrogen protection, the reaction was detected by TLC, After completion of the reaction, mixture was poured in ice-water, then the solvent was neutralized by Na₂CO₃, then extract by EtOAc, The product was purified by chromatography column to give 2.5 g of compound 4 (53% yield).

### Preparation of Compound 6

Compound 5 (2 g, 1 eq.), 2-(bromomethyl)-5-chlorobenzo[b]thiophene (4.0 g, 1 eq.) and Cs₂CO₃ (6.43 g, 1.5 eq.) in CH₃CN (30 mL) was heated to reflux for about 5 h. After cooling the solid was filtered off, the solvent was removed under reduced pressure. The resulting crude was purified by chromatography column to give 3.0 g of compound 6 (56% yield).

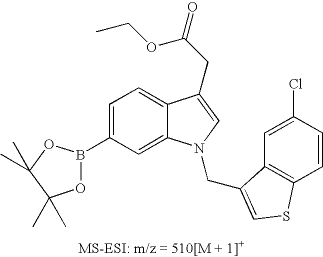

### Preparation of Compound 7

A mixture of compound 6 (3 g), in anhydrous DCM (5 mL), then the temperature was kept at −78° C. BBr₃ (2 g) was added under nitrogen protection, the reaction was detected by TLC, After completion of the reaction, mixture was poured in ice-water, then the solvent was neutralized by Na₂CO₃, then extract by EtOAc, The product was purified by chromatography column to give 2.5 g of compound 7 (50% yield).

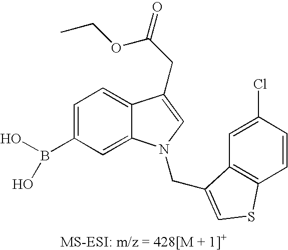

### Preparation of Compound 8

A mixture of compound 4 (286 mg, 1 mmol), compound 7 (427 mg, 1 mmol), Pd(PPh₃)₄ (10 mg, 0.1 mmol) and Na₂CO₃ (50 mg) in DMF was heated to 60° C., the reaction was detected by TLC. After the reaction was completed, the mixture was filtrated and extracted by EtOAc, then purified by Prep-HPLC to give 20 mg of compound 8 (10% yield).

### Preparation of Compound 621

A mixture of compound 8 (50 mg), and NaOH (30 mg) in EtOH was refluxed, after completion of the reaction, the reaction mixture was purified by prep-HPLC to give 20 mg of compound 621 (50%).

### Preparation of Compound 10

A mixture of compound 7 (200 mg), and NaOH (100 mg) was refluxed in EtOH, after completion of the reaction, the reaction mixture was neutralized by HCl, the reaction mixture was purified by prep-HPLC to give 180 mg of compound 10 (90%).

### Preparation of Compound 620

A mixture of compound 4 (286 mg, 1 mmol), compound 10 (400 mg, 1 mmol), Pd(PPh₃)₄ (10 mg, 0.1 mmol) and Na₂CO₃ (50 mg) in DMF was heated to 60° C., and the reaction was detected by TLC. After the completion of the reaction, the mixture was filtrated and extracted by EtOAc, then purified by Prep-HPLC to give 50 mg of compound 620 (30% yield).

### Preparation of Compound 13

A mixture of diisopropylamine (22 ml) and THF (100 ml) was kept at −78° C., then the n-butyllithium (1.6M, 48 mL) was added. After 3 h, a mixture of compound 12 (5.6 mL), ethyl formate (4.8 mL), and K₂CO₃ (13 g) was added. Then acetone (30 mL) was added, and the reaction mixture was poured into water and extracted by EtOAc. The EtOAc layer was concentrated and purified by chromatography column to give 3 g of compound 13 (50% yield).

### Preparation of Compound 14

A mixture of compound 13 (3 g, 1 eq.), guanidine (9.7 g, 1 eq.), sodium methanolate (5.4 g, 1 eq.) in methanol was heated to 60° C., and the reaction was detected by TLC. After the completion of the reaction, the solvent was neutralized by HCl. The precipitation was filtered, then dried to give 16 g of compound 14 (87% yield).

### Preparation of Compound 15

A mixture of compound 14 (1 g, 1 eq.), acetic anhydride (5 g.) and pyridine (3 g) was heated to reflux for about 3 h. After cooling the reaction mixture was concentrated, the crude was purified by chromatography column to give 400 mg of compound 3 (30% yield).

### Preparation of Compound 16

A mixture of compound 15 (400 mg), in POBr₃ (3 g) was heated to 80° C. under nitrogen protection, the reaction was detected by TLC. After the completion of the reaction, the mixture was poured in ice-water, then the solvent was neutralized by Na₂CO₃ and extract by EA, The product was purified by chromatography column to give 200 g of compound 16 (37% yield).

### Preparation of Compound 623

A mixture of compound 16 (200 mg, 0.86 mmol), compound 7 (427 mg, 1 mmol), Pd(PPh₃)₄ (20 mg, 0.1 mmol) and Na₂CO₃ (50 mg) in DMF was heated to 60° C., and the reaction was detected by TLC. After the reaction, the mixture was filtrated and then extracted by EtOAc, then purified by Prep-HPLC to give 30 mg of compound 623 (6.5% yield).

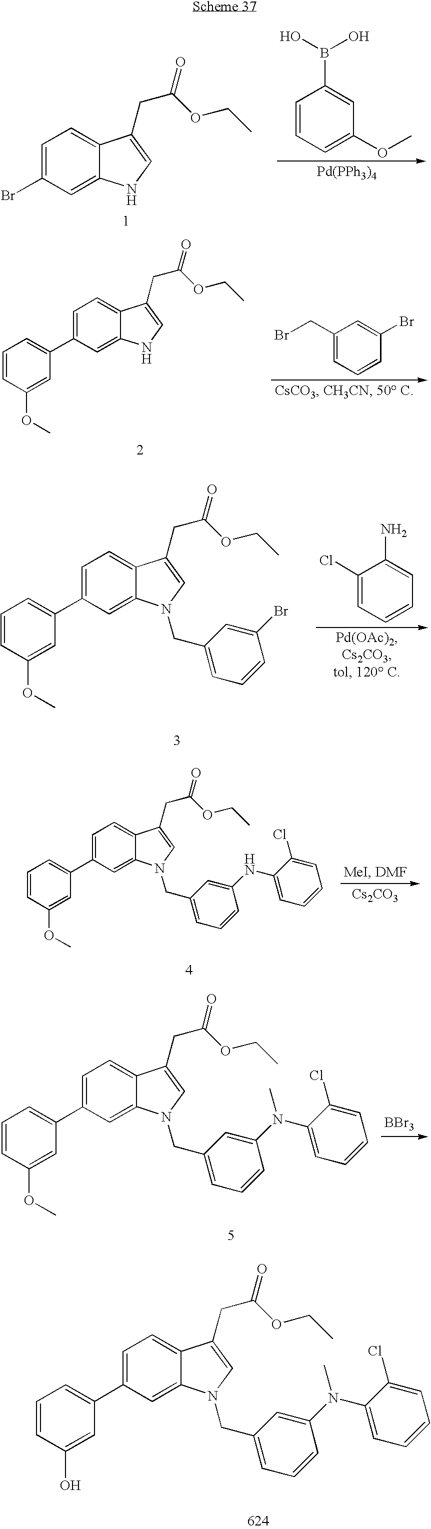

### Preparation of Compound 2

A mixture of compound 1 (4.0 g, 1 eq), (4.3 g, 2 eq), Pd(PPh₃)₄ (1.6 g, 0.1 eq), aqueous Na₂CO₃ (3.7 g, 2.5 eq) in 1:1 toluene-ethanol was heated to reflux under nitrogen protection, the reaction was detected by TLC. After the completion of the reaction, the solid was filtered off, the solvent was removed under reduced pressure. The product was purified by column and compound 2 was obtained in 3.6 g (63% yield).

### Preparation of Compound 3

A mixture of compound 2 (1 eq.), 1-bromomethyl-4-bromobenzene (1 eq.) and Cs₂CO₃ (1.5 eq.) in CH₃CN (5 mL/l) was heated to reflux for about 12 h. After cooling the solid was filtered off, the solvent was removed under reduced pressure. The resulting crude was purified by chromatography column to give compound 3.

### Preparation of Compound 4

A mixture of compound 3 (1 eq.), phenyl-amine (1.5 eq.) and cesium carbonate (2 eq.) in toluene (5 mL/1 mmol reagent 2) was stirred at 110° C. in nitrogen atmosphere, then palladium acetate and xanphos were added. The mixture was stirred at the temperature overnight and monitored by TLC. Compound 4 was isolated by pre-TLC (PE/EA=5:1)

### Preparation of Compound 5

A mixture of compound 4 (1 eq.), Cs₂CO₃ and iodomethane (3 eq.) in DMF was stirred at room temperature over night. The reaction was monitored by TLC. When the starting material was consumed, the mixture was extracted with EtOAc, washed with saturated brine, dried over anhydrous sodium sulfate, concentrated in vacuum and the target product was isolated by pre-TLC. (PE/EA=5:1)

### Preparation of Compound 624

Compound 5 (1 eq.) was dissolved in anhydrous dichloromethane (5 mL/mmol compound 5). Boron tribromide (3 eq.) was added to this mixture under ice-cooling and the whole was stirred at room temperature for 14 hours. Then 1N aqueous sodium hydroxide was added to the reaction mixture, the whole was extracted with ethyl acetate. The organic layer was washed with brine and then dried over anhydrous Na₂SO₄. The solvent was removed under reduced pressure. The crude was purified by chromatography column to give compound 624.

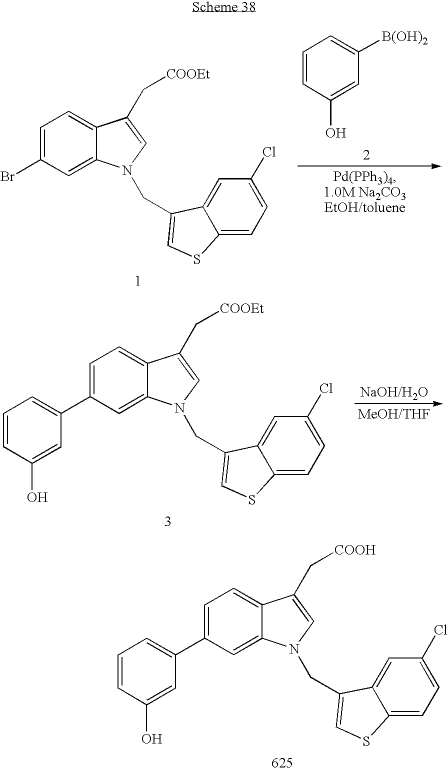

### Synthesis of Compound 625

A mixture of compound 1 (139 mg, 0.3 mmol), compound 2 (83 mg, 0.6 mmol), Pd(PPh₃)₄ (17.4 mg, 0.015 mmol) and sodium carbonate (1.0 M, 0.75 mL) was stirred at 82° C. under argon overnight. The mixture was diluted with ethyl acetate, washed with brine four times, dried (Na₂SO₄) and concentrated. Chromatography on silica gel with 1-5% EtOAc in DCM gave 84 mg of compound 3 as colorless solid.

Compound 3 (71 mg) was dissolved in MeOH (4 mL) and THF (1 mL) and sodium hydroxide (1.0 M, 0.6 mL) and water (1.9 mL) were added. The resulting mixture was stirred at room temperature overnight, acidified with 2N HCl to pH 2, and then concentrated. Water was added and the resulting precipitate was filtered and washed with water three times. Recrystallization from DCM gave 57 mg of compound 625 as faint-amber solid; ¹H NMR (DMSO-d₆) δ 3.67 (s, 2H), 5.72 (s, 2H), 6.7 (m, 1H), 7.01 (t, J=2.5 Hz, 1H), 7.1 (m, 1H), 7.22 (t, J=9.5 Hz, 1H), 7.28 (dd, J=10.5, 2.0 Hz, 1H), 7.41 (dd, J=10.5, 2.5 Hz, 1H), 7.43 (s, 1H), 7.56 (s, 1H), 7.57 (d, J=10 Hz, 1H), 7.8 (m, 1H), 8.01 (d, J=2.5 Hz, 1H), 8.03 (d, J=11.0 Hz, 1H), 9.43 (s, 1H), 12.24 (s, 1H).

### Synthesis of Compound 626

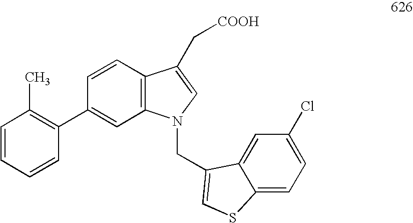

By a similar procedure as described for compound 625, compounds 626 (30 mg) was prepared from compound 1 (46 mg, 0.1 mmol) and 2-methylbenzeneboronic acid (27 mg, 0.2 mmol); ¹H NMR (DMSO-d₆) δ 2.21 (s, 3H), 3.67 (s, 2H), 5.67 (s, 2H), 7.00 (dd, J=10.0 Hz, 1H), 7.19-7.29 (m, 4H), 7.41 (dd, 1H), 7.44 (s, 1H), 7.55 (d, J=10.0 Hz, 1H), 7.6 (m, 1H), 7.68 (s, 1H), 7.95 (d, J=2.5 Hz, 1H), 8.02 (d, J=10.5 Hz, 1H), 12.24 (s, 1H).

### Synthesis of Compound 627

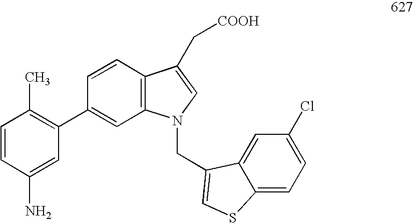

By a similar procedure as described for compound 625, compounds 627 (52 mg) was prepared from compound 1 (46 mg, 0.1 mmol) and 2-methyl-5-aminobenzeneboronic acid pinacol ester (28 mg 0.12 mmol); ¹H NMR (DMSO-d₆) δ 2.25 (s, 3H), 3.68 (s, 2H), 5.67 (s, 2H), 6.95-7.05 (m, 3H), 7.26 (d, J=9.5 Hz, 4H), 7.40 (dd, J=10.5, 2.0 Hz, 1H), 7.46 (s, 1H), 7.55 (s, 1H), 7.58 (d, J=10.0 Hz, 1H), 7.64 (s, 1H), 7.93 (d, J=2.5 Hz, 1H), 8.03 (d, J=10.5 Hz, 1H), 9.9 (bs, 2H), 12.2 (bs, 1H).

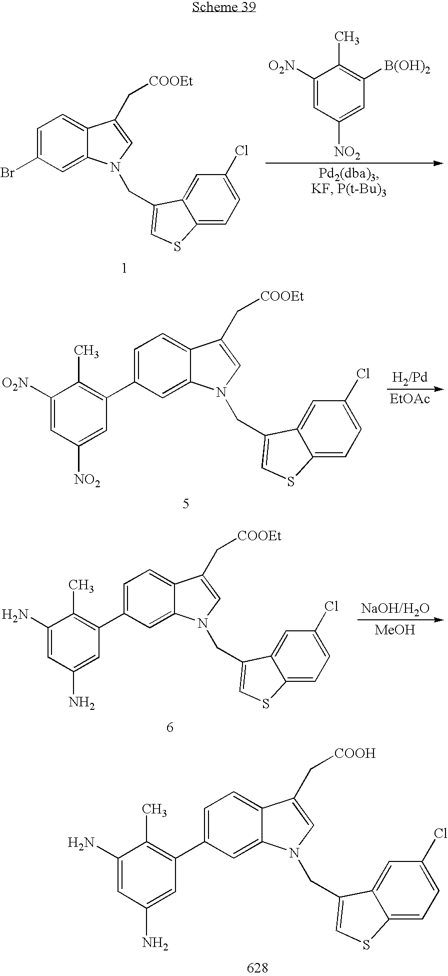

### Synthesis of Compound 628

A reaction mixture of compound 1 (92 mg, 0.2 mmol), 2-methyl-3,5-dinitrobenzeneboronic acid (68 mg 0.3 mmol), potassium fluoride (58 mg, 1.0 mmol), Pd₂(dba)₃ (8 mg) and tri(t-butyl)phosphine (0.2M in THF, 0.02 mL, 0.004 mmol) in THF (1 mL) under argon was stirred at room temperature overnight. The mixture was diluted with ethyl acetate, filtered, washed with brine three times, dried (Na₂SO₄), and concentrated. Chromatography on silica gel with DCM/hexanes (1:3 to 3:1) gave 98 mg of compound 5 as yellow solid.

Compound 5 (100 mg) in EtOAc (40 mL) was reduced by catalytic hydrogenation over 10% Pd/C with hydrogen gas (55 psi) during 4 h at rt. Chromatography on silica gel with 1.5-3% MeOH in DCM gave 55 mg of compound 6.

A solution of compound 6 (52 mg) in MeOH (3 mL), water (0.5 mL) and 2 N NaOH (0.2 mL) was stirred at room temperature under argon for 2 days. The solution was concentrated, acidified with 2 N HCl to pH 2, diluted with water, and concentrated to about 1 mL. Precipitate was filtered, washed thoroughly with water, and dried under vacuum to give 52 mg of compound 628 as faint-yellow solid; ¹H NMR (DMSO-d₆) δ 1.84 (s, 3H), 3.67 (s, 2H), 5.65 (s, 2H), 6.14 (s, 1H), 6.30 (s, 1H), 6.90 (dd, J=10.0, 1.5 Hz, 1H), 7.40 (dd, J=11.0, 2.0 Hz, 1H), 7.42 (s, 2H), 7.53 (d, J=10.0 Hz, 1H), 7.60 (s, 1H), 7.92 (d, J=2.5 Hz, 1H), 8.03 (d, J=10.5 Hz, 1H).

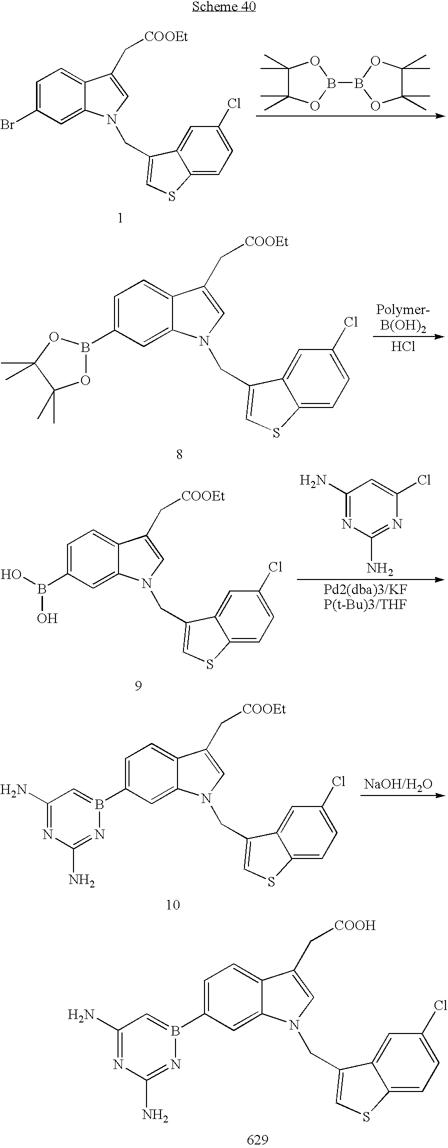

### Synthesis of Compound 629

A mixture of compound 1 (2.16 g, 4.68 mmol), KOAc (1.59 g, 16.2 mmol), bis(pinacolato)diboron (1.37 g, 5.38 mmol) and PdCl₂(dppf) (191 mg, 0.23 mmol) was stirred at 80° C. overnight. The mixture was diluted with EtOAc, filtered on celite cake, washed thoroughly with EtOAc. The filtrate was washed with brine four times, dried (Na₂SO₄), and concentrated. Chromatography on silica gel with DCM-hexanes (2:1) gave 1.84 g of compound 8 as foam.

A mixture of compound 8 (0.82 g, 1.6 mmol) and polymer-bound boronic acid (1-2 mmol/g, 8.0 g) in acetonitrile (35 mL) and 1.0 N HCL (3.5 mL) was stirred at room temperature for 24 h. Polymer reagent was filtered and the filtrate concentrated to dryness. Chromatography on silica gel with 1-2% MeOH in DCM gave 0.41 g of compound 9 as white solid.

A mixture of compound 9 (43 mg, 0.1 mmol), 4-chloro-2,6-diaminopyrimidine (22 mg, 0.15 mmol), KF (20 mg, 0.33 mmol), Pd₂(dba)₃ (9.2 mg, 0.01 mmol), and P(t-Bu)₃ (0.2 M in DMF, 0.09 mL) was stirred at 80° C. under argon for 3 days. The mixture was diluted with DCM, filtered, and concentrated. Chromatography on silica gel with 5-8% MeOH in DCM gave 19 mg of compound 10.

A solution of compound 10 (19 mg) in MeOH (2 mL) and 1 N NaOH (0.5 mL) was stirred overnight, diluted with more water, and concentrated to remove MeOH. The aqueous solution was acidified with AcOH and resulting precipitate filtered and washed thoroughly with water to give 8.1 mg of compound 629 as faint-yellow solid; ¹H NMR (DMSO-d₆) δ 3.68 (s, 2H), 5.71 (s, 2H), 5.84 (bs, 2H), 6.22 (bs, 2H), 6.25 (s, 1H), 7.33 (s, 1H), 7.43 (dd, J=10.5, 2.0 Hz, 1H), 7.45 (s, 1H), 7.57 (d, J=10.5 Hz, 1H), 7.62 (dd, J=10.5, 2.0 Hz, 1H), 8.00 (d, J=2.5 Hz, 1H), 8.05 (d, J=10.5 Hz, 1H), 8.10 (s, 1H), 12.2 (bs, 1H).

### Synthesis of Compound 630

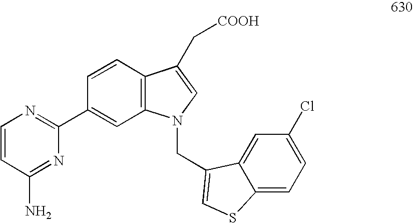

By a similar procedure as described for compound 629, compound 630 (27 mg) was prepared from compound 9 (86 mg, 0.2 mmol) and 4-amino-2-chloropyrimidine (38.8 mg 0.3 mmol); ¹H NMR (TEA salt, DMSO-d₆) δ 0.90 (t, J=9.0 Hz, 7.1H), 2.43 (q, J=9.0 Hz, 4.8H), 3.62 (s, 2H), 5.68 (s, 2H), 6.26 (d, J=7.0 Hz, 1H), 6.74 (bs, 2H), 7.23 (s, 1H), 7.40 (dd, J=10.5, 2.0 Hz, 1H), 7.43 (s, 1H), 7.54 (d, J=10.5 Hz, 1H), 7.98 (d, J=2.5 Hz, 1H), 8.01 (d, J=10.5 Hz, 1H), 8.05 (dd, J=10.5, 2.0 Hz, 1H), 8.09 (d, J=7.5 Hz, 1H), 8.39 (s, 1H), 12.2 (bs, 1H).

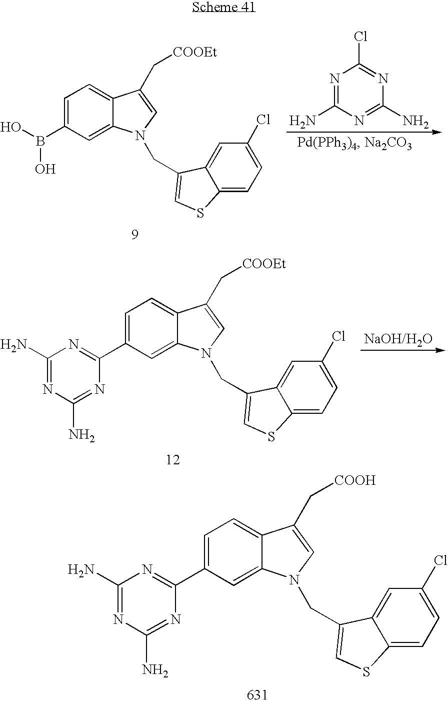

### Synthesis of Compound 631

A mixture of compound 9 (86 mg, 0.2 mmol), chlorodiaminotriazine (44 mg, 0.3 mmol), sodium carbonate (1 M, 0.5 mL) and Pd(PPh₃)₄ (24 mg, 0.02 mmol) in DMF (1.2 mL) under argon was stirred at 82° C. for 2 days. Solvent was evaporated and the residue extracted with a mixture of MeOH and DCM. Chromatography on silica gel with 5-7% MeOH in DCM gave 47 mg of compound 12, which was hydrolyzed with NaOH/H₂O to give compound 631 (31 mg) as off-white solid; ¹H NMR (DMSO-d₆+D₂O) δ 3.69 (s, 2H), 5.70 (s, 2H), 7.33 (s, 1H), 7.40 (dd, J=11.0, 2.5 Hz, 1H), 7.61 (s, 1H), 7.67 (d, J=11.0 Hz, 1H), 7.88 (d8d, J=10.5 Hz, 1H), 7.95 (d, J=2.5 Hz, 1H), 8.00 (d, J=10.5 Hz, 1H), 8.41 (s, 1H).

### Synthesis of Compound 632

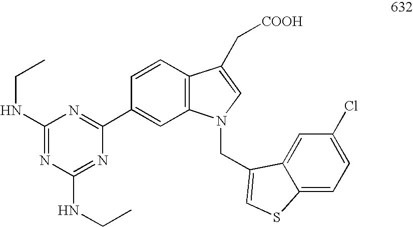

By a similar procedure as described for compound 631, compound 632 (18 mg) was prepared from compound 9 (43 mg, 0.2 mmol) and chlorodi(ethylamino)triazine (30 mg, 0.15 mmol); ¹H NMR (DMSO-d₆+D₂O) δ 1.13 (t, J=9.0 Hz, 6H), 3.37 (q, J=9.0 Hz, 4H), 3.69 (s, 2H), 5.70 (s, 2H), 7.34-7.70 (m, 4H), 7.81-7.95 (m, 2H), 7.99 (d, J=10.5 Hz, 1H), 8.37 (s, 1H).

### Synthesis of Compound 633

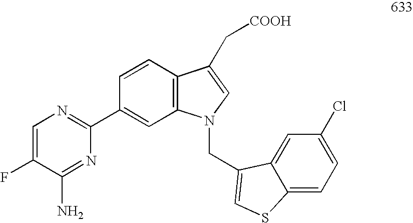

By a similar procedure as described for compound 631, compound 633 (21 mg) as off-white solid was prepared from compound 9 (64 mg, 0.2 mmol) and 4-amino-2-chloro-5-fluoropyrimidine (33 mg 0.225 mmol); ¹H NMR (DMSO-d₆) δ 3.67 (s, 2H), 5.69 (s, 2H), 7.29 (s, 1H), 7.40 (dd, J=10.5, 2.5 Hz, 1H), 7.50 (s, 1H), 7.59 (d, J=10.5 Hz, 1H), 7.95 (dd, J=10.5, 1.5 Hz, 1H), 7.98 (d, J=2.5 Hz, 1H), 8.01 (d, J=10.5 Hz, 1H), 8.25 (m, 1H), 8.35 (s, 1H).

### Synthesis of Compound 634

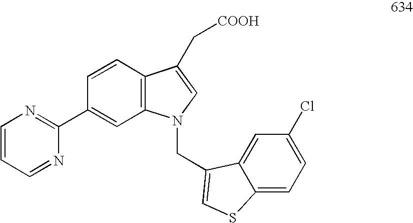

By a similar procedure as described for compound 631, compound 634 (41 mg) as faint-yellow solid was prepared from compound 9 (64 mg, 0.2 mmol) and 2-bromopyrimidine (36 mg 0.225 mmol); ¹H NMR (CDCl₃) δ 3.82 (s, 2H), 5.54 (s, 2H), 7.06 (s, 1H), 7.15 (t, J=6.0 Hz, 1H), 7.18 (s, 1H), 7.32 (dd, J=10.5, 2.5 Hz, 1H), 7.67 (d, J=2.5.0 Hz, 1H), 7.74 (d, J=11.0 Hz, 1H), 8.27 (dd, J=10.5, 1.5 Hz, 2H), 8.55 (s, 1H), 8.80 (d, J=6.0 Hz, 2H).

### Synthesis of Compound 635

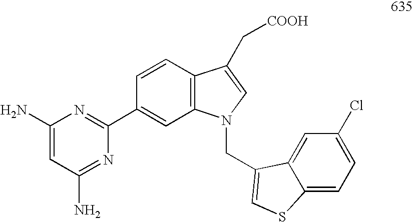

By a similar procedure as described for compound 631, compound 635 (15 mg) as faint-yellow solid was prepared from compound 9 (86 mg, 0.2 mmol) and 2-bromo-4,6-diaminopyrimidine (57 mg 0.3 mmol); ¹H NMR (DMSO-d₆) δ 3.62 (s, 2H), 5.63 (s, 2H), 5.97 (s, 4H), 7.17 (s, 1H), 7.39 (s, 1H), 7.41 (dd, J=11.0, 2.5 Hz, 1H), 7.50 (d, J=10.5 Hz, 1H), 7.99 (d, J=2.5 Hz, 1H), 8.0 (m, 2H), 8.27 (s, 1H), 8.31 (s, 1H), 8.36 (d, J=1.5 Hz, 1H).

### Synthesis of Compound 636

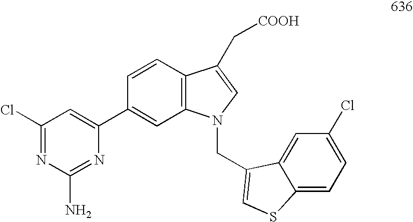

By a similar procedure as described for compound 631, compound 636 (16 mg) as faint-yellow solid was prepared from compound 9 (64 mg, 0.15 mmol) and 2-amino-4,6-dichloropyrimidine (37 mg 0.225 mmol); ¹H NMR (DMSO-d₆) δ 3.65 (s, 2H), 5.73 (s, 2H), 7.31 (s, 1H), 7.39 (dd, J=10.5, 2.0 Hz, 1H), 7.45 (s, 1H), 7.50 (s, 1H), 7.60 (d, J=11.0 Hz, 1H), 7.84 (dd, J=10.5, 1.5 Hz, 1H), 8.01 (m, 2H), 8.36 (d, J=1.5 Hz, 1H).

### Synthesis of Compound 637

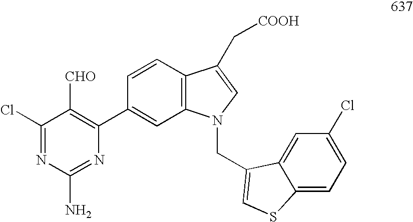

By a similar procedure as described for compound 631, compound 637 (18 mg) as faint-yellow solid was prepared from compound 9 (86 mg, 0.2 mmol) and 2-amino-4,6-dichloro-5-formylpyrimidine (58 mg 0.3 mmol); ¹H NMR (DMSO-d₆) δ 3.69 (s, 2H), 5.70 (s, 2H), 7.33 (dd, J=10.0, 1.5 Hz, 1H), 7.40 (dd, J=10.0, 2.0 Hz, 1H), 7.49 (s, 1H), 7.52 (s, 1H), 7.55 (bs, 2H), 7.60 (d, J=10.0 Hz, 1H), 7.87 (s, 1H), 7.99 (d, J=21.5 Hz, 1H), 8.01 (d, J=10.5 Hz, 1H), 9.65 (s, 1H), 12.2 (bs, 1H).

### Synthesis of Compound 638

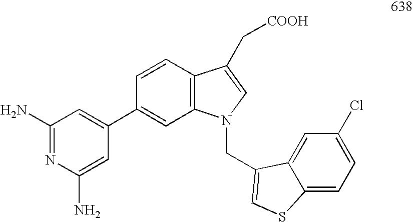

By a similar procedure as described for compound 631, compound 638 (26 mg) as faint-yellow solid was prepared from compound 9 (86 mg, 0.2 mmol) and 4-bromo-2,6-diaminopyridine (56.4 mg 0.3 mmol); ¹H NMR (DMSO-d₆) δ 3.66 (s, 2H), 5.36 (bs, 4H), 5.70 (s, 2H), 5.95 (s, 2H), 7.23 (d, J=10.0 1H), 7.39-7.46 (m, 3H), 7.56 (d, J=10.0 Hz, 1H), 7.72 (s, 1H), 7.98 (s, 1H), 8.03 (d, J=11.0 Hz, 1H), 9.65 (s, 1H), 12.2 (bs, 1H).

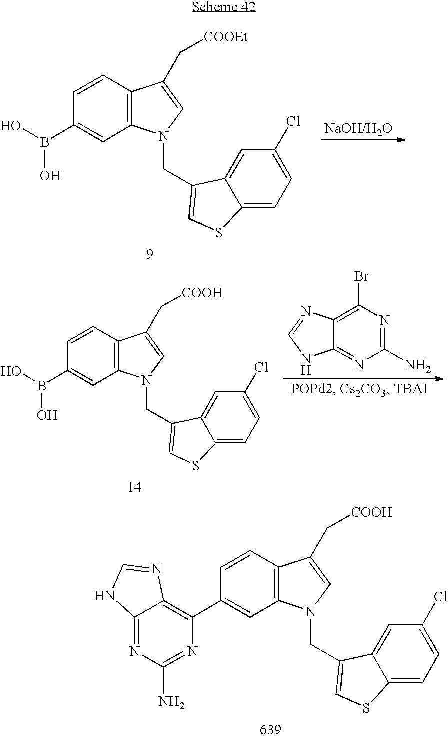

### Synthesis of Compound 639

Sodium hydroxide (1 N, 1 mL) was added to a solution of compound 9 (0.47 g) in a mixture of methanol, 1,4-dioxane and water. The resulting solution was stirred at room temperature for 4 h, acidified with 2 N HCl to pH 3, and concentrated to dryness. Chromatography on silica gel with 3-5% MeOH in DCM gave 0.41 g of compound 14 as colorless foam.

A mixture of compound 14 (55 mg, 0.136 mmol), 6-bromopurine (41.5 mg, 0.192 mmol), cesium carbonate (2.0 M, 0.19 mL, 0.38 mmol), POPd₂ (0.1 M in DMF, 0.077 mL, 0.0077 mmol) and TBAI (3.7 mg, 0.01 mmol) in DMF (1 mL) and water (0.19 mL) was heated at 150° C. on a Biotage microwave reactor with high power for 3 h. The mixture was acidified with AcOH and concentrated. Chromatography on silica gel with 2% TEA and 10-15% MeOH in DCM gave 31 mg of 639 as TEA salt. A second chromatography on silica gel with 10-15% MeOH in DCM gave 13 mg of compound 639 as yellow solid.

### Synthesis of Compound 640

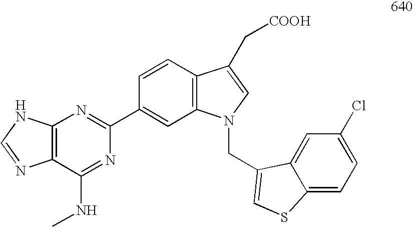

By a similar procedure as described for compound 639, compound 640 (6 mg) was prepared from compound 14 (65 mg, 0.16 mmol) and 2-chloro-6-methylaminopurine (59 mg 0.32 mmol).

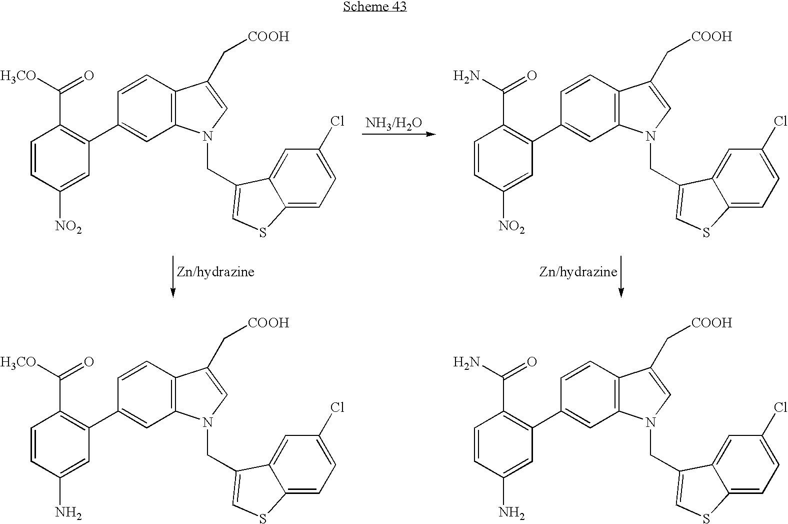

### Synthesis of Compounds 641 and 642

A solution of compound 14 (70 mg) in saturated aqueous ammonia (40 mL) was let stand at room temperature for 2 days and then concentrated to dryness. Chromatography was done on silica gel with 3-5% MeOH in DCM gave 34 mg of compound 16. Zinc powder (135 mg) and 65% hydrazine (0.5 mL) were added in sequence to a solution of compound 16 (23 mg) in MeOH (2 mL) and THF (1.5 mL). The resulting mixture was stirred at room temperature for 3 hours. Zinc powder was filtered and the filtrate concentrated. Water was added to the residue which was then acidified with 2 N HCl to pH 2. Precipitate was filtered, washed with water, and dried under vacuum to give 14.3 mg of compound 641 as pale-yellow solid; ¹H NMR (DMSO-d₆) δ 3.61 (s, 2H), 5.41 (s, 4H), 5.62 (s, 2H), 6.50 (m, 2H), 6.67 (s, 1H), 6.77 (s, 1H), 6.99 (d, J=10.0 1H), 7.26 (d, J=11.0 Hz, 1H), 7.37 (s, 1H), 7.41 (dd, J=10.5, 2.0 Hz, 1H), 7.51 (m, 3H), 8.01 (d, J=2.5 Hz, 1H), 8.03 (d, J=11.0 Hz, 1H), 12.2 (bs, 1H).

By a similar procedure as described for compound 641, compound 642 (19 mg) as white solid was prepared from compound 14 (36 mg); ¹H NMR (DMSO-d₆) δ 3.34 (s, 3H), 3.66 (s, 2H), 5.64 (s, 2H), 5.83 (bs, 2H), 6.47 (d, J=3.0 Hz, 1H), 6.52 (dd, J=10.5, 3.0 Hz, 1H), 6.88 (dd, J=10.0, 1.5 Hz, 1H), 7.38 (s, 1H), 7.39 (d, 1H), 7.41 (d, J=10.5, 2.0 Hz, 1H), 7.46 (s, 1H), 7.47 (d, J=10.0 Hz, 1H), 7.54 (d, J=10.5 Hz, 1H), 7.96 (d, J=2.5 Hz, 1H), 8.03 (d, J=10.5 Hz, 1H), 12.2 (bs, 1H).

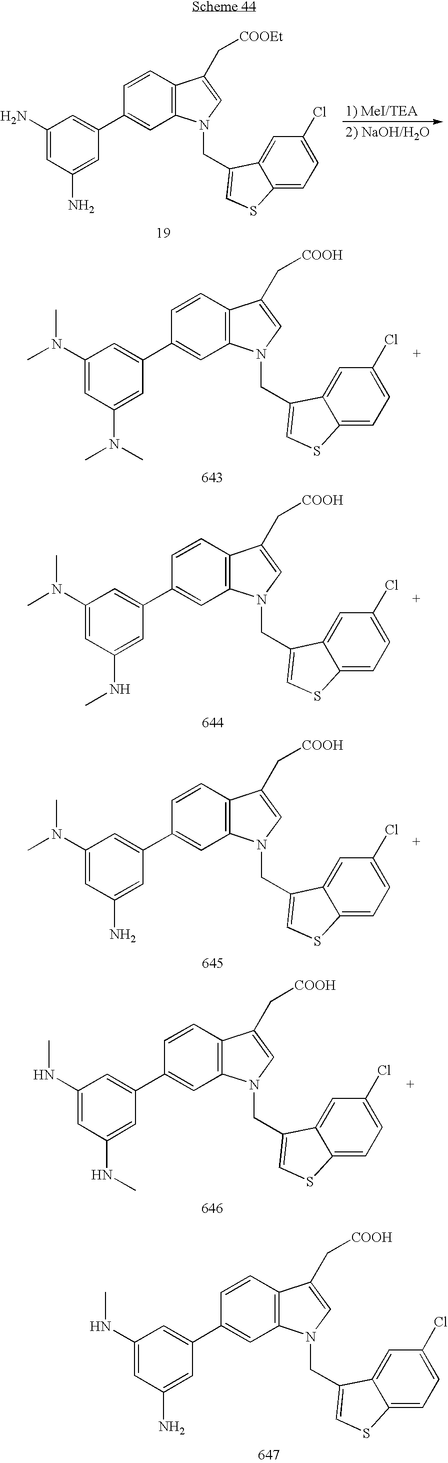

### Synthesis of Compounds 643, 644, 645, 646 and 647

A solution of compound 19 (80 mg, 0.163 mmol), MeI (0.26 mL) and TEA (0.136 mL, 0.98 mmol) in THF (2 mL) was stirred at 45° C. for 2 days and concentrated. Chromatography on silica gel with EtOAc in DCM-hexanes (1:1) gave five separated products: 6 mg of the ester of 643, 11 mg of the ester of 644, 4 mg of the ester of 645, 8 mg of the ester of 646, and 8 mg of the ester of 647). They were hydrolyzed with sodium hydroxide in MeOH/dioxane/H₂O at room temperature to give 643, 644, 645, 646 and 647, respectively, all as faint-brown solid.

¹H NMR of compound 643 (DMSO-d₆) δ 2.90 (s, 12H), 3.45 (s, 2H), 5.66 (s, 2H), 5.98 (s, 1H), 6.30 (d, J=2.5 Hz, 2H), 7.24 (dd, J=10.0, 1.5 Hz, 1H), 7.34 (s, 1H), 7.38 (dd, J=11.0, 2.5 Hz, 1H), 7.53 (d, J=10.5 Hz, 3H), 7.64 (s, 1H), 7.66 (s, 1H), 7.95 (d, J=2.5 Hz, 1H), 8.01 (d, J=10.5 Hz, 1H).

The ¹H NMR of compound 644 (DMSO-d₆) δ 2.69 (s, 3H), 2.88 (s, 6H), 3.61 (s, 2H), 5.4 (bs, 1H), 5.68 (s, 2H), 5.86 (s, 1H), 6.15 (s, 1H), 6.19 (s, 1H), 7.24 (d, J=10.5 Hz, 1H), 7.4 (m, 2H), 7.52 (d, J=10.0 Hz, 1H), 7.63 (s, 1H), 7.66 (s, 1H), 7.95 (s, 1H), 8.02 (d, J=10.5 Hz, 1H).

The ¹H NMR of compound 645 (DMSO-d₆) δ 2.86 (s, 6H), 3.60 (s, 2H), 5.67 (s, 2H), 5.94 (t, J=2.5 Hz, 1H), 6.16 (t, J=2.0 Hz, 1H), 6.22 (t, J=2.0 Hz, 1H), 7.21 (dd, J=10.0, 2.0 Hz, 1H), 7.38 (s, 1H), 7.39 (dd, J=10.5, 2.5 Hz, 1H), 7.51 (d, J=10.0 Hz, 1H), 7.57 (s, 1H), 7.65 (s, 1H), 7.96 (d, J=2.0 Hz, 1H), 8.02 (d, J=1.0 Hz, 1H).

The ¹H NMR of compound 646 (DMSO-d₆) δ 2.66 (s, 6H), 3.63 (s, 2H), 5.3 (bs, 2H), 5.68 (s, 2H), 5.71 (t, 1H), 6.05 (d, J=2.5 Hz, 2H), 7.21 (dd, J=10.5 Hz, 1.5, 1H), 7.39 (s, 1H), 7.39 (dd, J=11.0, 2.5 Hz, 1H), 7.51 (d, J=10.0 Hz, 1H), 7.57 (s, 1H), 7.64 (s, 1H), 7.96 (d, J=2.5 Hz, 1H), 8.02 (d, J=1.0 Hz, 1H).

The ¹H NMR of compound 647 (DMSO-d₆) δ 2.64 (s, 3H), 3.62 (s, 2H), 5.67 (s, 2H), 5.76 (s, 1H), 6.04 (s, 1H), 6.12 (s, 1H), 7.19 (d, J=10.0 Hz, 1H), 7.37 (s, 1H), 7.40 (dd, J=10.5, 2.0 Hz, 1H), 7.5 (m, 2H), 7.62 (s, 1H), 7.97 (d, J=2.0 Hz, 1H), 8.02 (d, J=10.5 Hz, 1H).

### Synthesis of Compounds 648

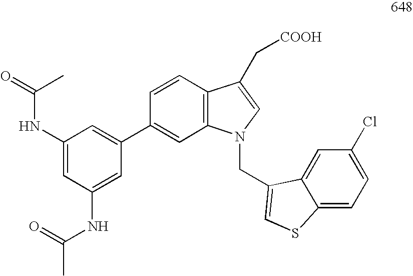

A mixture of compound 19 (75 mg, 0.15 mmol), acetic anhydride (0.043 mL), and pyridine (0.073 mL) in DCM (2 mL) was stirred at room temperature overnight. More acetic anhydride (0.2 mL) and pyridine (2 mL) were added, and the mixture was stirred for 3 h and concentrated. Chromatography on silica gel with 2-5% MeOH in DCM gave 69 mg of di-N-acetyl product, which was hydrolyzed with sodium hydroxide in MeOH/H₂O to give compound 648 as off-white solid; ¹H NMR (DMSO-d₆) δ 2.03 (s, 6H), 3.67 (s, 2H), 5.70 (s, 2H), 7.23 (d, J=10.0 Hz, 1H), 7.4 (m, 3H), 7.54 (s, 2H), 7.60 (d, J=10.5 Hz, 1H), 7.70 (s, 1H), 7.87 (s, 1H), 7.98 (d, J=2.0 Hz, 1H), 8.03 (d, J=10.5 Hz, 1H), 9.96 (s, 2H).

### Synthesis of Compounds 649 and 650

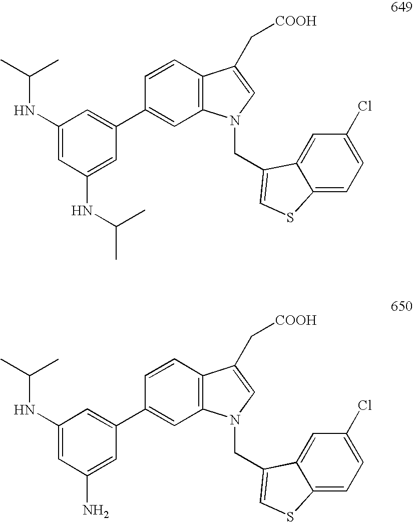

A mixture of compound 19 (159 mg, 0.165 mmol), 2-bromopropane (0.79 mL), and TEA (0.29 mL) in THF (2 mL) and dioxane (3 mL) was stirred at 60° C. for 3 days and concentrated. Chromatography on silica gel with 5-20% EtOAc in DCM-hexanes (1:1) gave two separated products (13 mg of the ester of 649 and 57 mg of the ester of 650), which were hydrolyzed with NaOH in MeOH, dioxane and water to give compounds 649 and 650, respectively, both as faint-brown solid.

Compound 649: ¹H NMR (DMSO-d₆+D₂O) δ 1.08 (d, J=7.5 Hz, 12H), 3.46 (m, 2H), 3.60 (s, 2H), 5.61 (s, 2H), 5.74 (s, 1H), 6.00 (s, 2H), 7.17 (d, J=10.0 Hz, 1H), 7.35-7.37 (m, 2H), 7.48 (d, J=10.0 Hz, 1H), 7.54 (s, 1H), 7.60 (s, 1H), 7.87 (s, 1H), 7.97 (d, J=11.0 Hz, 1H).

Compound 650: ¹H NMR (DMSO-d₆) δ 1.11 (d, J=8.0 Hz, 6H), 3.49 (m, 1H), 3.60 (s, 2H), 5.66 (s, 2H), 5.78 (s, 1H), 6.05 (s, 1H), 6.09 (s, 1H), 7.17 (d, J=10.0 Hz, 1H), 7.36 (s, 1H), 7.40 (d, 1H), 7.9-7.52 (m, 2H), 7.60 (s, 1H), 7.88 (s, 1H), 8.02 (d, J=11.0 Hz, 1H).

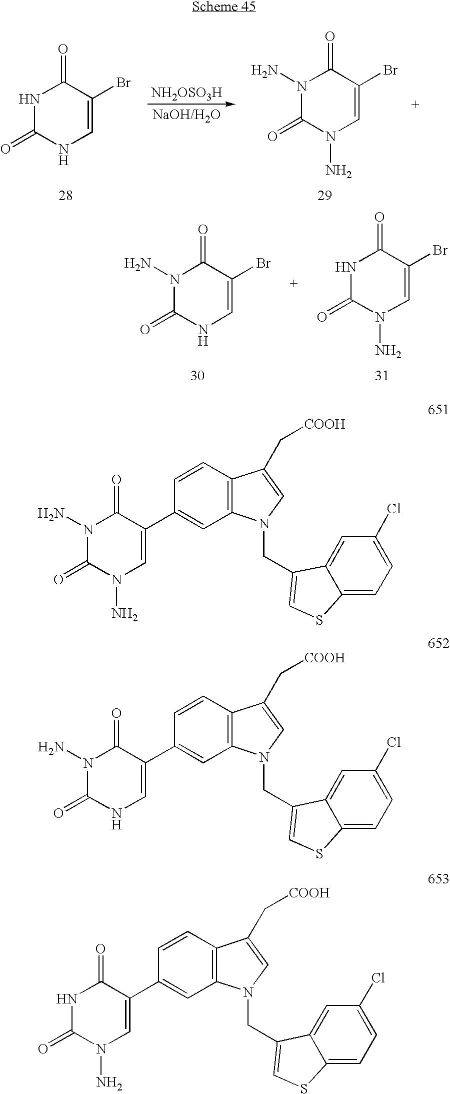

### Synthesis of Compound 651, 652, and 653

A mixture of 5-bromouracil 28 (2.30 g, 12 mmol), 2.0 N NaOH (43 mL), NH₂OSO₃ (4.52 g, 40 mmol) in water (40 mL) was stirred at 40° C. overnight. The mixture was cooled, acidified with AcOH to pH 4, and concentrated to dryness. The solid crude was extracted thoroughly with MeOH-DCM (2:1), and the extracts were concentrated. Chromatography on silica gel with 3-6% MeOH in DCM gave 0.69 g of the fraction 1: a mixture of 29 and 31 and 0.81 g of the fraction 2: a mixture of 28 and 30. Recrystallization of the fraction 1 from MeOH gave 0.12 g of compound 29, and recrystallization of the fraction 2 from MeOH gave 0.51 g of compound 30.

A mixture of compound 9 (86 mg, 0.2 mmol), compound 30 (50 mg, 0.24 mmol), sodium carbonate (1 M, 0.5 mL) and Pd(PPh₃)₄ (14.4 mg, 0.012 mmol) in DMF (1.2 mL) under argon was stirred at 82° C. for 2 days. Solvent was evaporated and the residue extracted with a mixture of MeOH and DCM. Chromatography on silica gel with 2-3% MeOH in DCM gave 26 mg of the ethyl ester of 652, which was hydrolyzed with NaOH/H₂O to give compound 652 (17 mg) as off-white solid.

A mixture of compound 9 (172 mg, 0.4 mmol), a mixture of compound 29 and 31 (104 mg, 0.48 mmol), sodium carbonate (1 M, 1 mL) and Pd(PPh₃)₄ (28.8 mg, 0.024 mmol) in DMF (2.4 mL) under argon was stirred at 82° C. for 4 days. POPD2 (15 mg) was added and the mixture was heated for one more day. The mixture was cooled, acidified with AcOH to pH 4, and concentrated. The residue was extracted with a mixture of MeOH and DCM. Chromatography on silica gel with 1-3% MeOH in DCM gave two separated products (the ester of 653: 11 mg and the ester of 651: 23 mg); which were hydrolyzed with NaOH/H₂O to give compound 653 and 651, respectively, both as off-white solid.

Compound 651: ¹H NMR (DMSO-d₆) δ 3.64 (s, 2H), 5.63 (s, 2H), 5.65 (s, 2H), 5.79 (s, 2H), 7.28 (dd, J=10.0, 2.0 Hz, 1H), 7.38 (s, 1H), 7.40 (dd, J=11.0, 2.5 Hz, 1H), 7.48 (s, 1H), 7.50 (d, J=10.5 Hz, 1H), 7.80 (s, 1H), 7.84 (s, 1H), 7.98 (d, J=2.5 Hz, 1H), 8.02 (d, J=10.5 Hz, 1H).

Compound 652: ¹H NMR (DMSO-d₆) δ 3.55 (s, 2H), 5.60 (s, 2H), 5.5 (br), 7.23 (dd, J=10.5 Hz, 1H), 7.33 (s, 1H), 7.40 (dd, J=10.5, 2.5 Hz, 1H), 7.47 (s, 1H), 7.49 (d, J=10.0 Hz, 1H), 7.55 (s, 1H), 7.77 (s, 1H), 7.99 (d, J=2.5 Hz, 1H), 8.01 (d, J=10.5 Hz, 1H).

Compound 653: ¹H NMR (DMSO-d₆) δ 3.63 (s, 2H), 5.58 (s, 2H), 5.64 (s, 2H), 7.24 (d, J=10.0 Hz, 1H), 7.37 (s, 1H), 7.40 (dd, J=10.5, 2.0 Hz, 1H), 7.48 (d, J=10.5 Hz, 1H), 7.50 (s, 1H), 7.77 (s, 1H), 7.80 (s, 1H), 7.97 (d, J=1.5 Hz, 1H), 8.02 (d, J=10.5 Hz, 1H), 11.48 (s, 1H).

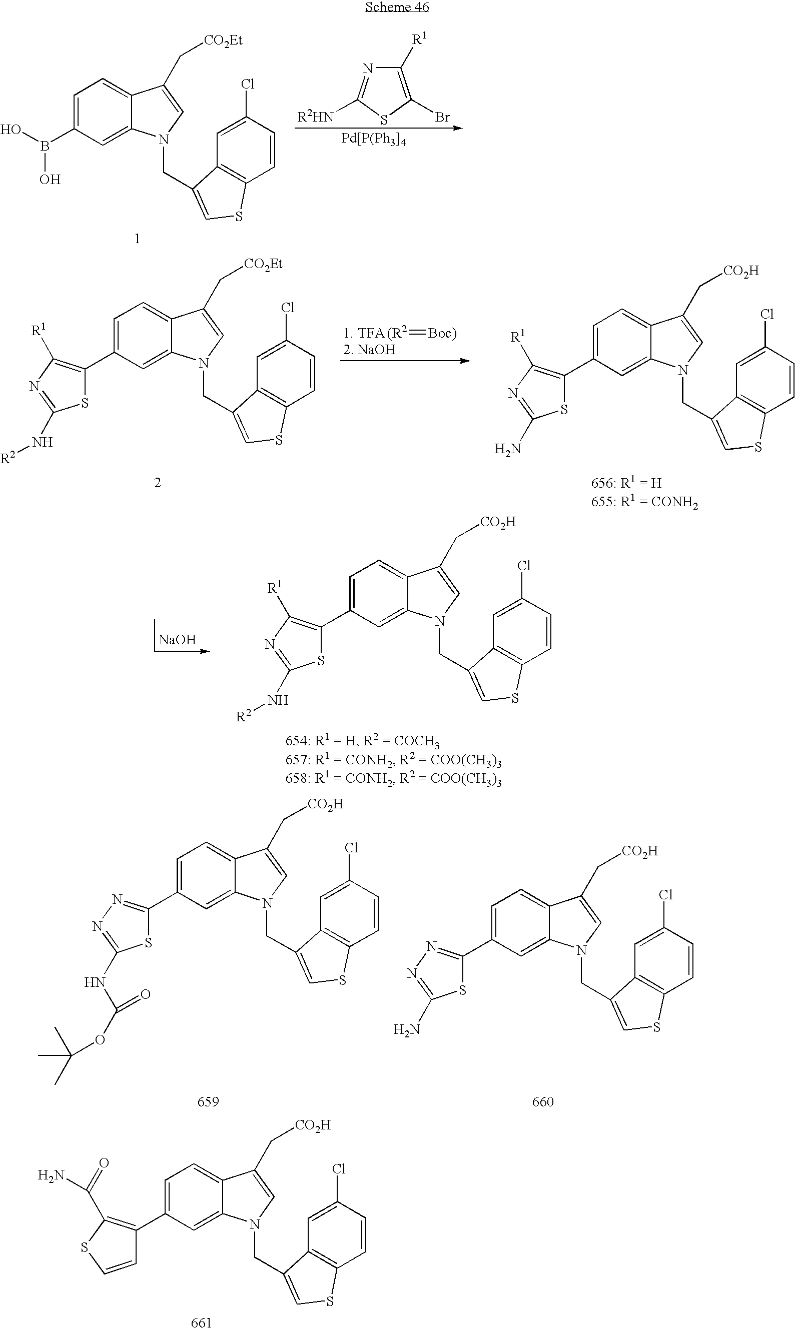

Synthesis of indole inhibitors bearing 5-membered heterocycles at position 6 is described.

The title compounds were prepared accordingly with the general scheme above as described as follow for compound 656 and compound 657:

A mixture of substituted indole 6-boronic acid 1 (220 mg, 0.514 mmol), N-Boc protected 2-amino-5-bromothiazole (287 mg, 1.03 mmol), toluene (5 ml), 1M sodium carbonate (1.54 ml) and Pd[P(Ph)₃]₄ was stirred under argon atmosphere overnight at 80° C. After cooling, insoluble material was filtered off and the filtrate was partitioned between saturated sodium bicarbonate and ethyl acetate. Organic phase was separated, dried over magnesium sulfate, and evaporated under vacuum. The residue was purified by column chromatography in 3-7% ethyl acetate-DCM to furnish fully protected intermediate 2 (R¹=H, R²=Boc), 40 mg (13%). ¹H-NMR (DMSO-d⁶), δ: 1.17 (t, 3H), 1.42 (s, 9H), 3.67 (s, 2H), 4.01 (q, 2H), 5.63 (s, 2H), 7.25 (dd, 1H), 7.38 (dd, 1H), 7.39 (s, 1H), 7.50 (d, 1H), 7.52 (s, 1H), 7.68 (s, 1H), 7.79 (s, 1H), 7.95 (d, 1H), 8.0 (d, 1H), 11.4 (br. s, 1H).

Compound 657: compound 2 (15 mg, 0.026 mmol) was dissolved in ethanol (2 ml) and dioxane (1 ml). 2N sodium hydroxide (0.1 ml) was added to the mixture and hydrolysis was carried out at 40° C. for two hours. The reaction mixture was evaporated; the residue was dissolved in water and 2N hydrochloric acid (0.1 ml) was added. The compound was extracted with ethyl acetate and isolated by precipitation from DCM. Yield 10 mg (69%). ¹H-NMR (DMSO-d⁶), δ: 1.42 (s, 9H), 3.61 (s, 2H), 5.63 (s, 2H), 7.24 (dd, 1H), 7.38 (s, 1H), 7.39 (d, 1H), 7.50 (d, 1H), 7.52 (s, 1H), 7.63 (s, 1H), 7.75 (s, 1H), 7.87 (d, 1H), 8.0 (d, 1H), 11.4 (br. s, 1H), 12.2 (br. s, 1H).

Compound 654: synthesized as compound 657. ¹H-NMR (DMSO-d⁶), δ: 2.12 (s, 3H), 3.61 (s, 2H), 5.68 (s, 2H), 7.26 (dd, 1H), 7.38 (s, 1H), 7.39 (dd, 1H), 7.51 (d, 1H), 7.55 (s, 1H), 7.77 (s, 1H), 7.82 (s, 1H), 7.96 (d, 1H), 8.0 (d, 1H), 12.0 (br. s, 1H), 12.2 (br. s, 1H).

Compound 658: synthesized similar to compound 657. ¹H-NMR (DMSO-d⁶), δ: 1.49 (s, 9H), 3.66 (s, 2H), 5.66 (s, 2H), 7.16 (dd, 1H), 7.20-7.23 (s and dd, 2H), 7.40-7.43 (s and dd, 2H), 7.45 (s, 1H), 7.50 (d, 1H), 7.58 (s, 1H), 7.82 (s, 1H), 8.02 (d, 1H), 8.04 (d, 1H), 11.6 (br. s, 1H), 12.2 (br. s, 1H).

Compound 659: Synthesized analogously to compound 657. ¹H-NMR (DMSO-d⁶), δ: 1.50 (s, 9H), 3.69 (s, 2H), 5.78 (s, 2H), 7.42 (dd, 1H), 7.51 (s, 1H), 7.53 (s, 1H), 7.62-7.64 (m, 2H), 7.99 (d, 1H), 8.04 (d, 1H), 8.15 (s, 1H), 11.9 (br s, 1H).

Compound 656: to a solution of compound 2 (12 mg, 0.021 mmol) in DCM (0.5 ml) was added triethyl silane (0.05 ml) followed by TFA (0.2 ml). After 3 hours at room temperature the reaction mixture was diluted with toluene (5 ml) end evaporated under vacuum. The co-evaporation with toluene was repeated one more time to afford white solid of the free amino intermediate. The latter was hydrolyzed to the title compound as described above and isolated by column chromatography in 5-20% MeOH-DCM as white foam. Yield 6 mg (63%). ¹H-NMR (DMSO-d⁶), δ: 3.62 (s, 2H), 5.65 (s, 2H), 6.98 (s, 2H), 7.12 (dd, 1H), 7.32 (s, 1H, 7.39 (s, 1H), 7.41 (dd, 1H), 7.47 (d, 1H), 7.61 (d, 1H), 7.62 (s, 1H), 7.97 (d, 1H), 8.04 (d, 1H), 12.2 (br. s, 1H).

Compound 655: synthesized as compound 656 and isolated as a salt with triethyl amine. ¹H-NMR (DMSO-d⁶), δ: 0.94 (t, 9H), 2.46 (q, 6H), 3.61 (s, 2H), 5.62 (s, 2H), 7.04 (br. s, 2H), 7.13 (dd, 1H), 7.17 (br. s, 1H), 7.23 (br. s, 1H), 7.40 (d, 1H), 7.42 (s, 1H), 7.44 (d, 1H), 7.62 (s, 1H), 7.73 (s, 1H), 8.01 (d, 1H), 8.03 (d, 1H).

Compound 660: synthesized similar to compound 656. ¹H-NMR (DMSO-d⁶), δ: 3.67 (s, 2H), 5.74 (s, 2H), 7.27 (s, 2H), 7.42 (dd, 1H), 7.45 (dd, 1H), 7.46 (s, 1H), 7.53 (s, 1H), 7.59 (d, 1H), 7.94 (s, 1H), 7.95 (d, 1H), 8.04 (d, 1H), 12.2 (br. s, 1H).

Compound 661: synthesized according to the general scheme. ¹H-NMR (DMSO-d⁶), δ: 3.67 (s, 2H), 5.67 (s, 2H), 6.61 (br. s, 1H), 7.12 (dd, 1H), 7.15 (d, 1H), 7.38 (br. s, 1H), 7.41 (dd, 1H), 7.46 (s, 1H), 7.57 (d, 1H), 7.60 (s, 1H), 7.71 (d, 1H), 7.76 (s, 1H), 8.01 (d, 1H), 8.03 (d, 1H), 12.2 (br. s, 1H).

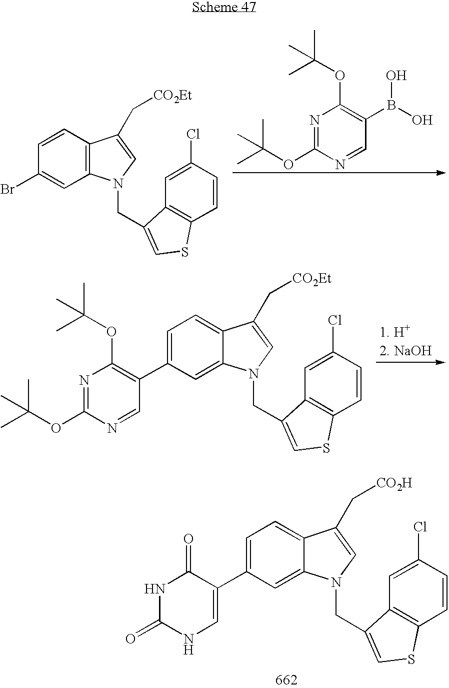

### Synthesis of Compound 662

A mixture of the starting bromoindole (151 mg, 0.33 mmol), boronic acid (187 mg, 0.65 mmol), ethanol (2 ml), toluene (2 ml), 1M sodium carbonate (0.7 ml) and Pd[P(Ph)₃]₄ (10 mg) was stirred under argon atmosphere at 80° C. overnight. After conventional extractive work-up the product was isolated by column chromatography in 2-3% ethyl acetate-DCM. Yield 180 mg (91%). ¹H-NMR (DMSO-d⁶), δ: 1.47 (s, 9H); 1.58 (s, 9H), 3.75 (s, 2H), 5.66 (s, 2H), 7.21 (dd, 1H), 7.40 (dd, 1H), 7.45 (s, 1H), 7.50 (s, 1H), 7.53 (d, 1H), 7.71 (s, 1H), 7.80 (d, 1H), 8.03 (d, 1H), 8.28 (s, 1H).

To a solution of protected compound from above (100 mg, 0.165 mmol) in methanol (3 ml) and THF (2 ml) was added hydrochloric acid (6N, 0.5 ml) and the mixture was stirred at 30° C. for 2 hours. The solid was filtered off and re-suspended in ethanol (2 ml). Sodium hydroxide (2N, 0.4 ml) was added and the hydrolysis was carried out for 1 hour at 40° C. The resulted solution was concentrated under vacuum, dissolved in a small amount of water and acidified to pH˜3 by 2N hydrochloric acid. The solid of 662 was filtered off, washed with water and dried under vacuum. Yield 30 mg (39%). ¹H-NMR (DMSO-d⁶), δ: 3.64 (s, 2H), 5.64 (s, 2H), 7.22 (d, 1H), 7.39 (s, 1H), 7.41 (d, 1H), 7.47 (d, 1H), 7.53 (s, 1H), 7.53 (d, 1H), 7.77 (s, 1H), 7.80 (s, 1H), 8.03 (d, 1H), 11.09 (s, 1H), 11.21 (s, 1H), 12.21 (br. s, 1H).

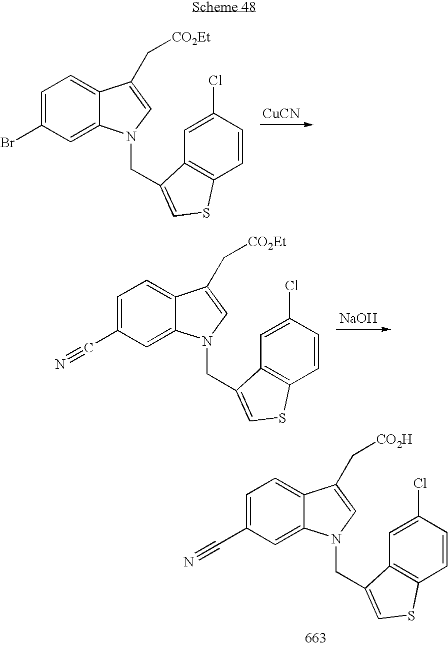

### Synthesis of Compound 663

A mixture of the starting bromoindole (462.8 mg, 1 mmol), cupper cyanide (723 mg, 8 mmol) and 1-methyl-2-pyrrolidinone (2 ml) was heated at 145° C. overnight. After cooling down to room temperature the reaction was partitioned between 10% ammonia and athylacetate, inorganic solids were filtered off and organic phase was separated, dried over magnesium sulphate and evaporated. The cyano ester intermediate was isolated by column chromatography (60-100% DCM-hexane) followed by crystallization from ethyl acetate-hexane. Yield: 294 mg (72%). ¹H-NMR (DMSO-d⁶), δ: 1.15 (t, 3H), 3.80 (s, 2H), 4.05 (q, 2H), 5.75 (s, 2H), 7.39 (dd, 1H), 7.42 (dd, 1H), 7.66 (s, 1H), 7.71 (d, 1H), 7.73 (s, 1H), 7.97 (d, 1H), 8.04 (d, 1H), 8.26 (s, 1H).

The ester intermediate (50 mg, 0.122 mmol) was hydrolyzed using a standard procedure to afford 663, isolated by column chromatography in 5% MeOH-DCM. Yield 45 mg (97%). ¹H-NMR (DMSO-d⁶), δ: 3.71 (s, 2H), 5.74 (s, 2H), 7.38 (dd, 1H), 7.42 (dd, 1H), 7.64 (s, 1H), 7.70 (dd, 1H), 7.72 (s, 1H), 7.98 (d, 1H), 8.04 (d, 1H), 8.25 (s, 1H), 12.3 (br. s, 1H).

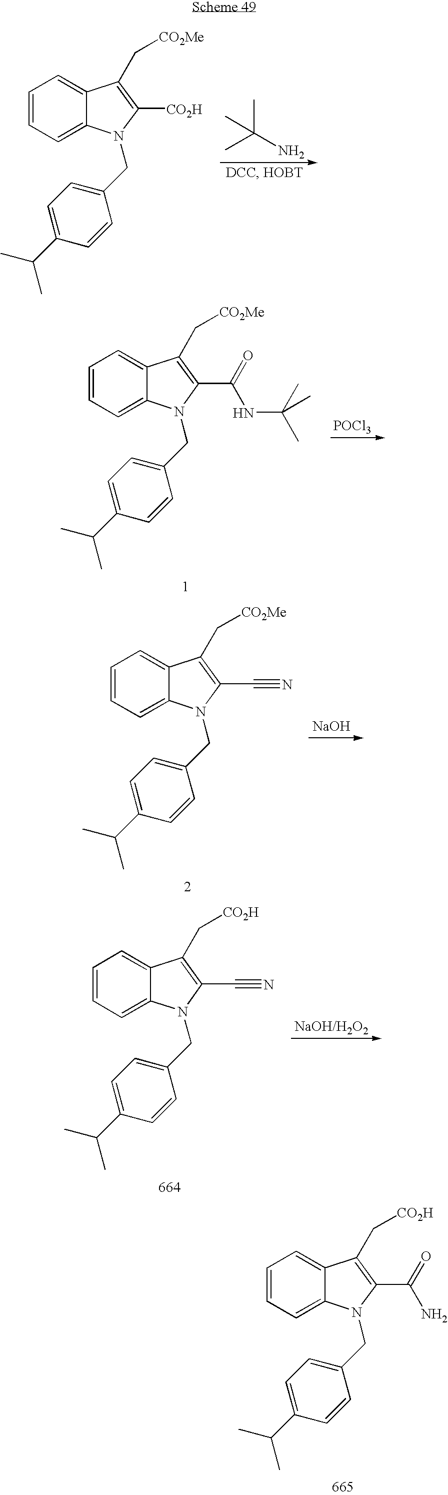

### Synthesis of Compounds 664 and 665

To a solution of starting acid (365 mg, 1 mmol) in DCM (4 ml) was added t-butyl anine (0.26 ml, 2.5 mmol) followed by HOBT (2 ml 0.5M solution in THF) and DCC (1 ml 1M THF solution). After stirring for 2 hours at room temperature and usual extractive work-up the amide 1 was isolated by column chromatography in 10-15% ethyl acetate-hexane. Yield 360 mg (86%). To a solution of this material (360 mg, 0.86 mmol) in benzene (3 ml), POCl₃ (0.42 ml, 4.6 mmol) was added and the reaction was carried out at 85° C. for 4 hours. After cooling, the reaction mixture was diluted with ethyl acetate, washed with saturated sodium bicarbonate, dried over magnesium sulfate and evaporated. Column chromatography (DCM to 2% EtOAc-DCM) afforded the nitrile 2 as a white solid. Yield 260 mg (87%). ¹H-NMR (DMSO-d⁶), δ: 1.13 (s, 3H), 1.15 (s, 3H), 2.81-2.84 (m, 1H), 3.64 (s, 3H), 4.01 (s, 2H), 5.52 (s, 2H), 7.10 (d, 1H), 7.12 (s, 1H), 7.19-7.23 (m, 3H), 7.39-7.43 (m, 1H), 7.70-7.72 (m, 2H).

Compound 2 (40 mg, 0.116 mmol) was hydrolyzed under a standard procedure to afford 32 mg (83%) of 664. ¹H-NMR (DMSO-d⁶), δ: 1.13 (s, 3H), 1.15 (s, 3H), 2.49-2.51 (m, 1H), 3.89 (s, 2H), 5.51 (s, 2H), 7.11 (d, 1H), 7.12 (s, 1H), 7.18-7.22 (m, 3H), 7.37-7.43 (m, 1H), 7.69 (d, 1H), 7.72 (d, 1H), 12.2 (br.s, 1H).

Alternatively, compound 2 (65 mg, 0.188 mmol) was dissolved in ethanol (2 ml) and hydrolyzed with 2N NaOH (0.2 ml) at 40° C. for 30 min. After the ester group hydrolysis was completed, hydrogen peroxide (30%, 0.2 ml) was added and the reaction was continued for 3 hours at the same temperature. Usual extractive work-up followed by column chromatography (10%-20% MeOH in DCM) afforded 665 as a white solid. Yield 30 mg (45%). ¹H-NMR (DMSO-d⁶), δ: 1.11 (s, 3H), 1.13 (s, 3H), 2.49-2.51 (m, 1H), 3.84 (s, 2H), 5.60 (s, 2H), 7.02 (d, 1H), 7.03 (s, 1H), 7.05-7.12 (m, 4H), 7.18-7.22 (m, 1H), 7.49 (d, 1H), 7.61 (d, 1H), 7.74 (br. s, 1H), 8.2 (br. s, 1H), 12.68 (br. s, 1H).

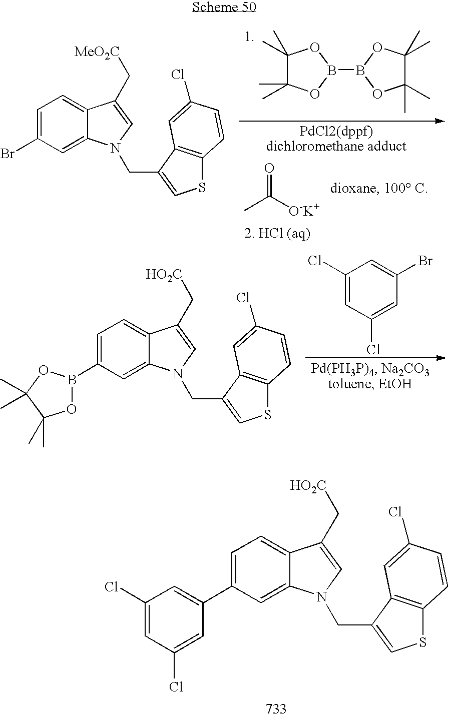

### Step 1: methyl 2-(1-((5-chlorobenzo[b]thiophen-3-yl)methyl)-6-(4,4,5,5-tetramethyl-1,3,2-dioxaborolan-2-yl)-1H-indol-3-yl)acetic acid

In a 100 mL round bottomed flask, 6-bromo-1-((5-chlorobenzo[b]thiophen-3-yl)methyl)-1H-indol-3-yl)acetate (6.685 ml, 0.6685 mmol) was suspended in dioxane (6 mL). To this was added 4,4,5,5-tetramethyl-2-(4,4,5,5-tetramethyl-1,3,2-dioxaborolan-2-yl)-1,3,2-dioxaborolane (220.7 mg, 0.8691 mmol), potassium acetate (196.8 mg, 2.006 mmol) and PdCl₂(dppf) dichloromethane adduct (16.50 mg, 0.02006 mmol). The reaction was purged with nitrogen and the reaction mixture was heated to 90° C. for 20 hours.

The reaction was worked up by diluting in EtOAc and washing with saturated Na₂CO₃ then was purified on a silica gel column using a gradient of 6:1 Hex/EtOAc->4:1 Hex/EtOAc to provide methyl 2-(1-((5-chlorobenzo[b]thiophen-3-yl)methyl)-6-(4,4,5,5-tetramethyl-1,3,2-dioxaborolan-2-yl)-1H-indol-3-yl)acetic acid as a yellow oil (0.316 g, 0.673 mmol, 95%).

### Step 2: 2-(1-((5-chlorobenzo[b]thiophen-3-yl)methyl)-6-(5,5-dimethyl-1,3,2-dioxaborinan-2-yl)-1H-indol-3-yl)acetate

To a solution of methyl 2-(1-((5-chlorobenzo[b]thiophen-3-yl)methyl)-6-(5,5-dimethyl-1,3,2-dioxaborinan-2-yl)-1H-indol-3-yl)acetate (0.227 g, 0.471 mmol) in 2 mL of THF was added 2N HCl (2.36 ml, 4.71 mmol) and the resulting mixture was stirred at stirred at 40° C. for 40 hrs to provide 2-(1-((5-chlorobenzo[b]thiophen-3-yl)methyl)-6-(5,5-dimethyl-1,3,2-dioxaborinan-2-yl)-1H-indol-3-yl)acetate as a yellow oil.

### Step 3: 2-(1-((5-chlorobenzo[b]thiophen-3-yl)methyl)-6-(3,5-dichlorophenyl)-1H-indol-3-yl)acetic acid (733)

To a solution of 2-(1-((5-chlorobenzo[b]thiophen-3-yl)methyl)-6-(5,5-dimethyl-1,3,2-dioxaborinan-2-yl)-1H-indol-3-yl)acetic acid (44.1 mg, 0.09428 mmol) and 1-bromo-3,5-dichlorobenzene (21.30 mg, 0.09428 mmol) in toluene (2 mL) was added Na₂CO₃ (0.2357 ml, 0.4714 mmol) and Pd(PPh₃)₄ (5.447 mg, 0.004714 mmol). To the solution was added 0.5 mL of EtOH to help solubility. After purging under N₂, the reaction mixture was heated to 90° C. overnight.

The reaction was then acidified with 2N HCl and extracted with EtOAc (3×). The combined organics and washed with brine, dried with MgSO₄ and concentrated down to light brown-orange solid. This solid was diluted in CH₂Cl₂ and Et₂O and the resulting solid was collected by filtration to afford 733 (15.8 mg, 33.46% yield). MS APCI (+) m/z 497.8 detected.

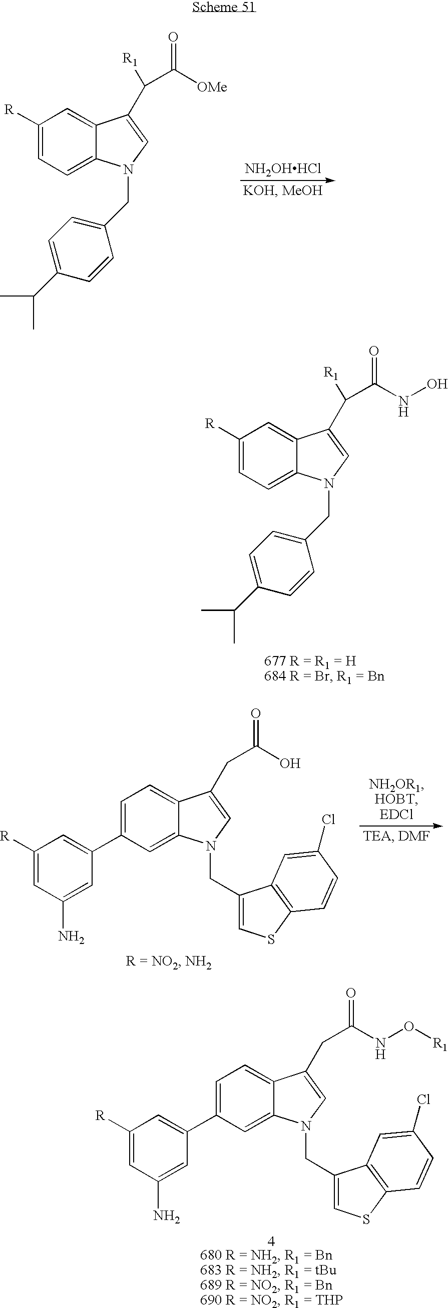

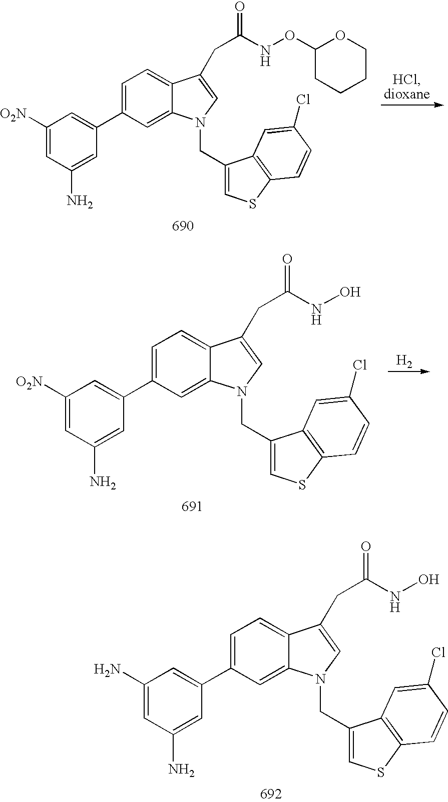

Indole hydroxamic acids were synthesized as bioisosteric replacements for indole acetic acid inhibitors. A general synthetic scheme for the preparation of hydroxamic acid derivatives is illustrated in Scheme 51 and exemplified by the description of the synthesis of compound 677 (R=R₁=H) and compound 692.

### Synthesis of N-1-(p-isopropylbenzyl)indole-3-methanehydroxamic acid (677)

To the stirred solution of methyl N-1-(p-isopropylbenzyl)indole-3-acetic acid (64 mg, 0.2 mml) in methanol (2 ml) was added hydroxylamine hydrochloride (56 mg, 0.8 mmol), followed by 5M KOH in methanol (0.2 ml). The reaction mixture was stirred at room temperature for 48 hours. Precipitate was filtered off and solvent removed under reduced pressure. The residue was partitioned between ethyl acetate and 1N HCl, organic layer washed with water, dried (MgSO₄) and concentrated under reduced pressure. Crystallization from DCM afforded 677 (25 mg). Mother liquor was chromatographed on the column of silica gel using 1-4% gradient of methanol in DCM to afford additional 10 mg of 677 (total yield 55%). ¹H NMR (DMSO-d₆): δ 1.13 (s, 3H), 1.15 (s, 3H), 2.82 (m, 1H), 3.38 (s, 2H), 5.31 (s, 2H), 6.96-7.59 (m, 9H), 8.76 (s, 1H), 10.61 (s, 1H).

Synthesis of 3-(tetrahydropyranyloxycarbamoylmethyl)-6-(3-amino-5-nitrophenyl)-N-1-((5-Cl-benzothiophen-3-yl)methyl)indole (690) and 6-(3-amino-5-nitrophenyl)-N-1-((5-Cl-benzothiophen-3-yl)methyl))indole-3-methanehydroxamic acid (692)

6-(3-amino-5-nitrophenyl)-N-1-((5-C₁-benzothiophen-3-yl)methyl)indole-3-acetic acid (98 mg, 0.2 mmol) was dissolved in DMF (4 ml) and to this solution was added HOBt hydrate (33 mg, 0.24 mmol), TEA (34 μL, 0.24 mmol) and O-(tetrahydro-2H-pyran-2-yl)hydroxylamine (28 mg, 0.24 mmol). The mixture was cooled in an ice bath and EDAC (46 mg, 0.24 mmol) was added. The reaction mixture was stirred at room temperature overnight, then diluted with ethyl acetate and extracted with water. Organic layer was washed with water, dried (MgSO₄) and concentrated under reduced pressure. Chromatography on the column of silica gel using 25-50% gradient of ethyl acetate in hexane afforded 690 (56 mg, 47%). ¹H NMR (DMSO-d₆): δ 1.21 (m, 2H), 1.47 (m, 2H), 1.60 (m, 2H), 3.44 (m, 3H), 3.90 (m, 1H), 4.79 (s, 1H), 5.71 (s, 2H), 5.82 (s, 2H), 7.23-8.02 (m, 11H), 11.19 (s, 1H).

Compound 690 (50 mg, 0.08 mmol) was dissolved in 4N HCl in dioxane (4 ml) and methanol (2 ml). The reaction mixture was stirred at room temperature for 2 hours, then evaporated to dryness and coevaporated twice with methanol. Crystallization from methanol afforded 691 (35 mg, 81%). ¹H NMR (DMSO-d₆): δ 3.38 (s, 2H), 5.71 (s, 2H), 7.28-8.02 (m, 13H), 10.59 (s, 1H).

Compound 691 (30 mg, 0.06 mmol) was suspended in methanol (4 ml) and hydrogenated in the presence of 10% Pd—C catalyst (25 mg) at 30 psi overnight. Catalyst was filtered off and the solvent removed under reduced pressure to afford 692 (25 mg, 83%). ¹H NMR (DMSO-d₆): δ 3.42 (m), 5.67 (s, 2H), 6.44 (s, 1H), 6.76 (s, 2H), 7.19-8.02 (m, 10H), 10.59 (s, 1H).

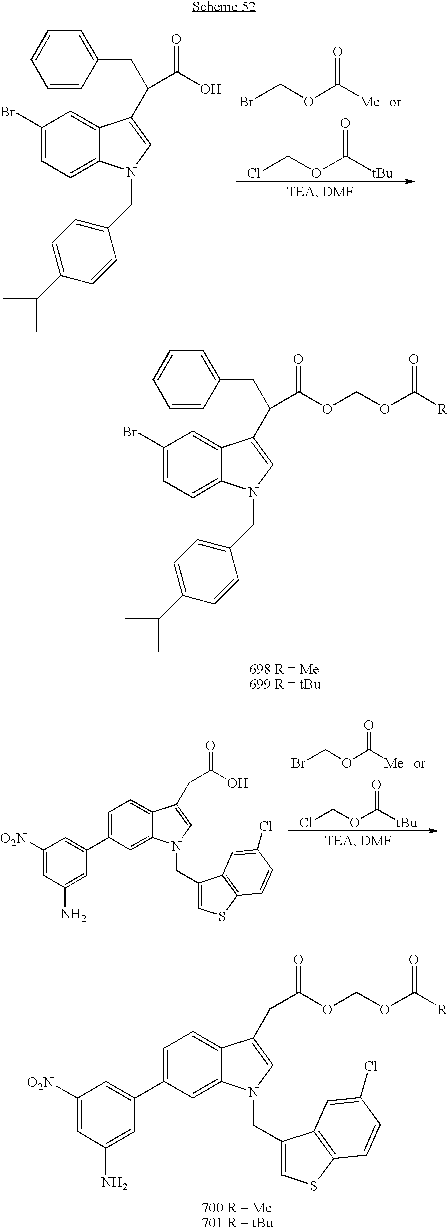

Prodrugs are utilized to deliver drugs that can not cross the cell membranes in vivo. Prodrug moieties like acetyloxymethyl (AOM) and pivaloyloxymethyl (POM) undergo hydrolytic cleavage catalyzed by cellular enzymes, thus releasing the active ingredient.

A general synthetic scheme for the preparation of acetyloxymethyl (AOM) and pivaloyloxymethyl (POM) prodrugs of indole 3-acetic acid inhibitors is illustrated in Scheme 52 and exemplified by the description of the synthesis of compounds 698 and 701.

### Synthesis of acetyloxymethyl 5-bromo-α-benzyl-N-1-(p-isopropylbenzyl)indole-3-acetic acid (698)

To the solution of 5-bromo-α-benzyl-N-1-(p-isopropylbenzyl)indole-3-acetic acid (95 mg, 0.2 mmol) in DMF (5 ml), TEA (56 μL, 0.4 mmol) and bromomethyl acetate (25 μL, 0.26 mmol) were added. The reaction mixture was stirred at room temperature overnight and then diluted with ethyl acetate and extracted with aqueous NH₄Cl. The organic layer was washed with brine, dried (MgSO₄) and evaporated to dryness. Column chromatography on silica gel using 20-30% gradient of ethyl acetate in hexane afforded 698 (80 mg, 83%). ¹H NMR (DMSO-d₆): δ 1.10 (s, 3H), 1.12 (s, 3H), 1.84 (s, 3H), 2.79 (m, 1H), 3.13 (m, 1H), 3.30 (m, 1H), 4.26 (m, 1H), 5.29 (s, 2H), 5.58 (dd, 2H), 6.95-7.72 (m, 13H).

### Synthesis of pivaloyloxymethyl 6-(3-amino-5-nitrophenyl)-N-1-((5-Cl-benzothiophen-3-yl)methyl))indole-3-acetic acid (701)

To the solution of 6-(3-amino-5-nitrophenyl)-N-1-((5-Cl-benzothiophen-3-yl)methyl))indole-3-acetic acid (98 mg, 0.2 mmol) in DMF (5 ml), TEA (56 μL, 0.4 mmol) and chloromethyl pivalate (38 μL, 0.26 mmol) were added. The reaction mixture was stirred at 55° C. for 10 hours, then diluted with ethyl acetate and extracted with aqueous NH₄Cl. The organic layer was washed with water, dried (MgSO₄) and evaporated to dryness. Chromatography on the column of silica gel using 25-30% gradient of ethyl acetate in hexane afforded 701 (50 mg, 42%). ¹H NMR (DMSO-d₆): δ 0.99 (s, 9H), 3.83 (s, 2H), 5.69 (s, 2H), 5.73 (s, 2H), 5.84 (s, 2H), 7.22-8.02 (m, 11H).

Compounds 670 to 674 were synthesized according to Scheme 19, and compounds 686 to 688 were synthesized according to Scheme 4.

Compound 670: ¹H NMR δ 2.24 (s, 3H), 5.88 (d, 1H), 7.11-8.41 (m, 13H), 9.04 (d, 1H), 12.89 (broad s, 1H).

Compound 672: (mixture of 2 stereoisomers) ¹H NMR (DMSO-d₆+D₂O) δ 1.7-2.4 (m, 7H), 3.24 (m, 1H), 3.673 (m, 1H), 4.26 (m, 1H), 5.66 (d, 1H), 7.11-8.40 (m, 8H).

Compound 674: ¹H NMR δ 1.67-1.97 (m, 4H), 2.20 (s, 3H), 2.60 (m, 1H), 2.84 (m, 1H), 3.43-3.74 (m, 1H), 5.61 (d, 1H), 7.10-8.79 (m, 12H), 12.79 (broad s, 1H).

Compound 675: ¹H NMR δ 3.63 (s, 2H), 3.77 (s, 3H), 4.80 (broad s, 4H), 5.81 (t, 1H), 6.15 (d, 2H), 7.16-7.50 (m, 4H).

Compound 676: ¹H NMR δ 3.65 (s, 2H), 4.85 (broad s, 4H), 5.41 (s, 2H), 5.79 (t, 1H), 6.08 (s, 2H), 7.15-7.54 (m, 9H).

Compound 678: (TEA⁺ salt): δ ¹H NMR δ 0.95 (t, 3H), 3.61 (s, 2H), 5.64 (s, 2H), 7.22-7.99 (m, 9H).

Compound 679: ¹H NMR δ 3.64 (s, 2H), 4.85 (broad s, 4H), 5.67 (s, 2H), 5.81 (t, 1H), 6.12 (d, 2H), 7.17-8.00 (m, 9H).

Compound 680: ¹H NMR δ 3.40 (s, 2H), 4.74 (s, 4H), 4.77 (s, 2H), 5.67 (s, 2H), 5.80 (t, 1H), 6.11 (d, 2H), 7.30-8.05 (m, 13H), 11.22 (broad s, 1H).

Compound 682: ¹H NMR δ 3.04 (s, 6H), 3.68 (s, 2H), 5.72 (s, 2H), 7.12-8.05 (m, 11H), 9.89 (broad s, 2H), 12.24 (broad s, 1H).

Compound 683: ¹H NMR δ 1.06 (s, 9H), 3.40 (s, 2H), 4.70 (broad s, 4H), 5.64 (s, 2H), 5.77 (s, 1H), 6.09 (s, 2H), 7.16-8.02 (m, 8H), 10.47 (broad s, 1H).

Compound 684: ¹H NMR δ 1.11 (s, 3H), 1.12 (s, 3H), 2.80 (m, 1H), 2.98 (m, 1H), 3.79 (m, 1H), 5.28 (s, 2H), 7.01-7.45 (m, 12H), 7.88 (s, 1H), 8.72 (s, 1H), 10.56 (s, 1H).

Compound 686: ¹H NMR δ 0.83 (m, 2H), 0.97 (m, 1H), 1.10 (s, 3H), 1.12 (s, 3H), 2.72 (m, 1H), 2.79 (m, 1H), 3.04 (m, 1H), 3.32 (m, 1H), 4.13 (m, 1H), 5.31 (s, 2H), 7.01-7.46 (m, 12H), 7.82 (s, 1H), 11.74 (s, 1H).

Compound 688: ¹H NMR δ 0.93-1.04 (m, 4H), 2.87 (m, 1H), 3.67 (s, 2H), 5.66 (s, 2H), 5.77 (s, 1H), 6.08 (s, 2H), 7.17-8.02 (m, 8H).

Compound 693: ¹H NMR δ 3.86 (s, 3H), 5.85 (s, 2H), 7.43 (m, 2H), 7.63 (s, 1H), 8.02-8.11 (m, 4H), 8.74 (s, 1H).

Compound 694: ¹H NMR δ 2.79 (t, 2H), 3.60 (m, 2H), 4.60 (t, 1H), 5.60 (s, 2H), 7.09-8.01 (m, 8H).

Compound 695: ¹H NMR δ 1.45-1.92 (m, 4H), 1.97 (s, 3H), 2.84 (m, 2H), 3.25 (m, 1H), 3.44 (m, 1H), 4.80 (m, 1H), 5.57 (s, 2H), 6.30-8.02 (m, 9H).

Compound 699: ¹H NMR δ 0.82 (s, 9H), 1.10 (s, 3H), 1.12 (s, 3H), 2.78 (m, 1H), 3.12 (m, 1H), 3.35 (m, 1H), 4.27 (m, 1H), 5.28 (s, 2H), 5.56 (d, 1H), 5.66 (d, 1H), 6.95-7.73 (m 13H).

Compound 700: ¹H NMR δ 2.00 (s, 3H), 3.84 (s, 2H), 5.67 (s, 2H), 5.84 (s, 2H), 5.89 (s, 2H), 7.23-8.02 (m, 11H).

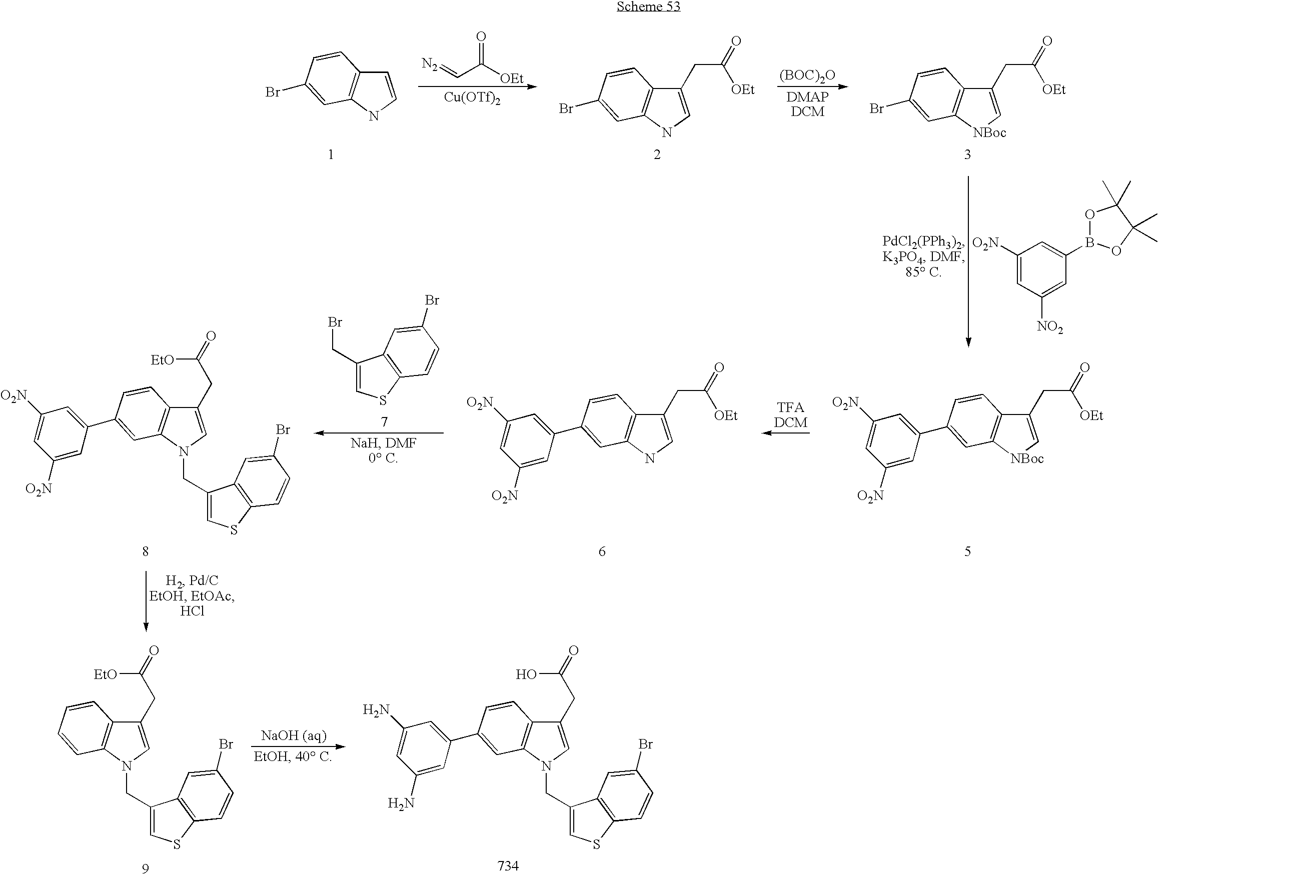

A general synthetic scheme for the preparation of Helicase inhibitors is illustrated in Scheme 53 and exemplified by the description of the synthesis of compound 734.

### Synthesis of (6-Bromo-1H-indol-3-yl)-acetic acid ethyl ester (2)

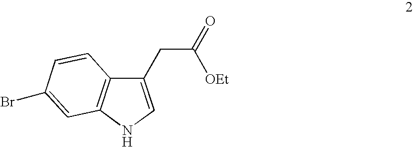

To a stirred solution of 6-bromoindole 1 (7.4 g, 37.8 mmol) in DCM (135 mL) under a nitrogen atmosphere was added copper (II) triflate (683 mg, 1.89 mmol) and the mixture cooled in an ice-bath (internal temperature 5° C.). A solution of ethyl diazoacetate (5.16 mL, 49.1 mmol, d 1.085) in DCM (50 mL) was added over 70 min causing nitrogen gas to be evolved. The reaction was allowed to slowly warm to room temperature and stirred for 16 h. The reaction was diluted with DCM (180 mL) and washed with water (350 mL). The organic phase was dried (Na₂SO₄), filtered and solvent evaporated. The crude was purified by column chromatography (silica, eluent 20% EtOAc in heptane) to give the title compound 2 as a brown oil (5.6 g, 53%). ¹H NMR (CDCl₃, 250 MHz) δ 8.17 (br s, 1H), 7.37 (d, 1H), 7.31 (d, 1H), 7.13 (dd, 1H), 6.91 (d, 1H), 4.10 (q, 2H), 3.65 (s, 2H), 1.19 (t, 3H).

### Synthesis of 6-Bromo-3-ethoxycarbonylmethyl-indole-1-carboxylic acid tert-butyl ester (3)

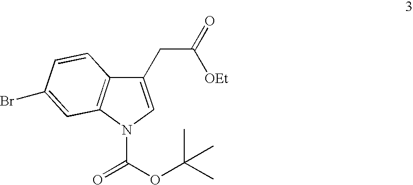

To a stirred solution of the indole 2 (7.9 g, 27.9 mmol) in THF (130 mL) was added Boc anhydride (12.8 mL, 55.9 mmol, d 0.95) followed by DMAP (5.11 g, 41.9 mmol). The reaction was stirred at room temperature and for 2 h, and then the THF evaporated. The crude was purified by column chromatography (silica, eluent 10% EtOAc in heptane) to give the title compound 3 as a pale yellow oil (8.2 g, 77%). ¹H NMR (CDCl₃, 250 MHz) δ 8.29 (br s, 1H), 7.46 (s, 1H), 7.30 (m, 2H), 4.10 (q, 2H), 3.60 (s, 2H), 1.59 (s, 9H), 1.19 (t, 3H).

### Synthesis of 2-(3,5-Dinitro-phenyl)-4,4,5,5-tetramethyl-[1,3,2]dioxaborolane (4)

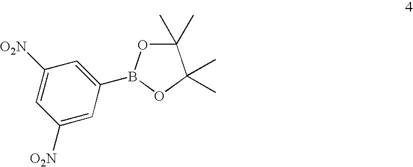

A flask was charged with 2,4-dinitroiodobenzene (2.00 g, 6.8 mmol), bispinacolato-diboron (2.59 g, 10.2 mmol), potassium acetate (2.00 g, 20.4 mmol), Pd(dppf)Cl₂ (500 mg, 0.6 mmol) and DMF (22 mL) and the mixture stirred for 1 h at 85° C. under a nitrogen atmosphere. More Pd(dppf)Cl₂ (100 mg, 0.12 mmol) was added and stirring continued at 85° C. for a further 2 h. The mixture was cooled to rt, diluted with EtOAc (300 mL) and washed with water (300 mL). The aqueous phase was then extracted with further portions of EtOAc (2×100 mL). The combined organic phases were washed with water (2×50 mL), brine (25 mL), dried (Na₂SO₄), filtered and solvent evaporated. The crude was purified by column chromatography (silica, eluent 20% EtOAc in heptane) followed by recrystallisation from heptane to give the title compound 4 as a white solid (685 mg, 34%). ¹H NMR (250 MHz, CDCl₃) δ 9.04 (t, 1H), 8.83 (d, 2H), 1.29 (s, 12H).

### Synthesis of 6-(3,5-Dinitro-phenyl)-3-ethoxycarbonylmethyl-indole-1-carboxylic acid tert-butyl ester (5)

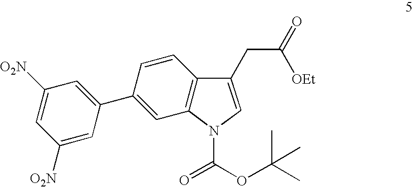

A stirred mixture of the indole 3 (2.0 g, 5.2 mmol), boronic ester 4 (2.3 g, 7.9 mmol), potassium phosphate tribasic (3.3 g, 15.7 mmol) and PdCl₂(PPh₃)₂ (110 mg, 0.16 mmol) in DMF (40 mL) was degassed with nitrogen for 5 min and then heated at 85° C. for 2 h. The reaction was cooled to rt, diluted with EtOAc (100 mL) and washed with 10% (w/v) aqueous citric acid solution (150 mL). The aqueous phase was extracted with EtOAc (3×150 mL). The combined organic phases were dried (MgSO₄), filtered and solvent evaporated. The crude was purified by column chromatography (silica, eluent 10% EtOAc in heptane) to give the title compound 5 as a yellow solid (2.0 g, 81%). ¹H NMR (CDCl₃, 360 MHz) δ 8.91 (t, 1H), 8.76 (d, 2H), 8.50 (m, 1H), 7.62 (m, 2H), 7.51 (d, 1H), 4.14 (q, 2H), 3.68 (s, 2H), 1.63 (s, 9H), 1.22 (t, 3H).

### Synthesis of [6-(3,5-Dinitro-phenyl)-1H-indol-3-yl]-acetic acid ethyl ester (6)

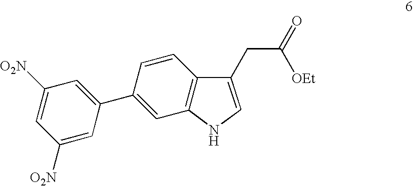

To a stirred solution of the Boc-indole 5 (2 g, 4.2 mmol) in DCM (40 mL) was added TFA (22 mL), and the reaction stirred at room temperature for 2 h. The solvent was then evaporated and the residue azeotroped with a mixture of heptane and DCM. It was then suspended in DCM (5 mL) and heptane (30 mL) and the mixture heated to reflux. The mixture was cooled to room temperature and filtered, discarding the collected solid. The filtrate was evaporated, dissolved in EtOAc (100 mL), and washed with satd NaHCO₃ (100 mL). A precipitate formed upon addition of NaHCO₃, therefore, the aqueous phase was extracted with EtOAc (5×250 mL) until all the precipitate had dissolved. The combined organic phases were dried (MgSO₄), filtered and evaporated to give the title compound 6 as a yellow brown solid (1.3 g, 83%). ¹H NMR (CDCl₃, 250 MHz) δ 8.97 (t, 1H), 8.81 (d, 2H), 8.35 (br s, 1H), 7.79 (d, 1H), 7.69 (d, 1H), 7.45 (d, 1H), 7.34 (d, 1H), 4.20 (q, 2H), 3.82 (s, 2H), 1.29 (t, 3H).

### Synthesis of [1-(5-Bromo-benzo[b]thiophen-3-ylmethyl)-6-(3,5-dinitro-phenyl)-1H-indol-3-yl]-acetic acid ethyl ester (8)

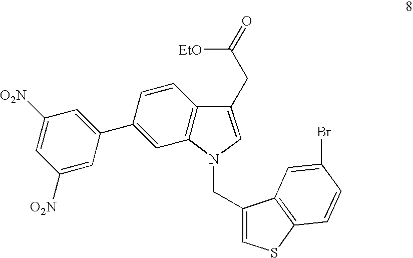

To a stirred solution of the indole 6 (50 mg, 0.14 mmol) in DMF (2 mL) at 0° C. was added a suspension of sodium hydride (60% in oil, 6 mg, 0.15 mmol) in DMF (1.5 mL), and the reaction stirred at 0° C. for 10 min. A solution of alkyl bromide 7 (41 mg, 0.14 mmol) in DMF (1.5 mL) was added at 0° C., and the reaction allowed to warm to room temperature over 4 h. The reaction was diluted with EtOAc (5 mL) and washed with 10% (w/v) aqueous citric acid solution (2×8 mL). The aqueous phase was then extracted with EtOAc (2×10 mL) and the combined organic phases dried (MgSO₄), filtered and evaporated. The crude was purified by column chromatography (silica, eluent 20% EtOAc in heptane) followed by trituration with heptane to give the title compound 8 as a yellow solid (20 mg, 25%). ¹H NMR (CDCl₃, 250 MHz) δ 8.94 (t, 1H), 8.75 (d, 2H), 7.87-7.73 (m, 3H), 7.60 (s, 1H), 7.48 (m, 2H), 7.26 (s, 1H), 7.00 (s, 1H), 5.58 (s, 2H), 4.19 (q, 2H), 3.81 (s, 2H), 1.28 (t, 3H).

### Synthesis of [1-(5-Bromo-benzo[b]thiophen-3-ylmethyl)-6-(3,5-diamino-phenyl)-1H-indol-3-yl]-acetic acid ethyl ester (9)

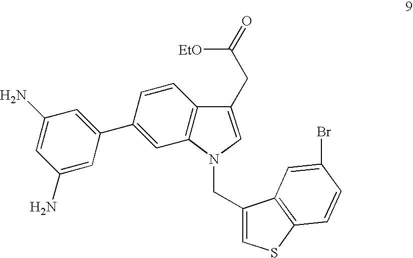

The dinitro compound 8 (20 mg, 0.034 mmol) was partially dissolved in EtOH (10 mL) with heating and then allowed to cool to rt. The mixture was then treated with concentrated HCl (0.5 mL) and 10% palladium on carbon (5 mg), and stirred under a hydrogen atmosphere for 1 h 40 min. The reaction was filtered through celite, washed with EtOH (20 mL), and the filtrate evaporated to give the title compound 9 as a cream solid (17 mg, 64%). MS m/e 535 (M⁺+1).

### Synthesis of [1-(5-Bromo-benzo[b]thiophen-3-ylmethyl)-6-(3,5-diamino-phenyl)-1H-indol-3-yl]-acetic acid (734)

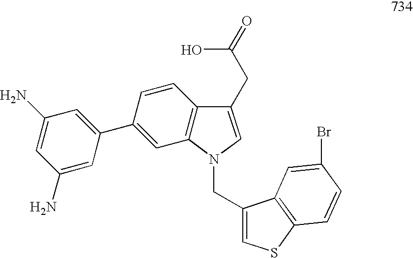

To a stirred solution of ethyl ester 9 (17 mg, 0.028 mmol) in EtOH (3 mL) at room temperature was added 2 M NaOH (112 μl, 0.22 mmol) and the reaction stirred at 40° C. for 2 h. The reaction was cooled to room temperature and the solvent evaporated. The residue was dissolved in water (pH 8) and extracted with EtOAc (2 mL). The pH of the aqueous solution was then adjusted to pH 5 with 1 M HCl and the solution extracted with EtOAc (3×2 mL). The combined organic phases from the acidic extraction were dried (MgSO₄), filtered and solvent evaporated. The crude was purified by column chromatography (silica, eluent 1-10% MeOH in DCM) to give the title compound 734 as a cream solid (3 mg, 21%). ¹H NMR (MeOD, 360 MHz) δ 7.91 (d, 1H), 7.70 (d, 1H), 7.52 (d, 1H), 7.43 (d, 1H), 7.37 (dd, 1H), 7.21 (dd, 1H), 7.10 (d, 2H), 6.36 (d, 2H part exchanged), 6.03 (s, 1H part exchanged), 5.48 (s, 2H), 3.59 (s, 2H). MS m/e 506, 508 (M⁺+1).

### Synthesis of Compound 709

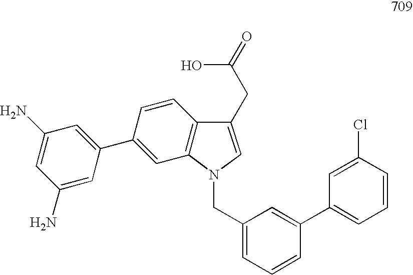

[1-(3-Bromo-benzyl)-6-(3,5-dinitro-phenyl)-1H-indol-3-yl]-acetic acid ethyl ester (10) was prepared as an orange solid (43 mg, 19%) by alkylation of indole 6 (200 mg, 0.42 mmol) with 3-bromobenzyl bromide (104 mg, 0.42 mmol) following the procedure described in Scheme 53. MS m/e 538, 540 (M⁺+1).

A mixture of aryl bromide 10 (43 mg, 0.08 mmol), 3-chlorophenylboronic acid (19 mg, 0.12 mmol), potassium phosphate tribasic (51 mg, 0.24 mmol) and PdCl₂(dppf) (20 mg, 0.03 mmol) in DMF (2 mL) was degassed with nitrogen for 2 min and then heated at 75° C. for 1.5 h. The reaction was cooled to room temperature, diluted with EtOAc (8 mL) and washed with 10% (w/v) aqueous citric acid solution (10 mL). The aqueous phase was then extracted with EtOAc (3×10 mL) and the combined organic phases dried (Na₂SO₄), filtered and evaporated. The crude was purified by column chromatography (silica, eluent 10% EtOAc in heptane) to give [1-(3′-Chloro-biphenyl-3-ylmethyl)-6-(3,5-dinitro-phenyl)-1H-indol-3-yl]-acetic acid ethyl ester (11) as a yellow oil (35 mg, 77%). MS m/e 570, 572 (M⁺+1).

The compound 709 was then obtained by hydrogenation and saponification of compound 11 according to the procedures described in Scheme 53. ¹H NMR (MeOH, 360 MHz) δ 7.48-7.01 (m, 12H), 5.28 (s, 2H), 3.63 (s, 2H). MS m/e 482, 484 (M⁺+1).

Additional compounds were synthesized using Scheme 53:

Compound 702: ¹H NMR (MeOD, 360 MHz) δ 7.98 (d, 1H), 7.56-7.63 (m, 4H), 7.25-7.53 (m, 3H), 6.72 (d, 2H part exchanged), 6.56 (d, 1H part exchanged), 5.66 (s, 2H), 3.75 (s, 2H). MS m/e 443 (M⁺+1).

Compound 703: ¹H NMR (MeOD, 360 MHz) δ 8.20 (s, 1H), 7.97 (d, 1H), 7.55-7.51 (m, 2H), 7.43 (s, 1H), 7.25 (s, 1H), 7.19 (dd, 1H), 7.12 (s, 1H), 6.34 (s, 2H part exchanged), 6.02 (s, 1H, part exchanged), 5.56 (s, 2H), 3.55 (s, 2H). MS m/e 453 (M⁺+1).

Compound 704: ¹H NMR (MeOD, 360 MHz) δ 7.96 (d, 1H), 7.77 (d, 1H), 7.44-7.51 (m, 3H), 7.36 (s, 1H), 7.17-7.24 (m, 3H), 6.30 (d, 2H part exchanged), 6.02 (s, 1H part exchanged), 5.45 (s, 2H), 3.67 (s, 2H), 2.56 (s, 3H). MS m/e 437 (M⁺+1).

Compound 705: ¹H NMR (DMSO, 360 MHz) δ 8.06 (dd, 1H), 7.75-7.68 (m, 2H), 7.63-7.55 (m, 2H), 7.47 (s, 1H), 7.32-7.20 (m, 2H), 6.76 (s, 2H part exchanged), 6.42 (s, 1H part exchanged), 5.70 (s, 2H), 3.67 (s, 2H). MS m/e 446 (M⁺+1).

Compound 706: ¹H NMR (MeOH, 360 MHz) δ 7.93 (m, 2H), 7.66 (s, 1H), 7.59 (d, 1H), 7.45-7.41 (m, 2H), 7.32-7.26 (m, 3H), 6.88 (s, 2H part exchanged), 6.53 (s, 1H part exchanged), 5.52 (s, 2H), 3.72 (s, 2H), 2.10 (s, 3H). MS m/e 453 (M⁺+1).

Compound 707: ¹H NMR (MeOH, 360 MHz) δ 7.65 (d, 1H), 7.50-7.45 (m, 3H), 7.21 (d, 1H), 7.14 (d, 1H), 6.89 (s, 1H), 6.38 (s, 2H part exchanged), 6.08 (s, 1H part exchanged) 5.37 (s, 2H), 3.57 (s, 2H), 2.46 (s, 3H). MS m/e 476, 478 (M⁺+1).

Compound 708: ¹H NMR (MeOH, 360 MHz) δ 7.74 (d, 1H), 7.52 (s, 1H), 7.46-7.38 (m, 2H), 7.37 (s, 1H), 7.05 (dd, 1H), 6.67 (d, 1H), 5.54 (s, 2H), 3.84 (s, 2H). MS m/e 445 (M⁺+1).

Compound 710: ¹H NMR (MeOH, 360 MHz) δ 7.72-7.69 (m, 2H), 7.62-7.55 (m, 2H), 7.30-7.21 (m, 3H), 7.15 (s, 1H), 6.45 (s, 2H part exchanged), 6.13 (s, 1H part exchanged), 5.63 (s, 2H), 3.69 (s, 2H). MS m/e 462,464 (M⁺+1).

Compound 791: ¹H NMR (MeOD, 360 MHz) δ 8.85 (br s, 1H), 8.05-7.78 (br m, 4H), 7.62-7.53 (br m, 3H), 7.39-7.30 (br m, 2H), 5.69 (br s, 2H), 4.08 (q, 2H), 3.75 (br s, 2H), 2.88 (br s, 3H), 1.17 (t, 3H). MS m/e 465 (M⁺+1).

Compound 792: ¹H NMR (CDCl₃, 250 MHz) δ 8.95 (t, 1H), 8.76 (d, 2H), 7.87-7.77 (m, 2H), 7.59 (s, 1H), 7.47 (dd, 1H), 7.37-7.23 (m, 2H), 7.22-7.08 (m, 2H), 5.57 (s, 2H), 4.20 (q, 2H), 3.81 (s, 2H), 1.28 (t, 3H).

Compound 793: ¹H NMR (CDCl₃, 250 MHz) δ 8.94 (t, 1H), 8.75 (d, 2H), 7.83-7.68 (m, 3H), 7.48-7.38 (m, 2H), 7.35-7.25 (m, 1H), 7.02 (d, 1H), 5.87 (s, 2H), 4.21 (q, 2H), 3.83 (s, 2H), 1.30 (t, 3H). MS m/e 551 (M⁺+1).

Compound 794: ¹H NMR (CDCl₃, 360 MHz) δ 7.62-7.55 (m, 2H), 7.41 (s, 1H), 7.27 (d, 1H), 7.19 (1H, obs), 7.17-7.12 (m, 1H), 6.81 (d, 1H), 6.31-6.28 (m, 2H), 5.94-5.91 (m, 1H), 5.74 (s, 2H), 4.11 (q, 2H), 3.72 (s, 2H), 3.53 (br s, 4H), 1.20 (t, 3H). MS m/e 491, 493 (M⁺+1).

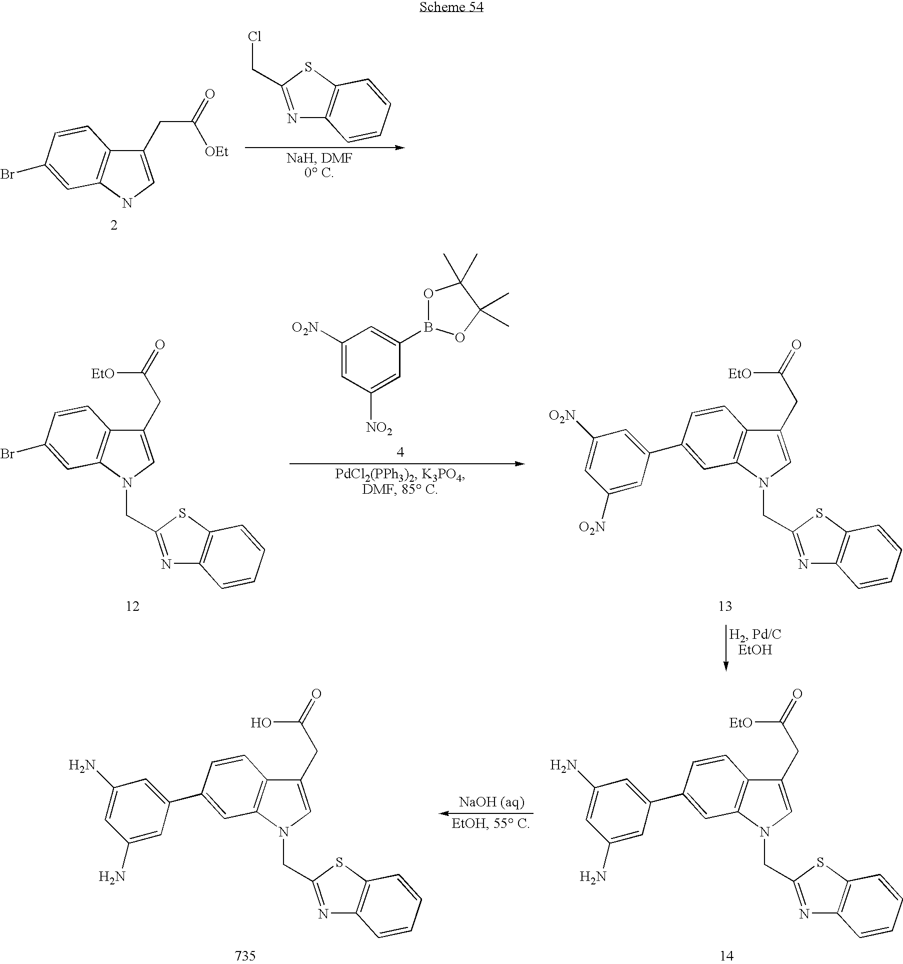

Another general route for the preparation of Helicase inhibitors is illustrated in Scheme 54 and exemplified by the description of the synthesis of compound 735.

### Synthesis of (1-Benzothiazol-2-ylmethyl-6-bromo-1H-indol-3-yl)-acetic acid ethyl ester (12)

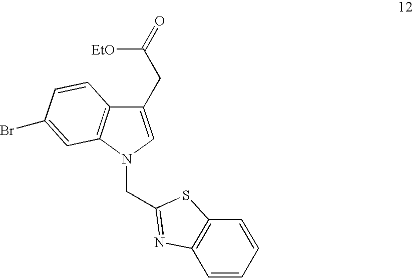

To a suspension of sodium hydride (60% in oil, 28 mg, 0.70 mmol) in DMF (3 mL) at 0° C. was added a solution of indole 2 (180 mg, 0.64 mmol) in DMF (1 mL), dropwise over 5 min. The reaction mixture was stirred at 0° C. for 5 min and then 2-(bromomethyl)-1,3-benzothiazole (160 mg, 0.70 mmol) in DMF (1 mL) was added, and the reaction stirred at 0° C. for 10 min. The reaction mixture was poured into saturated NaHCO₃ and extracted with EtOAc (4×10 mL). The combined organic phases were washed with water (2×5 mL) and brine (5 mL), dried (Na₂SO₄), filtered and evaporated. The crude was purified by column chromatography (silica, eluent 25% EtOAc in heptane) to give the indole compound 12 as a brown oil (130 mg, 48%). MS m/e 429, 431 (M⁺+1).

### Synthesis of ([1-Benzothiazol-2-ylmethyl-6-(3,5-dinitro-phenyl)-1H-indol-3-yl]-acetic acid ethyl ester (13)

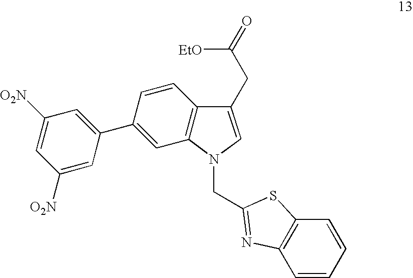

A mixture of the indole 12 (58 mg, 0.14 mmol), boronic ester 4 (59 mg, 0.20 mmol), potassium phosphate tribasic (85 mg, 0.40 mmol) and PdCl₂(PPh₃)₂ (30 mg, 0.04 mmol) in DMF (2 mL) was degassed with nitrogen for 5 min and then heated at 85° C. for 1 h. The reaction was cooled to rt, diluted with EtOAc (15 mL) and washed with 10% (w/v) aqueous citric acid solution (2×5 mL). The combined aqueous phases were then extracted with EtOAc (3×10 mL). The combined organic phases were washed with water (5 mL), brine (5 mL), dried (Na₂SO₄), filtered and evaporated. The crude was purified by column chromatography (silica, eluent 17-25% EtOAc in heptane) to give the dinitro compound 13 as a brown solid (28 mg, 52%). MS m/e 517 (M⁺+1).

### Synthesis of ([1-Benzothiazol-2-ylmethyl-6-(3,5-diamino-phenyl)-1H-indol-3-yl]-acetic acid ethyl ester (14)

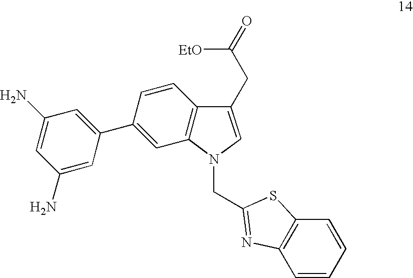

The dinitro compound 13 (33 mg, 0.072 mmol) was dissolved in EtOH (8 mL) with heating, and then allowed to cool to rt. The mixture was then treated with 10% palladium on carbon (7 mg) and concentrated HCl (8 drops) and stirred under a hydrogen atmosphere for 2 h. The solution was filtered through Celite, washed with EtOH (25 mL), and the filtrate evaporated to an oily residue. The residue was dissolved in EtOAc (15 mL) and washed with satd NaHCO₃ (15 mL). The aqueous phase was then extracted with further portions of EtOAc (3×10 mL) and the combined organic phases washed with water (5 mL), brine (5 mL), dried (Na₂SO₄), filtered and evaporated. The crude was purified by column chromatography (silica, eluent 17-25% EtOAc in heptane) to give the ester 14 as a brown oil (12 mg, 42%). MS m/e 457 (M⁺+1).

### Synthesis of [1-Benzothiazol-2-ylmethyl-6-(3,5-diamino-phenyl)-1H-indol-3-yl]-acetic acid (735)

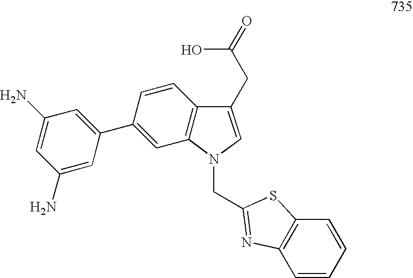

To a stirred solution of the ester 14 (12 mg, 0.42 mmol) in EtOH (3 mL) was added 2 M NaOH (0.5 mL, 1 mmol) and the solution stirred at 55° C. for 4 h. The EtOH was evaporated, the residue diluted with water (5 mL) adjusted to pH 5 with 1 M HCl and solid NaHCO₃ and extracted with EtOAc (3×10 mL). The combined organic phases were washed with brine (5 mL), dried (Na2SO4), filtered and evaporated. The crude was purified by column chromatography (silica, eluent 5% MeOH in DCM) to give the title compound 735 as an off-white solid (1.7 mg, 11%). ¹H NMR (MeOD, 360 MHz) δ 8.05 (d, 1H), 7.93 (d, 1H), 7.68 (d, 1H), 7.64 (s, 1H), 7.57 (t, 1H), 7.48-7.40 (m, 3H), 6.52 (d, 1H part exchanged), 6.21 (s, 2H part exchanged), 5.91 (s, 2H), 3.84 (s, 2H). MS m/e 429 (M⁺+1).

Additional compounds were synthesized using Scheme 54:

Compound 711: ¹H NMR (CDCl₃, 360 MHz) δ 9.15 (s, 1H), 8.91 (d, 2H), 8.08 (d, 1H), 7.94 (d, 1H), 7.74 (d, 1H), 7.44-7.73 (m, 9H), 7.28 (s, 1H), 5.74 (s, 2H), 4.31 (q, 2H), 3.89 (s, 2H), 1.39 (t, 3H).

Compound 712: ¹H NMR (MeOD, 250 MHz) δ 7.94 (d, 1H), 7.24-7.68 (m, 14H), 7.02 (s, 1H), 5.60 (s, 2H), 4.07 (q, 2H), 3.68 (s, 2H), 1.15 (t, 3H). MS m/e 532 (M⁺+1).

Compound 713: ¹H NMR (MeOD, 360 MHz) δ 7.78 (d, 1H), 7.16-7.76 (m, 11H), 6.84 (s, 1H), 6.32 (s, 2H part exchanged), 6.08 (s, 1H part exchanged), 5.34 (s, 2H), 3.52 (s, 2H). MS m/e 504 (M⁺+1).

Compound 714: ¹H NMR (CDCl₃, 360 MHz) δ 8.90 (s, 1H), 8.65 (d, 2H), 7.83 (d, 1H), 7.68 (d, 1H), 7.54 (d, 1H), 7.37-7.26 (m, 8H), 7.00 (s, 1H), 5.45 (s, 2H), 4.06 (q, 2H), 3.63 (s, 2H), 1.14 (t, 3H).

Compound 715: ¹H NMR (MeOD, 250 MHz) δ 7.93 (d, 1H), 7.72 (d, 1H), 7.56 (d, 1H), 7.28-7.48 (m, 8H), 6.95 (s, 1H), 6.43 (s, 2H, part exchanged), 6.19 (s, 1H part exchanged), 5.57 (s, 2H), 3.63 (s, 2H). MS m/e 538, 540 (M⁺+1).

Compound 716: ¹H NMR (DMSO, 250 MHz) δ 8.11 (s, 1H), 8.02 (s, 1H), 7.86 (d, 1H), 7.69 (t, 2H), 7.52 (m, 1H), 7.44 (s, 1H), 7.34-7.21 (m, 5H), 6.81 (s, 2H part exchanged), 6.45 (s, 4H, part exchanged), 6.15 (s, 2H part exchanged), 5.96 (s, 2H part exchanged), 5.61 (s, 2H), 3.71 (s, 2H), 3.67 (s, 2H) [1:1 mixture of 5,6- and 4,5-isomers]. MS m/e 496 (M⁺+1).

Compound 717: ¹H NMR (CDCl₃, 250 MHz) δ 8.95 (s, 1H), 8.77 (d, 2H), 7.91-7.80 (m, 2H), 7.58 (s, 1H), 7.50 (m, 2H), 7.28 (m, 2H), 7.11 (s, 1H), 5.61 (s, 2H), 4.20 (q, 2H), 3.81 (s, 2H), 1.28 (t, 3H). MS m/e 560 (M⁺+1).

Compound 718: ¹H NMR (MeOD, 250 MHz) δ 7.96 (d, 1H), 7.70 (m, 2H), 7.61 (s, 1H), 7.45-7.26 (m, 6H), 7.12 (s, 1H), 5.70 (s, 2H), 4.14 (q, 2H), 3.80 (s, 2H), 1.20 (t, 3H). MS m/e 540 (M⁺+1).

Compound 719: ¹H NMR (MeOD, 360 MHz) δ 7.85 (d, 1H), 7.57 (s, 1H), 7.49 (d, 1H), 7.44 (s, 1H), 7.23-7.14 (m, 4H), 6.36 (s, 2H part exchanged), 6.10 (s, 1H part exchanged), 5.51 (s, 2H), 3.64 (s, 2H). MS m/e 512 (M⁺+1).

Compound 720: ¹H NMR (CDCl₃, 250 MHz) δ 8.97 (s, 1H), 8.79 (d, 2H), 7.77 (m, 2H), 7.67 (s, 1H), 7.46 (m, 2H), 7.29 (m, 1H), 6.96 (s, 1H), 5.45 (s, 2H), 4.14 (q, 2H), 3.72 (s, 2H), 3.00 (q, 2H), 1.34 (t, 3H), 1.22 (t, 3H).

Compound 721: ¹H NMR (MeOD, 360 MHz) δ 7.78 (d, 1H), 7.63 (s, 1H), 7.56 (m, 2H), 7.31 (dd, 1H), 7.25 (dd, 1H), 6.95 (s, 1H), 6.50 (s, 2H part exchanged), 6.17 (s, 1H part exchanged) 5.48 (s, 2H), 3.66 (s, 2H), 3.00 (q, 2H), 1.26 (t, 3H). MS m/e 490, 492 (M⁺+1).

Compound 722: ¹H NMR (MeOD, 360 MHz) δ 7.63 (d, 1H), 7.57 (s, 1H), 7.46 (s, 1H), 7.36 (dd, 1H), 7.25 (s, 1H), 7.20 (s, 1H), 6.94 (s, 1H), 6.51 (s, 2H part exchanged) 6.20 (s, 1H part exchanged), 5.56 (s, 2H), 4.01 (s, 3H), 3.78 (s, 2H). MS m/e 492, 494 (M⁺+1).

Compound 723: ¹H NMR (MeOD, 360 MHz) δ 8.27 (d, 1H), 7.81 (m, 3H), 7.53 (m, 3H), 6.69 (s, 2H part exchanged), 6.39 (s, 1H part exchanged), 5.82 (s, 2H), 3.98 (s, 2H). MS m/e 480 (M⁺+1).

Compound 724: ¹H NMR (DMSO, 360 MHz) δ 7.60 (d, 1H), 7.49 (m, 2H), 7.41 (d, 1H), 7.24 (s, 2H), 7.13 (s, 1H), 6.35 (s, 2H), 6.05 (s, 1H), 5.53 (s, 2H), 3.63 (s, 2H). MS m/e 496 (M⁺+1).

Compound 725: ¹H NMR (DMSO, 360 MHz) δ 7.95 (d, 1H), 7.70 (s, 1H), 7.63 (s, 1H), 7.59-7.52 (m, 2H), 7.50 (m, 1H), 7.43 (s, 1H), 7.25 (dd, 1H), 6.16 (s, 2H), 5.86 (s, 1H), 5.75 (s, 2H), 3.70 (s, 2H). MS m/e 462 (M⁺+1).

Compound 726: ¹H NMR (MeOD, 250 MHz) δ 7.61 (m, 2H), 7.55 (dd, 1H), 7.38-7.32 (m, 3H), 7.29 (s, 1H), 6.52 (s, 2H part exchanged), 6.22 (s, 2H, part exchanged), 5.63 (s, 2H), 3.79 (s, 2H). MS m/e 480, 482 (M⁺+1).

Compound 727: ¹H NMR (MeOD, 360 MHz) δ 7.92 (s, 1H), 7.83 (d, 1H), 7.72-7.59 (m, 5H), 7.49 (s, 1H), 7.45 (d, 1H), 7.40-7.34 (m, 4H), 7.32-7.21 (m, 3H), 6.85 (s, 2H part exchanged), 6.84 (s, 2H part exchanged), 6.69 (s, 1H part exchanged), 6.36 (s, 1H, part exchanged), 5.93 (s, 2H), 5.57 (s, 2H), 3.81 (s, 2H), 3.77 (s, 2H) [1:1 mixture of 4- and 6-isomers]. MS m/e 462, 464 (M⁺+1).

Compound 728: ¹H NMR (MeOD, 360 MHz) δ 7.78 (d, 1H), 7.56 (d, 1H), 7.46 (m, 2H), 7.19 (dd, 2H), 7.07 (d, 2H), 6.50 (s, 2H part exchanged), 6.07 (s, 1H part exchanged), 5.42 (s, 2H), 3.62 (s, 2H). MS m/e 462, 464 (M⁺+1).

Compound 775: ¹H NMR (CDCl₃, 360 MHz) 5,6-isomer: δ 7.87 (s, 1H), 7.59 (d, 1H), 7.46-7.42 (m, 2H), 7.19 (m, 2H), 7.03 (s, 1H), 5.31 (s, 2H), 4.09 (m, 2H) 3.65 (s, 2H), 1.19 (t, 3H). 4,5-isomer: δ 7.65 (s, 1H), 7.39-7.35 (m, 2H), 7.19 (m, 1H), 7.07 (s, 1H), 6.99 (s, 1H), 6.45 (s, 1H), 5.74 (s, 2H), 4.09 (m, 2H), 3.68 (s, 1H), 1.19 (t, 3H) [3:2 mixture of 5,6- and 4,5-isomers]. MS m/e 497, 499 (M⁺+1).

Compound 776: ¹H NMR (DMSO, 250 MHz) 5,6-isomer: δ 8.39 (s, 1H), 8.18 (s, 1H), 7.63-7.16 (m, 5H), 6.09-5.79 (m, 3H), 5.67 (s, 2H), 3.64 (s, 2H). 4,5-isomer: δ 8.03 (d, 1H), 7.63-7.16 (m, 5H), 6.87 (s, 1H), 6.09-5.91 (m, 3H), 5.77 (s, 2H), 3.66 (s, 2H). [2:1 mixture of 5,6- and 4,5-isomers]. MS m/e 496 (M⁺+1).

Compound 777: ¹H NMR (MeOH, 360 MHz) δ 7.67 (d, 1H), 7.58 (s, 1H), 7.44 (d, 1H), 7.38 (s, 1H), 7.21-7.16 (m, 2H), 6.79 (s, 1H), 5.29 (s, 2H), 3.59 (s, 2H), 2.50 (s, 3H).

Compound 778: ¹H NMR (MeOH, 360 MHz) δ 7.65 (d, 1H), 7.50-7.45 (m, 3H), 7.21 (d, 1H), 7.14 (d, 1H), 6.89 (s, 1H), 6.38 (s, 2H part exchanged), 6.08 (s, 1H part exchanged) 5.37 (s, 2H), 3.57 (s, 2H), 2.46 (s, 3H). MS m/e 476, 478 (M⁺+1).

Compound 779: ¹H NMR (CDCl₃, 360 MHz) δ 7.43-7.35 (m, 4H), 7.17 (m, 1H), 7.02 (s, 1H), 6.97 (s, 1H), 5.33 (s, 2H), 4.06 (q, 2H), 3.63 (s, 2H), 1.16 (t, 3H). MS m/e 498-500 (M⁺+2).

Compound 780: ¹H NMR (MeOD, 360 MHz) δ 7.50 (d, 1H), 7.43 (s, 1H), 7.37-7.33 (m, 2H), 7.22 (s, 1H), 7.05 (s, 1H), 7.00 (d, 1H), 5.40 (s, 2H), 3.48 (s, 2H). MS m/e 467, 469 (M⁺+1).

Compound 782: ¹H NMR (MeOD, 360 MHz) δ 7.65 (d, 1H), 7.61 (d, 1H), 7.47 (d, 1H), 7.41-7.31 (m, 3H), 7.21 (s, 1H), 7.18 (dd, 1H), 5.55 (s, 2H), 3.70 (s, 2H). MS m/e 434, 436, 438 (M⁺+1).

Compound 783: ¹H NMR (MeOD, 360 MHz) δ 7.63 (s, 1H), 7.54-7.42 (m, 3H), 7.34 (dd, 1H), 7.28 (s, 1H), 7.21 (dd, 1H), 5.57 (s, 2H), 3.76 (s, 2H). m/e 453, 455 (M⁺+1).

Compound 784: ¹H NMR (MeOD, 360 MHz) δ 7.73 (d, 1H), 7.41 (m, 2H), 7.33 (d, 1H), 7.20 (dd, 1H), 7.09 (d, 1H), 7.06 (s, 1H), 5.75 (s, 2H), 3.63 (s, 2H). MS m/e 434, 436 (M⁺).

Compound 785: ¹H NMR (MeOD, 360 MHz) δ 7.80 (s, 1H), 7.54 (d, 1H), 7.50 (s, 1H), 7.38 (m, 1H), 7.20 (dd, 1H), 7.16 (s, 1H), 7.11 (s, 1H), 7.07 (d, 1H), 5.42 (s, 2H), 3.61 (s, 2H). MS m/e 434, 436, (M⁺).

Compound 786: ¹H NMR (DMSO, 360 MHz) δ 12.3 (bs, 1H), 8.29 (s, 1H), 7.85 (s, 1H), 7.68 (s, 1H), 7.58 (d, 1H), 7.34 (s, 1H), 7.24 (d, 1H), 6.90 (s, 1H), 5.92 (s, 2H), 3.71 (s, 2H).

Compound 787: ¹H NMR (CDCl₃, 360 MHz) δ 8.23 (d, 1H), 7.64 (d, 1H), 7.56 (d, 1H), 7.49 (s, 1H), 7.29-7.18 (m, 2H), 6.84 (s, 1H), 6.09 (d, 2H), 5.89 (s, 2H), 5.78 (s, 1H), 4.75 (br s, 1H), 3.66 (s, 2H). MS m/e 496, 498 (M⁺+1).

Compound 788: ¹H NMR (MeOD, 360 MHz) δ 7.93 (d, 1H), 7.62-7.59 (m, 2H), 7.38-7.30 (m, 2H), 7.00 (s, 1H), 6.48 (s, 2H part exchanged), 6.18 (s, 1H part exchanged) 5.50 (s, 2H), 3.64 (s, 2H), 2.60 (s, 3H). MS m/e 494, 496 (M⁺+1).

Compound 789: ¹H NMR (MeOD containing CDCl₃, 360 MHz) δ 7.90 (s, 1H), 7.68 (d, 1H), 7.52 (s, 1H), 7.31-7.29 (m, 2H), 7.26 (m, 1H), 7.15 (s, 1H), 6.94 (s, 1H), 5.39 (s, 2H), 3.24 (s, 2H). MS m/e 381, 383 (M⁺+1).

Compound 790: ¹H NMR (MeOD, 360 MHz) δ 7.92 (d, 1H), 7.71 (d, 1H), 7.65-7.62 (m, 2H), 7.41-7.33 (m, 2H), 7.24 (dd, 1H), 7.13 (s, 1H), 5.62 (s, 2H), 3.83 (s, 2H), 2.71 (s, 3H). MS m/e 370, 372 (M⁺+1).

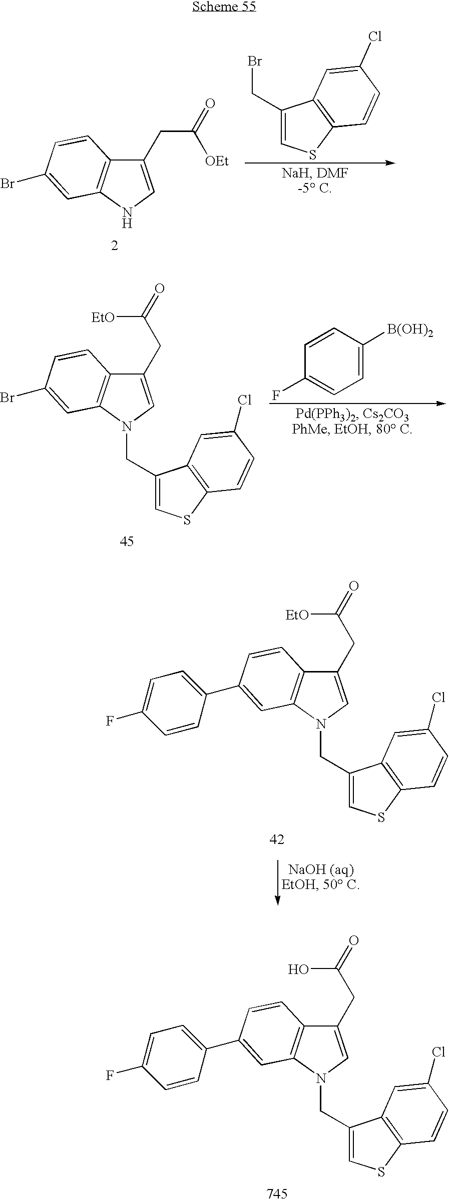

Another general route for the preparation of Helicase inhibitors is illustrated in Scheme 55 and exemplified by the description of the synthesis of compound 736.

### Synthesis of [6-Bromo-1-(5-chloro-benzo[b]thiophen-3-ylmethyl)-1H-indol-3-yl]-acetic acid ethyl ester (41)

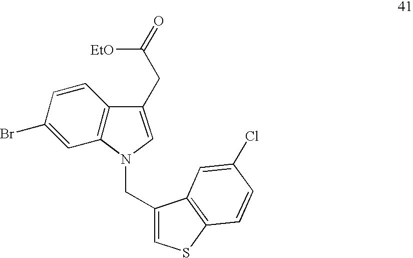

To a stirred solution of the indole 2 (1.0 g, 3.5 mmol) in DMF (35 mL) at −5° C. was added sodium hydride (60% in oil, 156 mg, 3.9 mmol) portion wise over 3 minutes. A solution of 3-bromomethyl-5-chlorobenzothiophene (926 mg, 3.5 mmol) in DMF (15 mL) was then added dropwise over 10 min and the reaction stirred at −5 to 5° C. for 1 h 30 min. The reaction was diluted with water (30 mL) and then made acidic with 10% (w/v) aqueous citric acid solution. The aqueous solution was extracted with EtOAc (2×50 mL), and the combined organic phases washed with brine (100 mL), dried (MgSO₄), filtered and solvent evaporated. The crude was purified by column chromatography (silica, eluent 10% EtOAc in heptane) to give the title compound 41 as a brown oil (1.07 g, 65%). ¹H NMR (CDCl₃, 250 MHz) δ 7.80 (d, 1H), 7.66 (d, 1H), 7.52 (d, 1H), 7.47 (d, 1H), 7.36 (dd, 1H), 7.26 (dd, 1H), 7.09 (s, 1H), 7.00 (s, 1H), 5.41 (d, 2H), 4.17 (q, 2H), 3.74 (d, 2H), 1.25 (t, 3H).

### Synthesis of [1-(5-Chloro-benzo[b]thiophen-3-ylmethyl)-6-(4-fluoro-phenyl)-1H-indol-3-yl]-acetic acid ethyl ester (42)

A stirred mixture of the indole 41 (73 mg, 0.16 mmol), 4-fluorophenylboronic acid (34 mg, 0.24 mmol), caesium carbonate (166 mg, 0.49 mmol) and Tetrakis Pd(PPh₃)₄ (19 mg, 0.016 mol) in EtOH (1 mL) and toluene (2 mL) was degassed with nitrogen for 1 min and then heated at 80° C. for 1 h 30 min. The reaction was cooled to rt, and filtered through Celite, washing with EtOAc (20 mL). The filtrate was evaporated and the crude purified by column chromatography (silica, eluent 30% heptane in DCM) to give the title compound 42 as a white solid (29 mg, 37%). ¹H NMR (CDCl₃, 250 MHz) δ 7.79 (d, 1H), 7.73-7.70 (m, 2H), 7.59-7.53 (m, 2H), 7.44 (d, 1H), 7.39-7.34 (m, 2H), 7.15-7.08 (m, 3H), 7.03 (s, 1H), 5.49 (d, 2H), 4.19 (q, 2H), 3.80 (s, 2H), 1.28 (t, 3H).

### Synthesis of 1-[1-(5-Chloro-benzo[b]thiophen-3-ylmethyl)-6-(4-fluoro-phenyl)-1H-indol-3-yl]-propan-2-one (745)

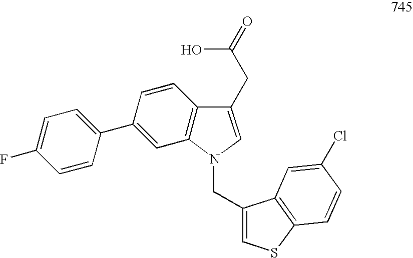

To a stirred solution of ethyl ester 42 (29 mg, 0.061 mmol) in EtOH (7 mL) at rt was added 2 M NaOH (152 μl, 0.30 mmol) and the reaction stirred at 50° C. for 2 h. The reaction was cooled to rt and the solvent evaporated. The residue was suspended in water (3 mL) and acidified to pH 1 with 1 M HCl. The mixture was then filtered, and the solid dried under vacuum, to give the target compound 745 as a pale yellow solid (22 mg, 81%). ¹H NMR (MeOD, 360 MHz) δ 7.88 (d, 2H), 7.71-7.62 (m, 4H), 7.39-7.33 (m, 3H), 7.29 (s, 1H), 7.15 (t, 2H), 5.63 (s, 2H), 3.77 (s, 2H). MS m/e 450, 452 (M⁺+1).

Additional compounds were synthesized using Scheme 55:

Compound 746: ¹H NMR (MeOD, 250 MHz) δ 7.86-7.81 (m, 2H), 7.68-7.62 (m, 2H), 7.40-7.27 (m, 5H), 7.21-7.14 (m, 2H), 6.86 (dd, 1H), 5.58 (s, 2H), 3.84 (s, 3H), 3.78 (s, 2H). MS m/e 484, 486 (M⁺+1).

Compound 747: ¹H NMR (CDCl₃, 360 MHz) δ 7.68 (d, 1H), 7.63 (t, 2H), 7.58 (s, 1H), 7.40-7.18 (m, 4H), 7.06 (s, 1H), 6.98-6.88 (m, 3H), 5.37 (d, 2H), 3.75 (s, 2H), 3.67 (s, 3H). MS m/e 484, 486 (M⁺+1).

Compound 748: ¹H NMR (CDCl₃, 360 MHz) δ 7.70 (d, 1H), 7.64 (d, 1H), 7.60 (d, 1H), 7.45 (dd, 2H), 7.37 (s, 1H), 7.33-7.26 (m, 2H), 7.06 (s, 1H), 6.96 (s, 1H), 6.88 (d, 2H), 5.42 (d, 2H), 3.78 (s, 5H). MS m/e 484, 486 (M⁺+1).

Compound 749: ¹H NMR (MeOD, 360 MHz) δ 7.70 (m, 2H), 7.54-7.48 (m, 4H), 7.28-7.13 (m, 7H), 5.45 (s, 2H), 3.64 (s, 2H). MS m/e 432, 434 (M⁺+1).

Compound 750: ¹H NMR (DMSO, 360 MHz) δ 8.03 (d, 1H), 7.95 (d, 1H), 7.68 (s, 1H), 7.56 (m, 2H), 7.45 (s, 1H), 7.40 (dd, 1H), 7.28-7.23 (m, 4H), 7.00 (dd, 1H), 5.67 (s, 2H), 3.67 (s, 2H), 2.21 (s, 3H). MS m/e 446, 448 (M⁺+1).

Compound 751: ¹H NMR (DMSO, 360 MHz) δ 8.04 (d, 2H), 7.87 (d, 1H), 7.61-7.57 (m, 4H), 7.42 (m, 2H), 7.34 (dd, 1H), 7.72 (d, 2H), 5.74 (s, 2H), 3.68 (s, 2H), 2.35 (s, 3H). MS m/e 446, 448 (M⁺+1).

Compound 752: ¹H NMR (CDCl₃, 250 MHz) δ 7.72-7.59 (m, 3H), 7.41 (s, 1H), 7.36-7.23 (m, 5H), 7.06 (s, 2H), 6.94 (s, 1H), 5.42 (s, 2H), 3.76 (s, 2H), 2.33 (s, 3H). MS m/e 446, 448 (M⁺+1).

Compound 753: ¹H NMR (MeOD, 360 MHz) δ 7.85 (m, 2H), 7.69-7.60 (m, 5H), 7.42-7.30 (m, 5H), 5.62 (s, 2H), 3.79 (s, 2H). MS m/e 466, 468 (M⁺+1).

Compound 754: ¹H NMR (MeOD, 360 MHz) δ 7.89 (m, 2H), 7.70-7.56 (m, 5H), 7.42-7.25 (m, 5H), 5.65 (s, 2H), 3.80 (s, 2H). MS m/e 466, 468 (M⁺+1).

Compound 755: ¹H NMR (DMSO, 250 MHz) δ 8.03 (m, 2H), 7.93 (dd, 1H), 7.87 (d, 1H), 7.78 (dt, 1H), 7.64 (m, 3H), 7.55 (m, 2H), 7.41 (dd, 1H), 7.26 (dd, 1H), 5.71 (s, 2H), 3.69 (s, 2H). MS m/e 457, 459 (M⁺+1).

Compound 756: ¹H NMR (CDCl₃, 360 MHz) δ 7.86 (m, 1H), 7.81 (m, 2H), 7.72 (m, 2H), 7.59 (m, 1H), 7.50 (m, 2H), 7.37 (dt, 2H), 7.20 (s, 1H), 7.02 (s, 1H), 5.52 (s, 2H), 3.85 (s, 2H). MS m/e 457, 459 (M⁺+1).

Compound 757: ¹H NMR (CDCl₃, 360 MHz) δ 7.80 (d, 1H), 7.75-7.69 (m, 6H), 7.51 (m, 1H), 7.41 (dd 1H), 7.37 (dd, 1H), 7.21 (s, 1H), 7.04 (s, 1H), 5.53 (s, 2H), 3.86 (s, 2H). MS m/e 457, 459 (M⁺+1).

Compound 758: ¹H NMR (MeOD, 360 MHz) δ 7.87 (m, 2H), 7.69 (d, 1H), 7.64 (s, 1H), 7.51 (td, 1H), 7.39-7.31 (m, 5H), 7.26-7.15 (m, 2H), 5.65 (s, 2H), 3.81 (s, 2H). MS m/e 450, 452 (M⁺+1).

Compound 759: ¹H NMR (MeOD, 360 MHz) δ 7.84 (m, 2H), 7.67 (d, 2H), 7.46-7.33 (m, 6H), 7.30 (s, 1H), 7.01 (t, 1H), 5.62 (s, 2H), 3.78 (s, 2H). MS m/e 450, 452 (M⁺+1).

Compound 760: ¹H NMR (MeOD, 250 MHz) δ 7.73 (d, 1H), 7.67 (d, 1H), 7.52 (d, 1H), 7.44 (d, 1H), 7.32 (d, 1H), 7.25-7.16 (m, 6H), 7.98 (dd, 1H), 5.47 (s, 2H), 4.40 (s, 2H), 3.67 (s, 2H). MS m/e 462, 464 (M⁺+1).

Compound 761: ¹H NMR (MeOD, 360 MHz) δ 8.11 (m, 2H), 7.99 (d, 1H), 7.92 (m, 3H), 7.74 (t, 1H), 7.65-7.56 (m, 5H), 5.90 (s, 2H), 4.01 (s, 2H), 3.37 (s, 3H), 3.28 (s, 3H). MS m/e 503, 505 (M⁺+1).

Compound 762: ¹H NMR (CDCl₃, 250 MHz) δ 7.89 (dd, 1H), 7.83-7.62 (m, 4H), 7.55-7.31 (m, 5H), 7.20 (dd, 1H), 7.11 (d, 2H), 5.47 (s, 2H), 5.31 (s, 1H), 3.82 (s, 2H). MS m/e 475, 477 (M⁺+1).

Compound 763: ¹H NMR (MeOD, 250 MHz) δ 8.15 (d, 1H), 7.86-7.70 (m, 6H), 7.48 (t, 1H), 7.42-7.21 (m, 4H), 5.62 (s, 2H), 3.61 (s, 2H). MS m/e 475, 477 (M⁺+1).

Compound 764: ¹H NMR (CDCl₃, 360 MHz) δ 8.50 (br s, 2H), 7.90-7.83 (m, 3H), 7.79-7.70 (m, 3H), 7.50 (d, 1H), 7.35 (br s, 3H), 5.68 (s, 2H), 3.73 (s, 2H). MS m/e 433, 435 (M⁺+1).

Compound 765: ¹H NMR (DMSO, 360 MHz) δ 12.25 (br s, 1H), 8.02 (m, 2H), 7.75 (s, 1H), 7.56 (m, 2H), 7.42 (m, 2H), 7.26 (dd, 1H), 7.07 (t, 1H), 6.87 (t, 1H), 6.81 (d, 1H), 6.52 (dd, 1H), 5.71 (s, 2H), 1.47 (br s, 2H), 3.66 (s, 2H). MS m/e 447, 449 (M⁺+1).

Compound 766: ¹H NMR (DMSO, 360 MHz) δ 12.29 (br s, 1H), 10.12 (s, 1H), 8.03 (m, 2H), 7.84 (d, 2H), 7.66-7.61 (m, 3H), 7.45 (s, 1H), 7.41 (dd, 1H), 7.34 (d, 2H), 7.28 (dd, 1H), 5.72 (s, 2H), 3.67 (s, 2H), 2.66 (s, 3H). MS m/e 489, 491 (M⁺+1).

Compound 767: ¹H NMR (DMSO, 250 MHz) δ 12.24 (br s, 1H), 8.59 (s, 1H), 8.03 (d, 2H), 7.82 (s, 1H), 7.64-7.56 (m, 3H), 7.44-7.38 (m, 3H), 7.31-7.19 (m, 3H), 5.87 (s, 2H), 5.72 (s, 2H), 3.67 (s, 2H). MS m/e 490, 492 (M⁺+1).

Compound 768: ¹H NMR (DMSO, 360 MHz) δ 12.26 (br s, 1H), 9.09 (m, 1H), 8.04 (m, 2H), 7.61-7.41 (m, 6H), 7.34-7.24 (m, 3H), 7.07 (d, 1H), 5.67 (s, 2H), 3.68 (s, 2H), 1.76 (s, 3H). MS m/e 489, 491 (M⁺+1).

Compound 769: ¹H NMR (CDCl₃, 360 MHz) δ 9.07 (s, 1H), 8.06-7.99 (m, 2H), 7.96 (dd, 1H), 7.65 (s, 1H), 7.63-7.58 (m, 2H), 7.48-7.36 (m, 3H), 7.27-7.14 (m, 2H), 7.06-6.98 (m, 2H), 6.02 (s, 2H), 5.67 (s, 2H), 3.68 (s, 2H). MS m/e 490, 492 (M⁺+1).

Compound 770: ¹H NMR (MeOD containing CDCl₃, 360 MHz) δ 7.70 (d, 2H), 7.62-7.54 (m, 5H), 7.39-7.33 (m, 2H), 7.24 (d, 1H), 7.20 (d, 1H), 6.95 (s, 1H), 5.34 (s, 2H), 3.66 (s, 2H), 2.54 (s, 3H). MS m/e 446, 448 (M⁺+1).

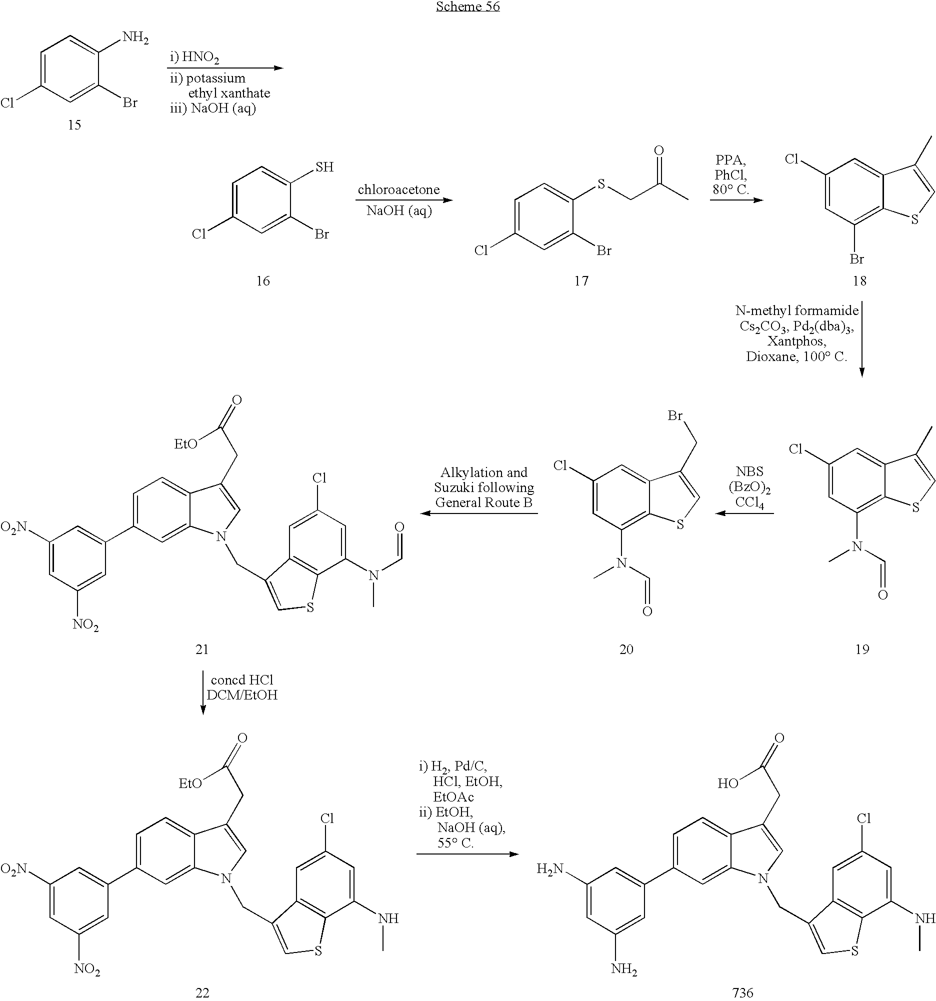

Another general route for the preparation of Helicase inhibitors is illustrated in Scheme 56 and exemplified by the description of the synthesis of compound 736.

### Synthesis of 2-Bromo-4-chloro-benzenethiol (16)

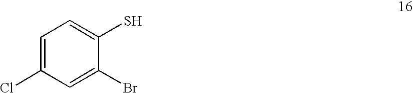

A suspension of 2-bromo-4-chloroaniline 15 (28.0 g, 136 mmol) in water (90 mL) was treated with concentrated HCl (40 mL) and the mixture cooled in ice. A solution of sodium nitrite (10.3 g, 150 mmol) in water (30 mL) was added dropwise over 45 min. The resultant diazonium solution was added in portions over 1 h to a stirred solution of potassium ethyl xanthate (38.0 g, 236 mmol) in water (100 mL) at 75° C. Stirring was continued at this temperature until nitrogen evolution ceased (about 1 h). The reaction mixture was cooled and the crude xanthate ester extracted with DCM (3×100 mL). The combined organic phases were evaporated, dissolved in EtOH (120 mL) and a solution of potassium hydroxide (40 g, 714 mmol) in water (40 mL) added. The solution was refluxed overnight, the EtOH evaporated and the mixture extracted with DCM (3×100 mL). The combined organic layers were dried (Na₂SO₄), filtered and evaporated to give the thiophenol compound 16 as a yellow oil (25.2 g, 83%). MS m/e 221 223 225 (M⁻−1) (negative ion electrospray).

### Synthesis of 1-(2-Bromo-4-chloro-phenylsulfanyl)-propan-2-one (17)

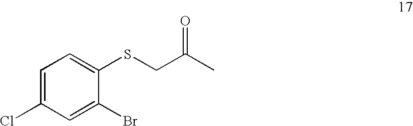

To a stirred solution of NaOH (4.6 g, 114 mmol) in water (250 mL) was added thiophenol 16 (25.0 g, 112 mmol). Chloroacetone (10.5 g, 114 mmol) was then added and the resultant cloudy solution stirred for 3.5 h then extracted with DCM (3×100 mL). The combined organic phases were washed with brine (50 mL), dried (Na₂SO₄), filtered and solvent evaporated. The crude was purified by column chromatography (silica, eluent 16-33% TBME in heptane) to give the thioether compound 17 as a yellow oil (14.5 g, 46%). ¹H NMR (CDCl₃, 250 MHz) δ 7.56 (d, 1H), 7.25-7.18 (m, 2H), 3.70 (s, 2H), 2.32 (s, 3H).

### Synthesis of 7-Bromo-5-chloro-3-methyl-benzo[b]thiophene (18)

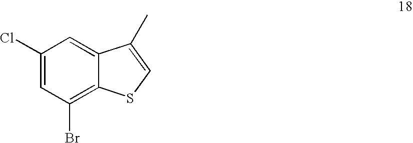

A solution of the thioether 17 (5.0 g, 19 mmol) in chlorobenzene (125 mL) was added to polyphosphoric acid (PPA, 18.0 g), and the mixture stirred at 80° C. for 48 h. The mixture was cooled and then poured into water (400 mL). The mixture was basified with solid sodium carbonate and extracted with DCM (3×100 mL). The combined organic phases were washed with brine (50 mL), dried (Na₂SO₄), filtered and the solvent evaporated. The crude was purified by column chromatography (silica, eluent 100% heptane) to give the bromide compound 18 as a white solid (2.3 g, 64%). ¹H NMR (CDCl₃, 360 MHz) δ 7.45 (s, 1H), 7.32 (s, 1H), 6.98 (s, 1H), 2.2 (s, 3H).

### Synthesis of N-(5-Chloro-3-methyl-benzo[b]thiophen-7-yl)-N-methyl-formamide (19)

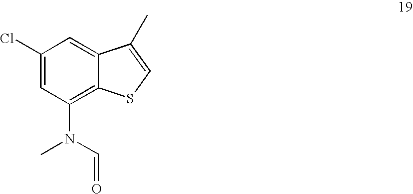

A flask was charged with bromide 18 (1.0 g, 5.5 mmol), N-methylformamide (390 mg, 6.6 mmol), caesium carbonate (2.7 g, 8.2 mmol), Pd₂(dba)₃ (100 mg, 0.11 mmol) and Xantphos (95 mg, 0.16 mmol). Dioxane (30 mL) was added and the mixture briefly degassed with nitrogen before refluxing under a nitrogen atmosphere for 16 h. The mixture was cooled to room temperature, diluted with DCM (100 mL) and washed with water (2×5 mL) and brine (10 mL). The organic phase was dried (Na₂SO₄), filtered and the solvent evaporated. The crude was purified by column chromatography (silica, eluent 20% EtOAc in heptane) to give the methylbenzothiophene compound 19 as a white solid (580 mg, 44%). ¹H NMR (CDCl₃, 360 MHz) δ 8.35 (s, 1H), 7.60 (s, 1H), 7.14 (s, 1H), 7.10 (s, 1H), 3.33 (s, 3H), 2.37 (s, 3H).

### Synthesis of N-(3-Bromomethyl-5-chloro-benzo[b]thiophen-7-yl)-N-methyl-formamide (20)

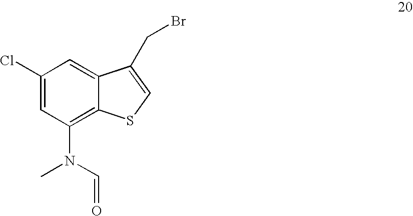

To a stirred solution of methylbenzothiophene 19 (640 mg, 2.7 mmol) in carbon tetrachloride (10 mL) was added N-bromosuccinimide (472 mg, 2.7 mmol) and benzoyl peroxide (70% in water, 20 mg, 0.083 mmol), and the mixture heated at 85° C. under a nitrogen atmosphere for 4 h. More N-bromosuccinimide (80 mg, 0.45 mmol) and benzoyl peroxide (20 mg, 0.083 mmol) were added and heating continued for a further 1 h. After cooling, the solvent was evaporated and the crude purified by column chromatography (silica, eluent 25-33% EtOAc in heptane) to give the bromide compound 20 as a white solid (695 mg, 82%). ¹H NMR (CDCl₃, 360 MHz) δ 8.36 (s, 1H), 7.81 (s, 1H), 7.52 (s, 1H), 7.12 (s, 1H), 4.65 (s, 2H), 3.33 (s, 3H).

### Synthesis of [1-[5-Chloro-7-(formyl-methyl-amino)-benzo[b]thiophen-3-ylmethyl]-6-(3,5-dinitro-phenyl)-1H-indol-3-yl]-acetic acid ethyl ester (21)

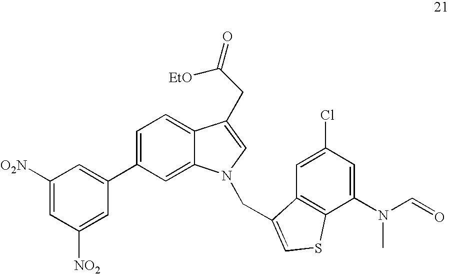

The formamide compound 21 was prepared by alkylation of indole 6 with bromide 20 followed by Suzuki coupling, following the procedures described in Scheme 54. MS m/e 607 (M⁺+1).

### Synthesis of [1-[5-Chloro-7-(methyl-amino)-benzo[b]thiophen-3-ylmethyl]-6-(3,5-dinitro-phenyl)-1H-indol-3-yl]-acetic acid ethyl ester (22)

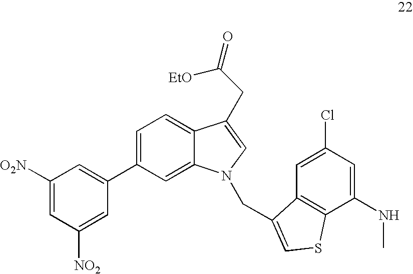

To a stirred solution of formamide 21 (200 mg, 0.33 mmol) in DCM (4 mL) and EtOH (4 mL)was added HCl (2 mL) and the orange solution stirred for 60 h. The solvent was then evaporated and the residue EtOAc (15 mL) and satd NaHCO₃ (5 mL). The aqueous phase extracted with EtOAc (3×15 mL), and the combined organic phases dried (Na₂SO₄), filtered and evaporated to give the dinitro compound 22 as orange oil (200 mg, quantitative yield). MS m/e 579, 581 (M⁺+1).

### Synthesis of [1-[5-Chloro-7-(methyl-amino)-benzo[b]thiophen-3-ylmethyl]-6-(3,5-diamino-phenyl)-1H-indol-3-yl]-acetic acid (736)

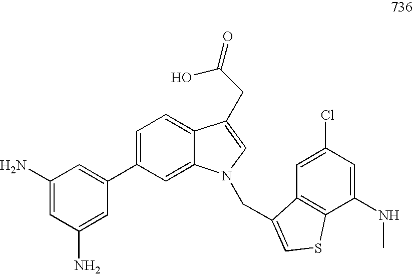

The dinitro compound 22 (60 mg, 0.103 mmol) was suspended in EtOH (8 mL) and EtOAc (4 mL) with heating, and allowed to cool to rt. The mixture was then treated with 10% palladium on carbon (10 mg) and concentrated HCl (4 drops) and stirred under a hydrogen atmosphere for 2 h. The solution was filtered through Celite, washed with EtOH (25 mL) and the filtrate evaporated. To a solution of the crude residue in EtOH (6 mL) and water (1 mL) was added 4 M NaOH (1 mL, 4 mmol) and the reaction stirred at 55° C. for 2 h. The EtOH was evaporated, and the residue diluted with water (5 mL) adjusted to pH 5 with 1 M HCl and solid NaHCO₃ and extracted with EtOAc (3×10 mL). The combined organic phases were washed with brine (5 mL), dried (Na₂SO₄), filtered and solvent evaporated. The solid was suspended in CHCl₃ (5 mL) containing a few drops of MeOH. The liquid was carefully decanted and the remaining solid dried under vacuum to give the compound 736 as a white solid (20 mg, 40%). ¹H NMR (MeOD, 360 MHz) δ 7.50 (d, 1H), 7.40 (s, 1H), 7.20 (d, 1H), 7.08 (d, 1H), 6.95 (s, 1H), 6.36 (m, H part exchanged), 6.02 (s, H part exchanged), 5.41 (s, 2H), 3.59 (s, 2H), 2.80 (s, 3H). MS m/e 491, 493 (M⁺+1).

### Synthesis of Compound 730

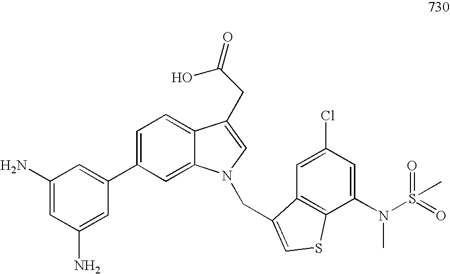

Amine 22 (40 mg, 0.07 mmol) was dissolved in a mixture of DCM (2 mL) and pyridine (1 mL), and 4 Å molecular sieves were added. Excess methane sulfonyl chloride was added (100-200 μl) and the mixture stirred at room temperature for 60 h. The mixture was diluted with DCM (20 mL) and filtered to remove the molecular sieves. The solvent was then evaporated and the crude purified by column chromatography (silica, eluent 25% EtOAc in heptane) to give the [1-[5-Chloro-7-(methanesulfonyl-methyl-amino)-benzo[b]thiophen-3-ylmethyl]-6-(3,5-dinitro-phenyl)-1H-indol-3-yl]-acetic acid ethyl ester (23) as a yellow solid (32 mg, 70%). MS m/e 679 (M⁺+23).

The compound 730 was then obtained by hydrogenation and saponification of 23 according to the procedures described in Scheme 53. Purification was done by HPLC. ¹H NMR (MeOD, 360 MHz) δ 7.83 (s, 1H), 7.65 (d, 1H), 7.55 (2×s, 2H), 7.35-7.30 (m, 3H), 6.91 (s, 2H part exchanged), 6.57 (s, 1H part exchanged), 5.66 (s, 2H), 3.78 (s, 2H), 3.32 (s, 3H), 3.09 (s, 3H). MS m/e 569, 571 (M⁺+1).

### Synthesis of Compounds 731

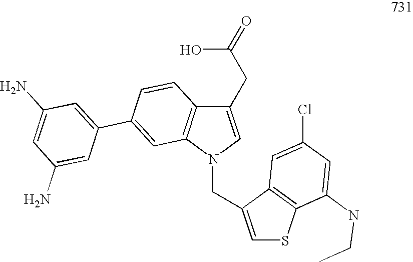

[1-[5-Chloro-7-(ethyl-amino)-benzo[b]thiophen-3-ylmethyl]-6-(3,5-dinitro-phenyl)-1H-indol-3-yl]-acetic acid ethyl ester (24) was synthesised from 18 using N-ethylformamide according to the procedures described in Scheme 56. MS m/e 593 (M⁺+1). The target compound 731 was then obtained by hydrogenation and saponification of 24 according to the procedures described in Scheme 54. ¹H NMR (MeOD containing CDCl₃, 360 MHz) δ 7.51 (d, 1H), 7.40 (s, 1H), 7.23 (d, 1H), 7.10 (s, 1H), 7.06 (s, 1H), 6.92 (s, 1H), 6.44 (s, 2H part exchanged), 6.35 (s, 1H part exchanged), 6.03 (s, 1H part exchanged) 5.41 (2H), 3.65 (s, 2H), 3.20 (q, 2H obs), 1.24 (t, 3H). MS m/e 505, 507 (M⁺+1).

### Synthesis of Compounds 732

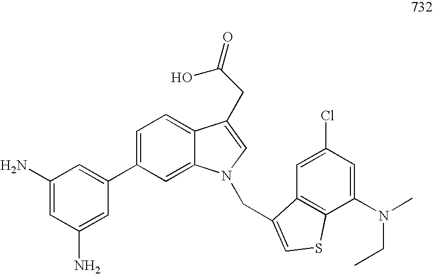

A mixture of 3 M aqueous sulfuric acid (28 μL, 0.084 mmol) and 12.3 M aqueous formaldehyde (16.4 μL, 0.20 mmol) was stirred at −10° C. A suspension of amine 24 (40 mg, 0.068 mmol) and sodium borohydride (9 mg, 0.24 mmol) in THF (1.5 mL) was added to the cooled mixture and the reaction stirred until complete conversion to product, as monitored by LCMS. Saturated NaHCO₃ (4 mL) was added and the mixture extracted with EtOAc (4×4 mL). The combined organic phases were dried (Na₂SO₄), filtered and evaporated to give the crude product which was purified by column chromatography (silica, eluent 25% EtOAc in heptane) to give [1-[5-Chloro-7-(ethyl-methyl-amino)-benzo[b]thiophen-3-ylmethyl]-6-(3,5-dinitro-phenyl)-1H-indol-3-yl]-acetic acid ethyl ester (25) as an orange solid (28 mg, 68%). MS m/e 607 (M⁺+1).

The compound 732 was then obtained by hydrogenation and saponification of 25 according to the procedures described in Scheme 54. Purification was by column chromatography (reverse phase silica (C18), eluent 0-70% MeOH in water). ¹H NMR (MeOD, 360 MHz) δ 7.75 (d, 1H), 7.70 (s, 1H), 7.60 (s, 1H), 7.48 (d, 1H), 7.38 (s, 1H), 7.29 (s, 1H), 7.05 (s, 1H), 6.62 (s, 2H part exchanged), 6.31 (s, 1H part exchanged), 5.72 (s, 2H), 3.90 (s, 2H), 3.45 (q, 2H obs), 3.05 (s, 3H), 1.30 (t, 3H). MS m/e 519, 521 (M⁺+1).

Additional compounds synthesized using Scheme 56 include the following:

Compound 729: ¹H NMR (MeOD, 360 MHz) δ 8.48 (s, 1H), 8.01 (s, 1H), 7.71 (d, 1H), 7.62 (s, 1H), 7.50 (s, 1H), 7.41 (m, 2H), 7.33 (s, 1H), 6.54 (s, 2H), 6.22 (s, 1H), 5.73 (s, 2H), 3.79 (s, 2H), 3.35 (s, 3H). MS m/e 519, 521 (M⁺+1).

Compound 771: ¹H NMR (CDCl₃, 360 MHz) δ 8.25 (s, 1H), 7.60 (s, 1H), 7.44 (d, 1H), 7.37 (s, 1H), 7.20 (d, 1H), 7.15 (s, 1H), 7.03 (s, 1H), 6.95 (s, 1H), 5.35 (s, 2H), 4.08 (q, 2H), 3.85 (q, 2H), 3.66 (s, 2H), 1.18 (t, 3H), 1.08 (t, 3H). MS m/e 534, 535 (M⁺+1).

Compound 772: ¹H NMR (MeOD, 360 MHz) δ 7.48 (s, 1H), 7.38 (d, 1H), 7.10-7.05 (m, 2H), 7.02 (s, 1H), 6.98 (s, 1H), 6.43 (s, 1H), 5.37 (s, 2H), 3.60 (s, 2H), 3.15 (q, 2H), 1.19 (s, 3H). MS m/e 478, 480 (M⁺+1).

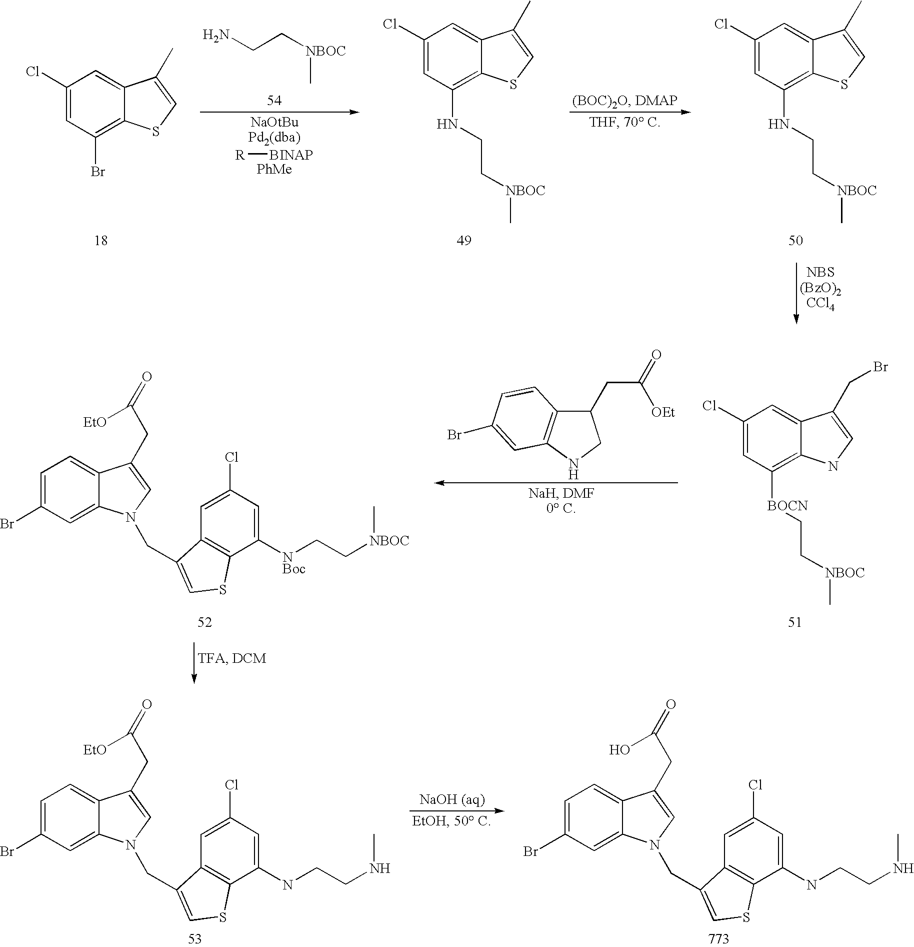

Another general route for the preparation of Helicase inhibitors is illustrated in Scheme 57 and exemplified by the description of the synthesis of compound 773.

### Synthesis of Compound 773

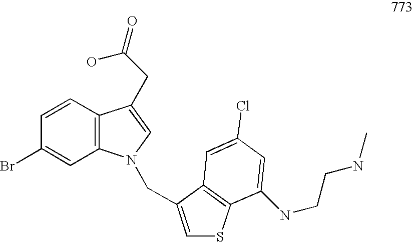

A stirred mixture of the aryl bromide 18 (500 mg, 2.74 mmol), R-BINAP (90 mg, 0.14 mmol), Pd₂(dba)₃ (50 mg, 3.33 mmol), amine 54 (565 mg, 3.27 mmol) and sodium tert-butoxide (445 mg, 4.64 mmol) in toluene (20 mL) was heated at 100° C. for 18 h. The reaction was then cooled to rt and the solvent evaporated. The crude was purified by column chromatography (silica, eluent 20% EtOAc in heptane) to give the title compound 49 as an oil (704 mg, 73%). MS m/e 355, 357 (M⁺+1). The (2-Amino-ethyl)-methyl-carbamic acid tert-butyl ester 54 used in this synthesis was prepared in a manner similar to that described in J. Med. Chem., 2000, 43, 3099.

To a stirred solution of the amine 49 (704 mg, 1.99 mmol) in THF (4 mL) was added Boc anhydride (433 mg, 1.99 mmol) and DMAP (242 mg, 1.99 mmol) and the reaction heated at 70° C. for 6 h. The reaction was then cooled to rt and the solvent evaporated. The crude was purified by column chromatography (silica, eluent 17% EtOAc in heptane) to give the title compound 50 as an oil (760 mg, 84%). MS m/e 477, 479 (M⁺+23).

The compound 51 was prepared as an oil (225 mg, 71%) by bromination of methylbenzothiophene 50 (370 mg, 0.82 mmol) following the procedure described in Scheme 58. ¹H NMR (CDCl₃, 360 MHz) δ 7.79 (s, 1H), 7.56 (br s, 1H), 7.27 (br s, 1H), 4.69 (s, 2H), 2.16 (s, 2H), 3.45 (br m, 2H), 2.89 (s, 3H), 1.42-1.25 (br m, 18H).

The compound 52 was prepared as an oil (135 mg, 43%) by alkylation of indole 2 (120 mg, 0.42 mmol) with the alkyl bromide 51 (225 mg, 0.42 mmol) following the procedure described in General Route B. ¹H NMR (CDCl₃, 250 MHz) δ 7.57-7.45 (m, 2H), 7.28-6.96 (m, 5H), 5.40 (s, 1H), 4.18 (q, 2H), 3.81-3.74 (m, 2H), 3.44 (br s, 1H), 2.88 (br s, 1H), 1.65 (br s, 1H), 1.57 (s, 1H), 1.42-1.24 (br m, 21H).

To a stirred solution of the Boc amine 52 (60 mg, 0.08 mmol) in DCM (5 mL) was added TFA (1 mL) and the reaction stirred at room temperature for 3 h. The solvents were then evaporated and the residue dissolved in DCM (10 mL). The organic solution was washed with satd NaHCO₃ (10 mL), brine (10 mL), dried (NaSO₄), filtered and the solvent evaporated. The crude was purified by column chromatography (silica, eluent 0.1% Et₃N and 10% MeOH in DCM) to give the title compound 53 as an oil (28 mg, 64%). MS m/e 535, 537 (M⁺+1). The target compound 773 was then obtained by saponification of 53 according to the procedure described in Scheme 54. ¹H NMR (DMSO, 360 MHz) δ 7.67 (s, 1H), 7.35-7.31 (m, 2H), 7.23 (s, 1H), 7.06 (s, 1H), 7.01 (d, 1H), 6.45 (s, 1H), 5.95 (bm, 1H), 5.44 (s, 2H), 3.49 (s, 2H), 2.85 (t, 2H) (two signals hidden under solvent peaks). MS m/e 507, 509 (M⁺+1).

### Synthesis of Compound 774

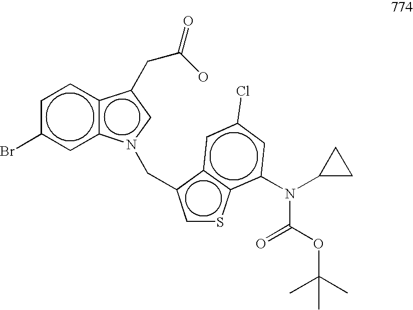

The compound 55 was prepared as an oil (100 mg, 17%) by alkylation of indole 2 (268 mg, 0.95 mmol) with (3-Bromomethyl-5-chloro-benzo[b]thiophen-7-yl)-cyclopropyl-carbamic acid tert-butyl ester (396 mg, 0.95 mmol) (synthesised from 18 using cyclopropylamine and General Route E) following the procedure described in Scheme 54. MS m/e 639-643 (M⁺+23). The target compound 774 was then obtained by saponification of 55 according to the procedure described in Scheme 54. ¹H NMR (CDCl₃, 360 MHz) δ 7.45-7.39 (m, 3H), 7.20 (m, 2H), 7.12 (s, 1H), 7.02 (s, 1H), 6.85 (s, 1H), 5.32 (s, 2H), 3.71 (s, 2H), 3.02 (quintet, 1H), 1.34 (s, 9H), 0.72 (m, 2H), 0.46 (m, 2H). MS m/e 613, 615 (M⁺+23).

### Benzothiaphene Synthesis

The synthesis of the alkyl bromides used in the preparation of the compounds via Schemes 53-57 are described in Scheme 58, Scheme 59 and Scheme 60.

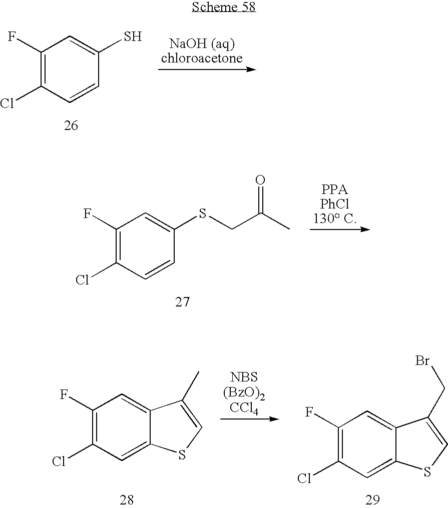

A general route the preparation of bromobenzothiaphenes is illustrated in Scheme 58 and exemplified by the description of the synthesis of compound 29.

To a stirred solution of sodium hydroxide (270 mg, 6.8 mmol) in water (15 mL) was added 3-chloro-4-fluorothiophenol 26 (1.00 g, 6.2 mmol). Chloroacetone (0.49 mL, 6.15 mmol, d 1.161) was added and the resultant cloudy solution stirred for 3.5 h then extracted with DCM (10 mL then 2×5 mL). The combined organic phases were washed with brine (5 mL), dried (MgSO₄), filtered and solvent evaporated. The crude was purified by column chromatography (silica, eluent 0-7% EtOAc in heptane) to give 1-(3-Chloro-4-fluoro-phenylsulfanyl)-propan-2-one (27) as a colourless oil (1.1 g, 83%). ¹H NMR (CDCl₃, 250 MHz) δ 7.43 (dd, 1H), 7.20 (m, 1H), 7.05 (dd, 1H), 3.52 (s, 2H), 2.28 (s, 3H).

A solution of the thioether 27 (1.08 g, 4.94 mmol) in chlorobenzene (30 mL) was added to PPA (4 mL), and the two-phase mixture stirred at 130° C. under a nitrogen atmosphere for 16 h. The mixture was cooled to room temperature and the chlorobenzene layer decanted off. The PPA layer was stirred with water (50 mL) and the resultant solution extracted with DCM (3×25 mL). The combined organic phases (including the chlorobenzene) were washed with satd NaHCO₃ (50 mL), dried (MgSO₄), filtered and solvents evaporated. The crude was purified by column chromatography (silica, eluent 100% heptane) to give 6-Chloro-5-fluoro-3-methyl-benzo[b]thiophene (28) as a white solid (570 mg, 58%) which was a roughly 1:1 mixture of regioisomers. ¹H NMR (CDCl₃, 250 MHz) desired isomer δ 7.85 (d, 1H), 7.44 (d, 1H), 7.12 (s, 1H), 2.72 (s, 3H), undesired isomer δ 7.66-7.50 (m, 2H), 7.12 (s, 1H), 2.39 (s, 3H).

Methylbenzothiophene 28 (560 mg, 2.8 mmol) was dissolved in carbon tetrachloride (2 mL), then N-bromosuccinimide (500 mg, 2.8 mmol) and benzoyl peroxide (70%, small spatula tip) was added and the solution heated at 80° C. under a nitrogen atmosphere for 8 h. The solvent was evaporated and the residue extracted with heptane (5×5 mL). The combined organic extracts were evaporated and the crude purified by column chromatography (silica, 0-5% EtOAc in heptane) to give 3-Bromomethyl-6-chloro-5-fluoro-benzo[b]thiophene (29) as a mixture of regioisomers (470 mg, 60%). ¹H NMR (CDCl₃, 250 MHz) desired isomer δ 7.89 (d, 1H), 7.58 (s 1H), 7.21 (s, 1H), 5.05 (s, 2H), undesired isomer δ 7.67-7.60 (m, 2H), 7.17 (s, 1H), 4.67 (s, 2H).

The compound 723 was then synthesised via Scheme 54. The desired 6-chloro-5-fluoro regioisomer could be purified away from its 4-chloro-5-fluoro regioisomer after the alkylation step.

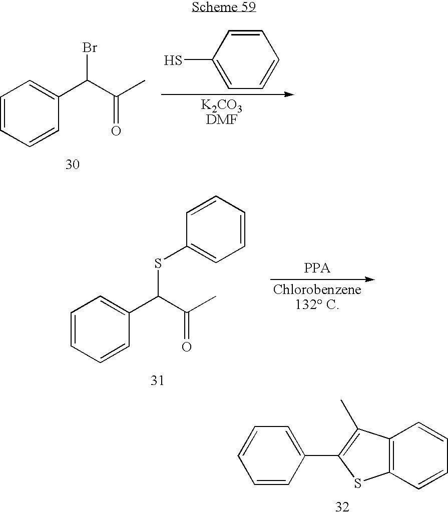

A general synthetic route for the preparation of 2-phenyl-benzothiaphenes is illustrated in Scheme 59 and exemplified by the description of the synthesis of compound 32.

To a stirred solution of thiophenol (241 μL 2.4 mmol, d 1.073) in DMF (5 mL) was added potassium carbonate (648 mg, 4.7 mmol) and the reaction stirred at room temperature for 2 min. A solution of 1-bromo-1-phenyl-propan-2-one (30) (500 mg, 2.4 mmol) in DMF (3 mL) was then added and the reaction stirred at room temperature overnight. After 18 h, the reaction was complete as monitored by TLC so water (10 mL) was added and the mixture extracted into EtOAc (3×20 mL). The combined organic phases were dried (Na₂SO₄), filtered and solvent evaporated. The crude was purified by column chromatography (silica, 5% EtOAc in heptane) to give 1-Phenyl-1-phenylsulfanyl-propan-2-one (31) as a yellow solid (172 mg, 30%). ¹H NMR (CDCl₃, 250 MHz) δ 7.15-7.40 (m, 10H), 5.0 (s, 1H), 2.25 (s, 3H).

The bromo-1-phenyl-propan-2-one (30) used in the above synthesis was prepared in a manner similar to that described in Tetrahedron Asymmetry, 1994, 5 (7), 1249-1268. Other substituted bromo-1-phenyl-propan-2-ones described herein were also prepared in a similar fashion.

3-Methyl-2-phenyl-benzo[b]thiophene (32) was prepared as a cream solid (113 mg, 71%) by cyclisation of thioether 31 (172 mg, 0.71 mmol) following the procedure described in Scheme 56. ¹H NMR (CDCl₃, 360 MHz) δ 7.85 (d, 1H), 7.74 (d, 1H), 7.57 (m, 2H), 7.50-7.34 (m, 5H), 2.49 (s, 3H).

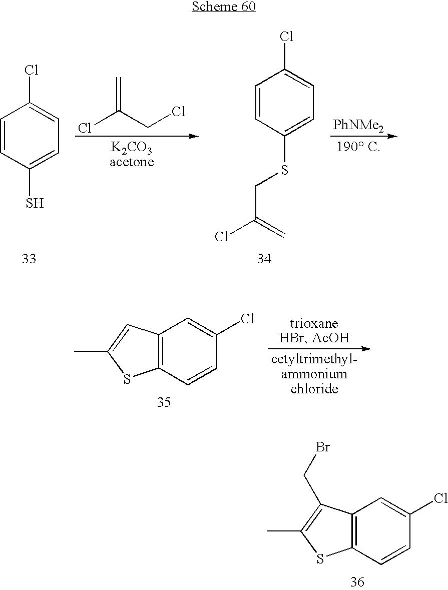

A general synthetic route for the preparation of 2-methyl-benzothiaphenes is illustrated in Scheme 60 and exemplified by the description of the synthesis of compound 36.

To a solution of 4-chlorothiophenol (2.88 g, 20 mmol) in acetone (60 mL) was added potassium carbonate (5.52 g, 40 mmol) followed by 2,3-dichloropropene (2.20 g, 20 mmol). The resulting solution was heated to 60° C. for 1 h, and then allowed to cool to rt. The acetone was evaporated and the crude residue dissolved in EtOAc (30 mL) and washed with water (30 mL). The aqueous layer was then extracted with EtOAc (2×20 mL). The combined organic layers were dried (Na₂SO₄), filtered and evaporated to give 1-chloro-4-(2-chloro-allylsulfanyl)-benzene (34) as a white solid (4.20 g, 96%). ¹H NMR (CDCl₃, 360 MHz) δ 7.25-7.40 (m, 4H), 5.28 (s, 2H), 3.70 (s, 2H).

The thioether 34 (2.00 g, 9.1 mmol) was dissolved in N,N-dimethylaniline (10 mL) and heated to 190° C. for 24 h, then allowed to cool to rt. TBME (30 mL) was added to the reaction mixture, which was then washed with 2 M HCl (3×30 mL). The organic phase was dried (Na₂SO₄), filtered and evaporated to give the crude residue. This was crystallised from 5% DCM in heptane to give 5-Chloro-2-methyl-benzo[b]thiophene (35) as white needles (200 mg, 12%). A further 950 mg (57%) of less pure product was recovered by evaporation of the mother liquors. ¹H NMR (CDCl₃, 360 MHz) δ 7.68 (m, 2H), 7.23 (d, 1H), 6.91 (s, 1H), 2.58 (s, 3H).

To a solution of acetic acid (100 μl) in hydrogen bromide (48% in H₂O, 4 mL) was added benzothiophene 35 (350 mg, 1.9 mmol) followed by trioxane (309 mg, 3.4 mmol) and hexadecyltrimethylammonium bromide (14 mg, 0.04 mmol). The resulting suspension was stirred at room temperature for 5 h. The reaction mixture was then diluted with water (15 mL) and filtered, washing with water (2×5 mL) to give 3-Bromomethyl-5-chloro-2-methyl-benzo[b]thiophene (36) as a white solid (310 mg, 59%). ¹H NMR (CDCl₃, 360 MHz) δ 7.66 (s, 1H), 7.61 (d, 1H), 7.25 (d, 1H), 4.60 (s, 2H), 2.52 (s, 3H).

### Other Benzothiaphene Synthesis

### 5-Chloro-7-methoxy-3-methyl-benzo[b]thiophene (37)

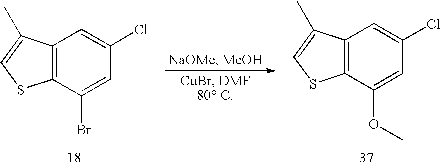

To a stirred suspension of the aryl bromide 18 (600 mg, 2.3 mmol) in MeOH (6 mL) and DMF (55 μL) was added sodium methoxide (1.24 g, 23 mmol) and the reaction heated to 80° C. Copper (I) bromide (32 mg, 0.23 mmol) was added and stirring continued at 80° C. for 6 hours. Reaction mixture turned dark blue. The reaction was cooled to room temperature overnight and then diluted with DCM (80 mL) and washed with water (80 mL). The green aqueous phase was extracted with DCM (2×80 mL) and the combined organic phases dried (Na₂SO₄), filtered and solvent evaporated. The crude was purified by column chromatography (silica, eluent 100% heptane) to give the title compound 37 as a colourless oil (346 mg, 71%). ¹H NMR (CDCl₃, 360 MHz) δ 7.33 (s, 1H), 7.11 (s, 1H), 6.78 (s, 1H), 4.00 (s, 3H), 2.40 (s, 3H).

### 5-Nitro-3-chloromethyl-benzo[b]thiophene (39)

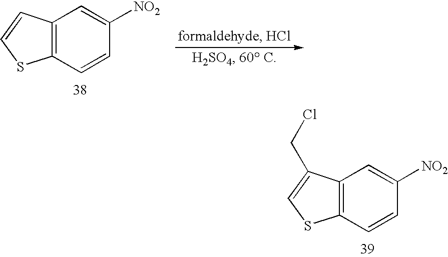

To a stirred solution of 5-nitrobenzothiophene 38 (800 mg, 4.48 mmol), concentrated HCl (1.04 mL) and formaldehyde (30% in water, 600 μL) at 60° C. was added concentrated sulfuric acid (680 μL) dropwise. The reaction was heated for a further 24 h and then cooled to room temperature and diluted with water (5 mL). The aqueous mixture was extracted with EtOAc (10 mL) and washed with water (10 mL), satd NaHCO₃ (10 mL), and water (10 mL). The organic phase was dried (MgSO₄), filtered and the solvent evaporated to give the title compound 39 as an orange solid (550 mg, 54%). ¹H NMR (CDCl₃, 360 MHz) δ 8.80 (d, 1H), 8.27 (dd, 1H), 8.00 (d, 1H), 7.70 (s, 1H), 4.91 s, 2H).

The 5-nitrobenzothiophene 38 used in the above synthesis was prepared in a manner similar to that described in J. Am. Chem. Soc., 1935, 57, 1611.

### 5-Bromo-3-bromomethyl-benzo[b]thiophene (7)

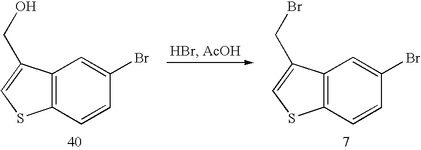

5-bromobenzothiophene-3-methanol 40 (49 mg, 0.20 mmol) was dissolved in 33 wt. % hydrogen bromide in acetic acid (2 mL) and the mixture stirred at room temperature for 20 min. The reaction was then diluted with diethyl ether (3 mL) and washed with water (3 mL) followed by satd NaHCO₃ (2×3 mL). The organic phase was dried (MgSO₄), filtered and evaporated to give the title compound 7 as a beige solid (49 mg, 79%). ¹H NMR (CDCl₃, 250 MHz) δ 8.04 (d, 1H), 7.74 (d, 1H), 7.55 (s, 1H), 7.50 (dd, 1H), 4.71 (s, 2H).

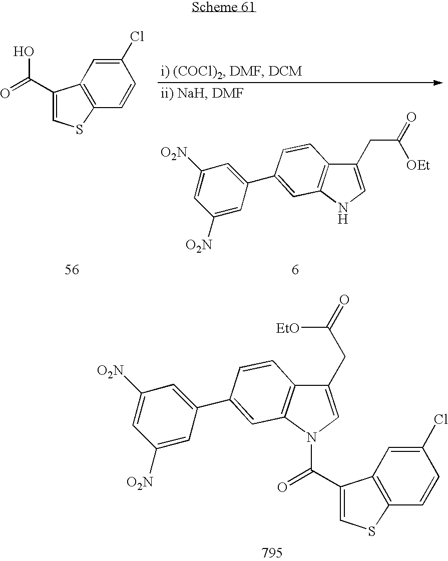

### Synthesis of [1-(5-Chloro-benzo[b]thiophene-3-carbonyl)-6-(3,5-dinitro-phenyl)-1H-indol-3-yl]-acetic acid ethyl ester (795)

To a stirred solution of 5-chloro-benzo[b]thiophene-3-carboxylic acid 56 (58 mg, 0.27 mmol) in DCM (2 mL) was added DMF (1 drop) followed by oxalyl chloride (46 μL, 0.54 mmol, d 1.478) and the reaction stirred at rt for 2 h 30 min. The solvent was then evaporated to leave the crude acid chloride.

To a stirred solution of sodium hydride (60% in oil, 12 mg, 0.30 mmol) in DMF (1.5 mL) at 0° C. was added a solution of the indole 6 (100 mg, 0.27 mmol) in DMF (2 mL) and the mixture stirred at 0° C. for 5 min. A solution of the acid chloride, as prepared above, in DMF (1 mL) was then added dropwise over 5 min and the reaction stirred at 0-8° C. for 2 h. The reaction was then diluted with EtOAc (10 mL) and washed with 10% (w/v) aqueous citric acid solution (10 mL). The aqueous phase was extracted with EtOAc (10 mL) and the combined organic phases dried (MgSO₄), filtered and solvent evaporated. The crude was purified by dry flash chromatography (silica, eluent 5-20% EtOAc in heptane) to give the title compound 795 as a yellow solid (30 mg, 20%). ¹H NMR (CDCl₃, 250 MHz) δ 9.03 (t, 1H), 8.90-8.83 (m, 3H), 8.20 (d, 1H), 8.11 (s, 1H), 7.89 (d, 1H), 7.83-7.76 (m, 1H), 7.73-7.61 (m, 2H), 7.48 (dd, 1H), 4.21 (q, 2H), 3.78 (s, 2H), 1.29 (t, 3H).

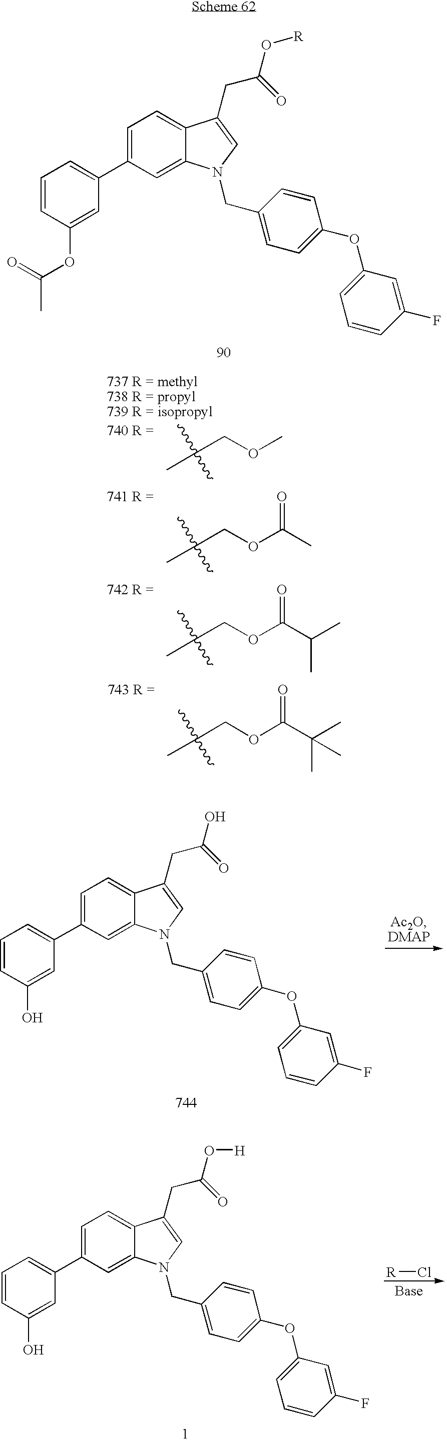

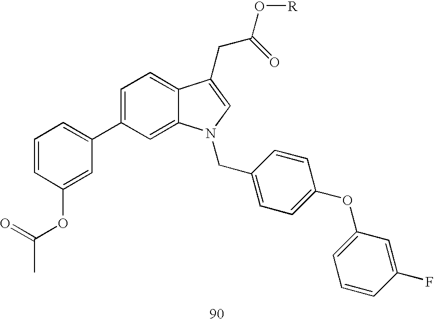

Seven ester prodrugs, compounds 737-743, of the indole target compound 744, can be synthesized via Scheme 62.

The synthesis of compound 744 is shown below:

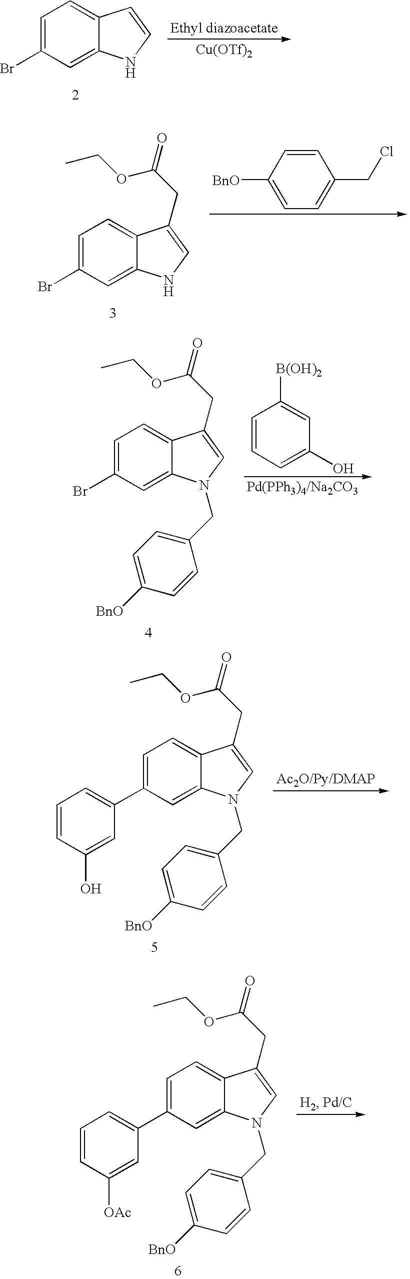

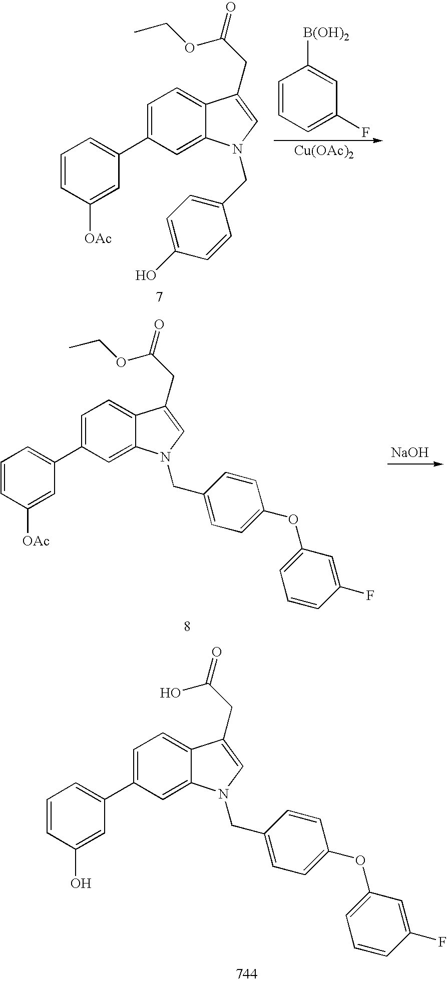

Additional compounds synthesised using any of the Schemes are listed in the Tables below under the section “example of activity.”

### Example A

### HCV Helicase Prompt FRET Assay

Test compounds were diluted to 2.5 mM by adding 6 uL of a 10 mM solution of the compound to 18 uL of DMSO in a 384-well Costar polypropylene plate. Serial dilutions (2.5×) were performed in the plate using DMSO as the diluent. 1 ul of solution was transferred from each well to a new 384-well Costar polypropylene plate.

A 2× mixture was prepared containing 100 nM helicase substrate, 500 nM helicase capture strand (CS), and 600 μM ATP by diluting stock solutions with assay buffer consisting of 25 mM MOPS, pH 7.0, and 1.5 mM MgCl₂, 0.005% (v/v) Triton X-100. Stock helicase substrate was prepared by annealing a FAM-labeled oligonucleotide to a BHQ-1 labeled oligonucleotide, which were both custom synthesized and HPLC-purified at Biosearch Technologies. The FAM-labeled and BHQ-labeled oligonucleotide had the following sequences:

(SEQ ID NO. 1)

5′ FAM d(TAGTACCGCCACCCTCAGAACCTTTTTTTTTTTTTT) 3′

(SEQ ID NO. 2)

3′ BHQ-1 (ATCATGGCGGTGGGAGTCTTGG)d 5′

The helicase capture strand was custom synthesized and purified at Integrated DNA Technologies. The helicase capture strand had the following sequence:

| | |
|---|---|
| 5′ d(TAGTACCGCCACCCTCAGAACC) 3′ | (SEQ ID NO. 3) |

The ATP solution was prepared in MilliQ water, pH adjusted to approximately 7 with NaOH. ATP powder was obtained from Sigma (A-7699). The concentration was determined via A₂₆₀ (ext. coeff=15,400 M⁻¹cm⁻¹).

11.5 μL of assay buffer was added to all wells of the plate containing luL of test compound. 5 μL of the Compound/assay buffer mixture was added to a black polystyrene 384-well Proxiplate. A 4× Enzyme solution was prepared by diluting enzyme stock containing full-length NS3 (1-631), purified at Array BioPharma, with assay buffer. 5 μL of the 4× Enzyme solution was added to the Proxiplate and incubated for 5 minutes (for control wells lacking enzyme, assay buffer was used instead). 10 μL of 2× substrate/CS/ATP mixture was then added to all wells of Proxiplate. The final assay conditions included 50 nM Substrate, 250 nM Capture Strand, 300 μM ATP, 5 or 6 nM Enzyme, and 2% (v/v) DMSO. The reaction proceeded for 70 cycles (˜30 min.) on an Envision at room temperature. The plate was read using the Envision (top mirror=FITC; Excitation filter=FITC 485; Emission filter=FITC 535). The fluorescence intensity was recorded for a total of 30 minutes. The initial rates of the reactions was calculated and used to calculate IC₅₀ values. The IC₅₀ curve fitting was performed using either a 4-parameter or 5-parameter logistic equation.

### Example B

### HCV Helicase TR-FRET Assay

A DMSO test compound plate starting with 10 mM stock solutions of the compounds in DMSO was prepared as described in Example A.

A 2× Helicase Substrate/Capture Strand/ATP mixture was prepared by diluting the stock solutions with assay buffer consisting of 25 mM MOPS, pH 7.0, and 500 μM MgCl₂, 0.005% (v/v) Triton X-100. The helicase substrate was TRUPOINT™ helicase assay reagent obtained from PerkinElmer, Inc. Substrate stock solution was prepared per the instruction booklet from Perkin Elmer; #AD0166 as 1 μM aliquots kept at −20° C. The capture strand was also TRUPOINT™ helicase assay reagent obtained from PerkinElmer, Inc. Capture strand stock solution was prepared per the instruction booklet from Perkin Elmer; #AD0164 as 15 μM aliquots kept at −20° C. ATP stock solution was prepared as in Example A. The resulting mixture contained 8 nM Helicase Substrate, 30 nM Capture Strand, and 200 μM ATP.

As in Example A, 11.5 μL of assay buffer was added to all wells of the plate containing 1 uL of test compound. 5 μL of the compound/assay buffer mixture was added to a white polystyrene 384-well Proxiplate. A 4× Enzyme solution was prepared by diluting enzyme stock containing full-length NS3 (1-631), purified at Array BioPharma, with assay buffer. 5 μL of the 4× Enzyme solution was added to the Proxiplate and incubated for 5 minutes (for control wells lacking enzyme, assay buffer was used instead). 10 μL of the 2× substrate/CS/ATP mixture was added to all wells. The final assay conditions included 4 nM Helicase Substrate, 15 nM Helicase Capture Strand, 100 μM ATP, 2.5 nM Enzyme, and 2% (v/v) DMSO. The reaction proceded for 25 cycles on an Envision at 22° C. (i.e., kinetic read for 30 min). The plate was read in the Envision (mirror=LANCE/DELFIA; Excitation filter=UV2 (TRF) 320; Emission filter=Europium 615; Delay=60 μs, window time=50 μs, 2000 μs between flashes). The fluorescence intensity was recorded for a total of 30 minutes. The initial rates of the reactions was calculated and used to determine IC₅₀ values. The IC₅₀ curve fitting was performed using either a 4-parameter or 5-parameter logistic equation.

### Examples of Activity

For the IC₅₀ activity in the following tables, A=10˜50 μM, B<10 μM, C>50 μM, and ND or nd=not determined.

TABLE 1
Examples of compounds prepared using Scheme 1 or Scheme 2.

| Example | Structure | IC50 Activity | LC/MS |
|---|---|---|---|
| 100 | 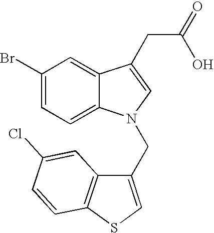 | A | 433.9 (neg APCI) |
| 101 | 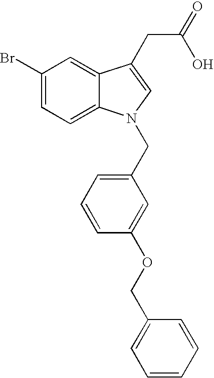 | B | 448.1, 450.0(neg APCI) |
| 102 | 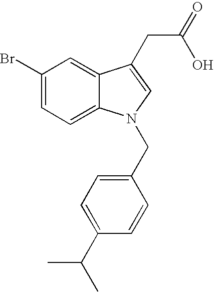 | A | 384.1, 386.0(neg APCI) |
| 103 | 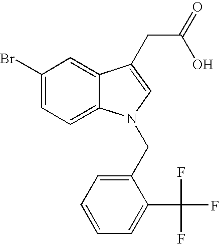 | A | 410.0, 411.9(neg APCI) |
| 104 | 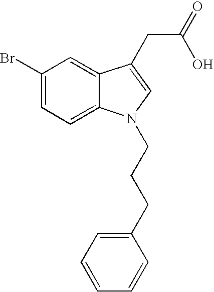 | A | 370.2, 372.0(neg APCI) |
| 105 | 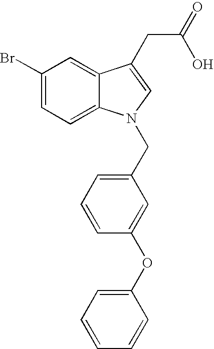 | B | 434.1, 436.0(neg APCI) |
| 106 | 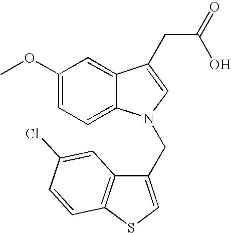 | A | 384.0 (neg APCI) |
| 107 | 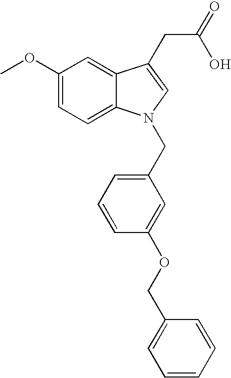 | A | 400.0 (neg APCI) |
| 108 | 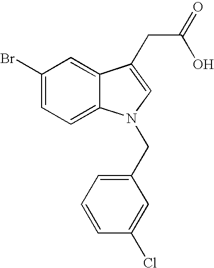 | A | 377.9 (neg APCI) |
| 109 | 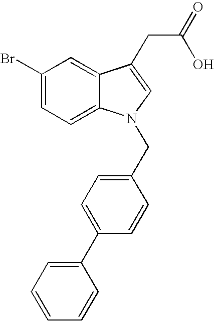 | B | 418.0, 419.9(neg APCI) |
| 110 | 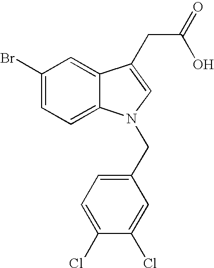 | A | 411.9 (neg APCI) |
| 111 | 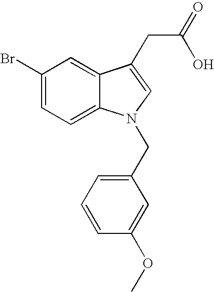 | A | 372.0, 374.0(neg APCI) |
| 112 | 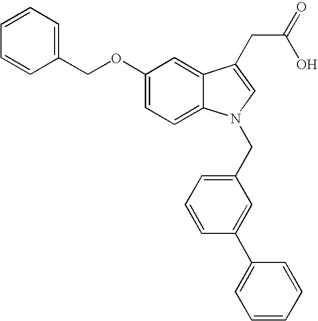 | B | 446.2 (neg APCI) |
| 113 | 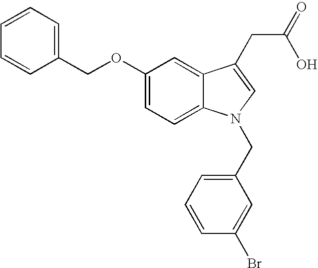 | A | 448.1, 450.0(neg APCI) |
| 114 | 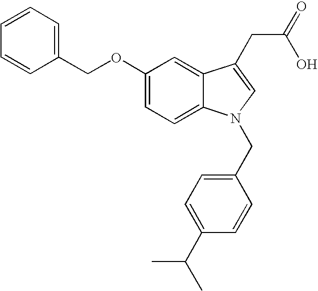 | A | 412.2 (neg APCI) |
| 115 | 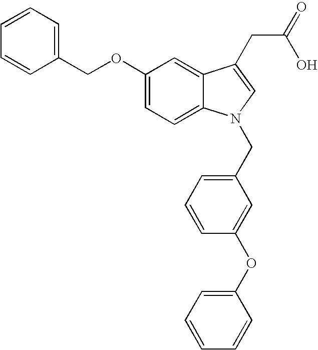 | B | 462.1 (neg APCI) |
| 116 | 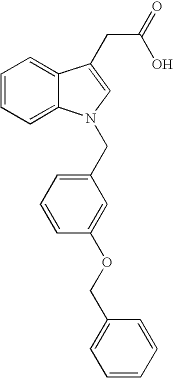 | A | 370.2 (neg APCI) |
| 117 | 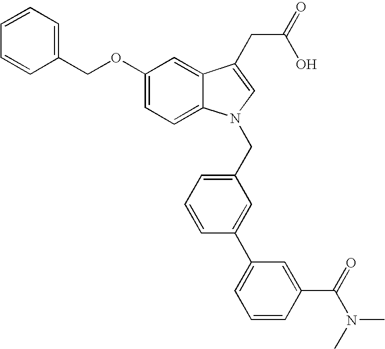 | A | 517.2 (neg APCI) |
| 118 | 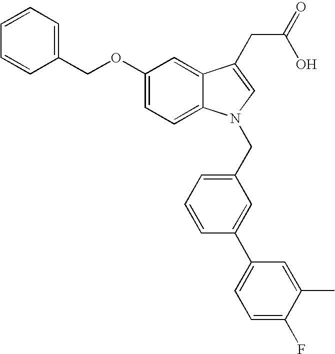 | B | 478.1 (neg APCI) |
| 119 | 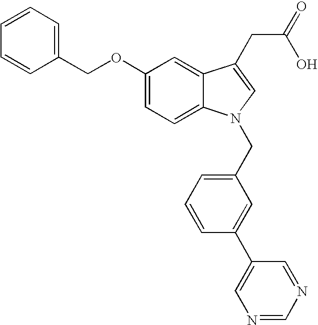 | A | 448.1 (neg APCI) |
| 120 | 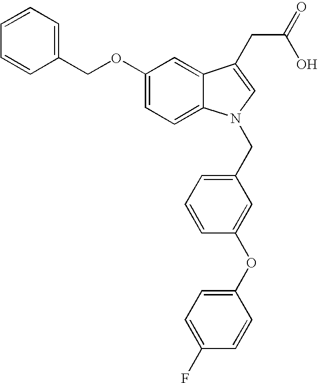 | B | 480.0 (neg APCI) |
| 121 | 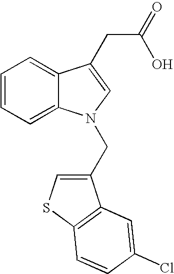 | A | 354.0 (neg APCI) |
| 122 | 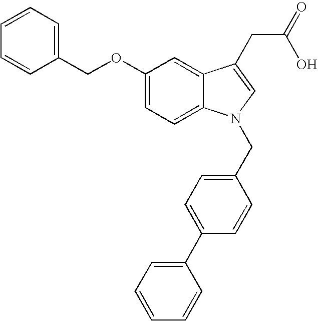 | B | 446.1 (neg APCI) |
| 123 | 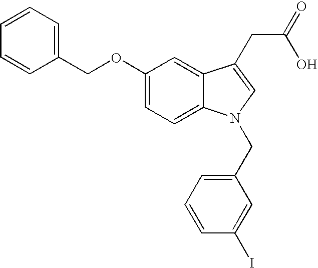 | B | 496.0 (neg APCI) |
| 124 | 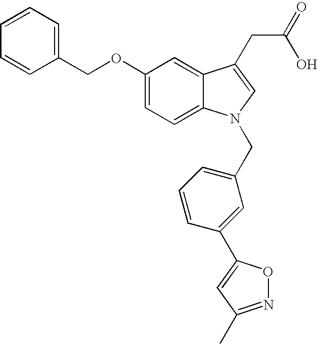 | A | 451.1 (neg APCI) |
| 125 | 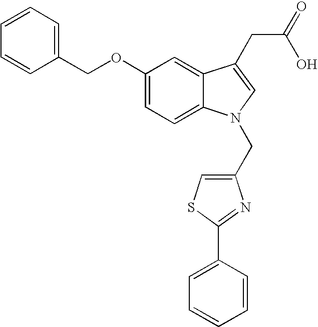 | A | 453.1 (neg APCI) |
| 126 | 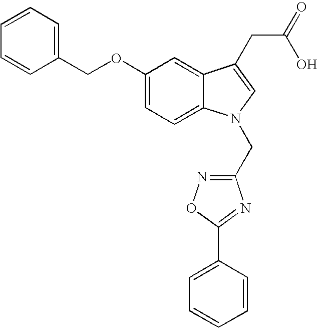 | A | 438.1 (neg APCI) |
| 127 | 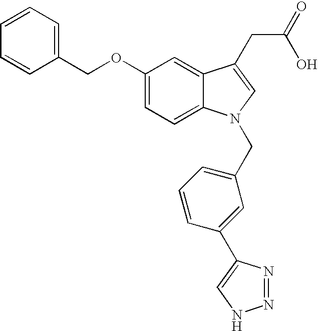 | A | 437.2 (neg APCI) |
| 128 | 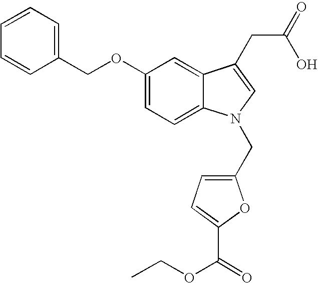 | A | 432.1 (neg APCI) |
| 129 | 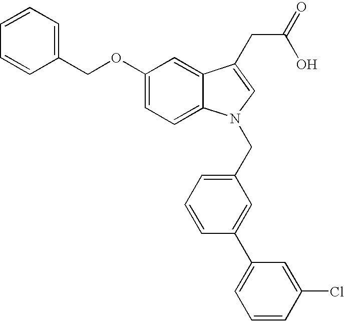 | B | 480.3 (neg APCI) |
| 130 | 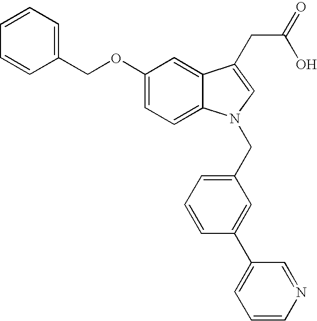 | A | 447.2 (neg APCI) |
| 131 | 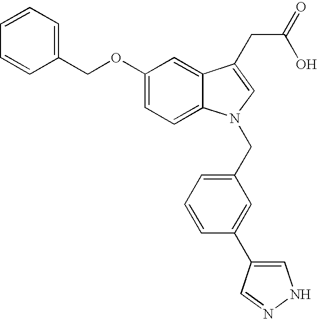 | B | 436.2 (neg APCI) |
| 132 | 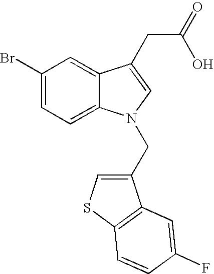 | A | 416.0, 417.9(neg APCI) |
| 133 | 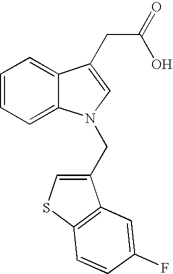 | A | 338.2 (neg APCI) |
| 134 | 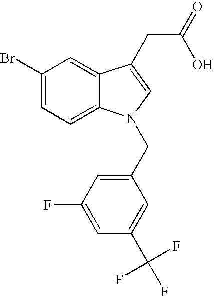 | A | 427.9, 429.9(neg APCI) |
| 135 | 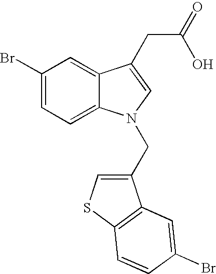 | B | 477.9 (neg APCI) |
| 136 | 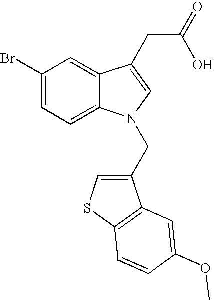 | B | 428.1, 430.0(neg APCI) |
| 137 | 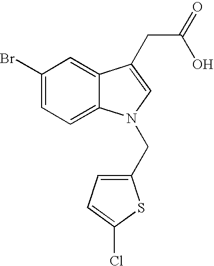 | A | 382.0, 384.0(neg APCI) |
| 138 | 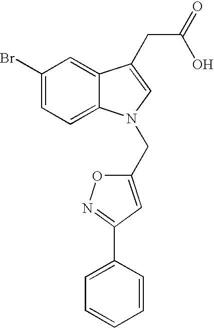 | A | 409.1, 411.1(neg APCI) |
| 139 | 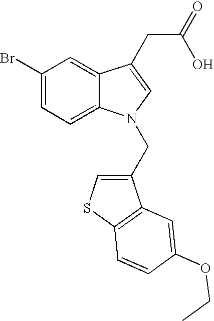 | B | 382.0, 384.0(neg APCI) |
| 140 | 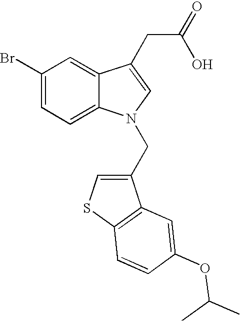 | B | 456.0, 458.00(neg APCI) |
| 141 | 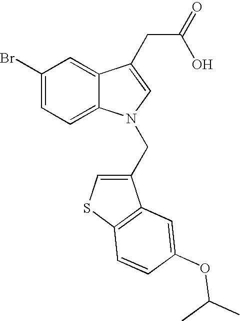 | A | |
| 142 | 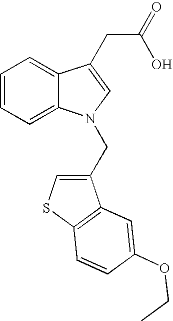 | A | 363.7 (neg APCI) |

A = 10~50 μM
B < 10 μM

TABLE 2
Examples of compounds prepared using Scheme 3.

| Example | Structure | IC50 Activity | LC/MS |
|---|---|---|---|
| 143 | 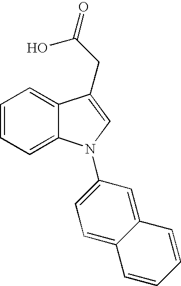 | A | 300.0 (neg APCI) |
| 144 | 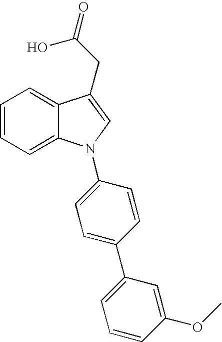 | B | 356.1 (neg APCI) |
| 145 | 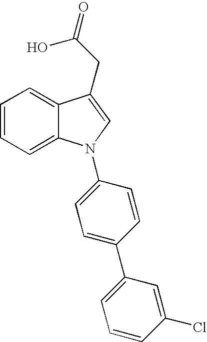 | B | 360.0 (neg APCI) |
| 146 | 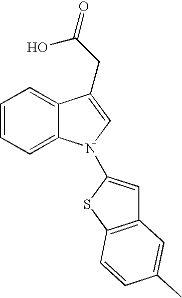 | B | 320.0 (neg APCI) |
| 147 | 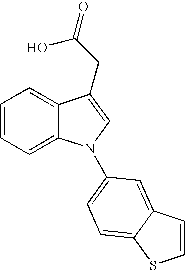 | A | 306.0 (neg APCI) |
| 148 | 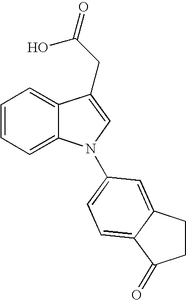 | A | 304.09 (neg APCI) |
| 149 | 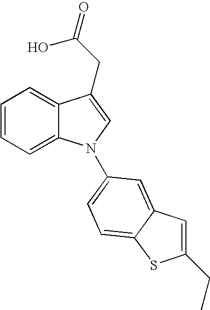 | B | 334.0 (neg APCI) |
| 150 | 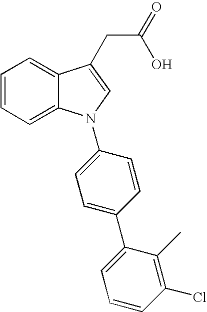 | B | 373.9 (neg APCI) |
| 151 | 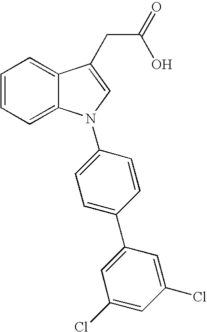 | B | 393.9 (neg APCI) |
| 152 | 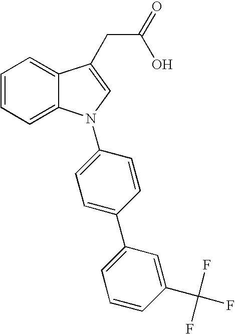 | B | 394.0 (neg APCI) |
| 153 | 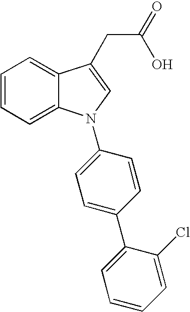 | B | 359.9 (neg APCI) |
| 154 | 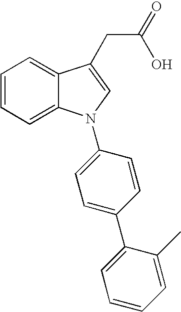 | B | 340.0 (neg APCI) |
| 155 | 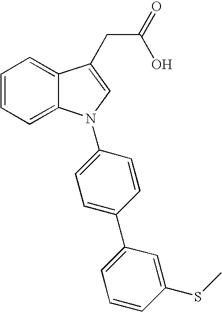 | B | 374.1 (pos, APCI) |
| 156 | 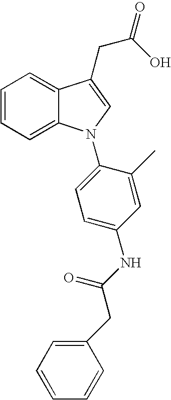 | A | 396.7 (neg APCI) |
| 157 | 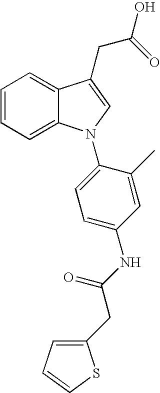 | A | 403.1 (neg APCI) |
| 158 | 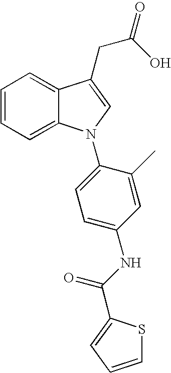 | A | 389.1 (neg APCI) |
| 159 | 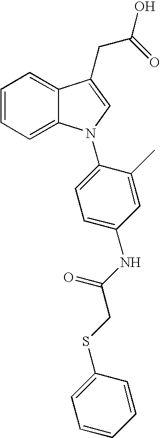 | A | 429.2 (neg APCI) |
| 160 | 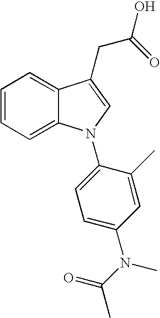 | A | 335.2 (neg APCI) |
| 161 | 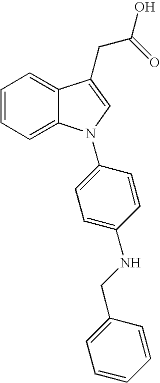 | B | 357.0 (pos APCI) |
| 162 | 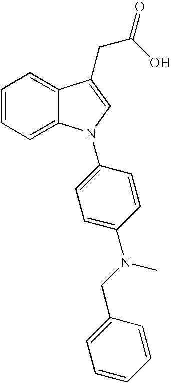 | B | 371.0 (pos APCI) |
| 163 | 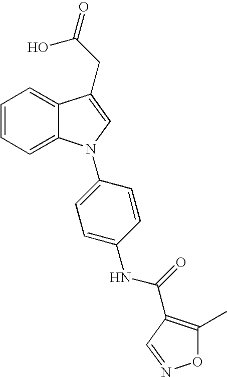 | A | 374.3 (neg APCI) |
| 164 | 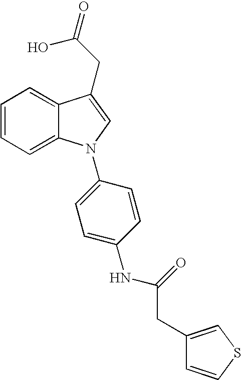 | A | 389.3 (neg APCI) |
| 165 | 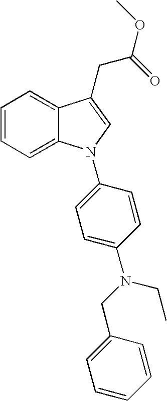 | A | 399.2 (pos APCI) |
| 166 | 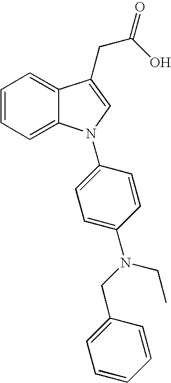 | B | 385.1 (pos APCI) |
| 167 | 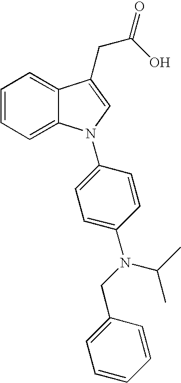 | B | 397.0 (neg APCI) |
| 168 | 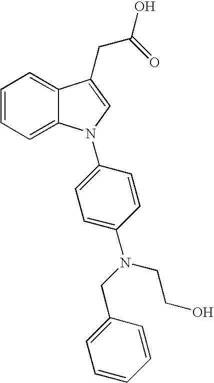 | A | 401.1 (pos APCI) |
| 169 | 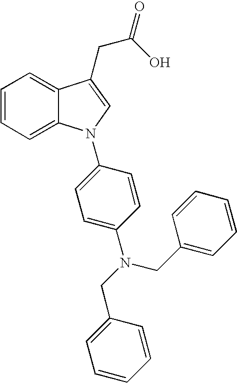 | B | 447.2 (pos APCI) |
| 170 | 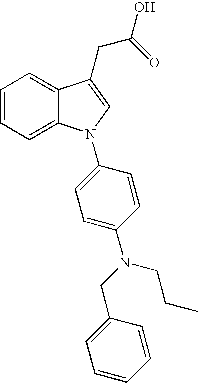 | A | 399.1 (pos APCI) |
| 171 | 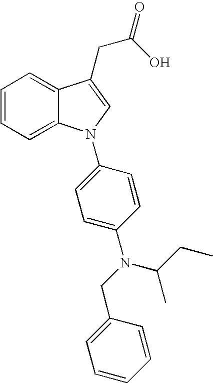 | B | 413.2 (pos APCI) |
| 172 | 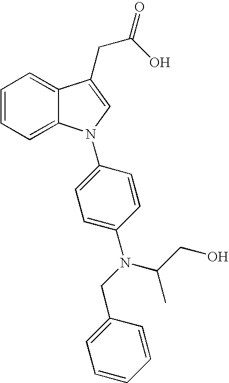 | B | 415.2 (pos APCI) |
| 173 | 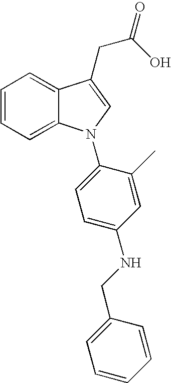 | A | 371.2 (pos APCI) |
| 174 | 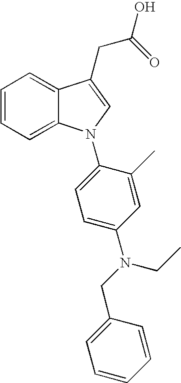 | B | 399.2 (pos APCI) |
| 175 | 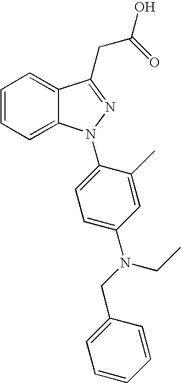 | A | 400.2 (pos APCI) |
| 176 | 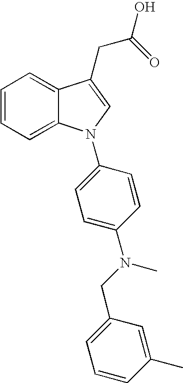 | B | 385.1 (pos APCI) |
| 177 | 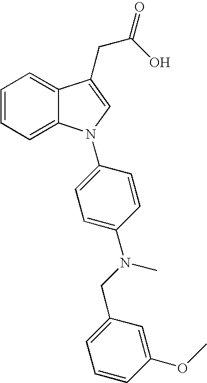 | B | 401.2 (pos APCI) |
| 178 | 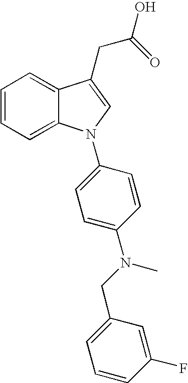 | B | 389.1 (pos APCI) |
| 179 | 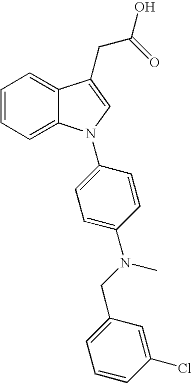 | B | 405.1 (pos APCI) |
| 180 | 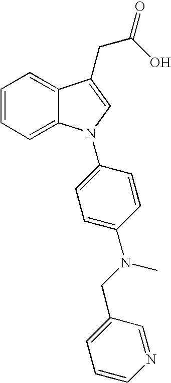 | A | 372.2 (pos APCI) |
| 181 | 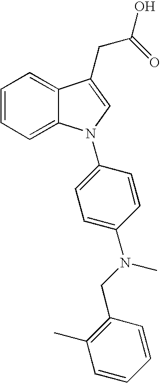 | B | 385.2 (pos APCI) |
| 182 | 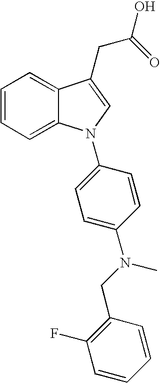 | B | 389.1 (pos APCI) |

A = 10~50 μM
B < 10 μM

TABLE 3
Examples of compounds made with Scheme 4 and Scheme 5.

| Example | Structure | IC50 Activity | LC/MS |
|---|---|---|---|
| 183 | 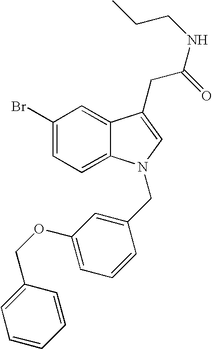 | A | 490.2 (neg APCI) |
| 184 | 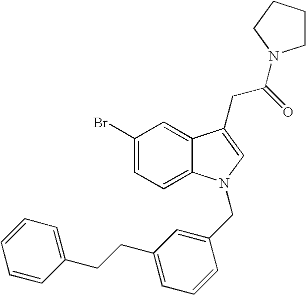 | A | 502.4 (neg APCI) |
| 185 | 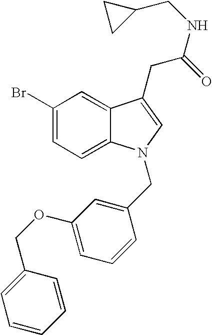 | A | 502.9 (neg APCI) |
| 186 | 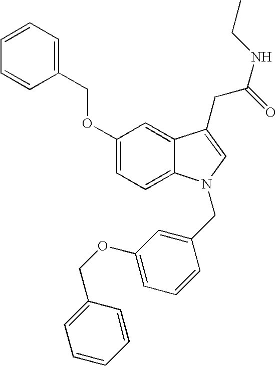 | A | 503.8 (neg APCI) |
| 187 | 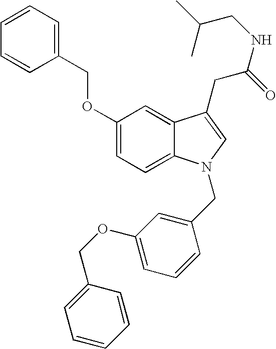 | A | 531.6 (neg APCI) |
| 188 | 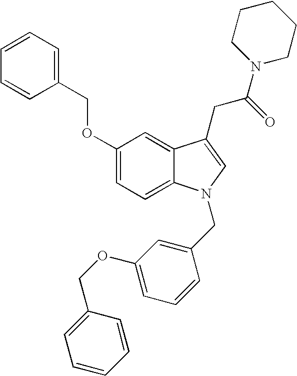 | A | 543.6 (neg APCI) |
| 189 | 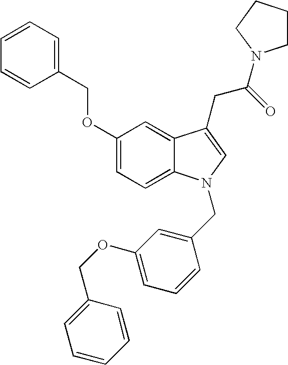 | A | 529.4 (neg APCI) |
| 190 | 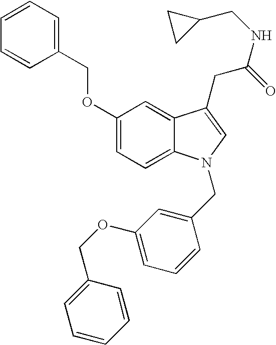 | A | 529.1 (neg APCI) |
| 191 | 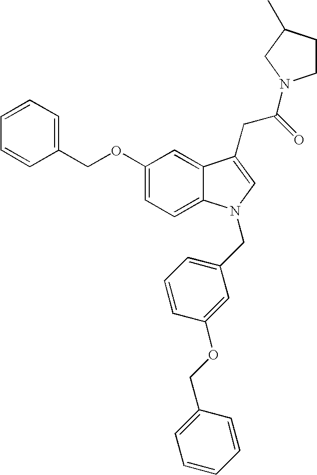 | A | 544.9 (neg APCI) |
| 192 | 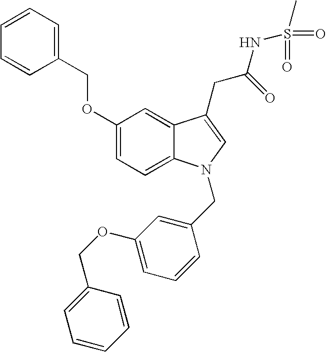 | B | 553.5 (neg APCI) |
| 193 | 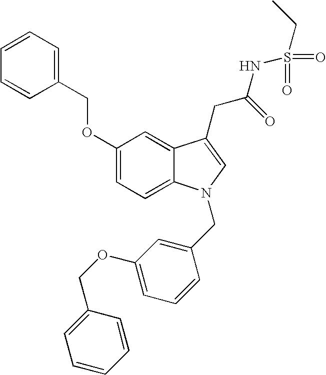 | B | 567.2 (neg APCI) |
| 194 | 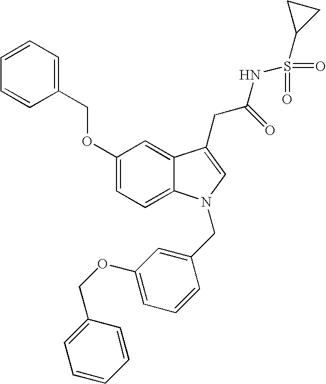 | B | 580 (neg APCI) |
| 195 | 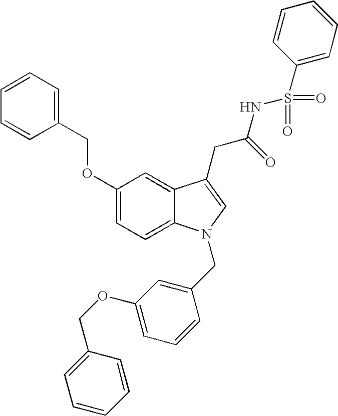 | B | 615.3 (neg APCI) |
| 196 | 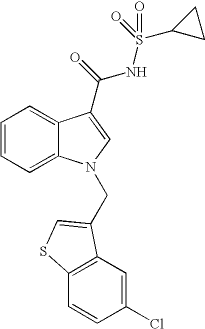 | A | 444 (neg APCI) |
| 197 | 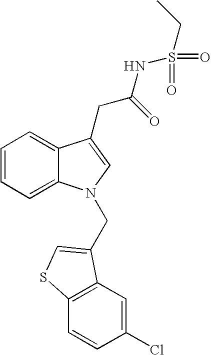 | A | 446.2 (neg APCI) |
| 198 | 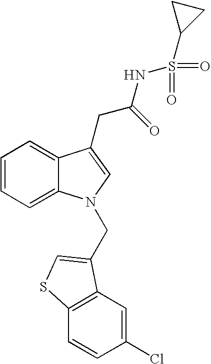 | A | 458 (neg APCI) |
| 199 | 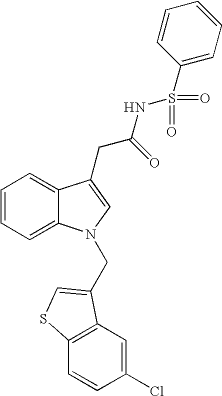 | A | 493.2 (neg APCI) |
| 200 | 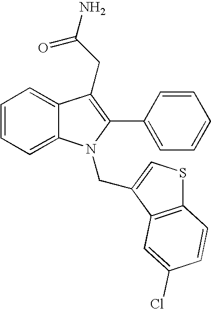 | A | 431.1 (pos APCI) |
| 201 | 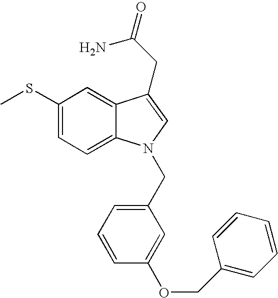 | B | 417.8 (pos APCI) |

A = 10~50 μM
B < 10 μM

TABLE 4
Examples of compounds made in Scheme 6.

| Example | Structure | IC50 Activity | LC/MS |
|---|---|---|---|
| 202 | 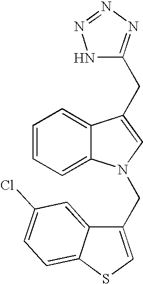 | B | 378.1 (neg APCI) |
| 203 | 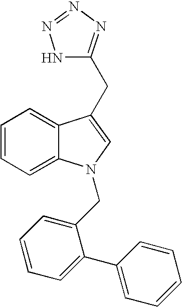 | B | 364.2 (neg APCI) |
| 204 | 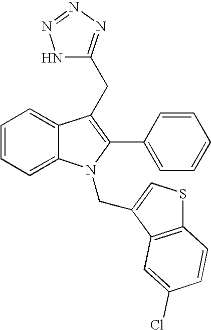 | B | 454.1 (neg APCI) |
| 205 | 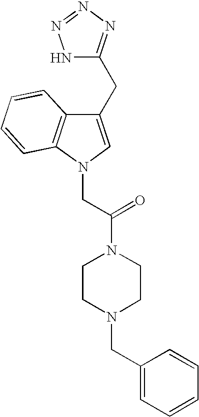 | A | 416.2 (pos APCI) |
| 206 | 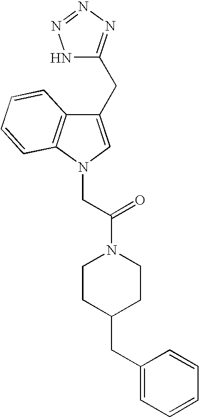 | A | 415.2 (pos APCI) |
| 207 | 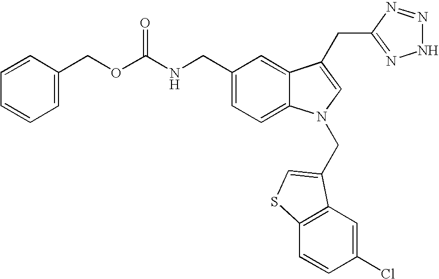 | B | 541.1 (neg APCI) |
| 208 | 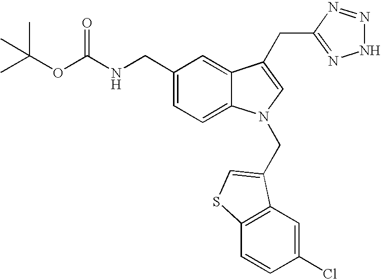 | B | 509.1 (pos APCI) |
| 209 | 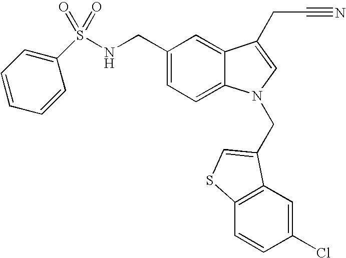 | A | 504.3 (neg APCI) |
| 210 | 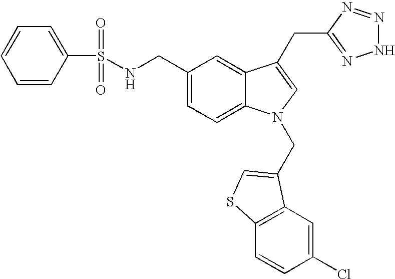 | B | 547.3 (neg APCI) |
| 211 | 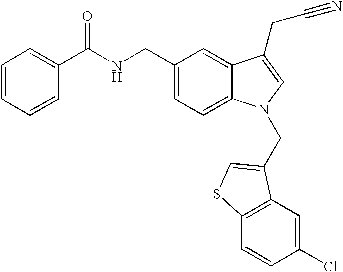 | A | 470.0 (pos APCI) |

A = 10~50 μM
B < 10 μM

TABLE 5
Examples of compounds made using Scheme 7.

| Example | Structure | IC50 Activity | LC/MS |
|---|---|---|---|
| 212 | 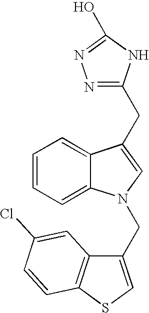 | A | 293.2 (neg APCI) |

A = 10~50 μM

TABLE 6
Examples of compounds made using Scheme 8.

| Example | Structure | IC50 Activity | LC/MS |
|---|---|---|---|
| 213 | 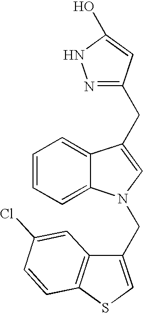 | A | 392.2 (neg APCI) |

A = 10~50 μM

TABLE 7
Examples of compounds made using Scheme 9.

| Example | Structure | IC50 Activity | LC/MS |
|---|---|---|---|
| 214 | 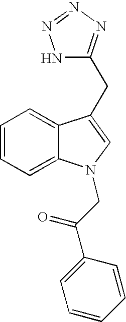 | A | 316.2 (neg APCI) |

A = 10~50 μM

TABLE 8
Examples of compounds made using Scheme 10.

| | | IC50 | |
|---|---|---|---|
| Example | Structure | Activity | LC/MS |
| 215 | 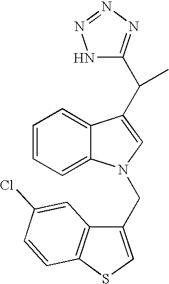 | A | 392.3 (neg APCI) |
| 216 | 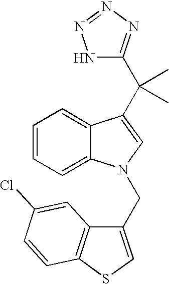 | A | 406.3 (neg APCI) |

A = 10~50 μM

TABLE 9
Examples of compounds made using Scheme 11.

| Example | Structure | IC50 Activity | LC/MS |
|---|---|---|---|
| 217 | 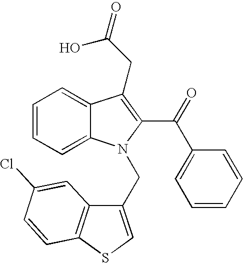 | B | 458.9 (neg APCI) |
| 218 | 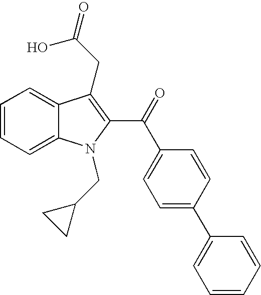 | B | 408.9 (neg APCI) |
| 219 | 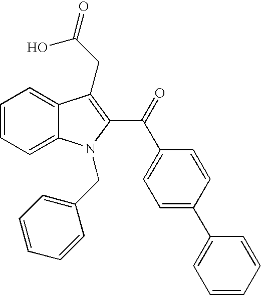 | B | 400.0(neg APCI-CO2) |
| 220 | 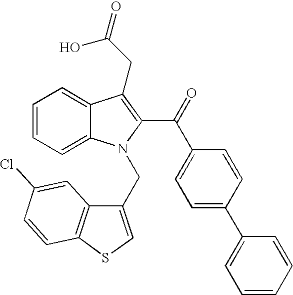 | B | 534.1 (neg APCI) |
| 221 | 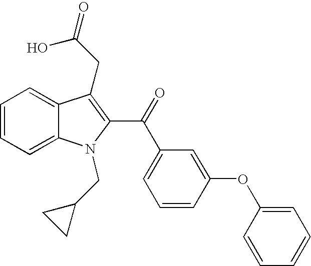 | B | 423.5 (neg APCI) |
| 222 | 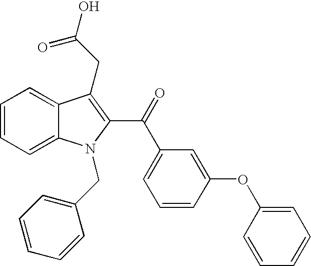 | B | 459.7 (neg APCI) |
| 223 | 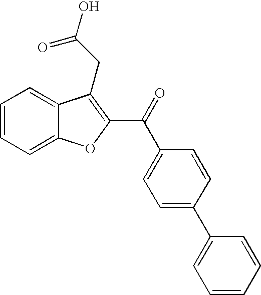 | A | 356.0 (neg APCI) |
| 224 | 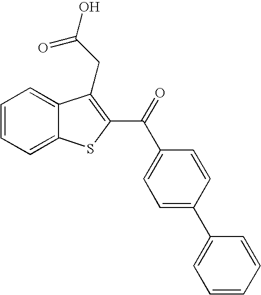 | B | 372.1 (neg APCI) |

A = 10~50 μM
B < 10 μM

TABLE 10
Examples of compounds made using Scheme 12.

| Example | Structure | IC50 Activity | LC/MS |
|---|---|---|---|
| 225 | 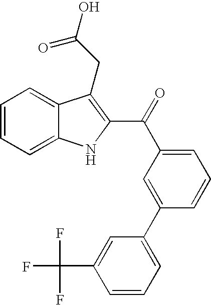 | A | 422.3 (neg APCI) |
| 226 | 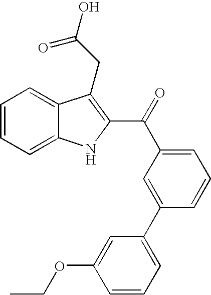 | A | 398.3 (neg APCI) |
| 227 | 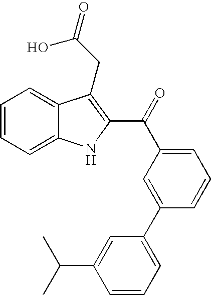 | B | 396.3 (neg APCI) |
| 228 | 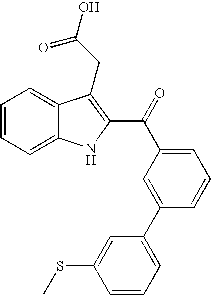 | A | 400.2 (neg APCI) |
| 229 | 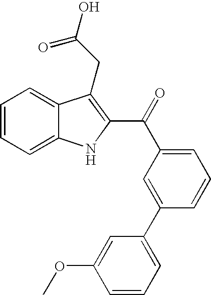 | A | 384.3 (neg APCI) |
| 230 | 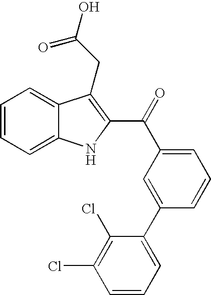 | B | 422.2 (neg APCI) |
| 231 | 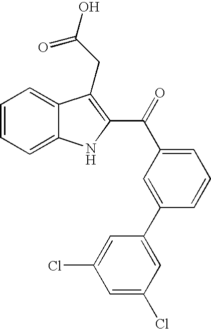 | B | 422.2 (neg APCI) |
| 232 | 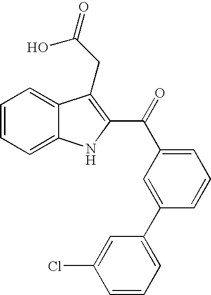 | A | 388.2 (neg APCI) |
| 233 | 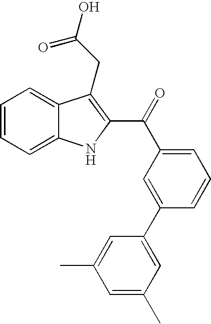 | B | 382.3 (neg APCI) |
| 234 | 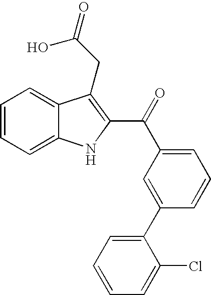 | A | 388.2 (neg APCI) |
| 235 | 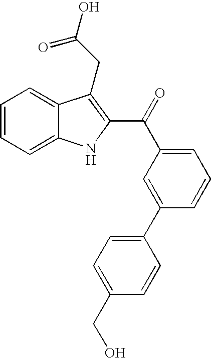 | A | 384.2 (neg APCI) |
| 236 | 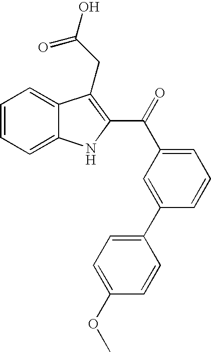 | A | 384.2 (neg APCI) |
| 237 | 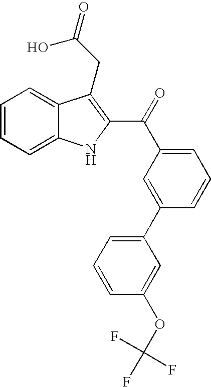 | A | 438.2 (neg APCI) |
| 238 | 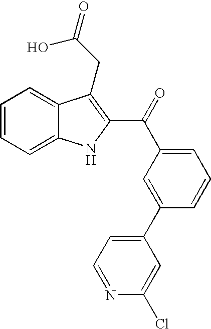 | A | 389.2 (neg APCI) |
| 239 | 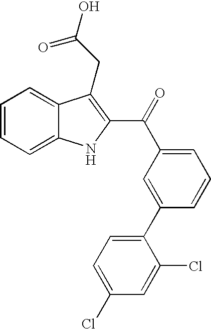 | B | 422.1 (neg APCI) |
| 240 | 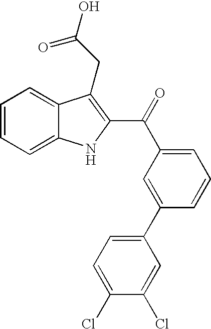 | B | 422.2 (neg APCI) |
| 241 | 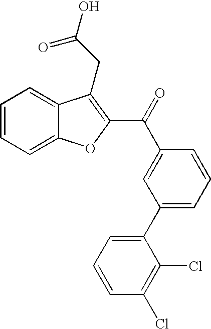 | B | 407.1 (negAPCI (—H2O)) |
| 242 | 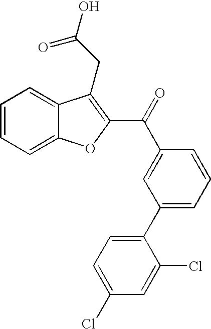 | B | 407.1 (negAPCI (—H2O)) |
| 243 | 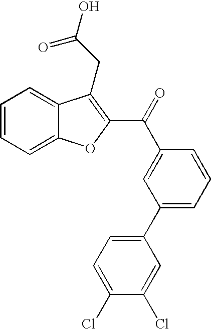 | B | 423.9 (neg APCI) |
| 244 | 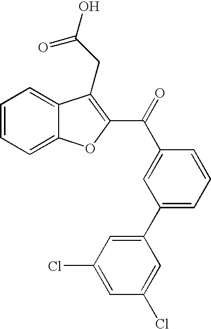 | B | 423.9 (neg APCI) |
| 245 | 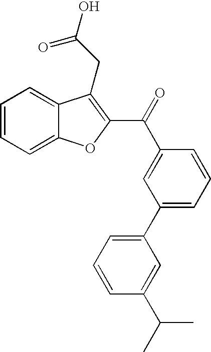 | B | 397.7 (neg APCI) |
| 246 | 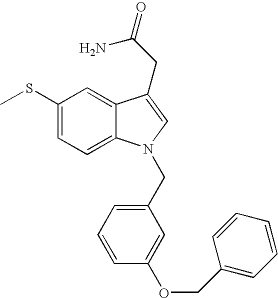 | B | 417.8 (pos APCI) |
| 247 | 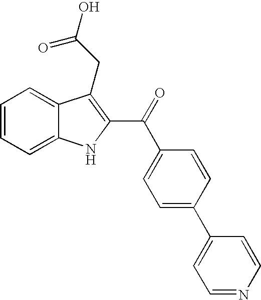 | A | 355.2 (neg APCI) |
| 248 | 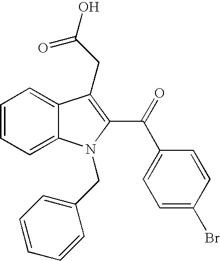 | A | 447.5 (neg APCI) |
| 249 | 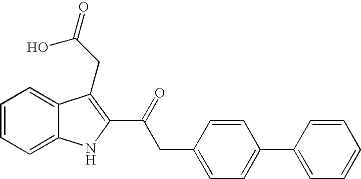 | A | 367.2 (neg APCI) |
| 250 | 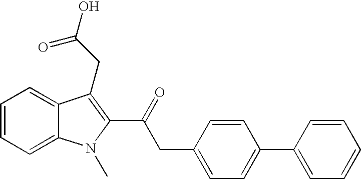 | A | 383.1 (pos APCI) |
| 251 | 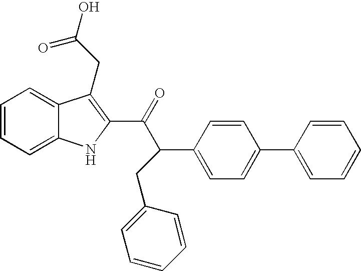 | B | 458.5 (neg APCI) |
| 252 | 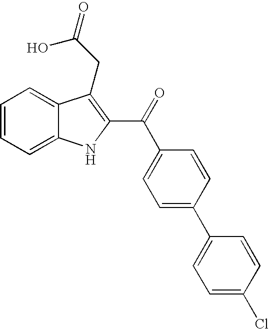 | A | 388.1 (neg APCI) |
| 253 | 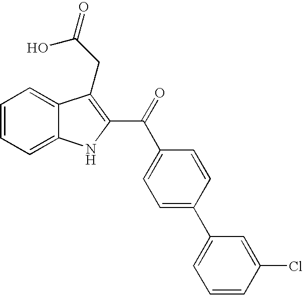 | A | 388.0 (neg APCI) |
| 254 | 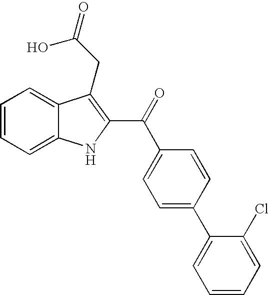 | B | 388.1 (neg APCI) |
| 255 | 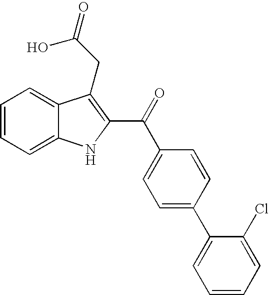 | A | 388.0 (neg APCI) |
| 256 | 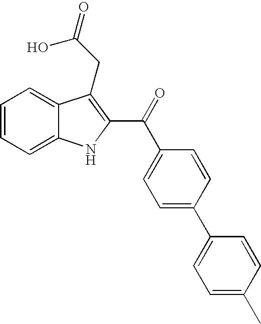 | A | 368.1 (neg APCI) |
| 257 | 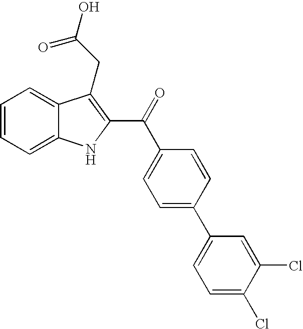 | B | 422.1 (neg APCI) |
| 258 | 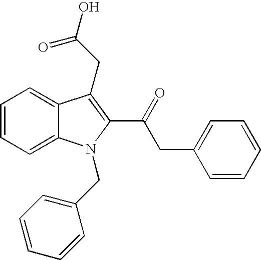 | A | 382.2 (neg APCI) |
| 259 | 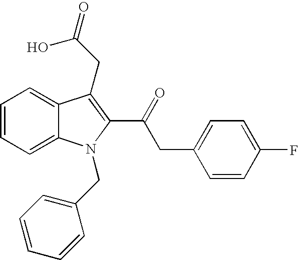 | A | 400.2 (neg APCI) |
| 260 | 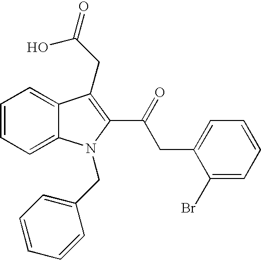 | B | 460.3 (neg APCI) |
| 261 | 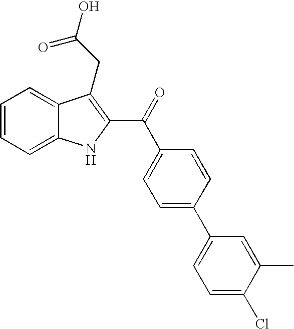 | B | 402.1 (neg APCI) |
| 262 | 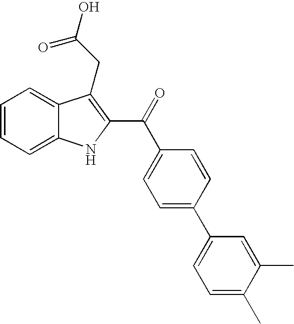 | A | 382.2 (neg APCI) |
| 263 | 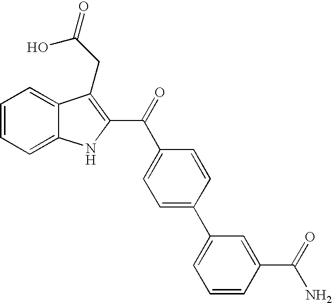 | A | 397.2 (neg APCI) |
| 264 | 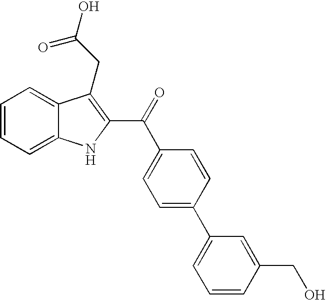 | A | 384.2 (neg APCI) |
| 265 | 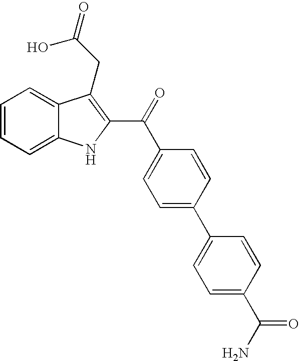 | A | 397.2 (neg APCI) |
| 266 | 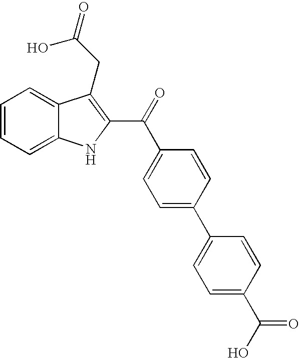 | A | 398.2 (neg APCI) |
| 267 | 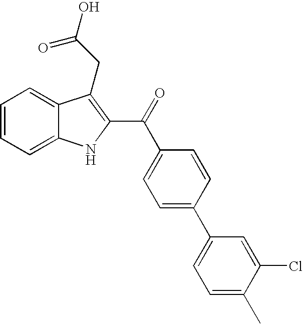 | B | 402.3 (neg APCI) |
| 268 | 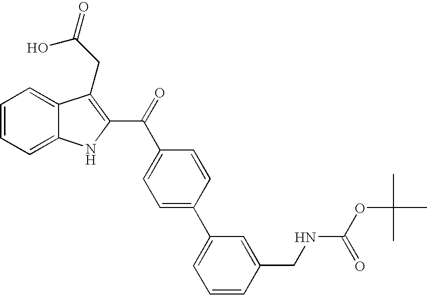 | B | 483.3 (neg APCI) |
| 269 | 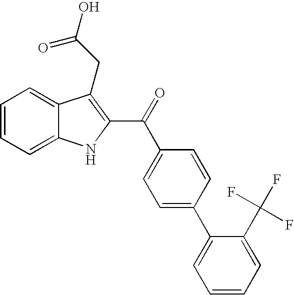 | A | 422.2 (neg APCI) |
| 270 | 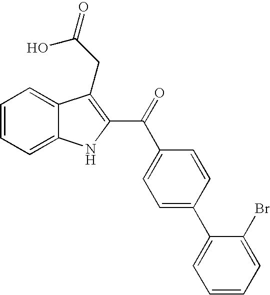 | A | 432.1 (neg APCI) |
| 271 | 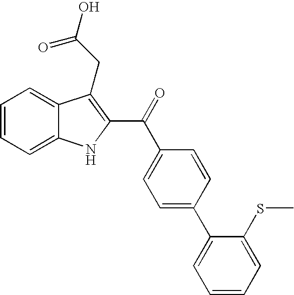 | A | 400.2 (neg APCI) |
| 272 | 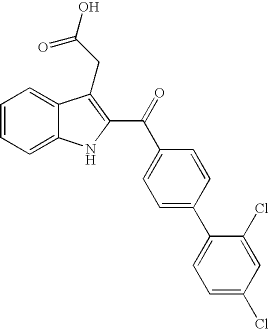 | B | 422.1 (neg APCI) |
| 273 | 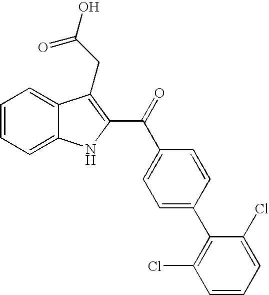 | A | 422.1 (neg APCI) |
| 274 | 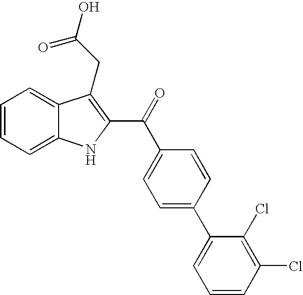 | B | 422.1 (neg APCI) |
| 275 | 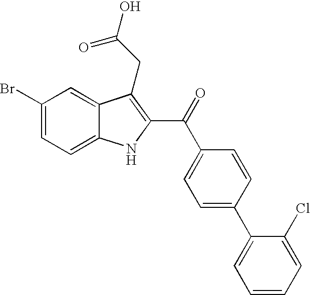 | B | 468.0 (neg APCI) |
| 276 | 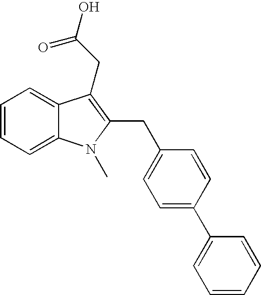 | A | 354.5 (neg APCI) |
| 277 | 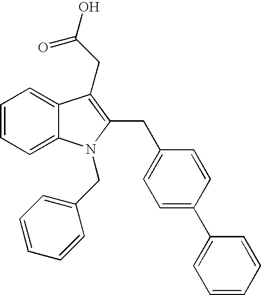 | A | 430.4 (neg APCI) |
| 278 | 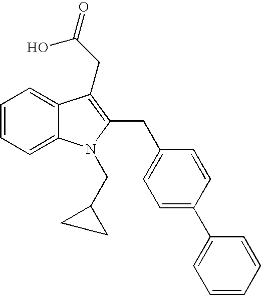 | B | 394.3 (neg APCI) |
| 279 | 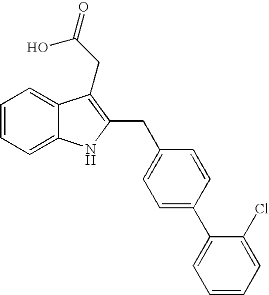 | B | 374.0 (neg APCI) |
| 280 | 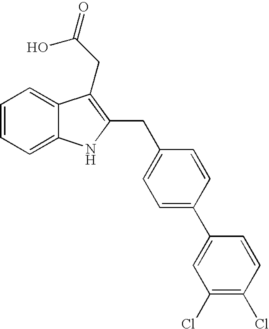 | B | 408.0 (neg APCI) |
| 281 | 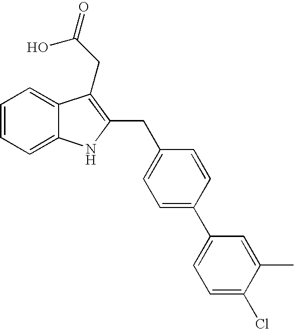 | B | 389.1 (neg APCI) |
| 282 | 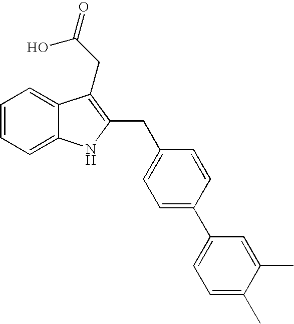 | B | 368.1 (neg APCI) |
| 283 | 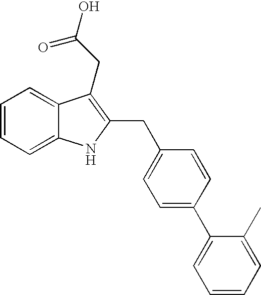 | A | 354.1 (neg APCI) |
| 284 | 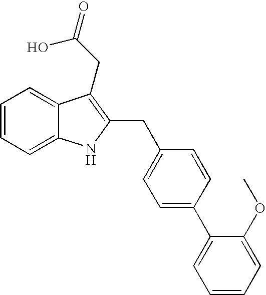 | A | 370.1 (neg APCI) |
| 285 | 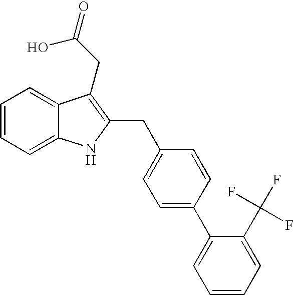 | B | 408.1 (neg APCI) |
| 286 | 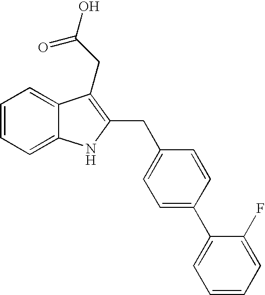 | A | 358.1 (neg APCI) |
| 287 | 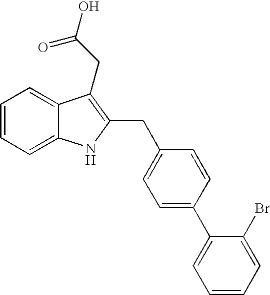 | B | 418.1 (neg APCI) |
| 288 | 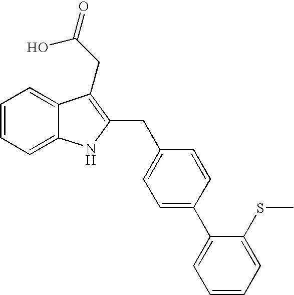 | B | 386.1 (neg APCI) |
| 289 | 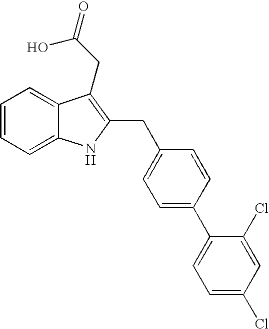 | B | 408.0 (neg APCI) |
| 290 | 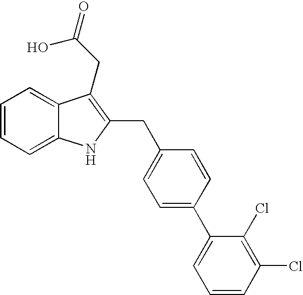 | B | 408.0 (neg APCI) |
| 291 | 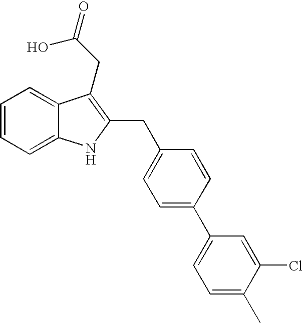 | B | 387.9 (neg APCI) |
| 292 | 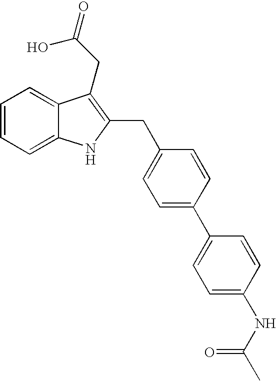 | A | 397.0 (neg APCI) |
| 293 | 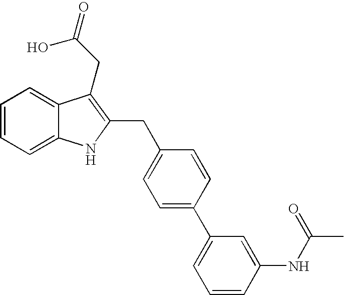 | A | 397.1 (neg APCI) |
| 294 | 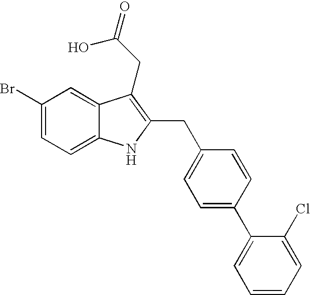 | B | 454.0 (neg APCI) |
| 737 | 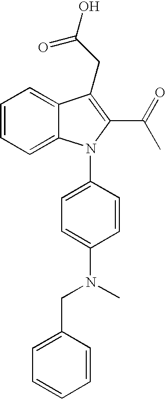 | A | 413.7 (neg APCI) |
| 738 | 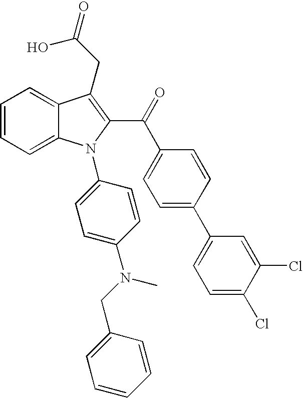 | A | 543.1 (pos APCI)M-C₆H₅ |
| 739 | 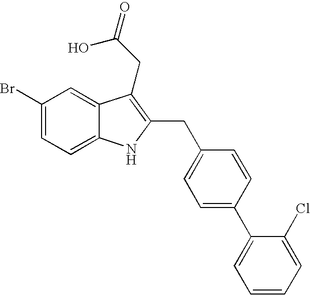 | A | 454.3 |

A = 10~50 μM
B < 10 μM

TABLE 11
Examples of compounds made using Scheme 13.

| Example | Structure | IC50 Activity | LC/MS |
|---|---|---|---|
| 295 | 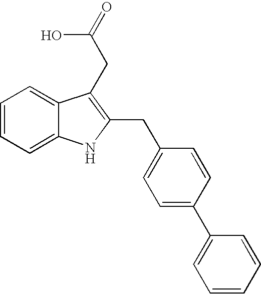 | B | 339.7 (neg APCI) |
| 296 | 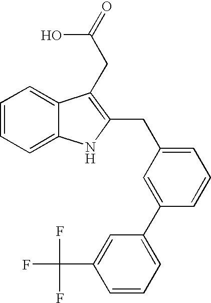 | B | 408.1 (neg APCI) |
| 297 | 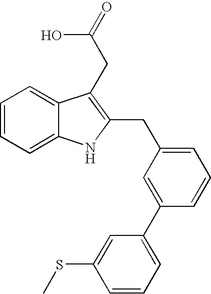 | B | 386.1 (neg APCI) |
| 298 | 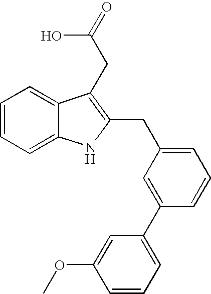 | B | 370.1 (neg APCI) |
| 299 | 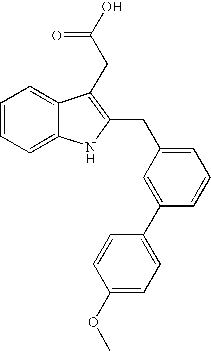 | B | 370.1 (neg APCI) |
| 300 | 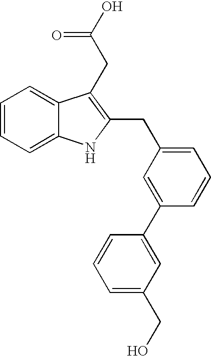 | B | 370.3 (neg APCI) |
| 301 | 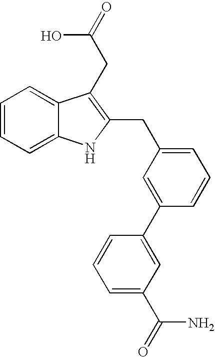 | A | 383.3 (neg APCI) |
| 302 | 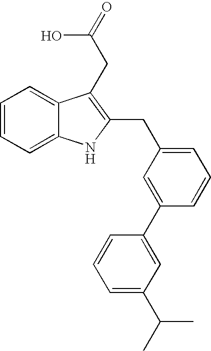 | B | 382.2 (neg APCI) |
| 303 | 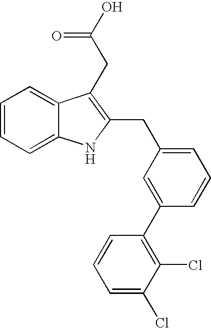 | B | 408.0 (neg APCI) |
| 304 | 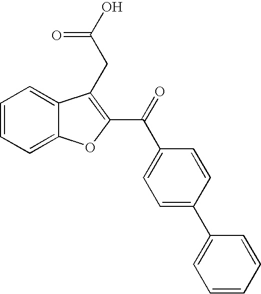 | A | 356.0 (neg APCI) |
| 305 | 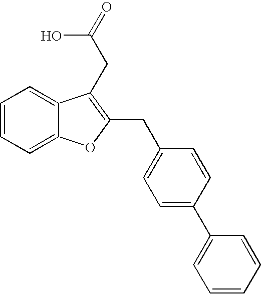 | A | 341.1 (neg APCI) |
| 306 | 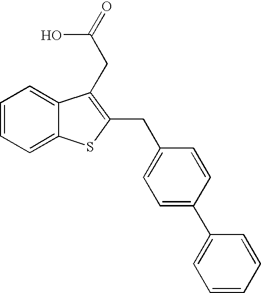 | B | 357.1 (neg APCI) |
| 307 | 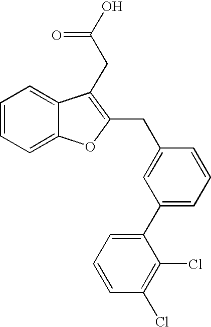 | B | 410.9 (neg APCI) |
| 308 | 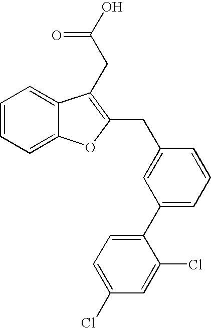 | B | 409.0 (neg APCI) |
| 309 | 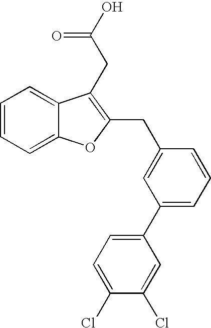 | B | 409.0 (neg APCI) |
| 310 | 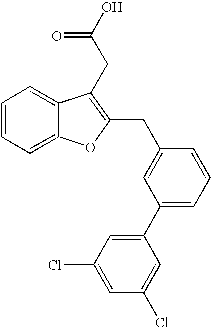 | B | 409.1 (neg APCI) |
| 311 | 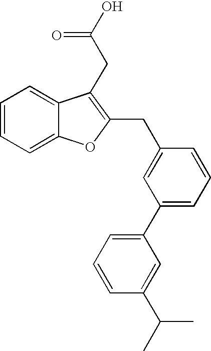 | B | 383.0 (neg APCI) |

TABLE 12
Examples of compounds made using Scheme 14.

| Example | Structure | IC50 Activity | LC/MS |
|---|---|---|---|
| 312 | 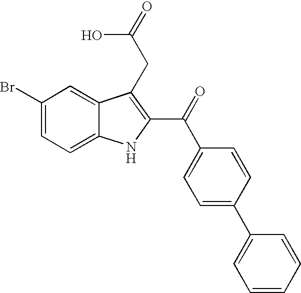 | A | 431.7, 433.7(neg APCI) |

TABLE 13
Examples of compounds made using Scheme 15.

| Example | Structure | IC50 Activity | LC/MS |
|---|---|---|---|
| 313 | 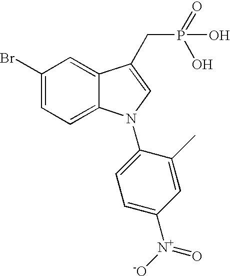 | A | 426.9 (posAPCI) |
| 314 | 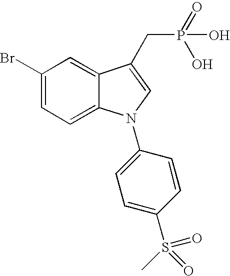 | A | 444.1 (negAPCI) |
| 315 | 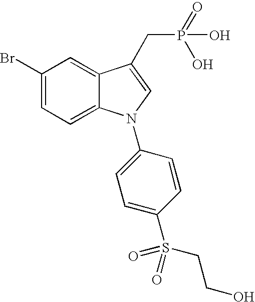 | A | 476.0 (posAPCI) |
| 316 | 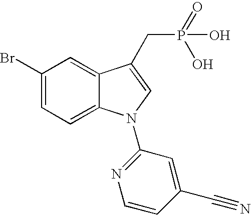 | A | 393.0 (negAPCI) |
| 317 | 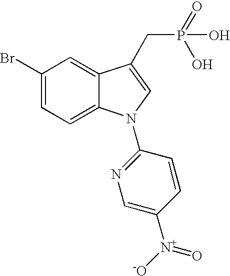 | B | 409.9, 411.9(negAPCI) |

TABLE 14
Examples of compounds made using Scheme 16 and Scheme 17.

| Example | Structure | IC50 Activity |
|---|---|---|
| 318 | 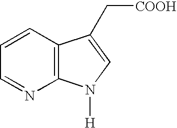 | C |
| 319 | 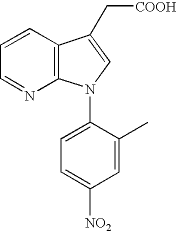 | C |
| 320 | 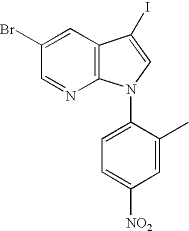 | C |
| 321 | 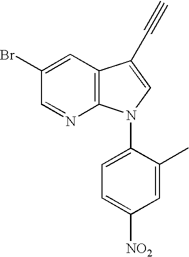 | C |
| 322 | 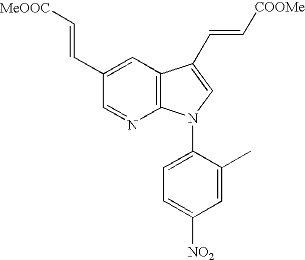 | A |
| 323 | 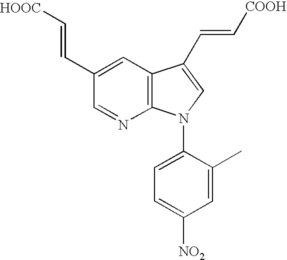 | C |
| 324 | 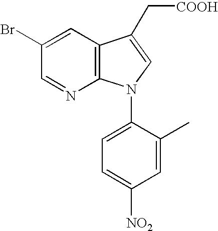 | C |
| 325 | 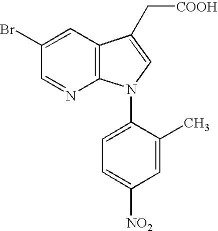 | C |
| 326 | 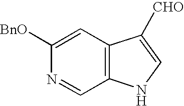 | C |
| 327 | 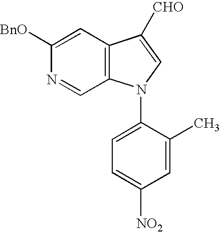 | C |
| 328 | 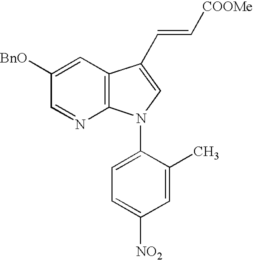 | C |
| 329 | 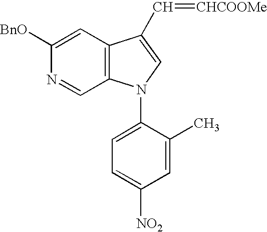 | C |
| 330 | 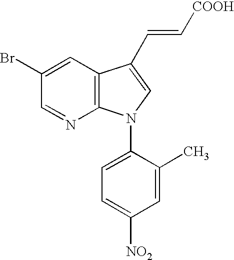 | C |
| 331 | 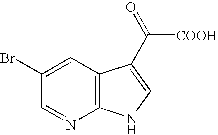 | C |
| 332 | 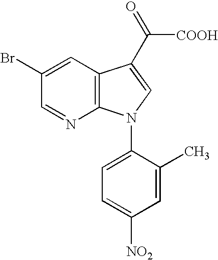 | A |
| 333 | 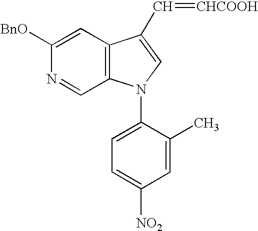 | C |
| 334 | 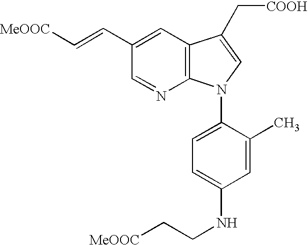 | A |
| 335 | 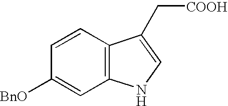 | ND |
| 336 | 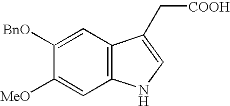 | ND |
| 337 | 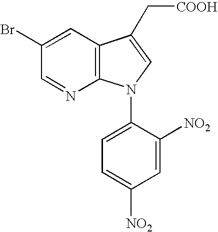 | A |
| 338 | 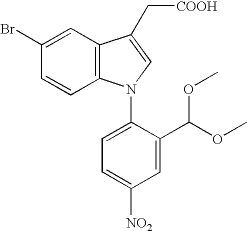 | C |
| 339 | 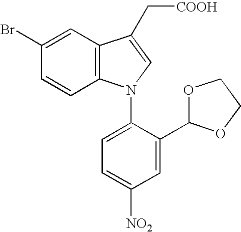 | A |
| 340 | 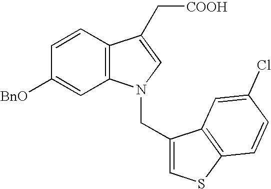 | B |
| 341 | 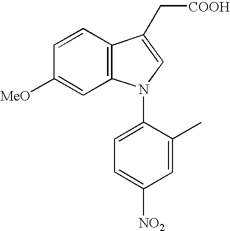 | C |
| 342 | 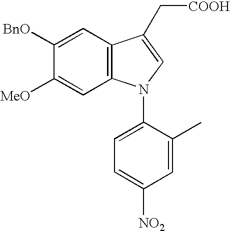 | A |
| 343 | 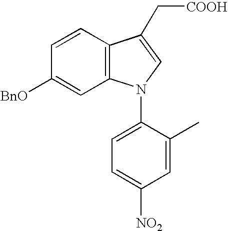 | A |
| 344 | 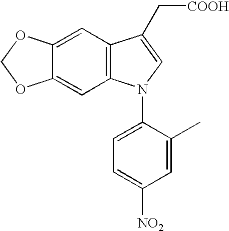 | C |
| 345 | 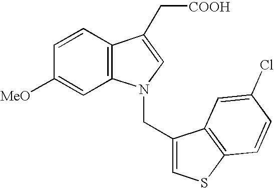 | A |
| 346 | 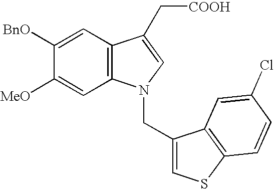 | A |
| 347 | 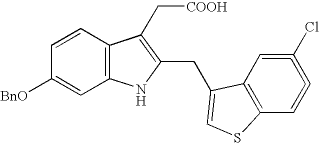 | B |
| 348 | 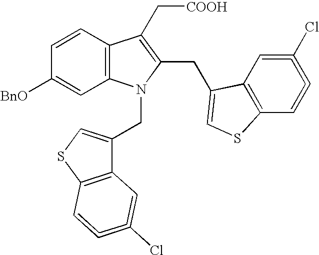 | B |
| 349 | 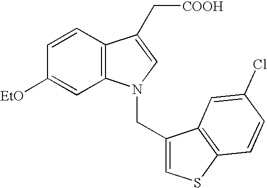 | A |
| 350 | 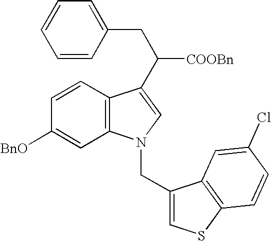 | C |
| 351 | 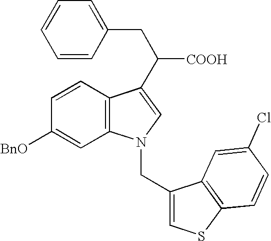 | B |
| 352 | 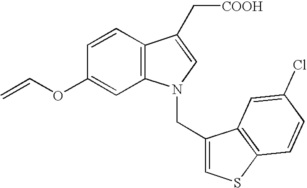 | B |
| 353 | 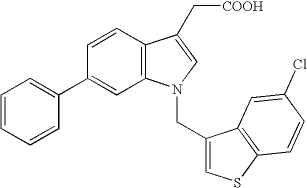 | B |
| 354 | 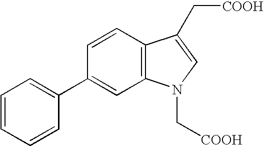 | C |
| 355 | 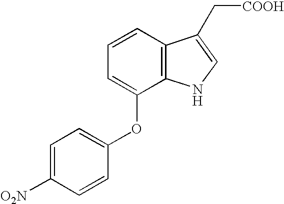 | C |
| 356 | 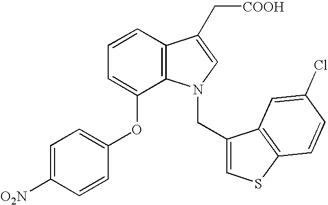 | B |
| 357 | 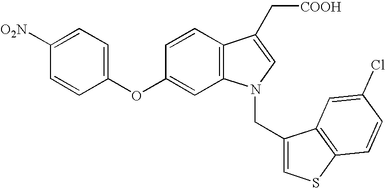 | B |
| 358 | 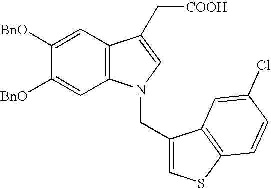 | B |
| 359 | 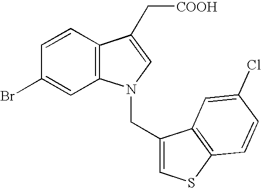 | A |
| 360 | 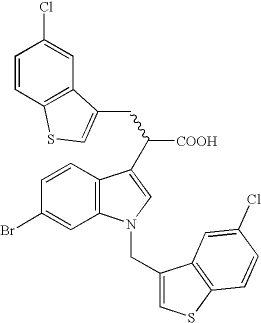 | B |
| 361 | 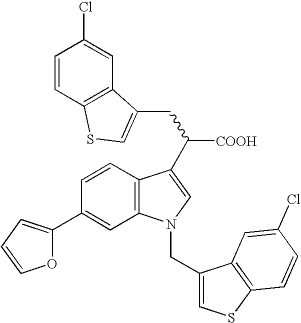 | B |
| 362 | | B |
| 363 | 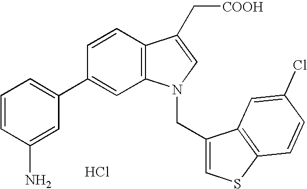 | B |
| 364 | 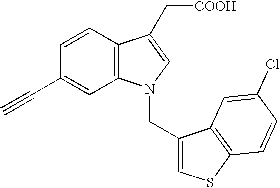 | B |
| 365 | 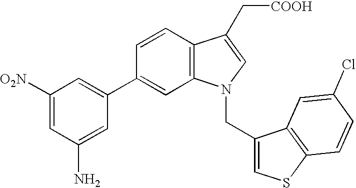 | B |
| 366 | 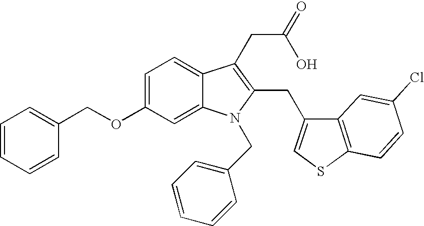 | B |
| 367 | 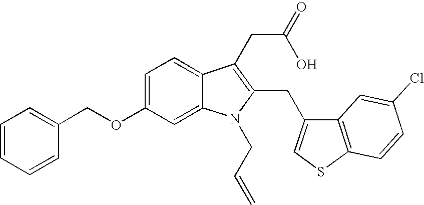 | B |
| 368 | 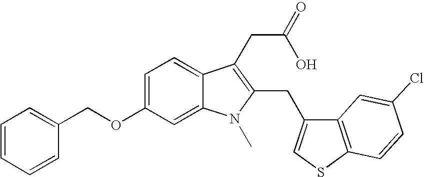 | B |
| 369 | 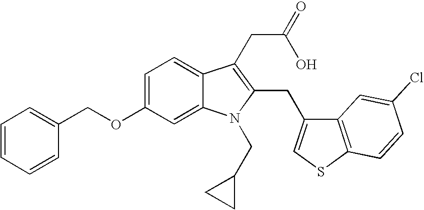 | B |
| 370 | 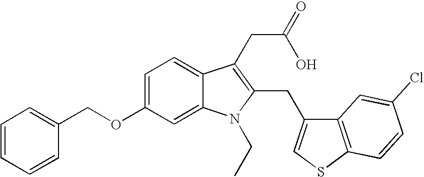 | B |
| 371 | 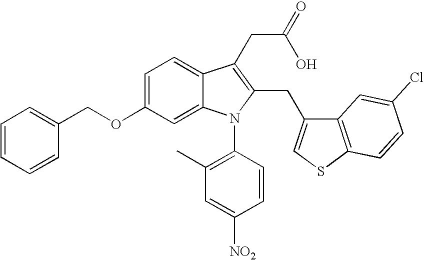 | B |
| 372 | 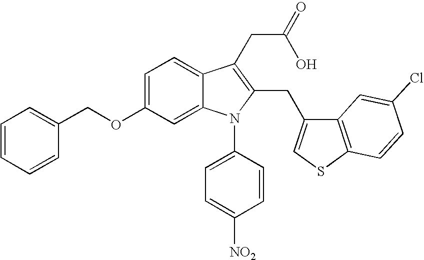 | B |
| 373 | 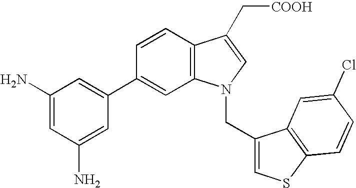 | B |
| 374 | 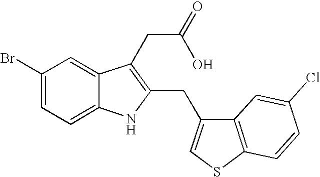 | A |
| 375 | 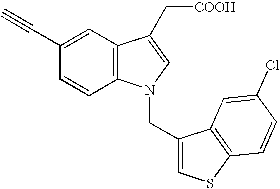 | ND |
| 740 | 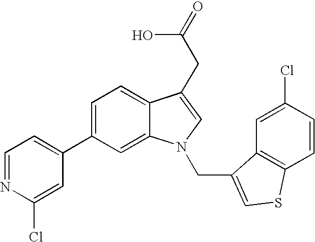 | A |
| 741 | 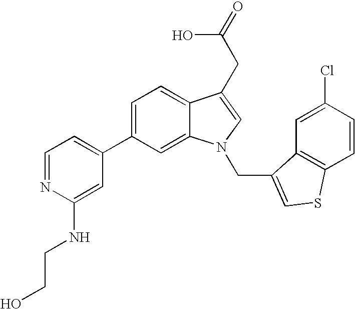 | B |
| 742 | 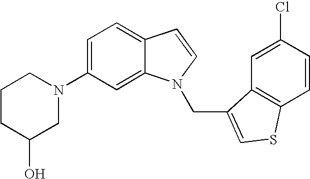 | A |
| 743 | 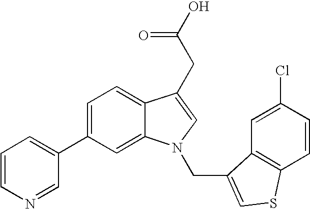 | A |
| 744 | 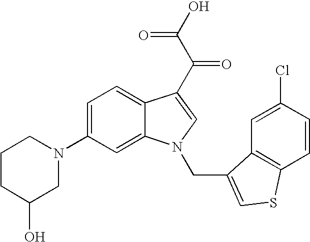 | B |
| 745 | 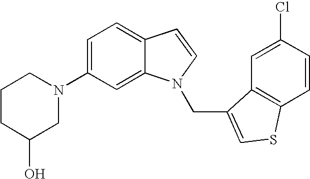 | A |
| 746 | 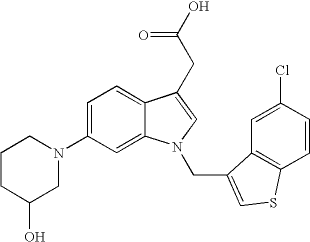 | A |
| 747 | 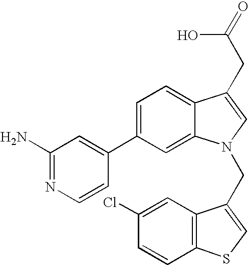 | A |
| 748 | 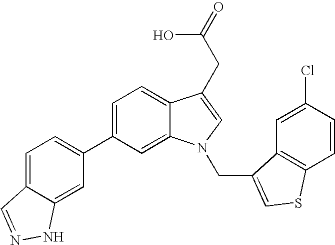 | A |
| 749 | 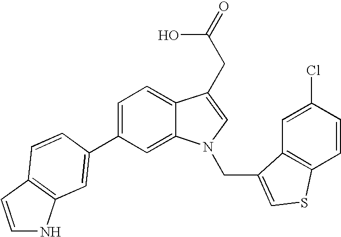 | A |
| 750 | 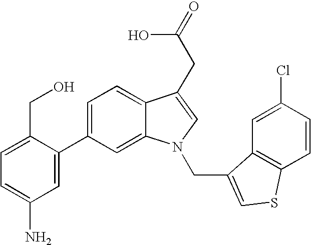 | A |

A = 10~50 μM
B < 10 μM
C > 50 μM
ND = not determined

TABLE 15
Examples of compounds made using Scheme 18,
Scheme 19, Scheme 20 and Scheme 21.

| Example | Structure | IC50 Activity |
|---|---|---|
| 376 | 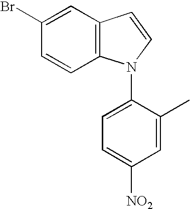 | C |
| 377 | 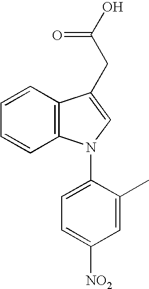 | C |
| 378 | 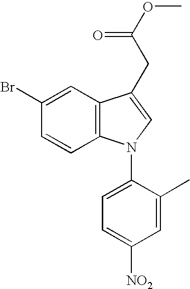 | C |
| 379 | 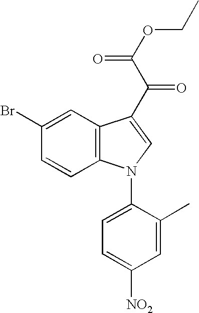 | A |
| 380 | 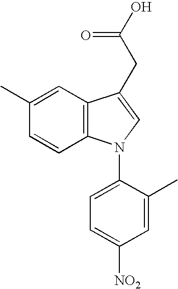 | C |
| 381 | 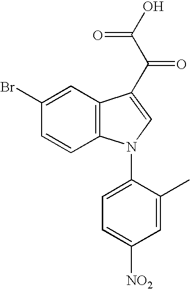 | A |
| 382 | 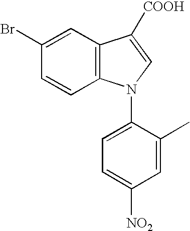 | C |
| 383 | 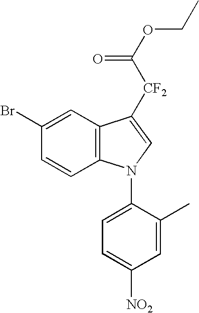 | A |
| 384 | 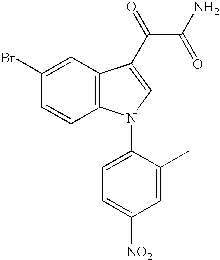 | C |
| 385 | 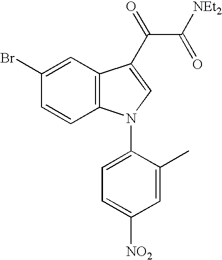 | C |
| 386 | 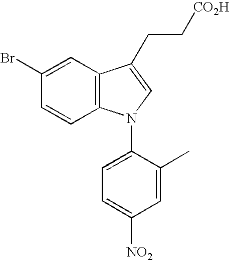 | A |
| 387 | 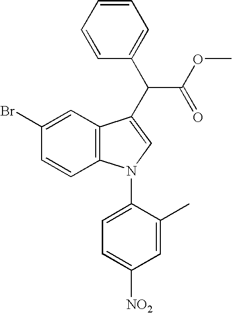 | A (C on 2^{nd} test) |
| 388 | 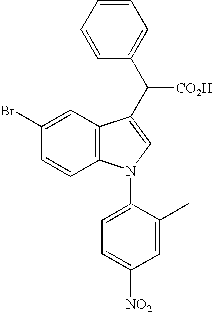 | B |
| 389 | 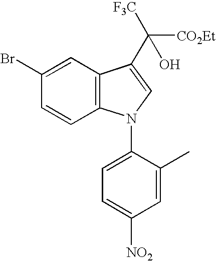 | C |
| 390 | 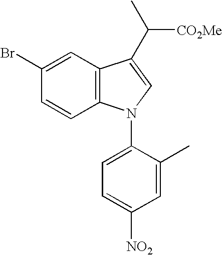 | C |
| 391 | 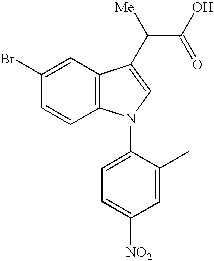 | A |
| 392 | 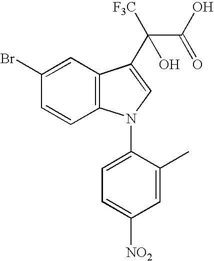 | B |
| 393 | 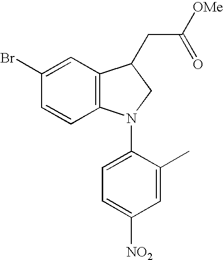 | A |
| 394 | 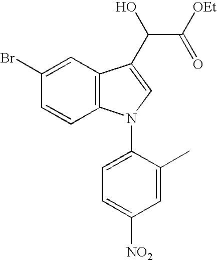 | C |
| 395 | 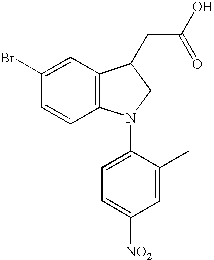 | A |
| 396 | 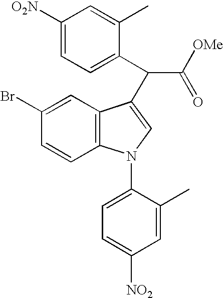 | B |
| 397 | 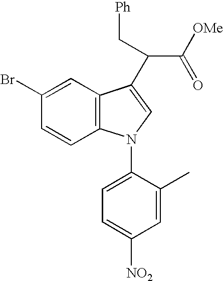 | C |
| 398 | 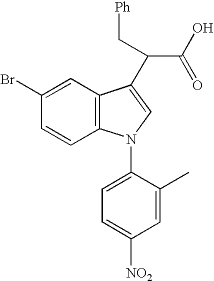 | B |
| 399 | 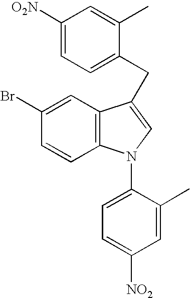 | A |
| 400 | 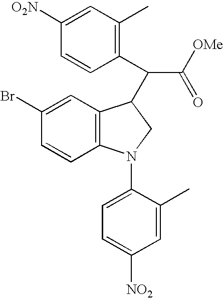 | A |
| 401 | 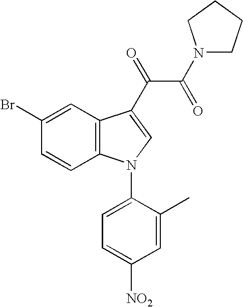 | C |
| 402 | 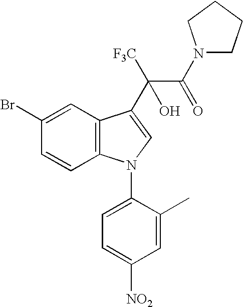 | C |
| 403 | 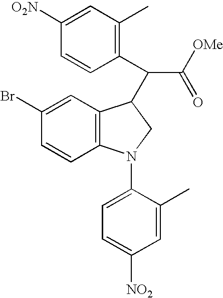 | >20 |
| 404 | 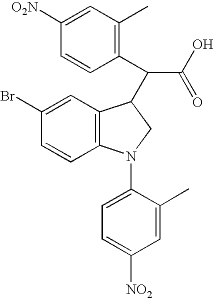 | A |
| 405 | 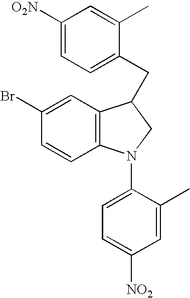 | A |
| 406 | 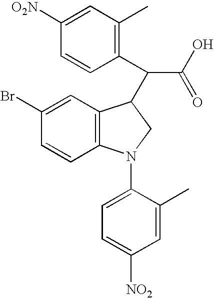 | A |
| 407 | 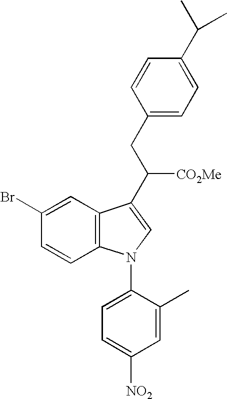 | C |
| 408 | 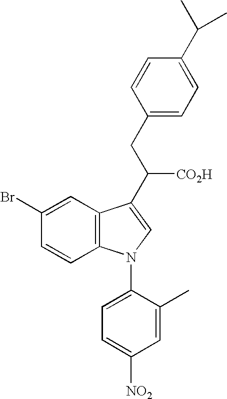 | A |
| 409 | 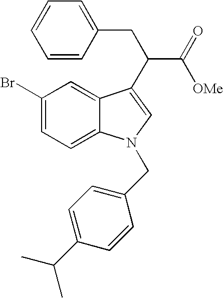 | C |
| 410 | 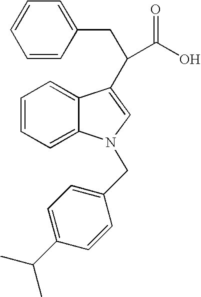 | B |
| 411 | 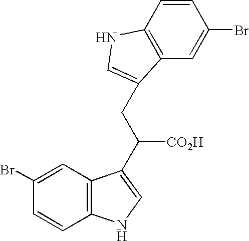 | A |
| 412 | 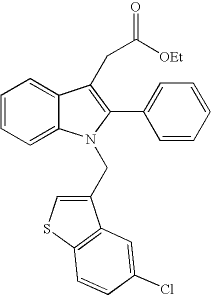 | C |
| 413 | 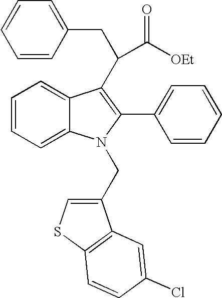 | C |
| 414 | 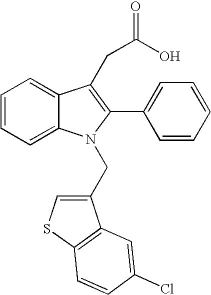 | B |
| 415 | 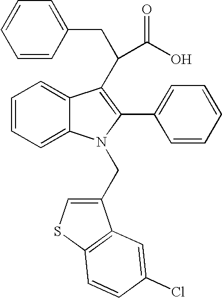 | B |
| 416 | 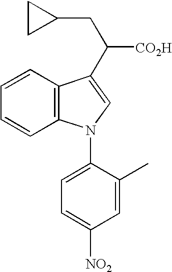 | A |
| 417 | 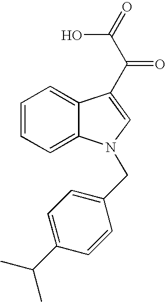 | A |
| 418 | 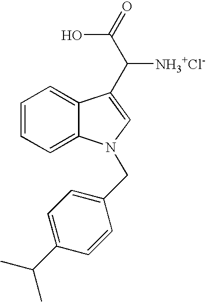 | C |
| 419 | 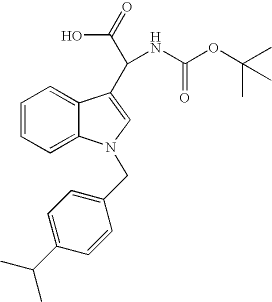 | A |
| 420 | 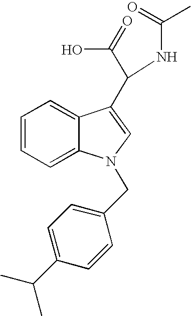 | A |
| 421 | 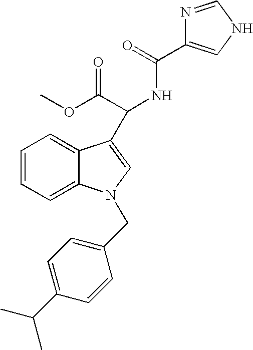 | C |
| 422 | 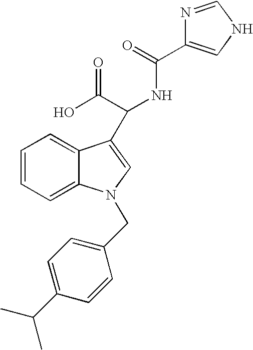 | A |
| 423 | 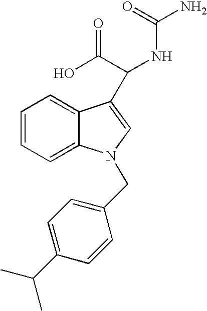 | A |
| 424 | 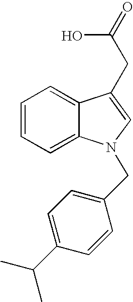 | A |
| 425 | 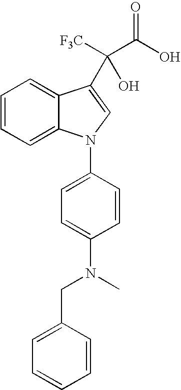 | B |
| 426 | 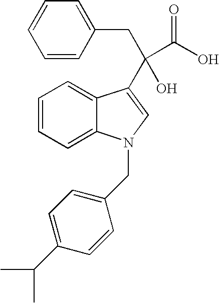 | ND |

A = 10~50 μM
B < 10 μM
C > 50 μM
ND = not determined

TABLE 16
Examples of compounds made using Scheme 22 and Scheme 23.

| | | |
|---|---|---|
| Example | Structure | IC50 Activity |

2-esters

| | | |
|---|---|---|
| 427 | 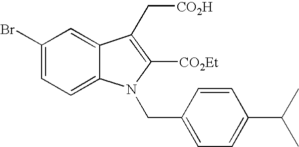 | A |

Tertially amides

| | | |
|---|---|---|
| 428 | 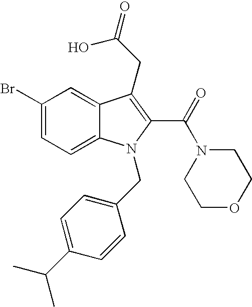 | C |
| 429 | 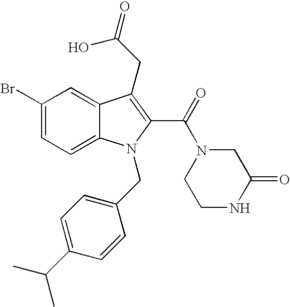 | C |
| 430 | 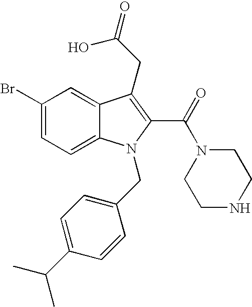 | C |
| 431 | 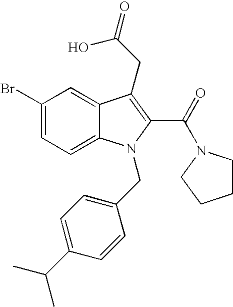 | A |
| 432 | 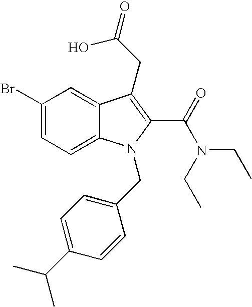 | A |

Monoalkylamides

| | | |
|---|---|---|
| 433 | 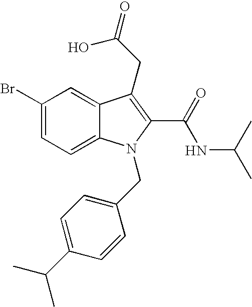 | A |
| 434 | 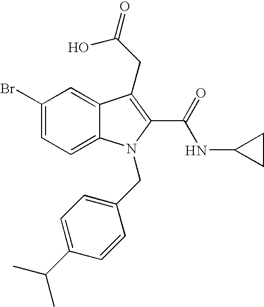 | A |
| 435 | 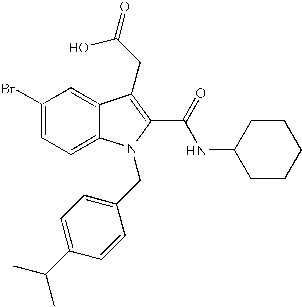 | A |
| 436 | 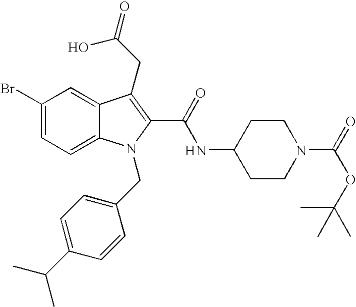 | B |
| 437 | 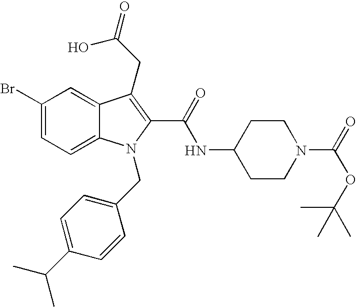 | B |
| 438 | 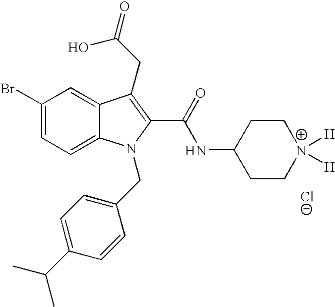 | A |
| 439 | 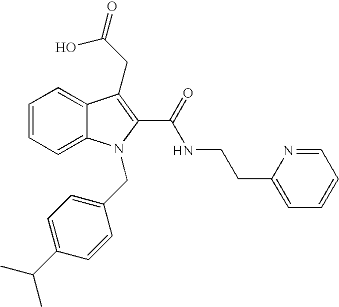 | A |

Benzylamides

| | | |
|---|---|---|
| 440 | 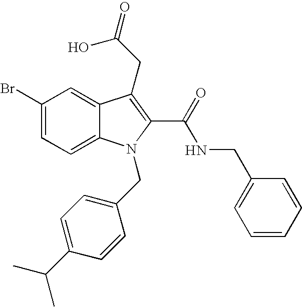 | A |
| 441 | 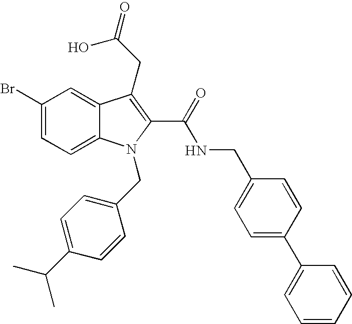 | A |
| 442 | 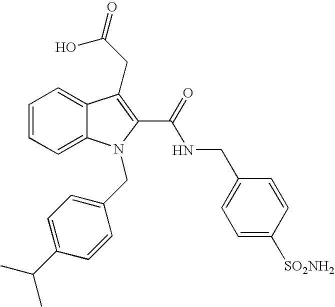 | A |
| 443 | 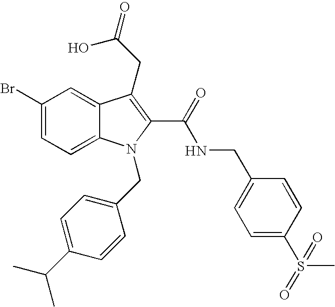 | B |
| 444 | 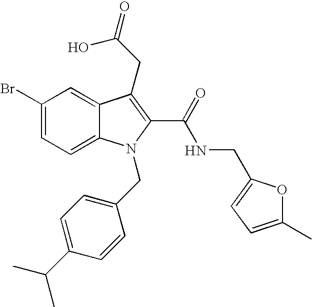 | A |
| 445 | 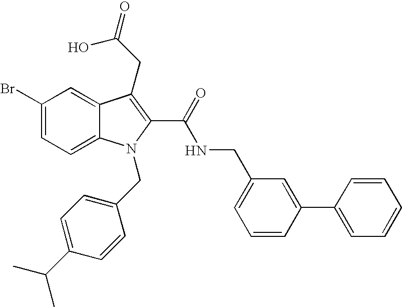 | B |
| 446 | 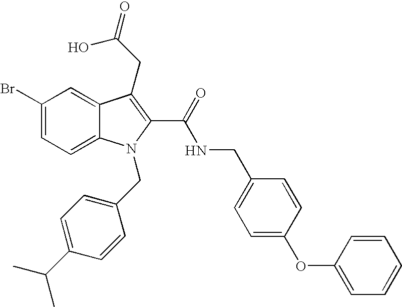 | B |
| 447 | 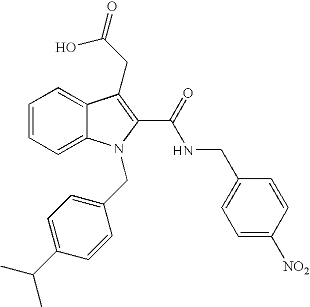 | B |
| 448 | 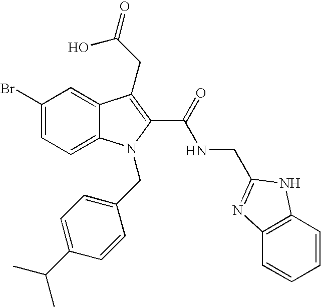 | B |
| 449 | 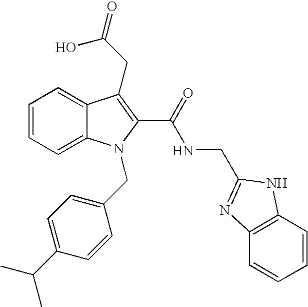 | B |
| 450 | 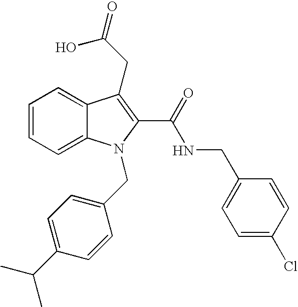 | B |
| 451 | 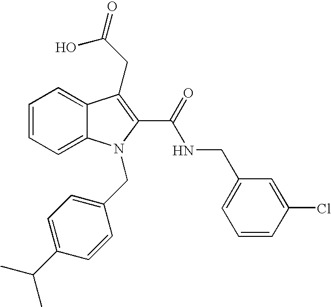 | B |
| 452 | 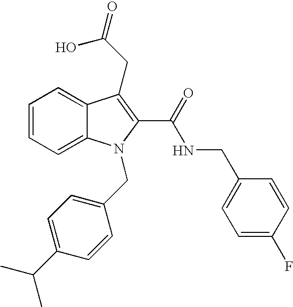 | B |
| 453 | 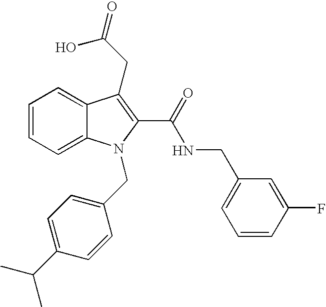 | B |
| 454 | 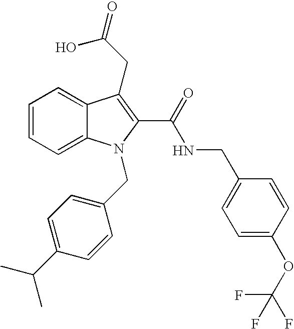 | B |
| 455 | 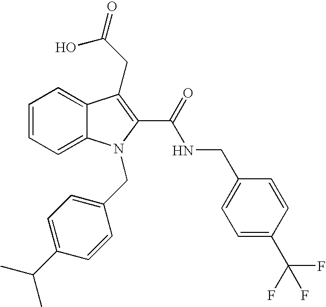 | B |

Arylamides

| | | |
|---|---|---|
| 456 | 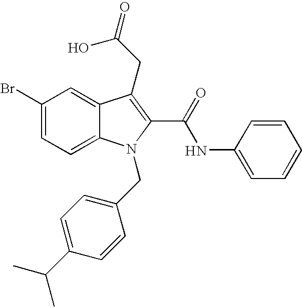 | B |
| 457 | 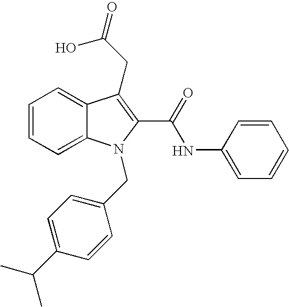 | ND |
| 458 | 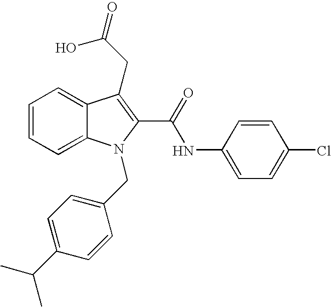 | ND |
| 459 | 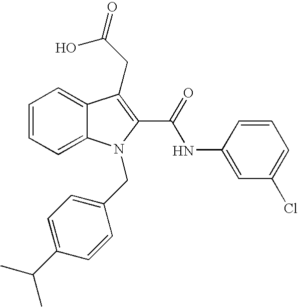 | ND |
| 460 | 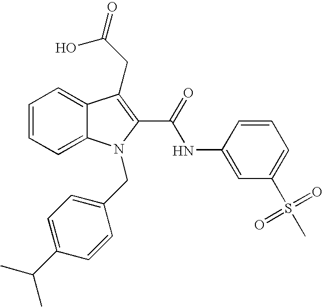 | B |
| 461 | 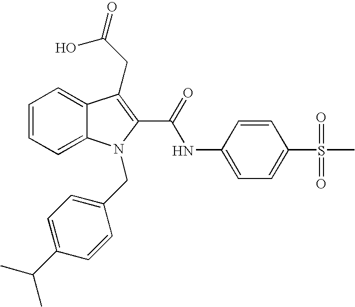 | B |
| 462 | 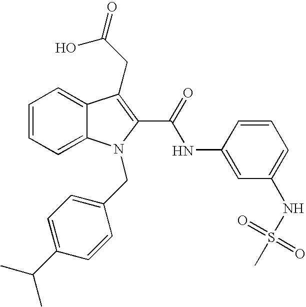 | B |
| 463 | 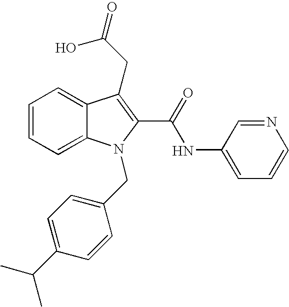 | A |

A = 10~50 μM
B < 10 μM
C > 50 μM
ND = not determined

TABLE 17
Examples of compounds made using Scheme 24.

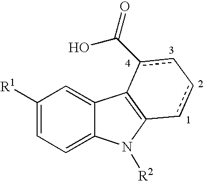

| | | | | IC₅₀ |
|---|---|---|---|---|
| Example | R¹ | R² | 1,2-3,4 | Activity |
| 464 | H | CH₂Ph | CH₂—CH₂—CH₂—CH₂ | C |
| 465 | H | CH₂Ph | CH═CH—CH═CH | C |
| 466 | H | H | CH₂—CH₂—CH₂—CH₂ | C |
| 467 | H | H | CH═CH—CH═CH | C |
| 468 | H | H | CH═CH—CH₂—CH₂ | C |
| 469 | H | 2-Me-4-NO₂—C₆H₃ | CH₂—CH₂—CH₂—CH₂ | C |
| 470 | H | 2-Me-4-NO₂—C₆H₃ | CH═CH—CH═CH | C |
| 471 | Br | 2-Me-4-NO₂—C₆H₃ | CH═CH—CH═CH | A |

A = 10~50 μM
B < 10 μM
C > 50 μM

TABLE 18
Examples of compounds made using Scheme 25.

| Example | Structure | IC50 Activity | MS-ESI: m/z |
|---|---|---|---|
| 472 | 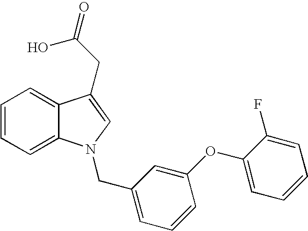 | B | 376[M + 1]⁺ |
| 473 | 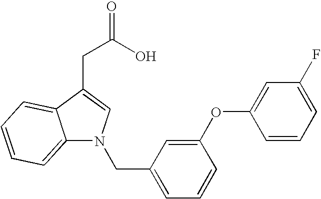 | B | 376[M + 1]⁺ |
| 474 | 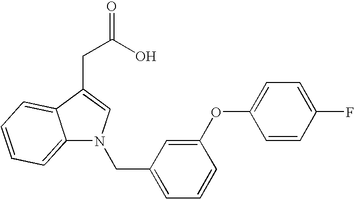 | B | 376[M + 1]⁺ |
| 475 | 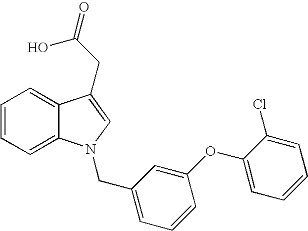 | B | 392[M + 1]⁺ |
| 476 | 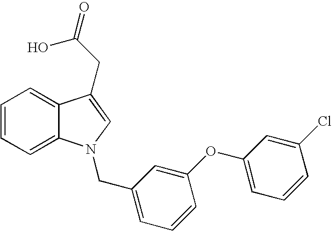 | B | 392[M + 1]⁺ |
| 477 | 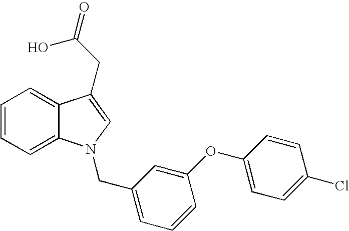 | B | 392[M + 1]⁺ |
| 478 | 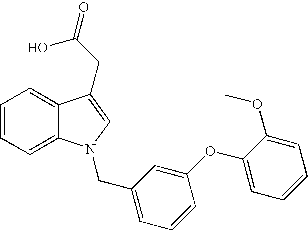 | A | 388[M + 1]⁺ |
| 479 | 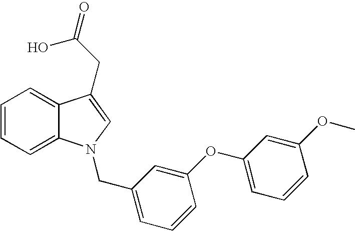 | A | 388[M + 1]⁺ |
| 480 | 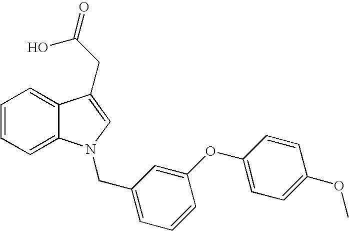 | B | 388[M + 1]⁺ |
| 481 | 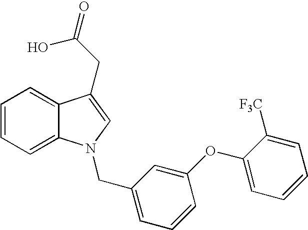 | B | 426[M + 1]⁺ |
| 482 | 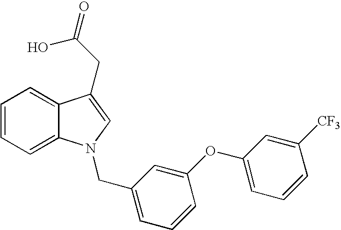 | B | 426[M + 1]⁺ |
| 483 | 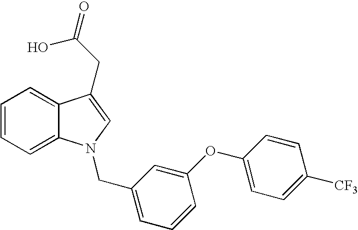 | B | 426[M + 1]⁺ |
| 484 | 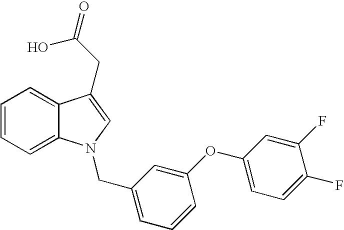 | B | 394[M + 1]⁺ |
| 485 | 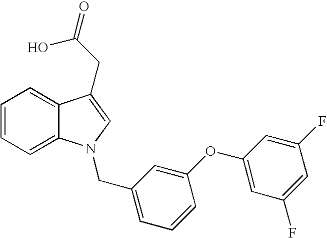 | A | 394[M + 1]⁺ |
| 486 | 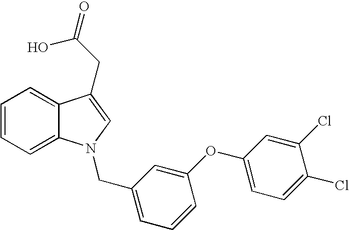 | B | 427[M + 1]⁺ |
| 487 | 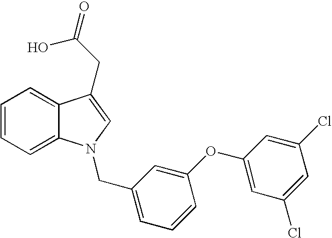 | B | 427[M + 1]⁺ |
| 488 | 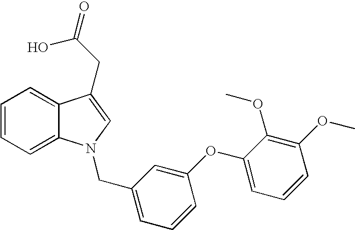 | B | 418[M + 1]⁺ |
| 489 | 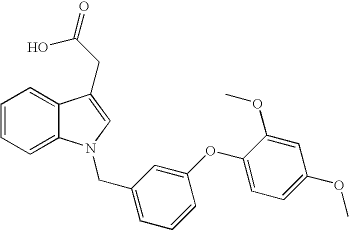 | A | 418[M + 1]⁺ |
| 490 | 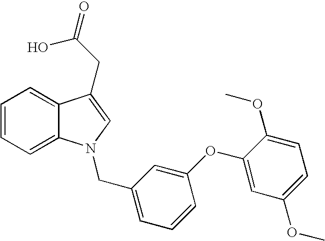 | A | 418[M + 1]⁺ |
| 491 | 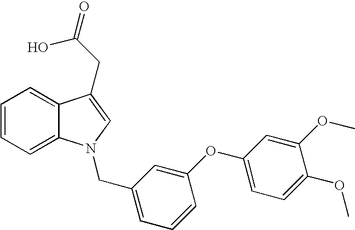 | A | 418[M + 1]⁺ |
| 492 | 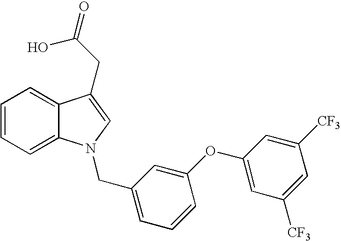 | B | 494[M + 1]⁺ |

TABLE 19
Examples of compounds made using Scheme 26.

| | | | |
|---|---|---|---|
| Example | Structure | IC50 Activity | MS-ESI: m/z |

| | | | |
|---|---|---|---|
| 493 | 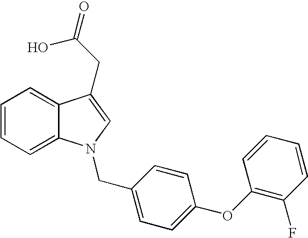 | B | 376[M + 1]⁺ |
| 494 | 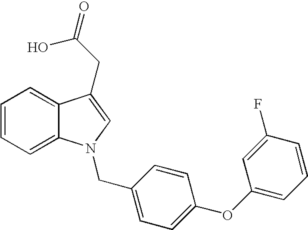 | B | 376[M + 1]⁺ |
| 495 | 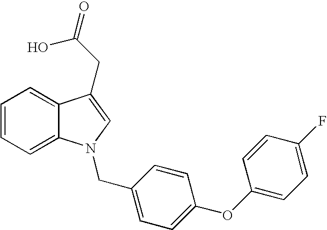 | B | 376[M + 1]⁺ |
| 496 | 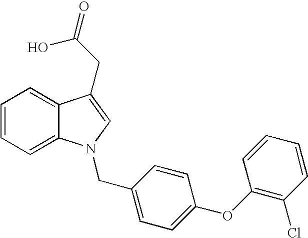 | B | 392[M + 1]⁺ |
| 497 | 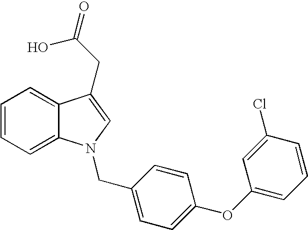 | B | 392.1[M + 1]⁺ |
| 498 | 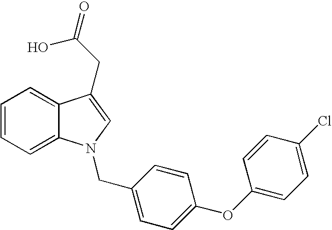 | B | 392[M + 1]⁺ |
| 499 | 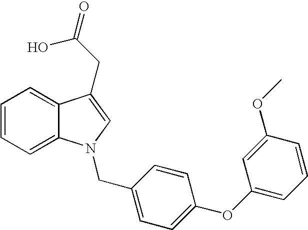 | A | 388.1[M + 1]⁺ |
| 500 | 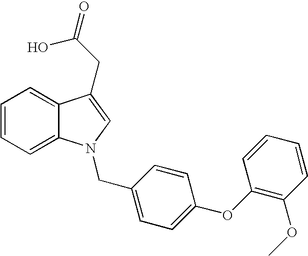 | B | 388.4[M + 1]⁺ |
| 501 | 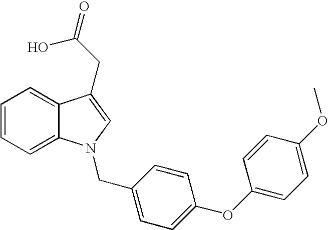 | B | 388.1[M + 1]⁺ |
| 502 | 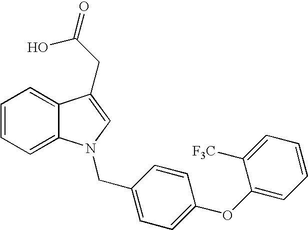 | B | 426.1[M + 1]⁺ |
| 503 | 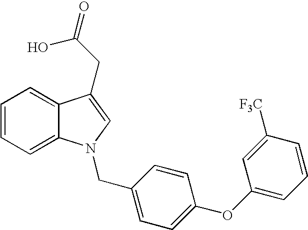 | B | 426.1[M + 1]⁺ |
| 504 | 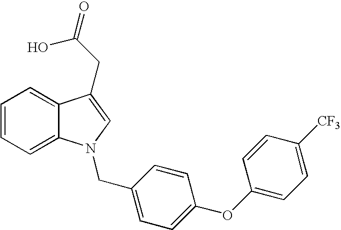 | B | 426[M + 1]⁺ |
| 505 | 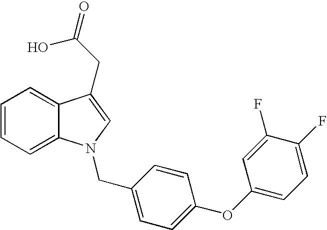 | B | 394[M + 1]⁺ |
| 506 | 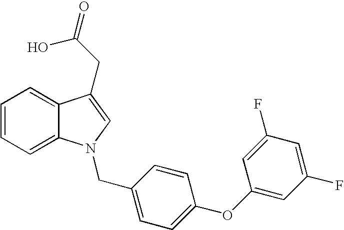 | B | 394[M + 1]⁺ |
| 507 | 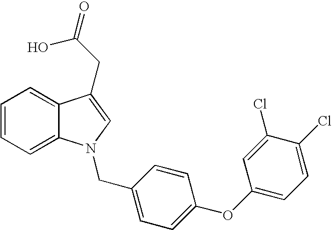 | B | 427[M + 1]⁺ |
| 508 | 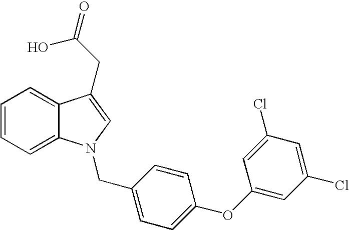 | B | 427[M + 1]⁺ |
| 509 | 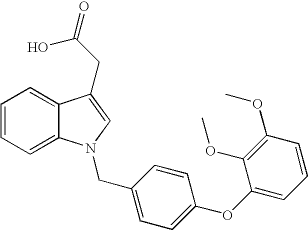 | B | 418[M + 1]⁺ |
| 510 | 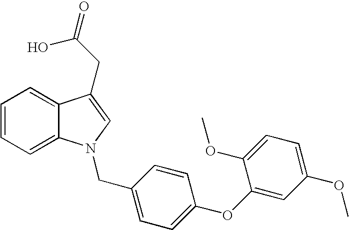 | B | 418[M + 1]⁺ |
| 511 | 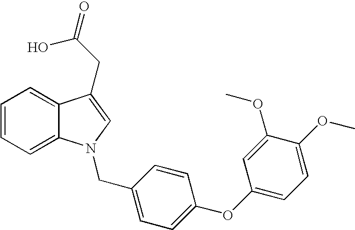 | B | 418[M + 1]⁺ |
| 512 | 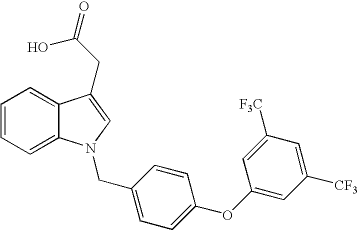 | B | 494[M + 1]⁺ |
| 513 | 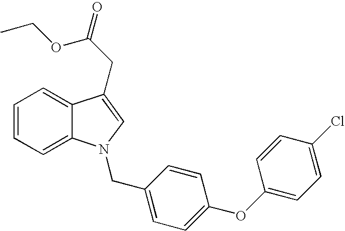 | | 420.9[M + 1]⁺ |
| 514 | 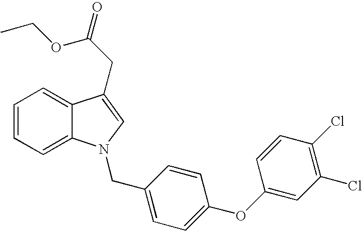 | | 455.3[M + 1]⁺ |
| 515 | 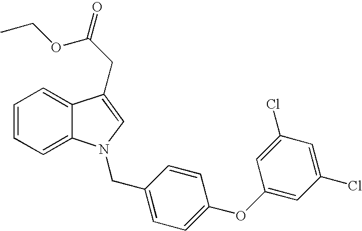 | B | 455.3[M + 1]⁺ |
| 516 | 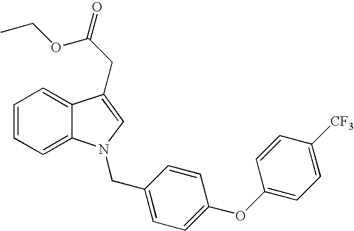 | A | 454[M + 1]⁺ |
| 517 | 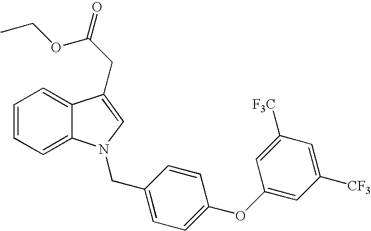 | A | 522.4[M + 1]⁺ |
| 518 | 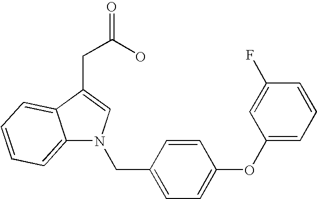 | B | 376[M + 1]⁺ |
| 519 | 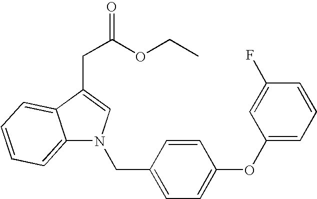 | | 404[M + 1]⁺ |

TABLE 20
Examples of compounds made using Scheme 27.

| Example | Structure | IC50 Activity | MS-ESI: m/z |
|---|---|---|---|
| 520 | 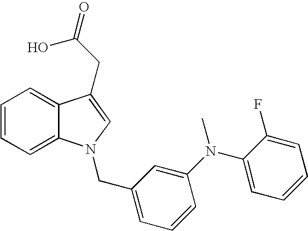 | B | 389.4[M + 1]⁺ |
| 521 | 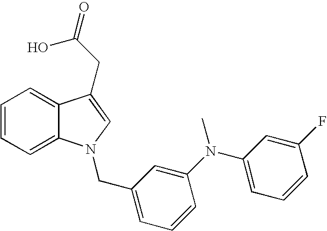 | B | 389.4[M + 1]⁺ |
| 522 | 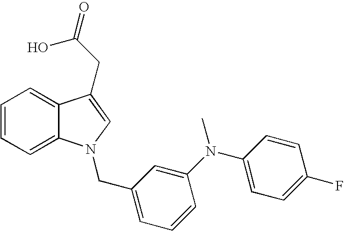 | B | 389.4[M + 1]⁺ |
| 523 | 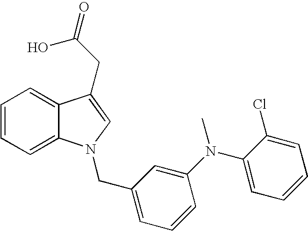 | B | 405.9[M + 1]⁺ |
| 524 | 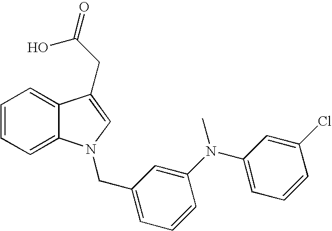 | B | 405.9[M + 1]⁺ |
| 525 | 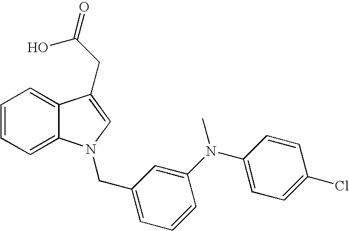 | B | 405.9[M + 1]⁺ |
| 526 | 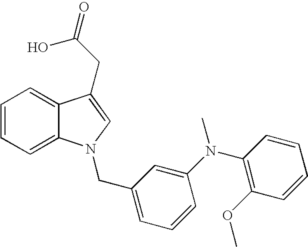 | A | 401.4[M + 1]⁺ |
| 527 | 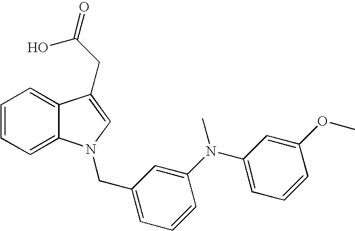 | B | 401.4[M + 1]⁺ |
| 528 | 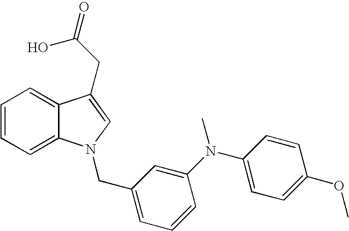 | B | 401.4[M + 1]⁺ |
| 529 | 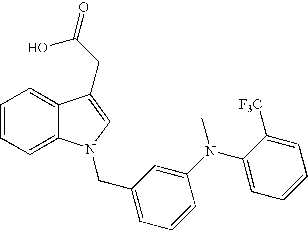 | B | 439.4[M + 1]⁺ |
| 530 | 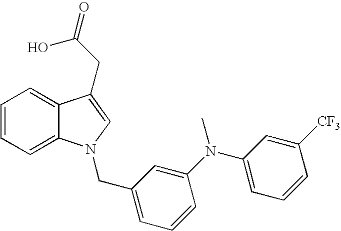 | B | 439.4[M + 1]⁺ |
| 531 | 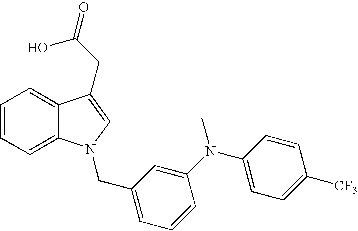 | B | 439.4[M + 1]⁺ |
| 532 | 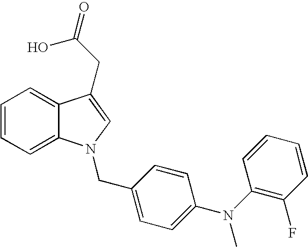 | | 389.4[M + 1]⁺ |
| 533 | 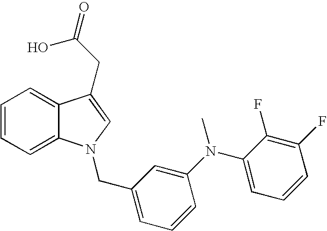 | B | 407.4[M + 1]⁺ |
| 534 | 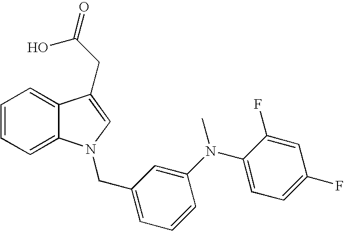 | A | 407.4[M + 1]⁺ |
| 535 | 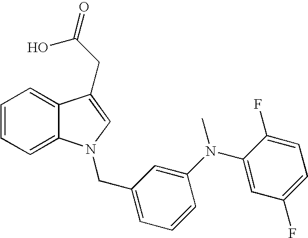 | A | 407.4[M + 1]⁺ |
| 536 | 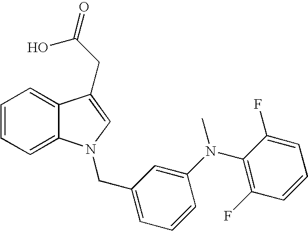 | A | 407.4[M + 1]⁺ |
| 537 | 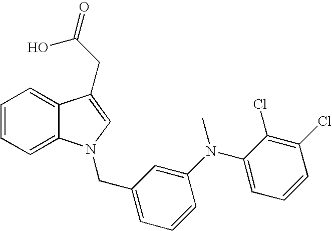 | B | 440.3[M + 1]⁺ |
| 538 | 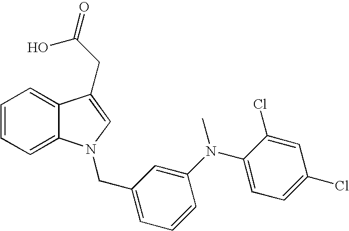 | B | 440.3[M + 1]⁺ |
| 539 | 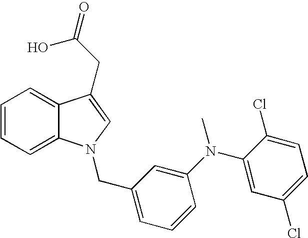 | B | 440.3[M + 1]⁺ |
| 540 | 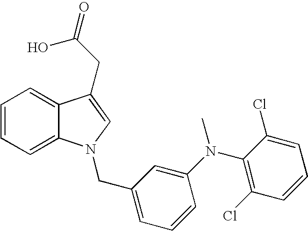 | B | 440.3[M + 1]⁺ |
| 541 | 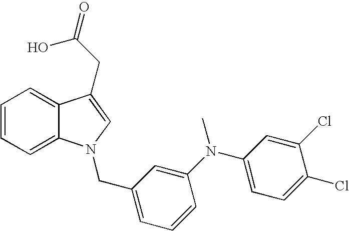 | B | 440.3[M + 1]⁺ |
| 542 | 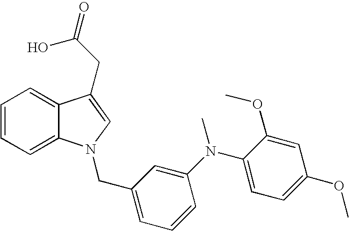 | B | 431.5[M + 1]⁺ |
| 543 | 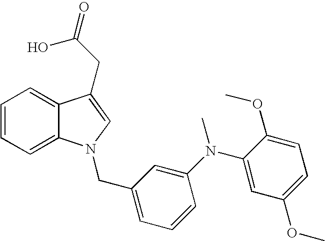 | B | 431.5[M + 1]⁺ |
| 544 | 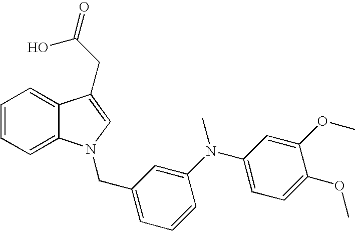 | B | 431.5[M + 1]⁺ |
| 545 | 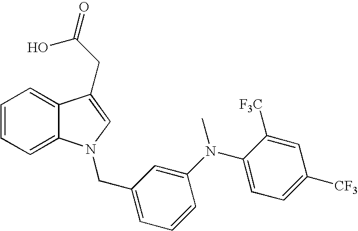 | A | 507.4[M + 1]⁺ |
| 546 | 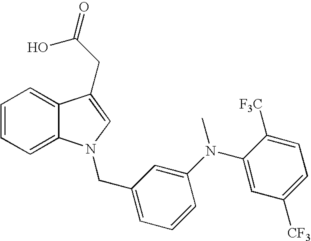 | B | 507.4[M + 1]⁺ |
| 547 | 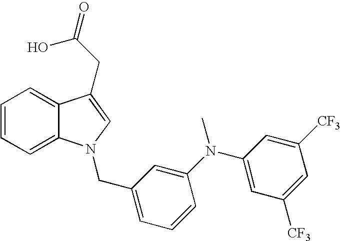 | B | 507.4[M + 1]⁺ |
| 548 | 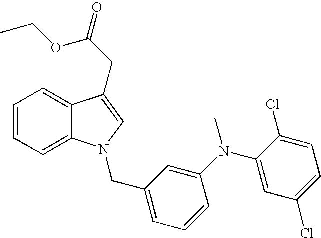 | B | 468.3[M + 1]⁺ |
| 549 | 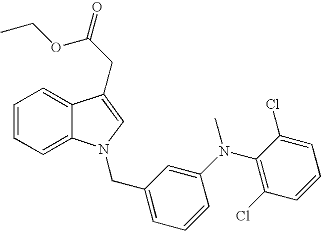 | | 468.3[M + 1]⁺ |
| 550 | 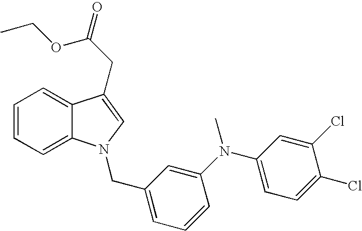 | C | 468.3[M + 1]⁺ |
| 551 | 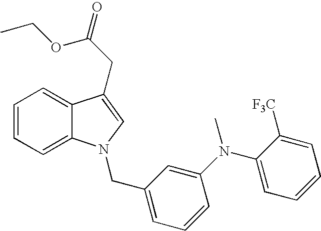 | | 467.5[M + 1]⁺ |
| 552 | 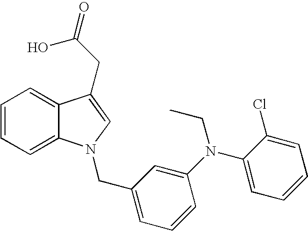 | B | 419.9[M + 1]⁺ |
| 553 | 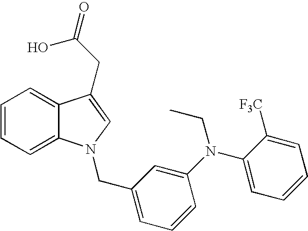 | A | 453.4[M + 1]⁺ |
| 554 | 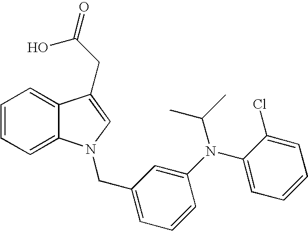 | B | 433[M + 1]⁺ |
| 555 | 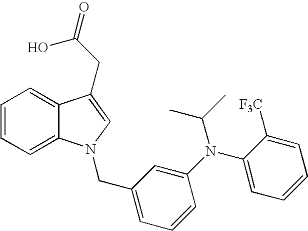 | B | 467[M + 1]⁺ |

TABLE 21
Examples of compounds made using Scheme 28.

| | | | MS-ESI |
|---|---|---|---|
| Example | Structure | IC50 Activity | m/z |
| 556 | 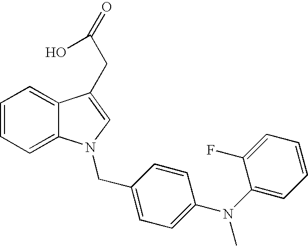 | | 389.4[M + 1]⁺ |
| 557 | 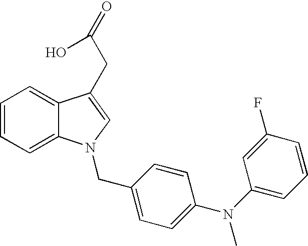 | B | 389.4[M + 1]⁺ |
| 558 | 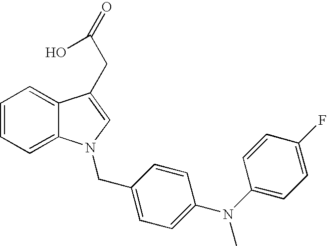 | A | 389.4[M + 1]⁺ |
| 559 | 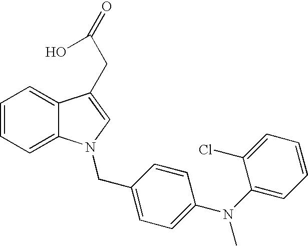 | B | 405.9[M + 1]⁺ |
| 560 | 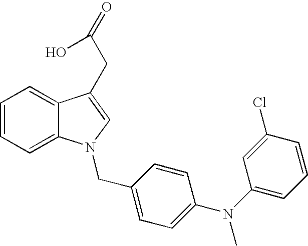 | B | 405.9[M + 1]⁺ |
| 561 | 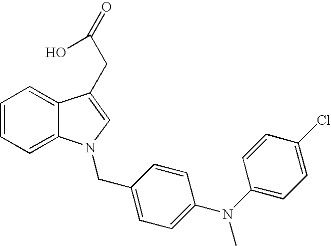 | B | 405.9[M + 1]⁺ |
| 562 | 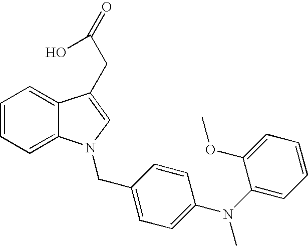 | A | 401.4[M + 1]⁺ |
| 563 | 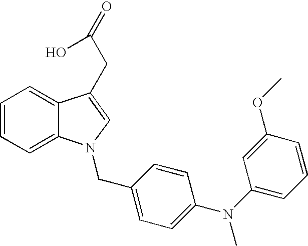 | B | 401.4[M + 1]⁺ |
| 564 | 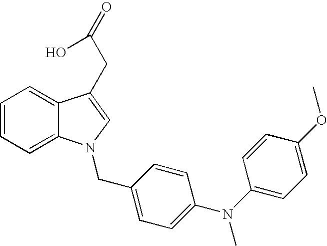 | B | 401.4[M + 1]⁺ |
| 565 | 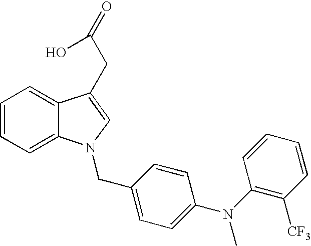 | B | 439.4[M + 1]⁺ |
| 566 | 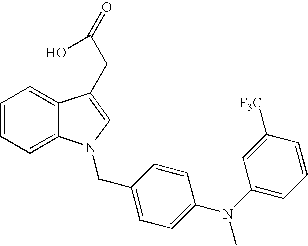 | B | 439.4[M + 1]⁺ |
| 567 | 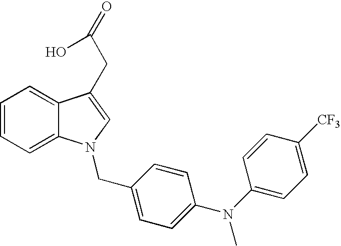 | B | 439.4[M + 1]⁺ |
| 568 | 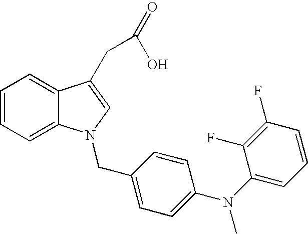 | B | 407.1[M + 1]⁺ |
| 569 | 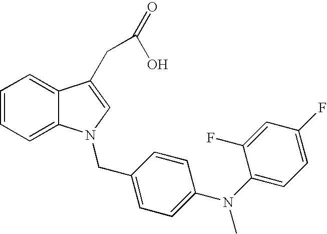 | B | 407.2[M + 1]⁺ |
| 570 | 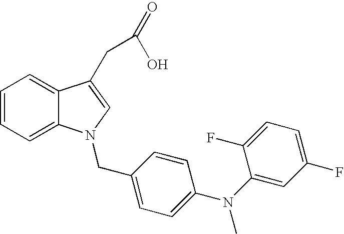 | B | 407.2[M + 1]⁺ |
| 571 | 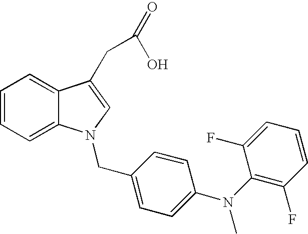 | A | 407.2[M + 1]⁺ |
| 572 | 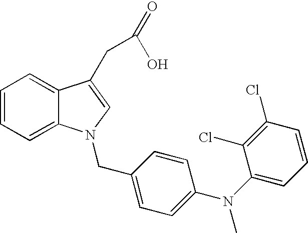 | B | 440[M + 1]⁺ |
| 573 | 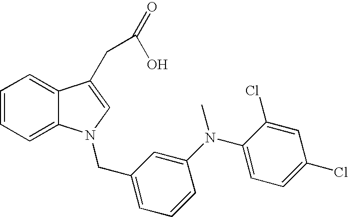 | B | 440[M + 1]⁺ |
| 574 | 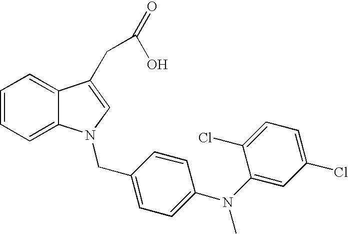 | B | 440[M + 1]⁺ |
| 575 | 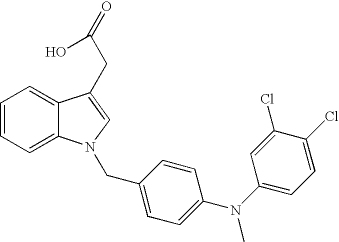 | B | 440[M + 1]⁺ |
| 576 | 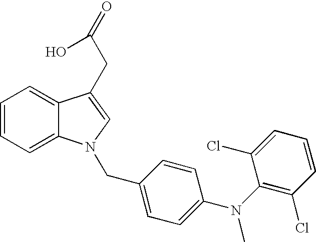 | B | 440.3[M + 1]⁺ |
| 577 | 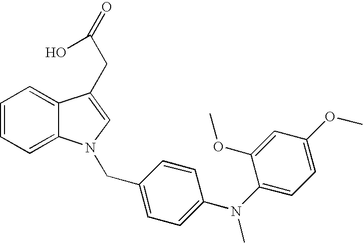 | A | 431.2[M + 1]⁺ |
| 578 | 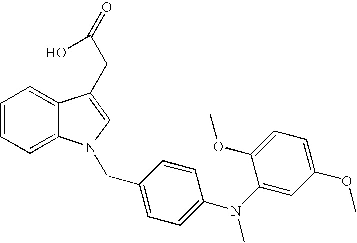 | B | 431.2[M + 1]⁺ |
| 579 | 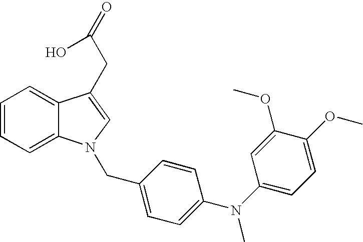 | A | 431.2[M + 1]⁺ |
| 580 | 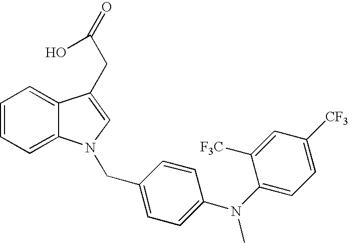 | B | 507.1[M + 1]⁺ |
| 581 | 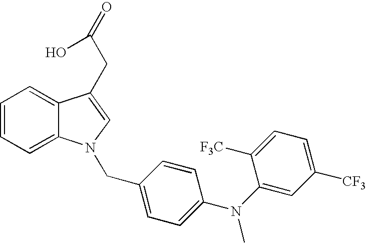 | B | 507.1[M + 1]⁺ |
| 582 | 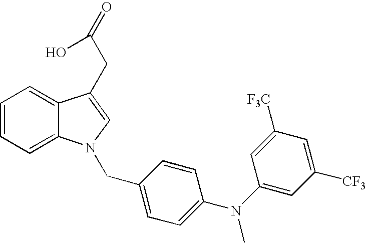 | B | 507.1[M + 1]⁺ |
| 583 | 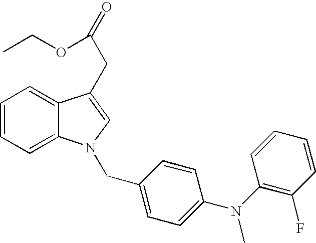 | A | 417.4[M + 1]⁺ |

TABLE 22
Examples of compounds made using Scheme 29.

| Example | Structure | IC50 Activity | MS-ESI: m/z |
|---|---|---|---|
| 584 | 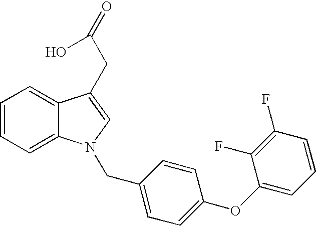 | B | 394.3[M + 1]⁺ |
| 585 | 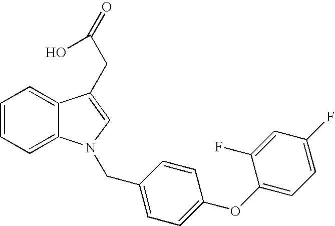 | B | 394.3[M + 1]⁺ |
| 586 | 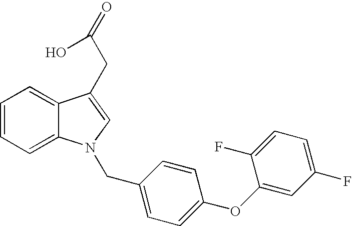 | B | 394.3[M + 1]⁺ |
| 587 | 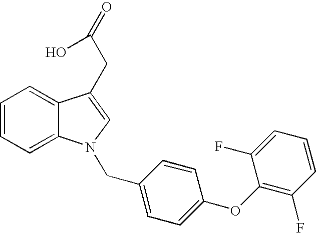 | B | 394.3[M + 1]⁺ |
| 588 | 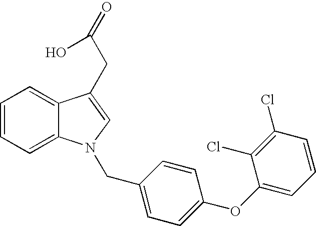 | B | 427[M + 1]⁺ |
| 589 | 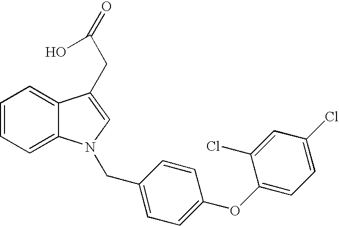 | B | 427.1[M + 1]⁺ |
| 590 | 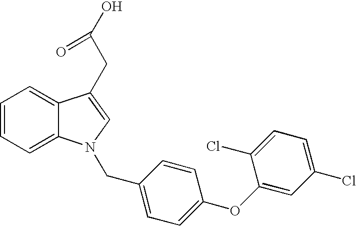 | B | 427.3[M + 1]⁺ |

TABLE 23
Examples of compounds made using Scheme 30.

| Example | Structure | IC50 Activity | MS-ESI: m/z |
|---|---|---|---|
| 591 | 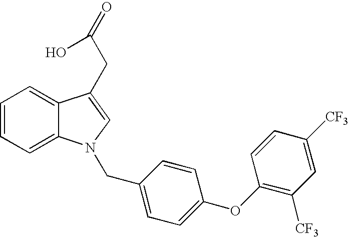 | B | 494[M + 1]⁺ |
| 592 | 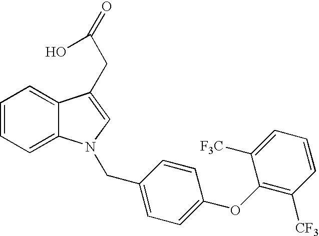 | A | 494[M + 1]⁺ |
| 593 | 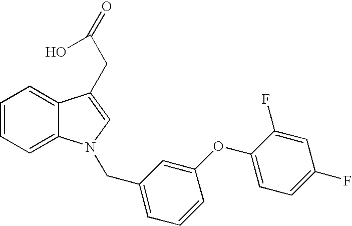 | B | 394[M + 1]⁺ |
| 594 | 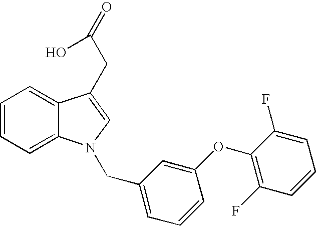 | B | 394[M + 1]⁺ |
| 595 | 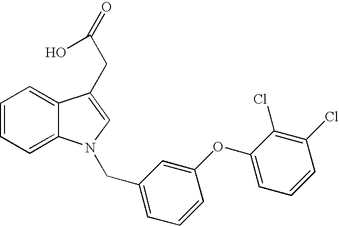 | B | 427[M + 1]⁺ |
| 596 | 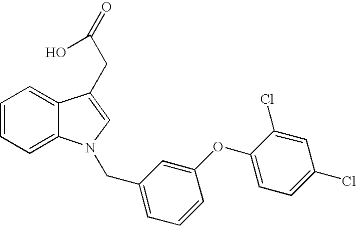 | B | 427[M + 1]⁺ |
| 597 | 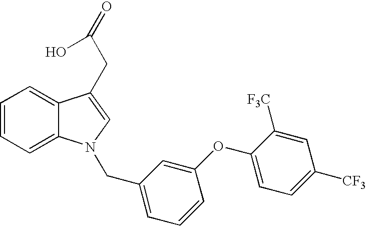 | B | 494[M + 1]⁺ |
| 598 | 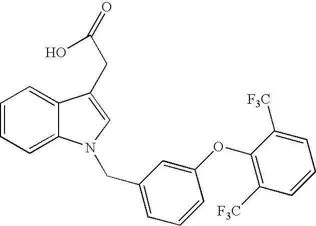 | B | 480[M + 1]⁺ |
| 599 | 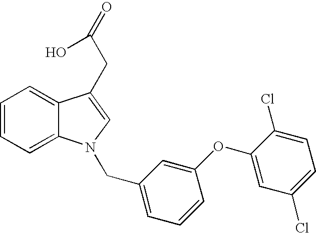 | A | 427[M + 1]⁺ |
| 600 | 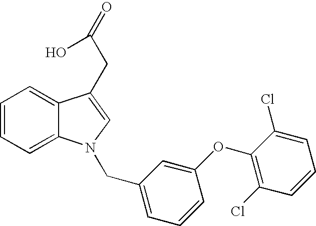 | A | 427[M + 1]⁺ |

TABLE 24
Examples of compounds made using Scheme 31.

| Example | Structure | IC50 Activity | MS-ESI: m/z |
|---|---|---|---|
| 601 | 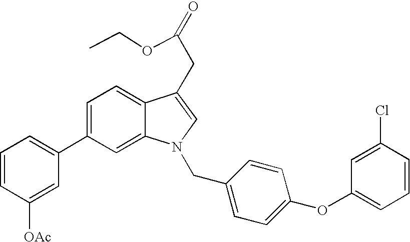 | C | 555.0[M + 1]⁺ |
| 602 | 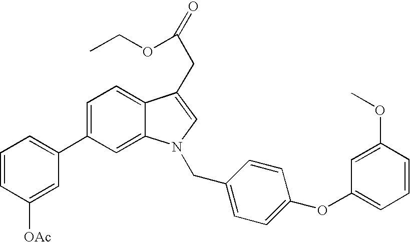 | A | 550[M + 1]⁺ |
| 603 | 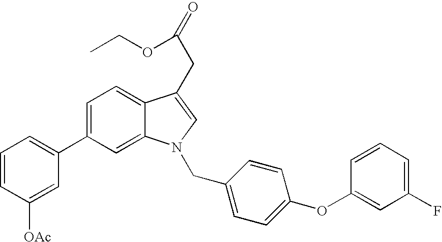 | C | 538[M + 1]⁺ |
| 604 | 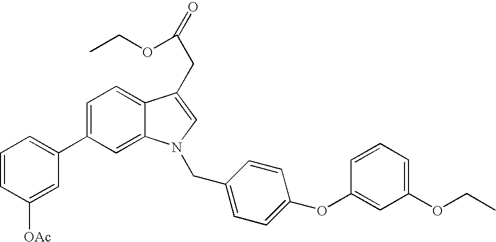 | C | 564[M + 1]⁺ |
| 605 | 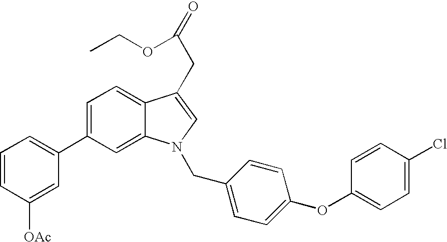 | C | 555[M + 1]⁺ |
| 606 | 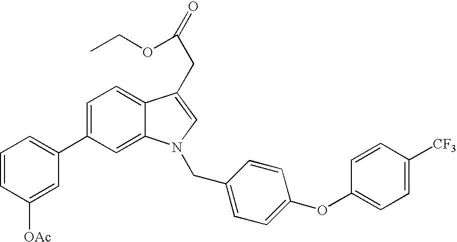 | A | 588[M + 1]⁺ |
| 607 | 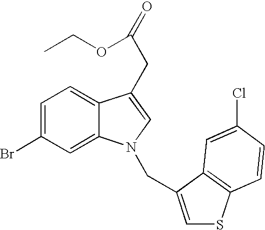 | | 463[M + 1]⁺ |

TABLE 25
Examples of compounds made using Scheme 32.

| Example | Structure | IC50 Activity | MS-ESI: m/z |
|---|---|---|---|
| 608 | 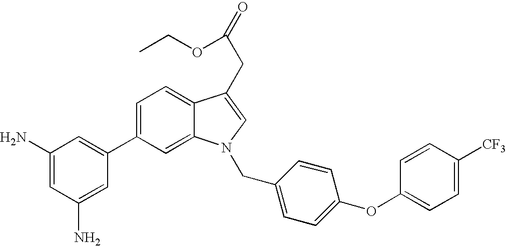 | A | 560[M + 1]⁺ |
| 609 | 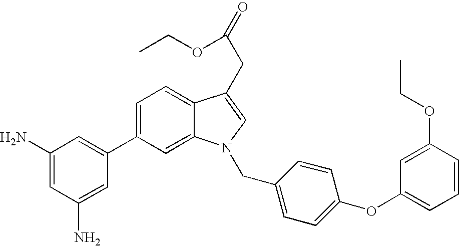 | A | 536[M + 1]⁺ |
| 610 | 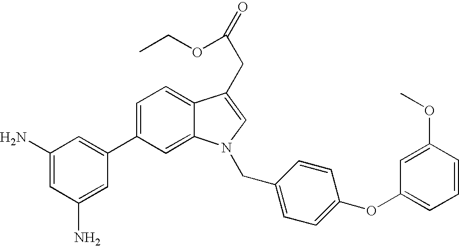 | A | 522[M + 1]⁺ |
| 611 | 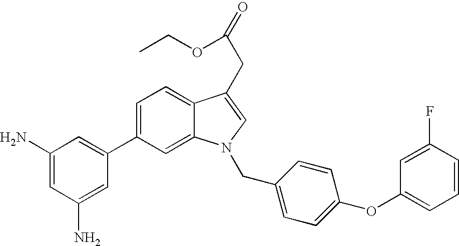 | | 510[M + 1]⁺ |
| 612 | 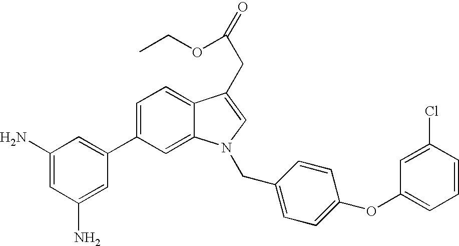 | B | 527[M + 1]⁺ |
| 613 | 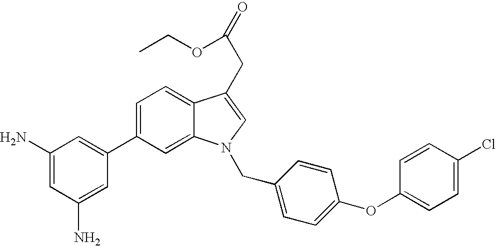 | A | 526[M + 1]⁺ |

TABLE 26
Examples of compounds made using Scheme 33.

| Example | Structure | IC50 Activity | MS-ESI: m/z |
|---|---|---|---|
| 614 | 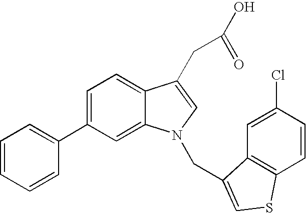 | B | 432[M + 1]⁺ |
| 615 | 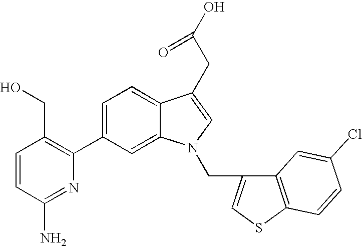 | A | 478[M + 1]⁺ |
| 616 | 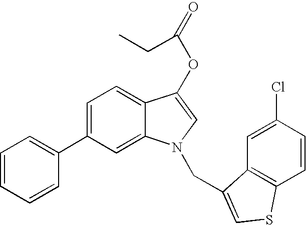 | C | 446[M + 1]⁺ |

TABLE 27
Examples of compounds made using Scheme 34.

| Example | Structure | IC50 Activity | MS-ESI: m/z |
|---|---|---|---|
| 617 | 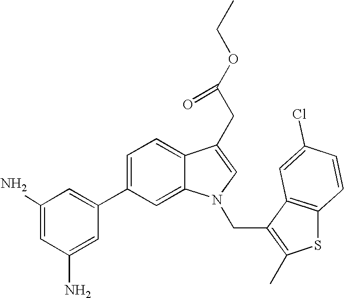 | B or C | 505.0[M + 1]⁺ |

TABLE 28
Examples of compounds made using Scheme 35.

| Example | Structure | IC50 Activity | MS-ESI: m/z |
|---|---|---|---|
| 618 | 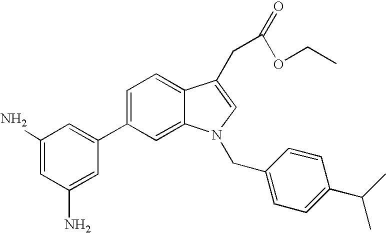 | | 442[M + 1]⁺ |
| 619 | 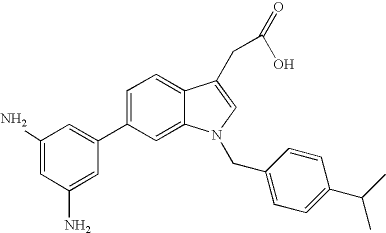 | B | 414.5[M + ]⁺ |

TABLE 29
Examples of compounds made using Scheme 36.

| Example | Structure | IC50 Activity | MS-ESI: m/z |
|---|---|---|---|
| 620 | 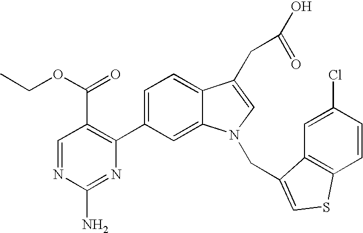 | | 521[M + 1]⁺ |
| 621 | 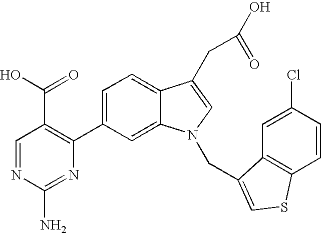 | C | 493[M + 1]⁺ |
| 622 | 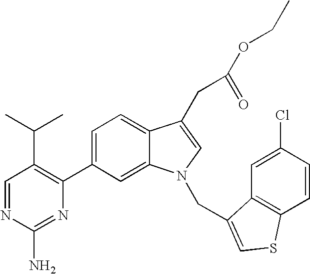 | | 520[M + 1]⁺ |
| 623 | 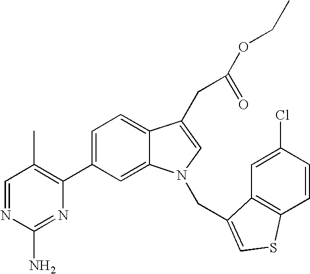 | | 492[M + 1]⁺ |

TABLE 30
Examples of compounds made using Scheme 37.

| Example | Structure | IC50 Activity | MS-ESI: m/z |
|---|---|---|---|
| 624 | 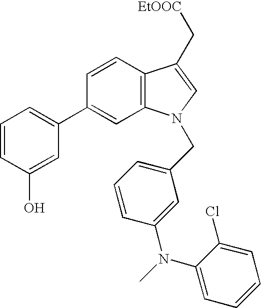 | A | 526[M + 1]⁺ |

TABLE 31
Examples of compounds made using Schemes 38-45.

| Example | Structure | IC50 Activity |
|---|---|---|
| 625 | 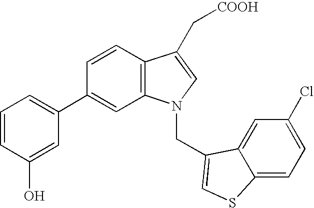 | B |
| 626 | 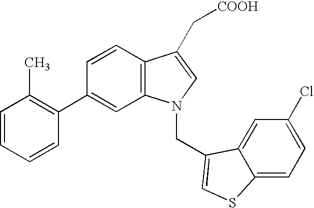 | B |
| 627 | 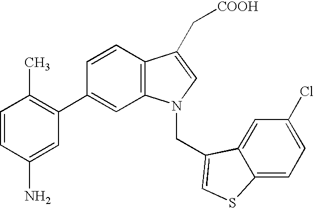 | B |
| 628 | 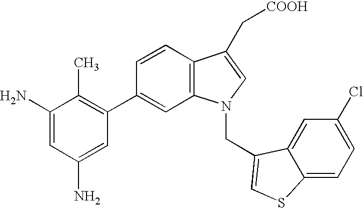 | B |
| 629 | 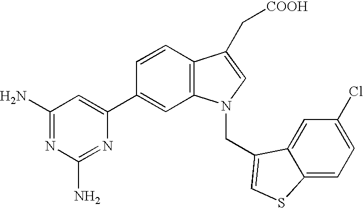 | B |
| 630 | 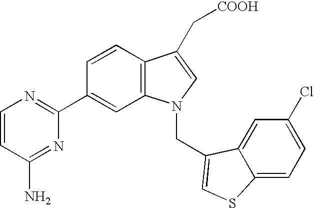 | B |
| 331 | 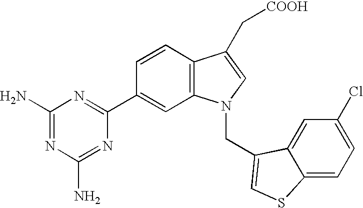 | B |
| 632 | 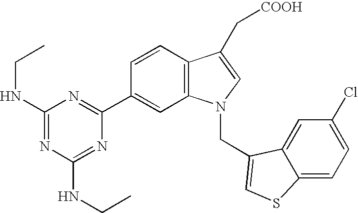 | B |
| 633 | 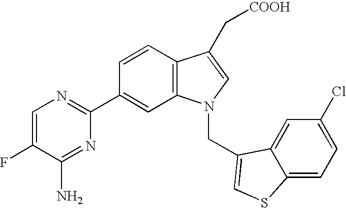 | A |
| 634 | 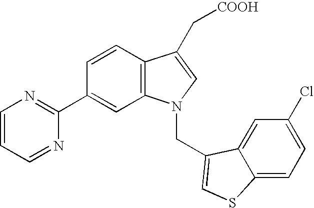 | na |
| 635 | 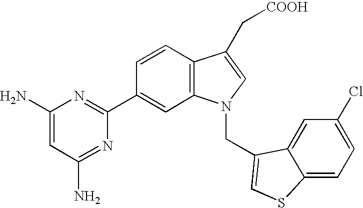 | A |
| 636 | 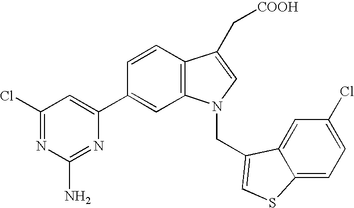 | B |
| 637 | 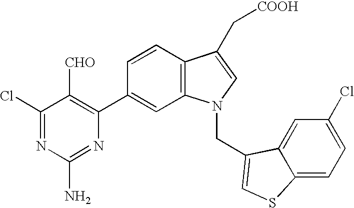 | B |
| 638 | 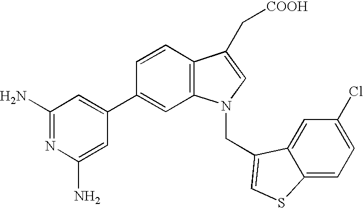 | A |
| 639 | 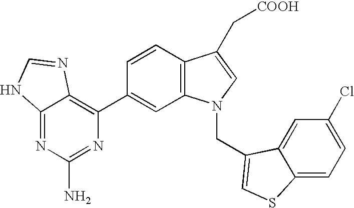 | A |
| 640 | 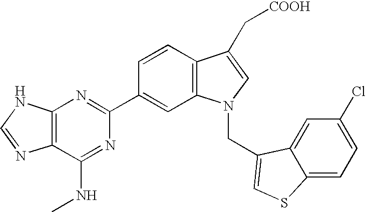 | B |
| 641 | 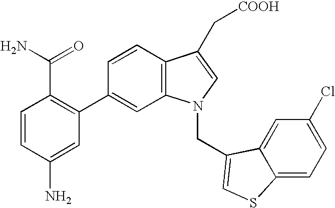 | A |
| 642 | 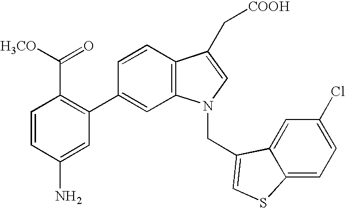 | B |
| 643 | 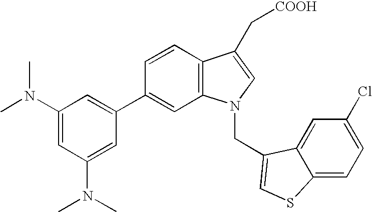 | na |
| 644 | 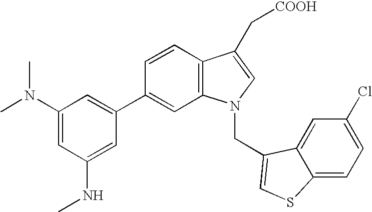 | na |
| 645 | 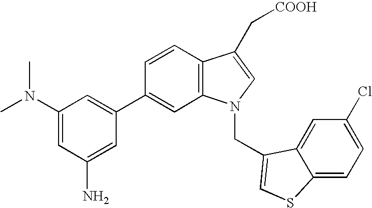 | na |
| 646 | 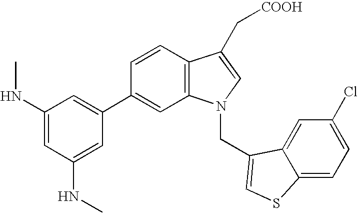 | B |
| 647 | 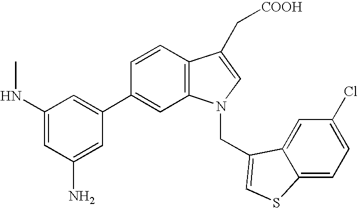 | B |
| 648 | 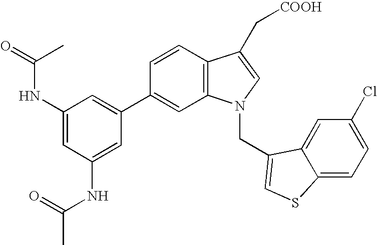 | B |
| 649 | 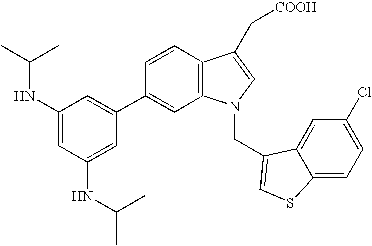 | B |
| 650 | 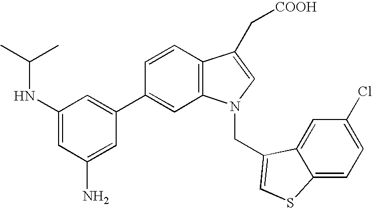 | B |
| 651 | 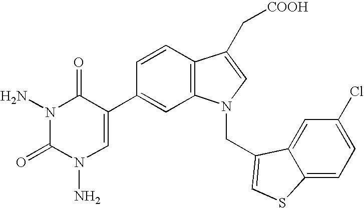 | C |
| 652 | 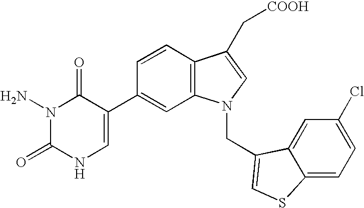 | A |
| 653 | 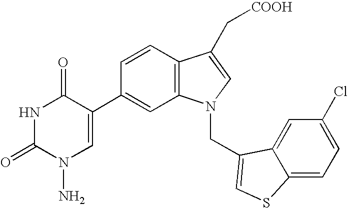 | A |

TABLE 32
Examples of compounds made using Schemes 46-49.

| Example | Structure | IC50 Activity |
|---|---|---|
| 654 | 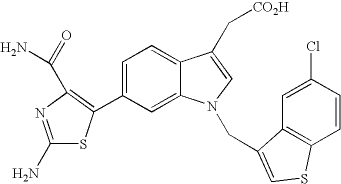 | B |
| 655 | 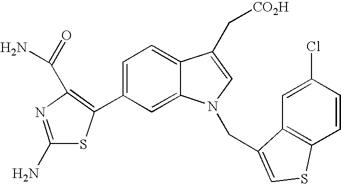 | A |
| 656 | 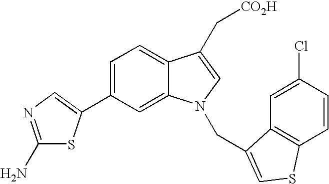 | A |
| 657 | 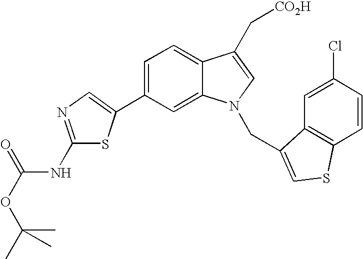 | B |
| 658 | 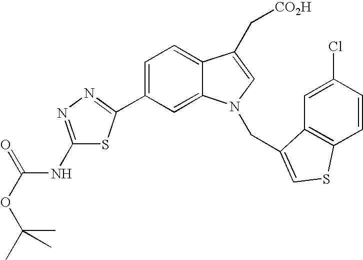 | B |
| 659 | 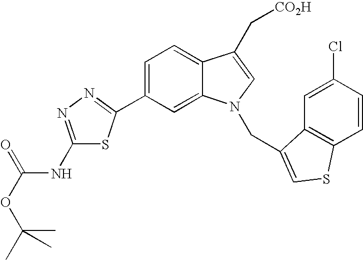 | A |
| 660 | 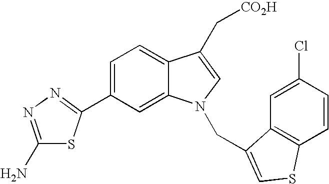 | A |
| 661 | 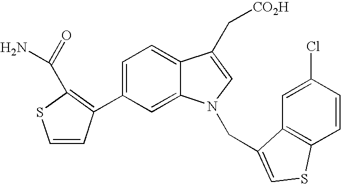 | A |
| 662 | 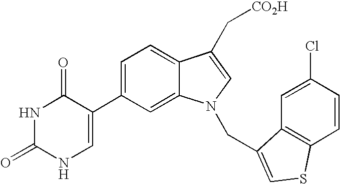 | A |
| 663 | 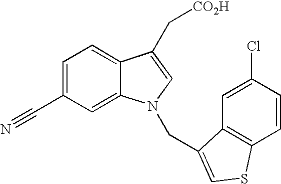 | A |
| 664 | 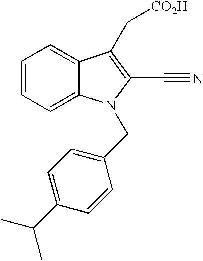 | A |
| 665 | 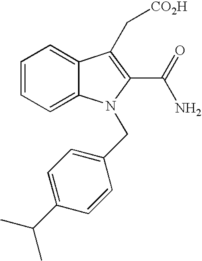 | A |

TABLE 33
Examples of compounds made using Scheme 50.

| Example | Structure | IC50 Activity | LC/MS |
|---|---|---|---|
| 666 | 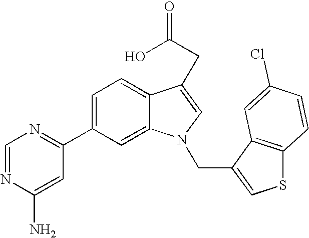 | A | 4447.0(pos APCI) |
| 667 | 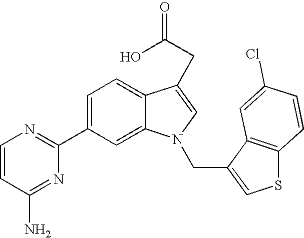 | A | 447.0(pos APCI) |
| 668 | 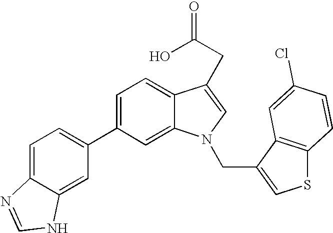 | A | 470.1(pos APCI) |
| 669 | 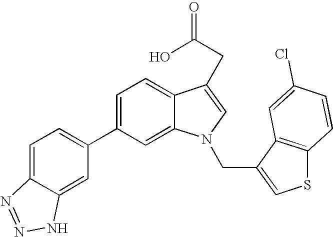 | A | 473.4(pos APCI) |

TABLE 34
Examples of compounds made using Schemes 51-52.

| Example | Structure | IC50 Activity |
|---|---|---|
| 670 | 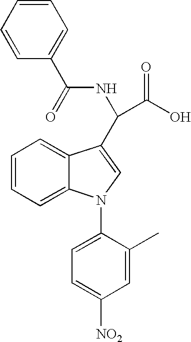 | A |
| 671 | 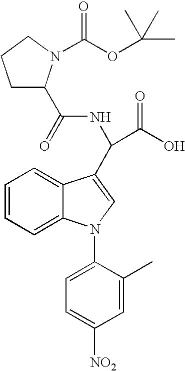 | A |
| 672 | 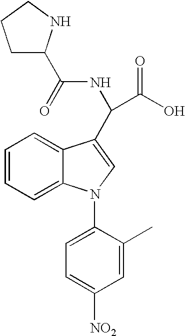 | C |
| 673 | 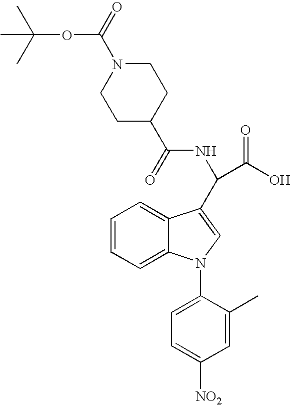 | A |
| 674 | 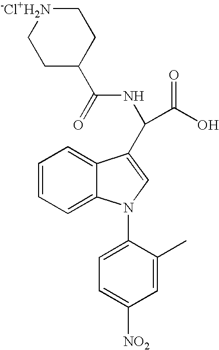 | C |
| 675 | 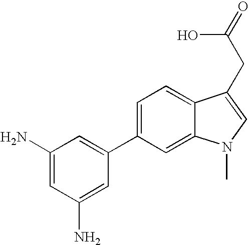 | C |
| 676 | 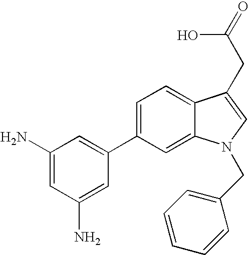 | A |
| 677 | 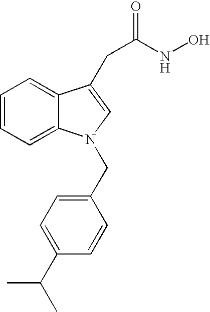 | A |
| 678 | 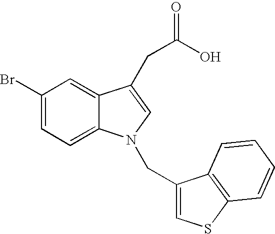 | A |
| 679 | 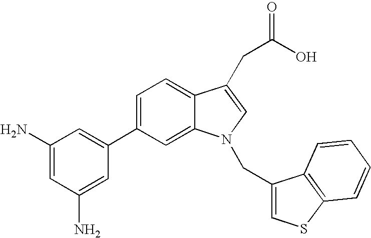 | B |
| 680 | 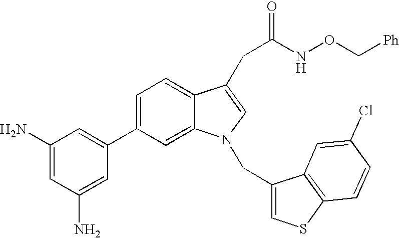 | B |
| 681 | 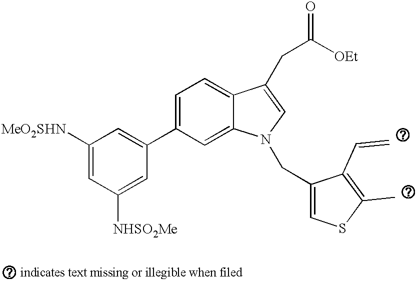 | C |
| 682 | 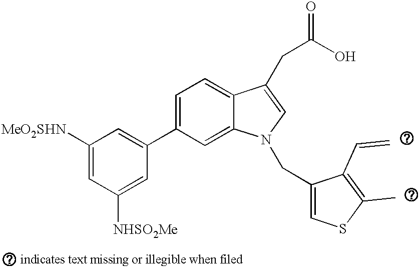 | B |
| 683 | 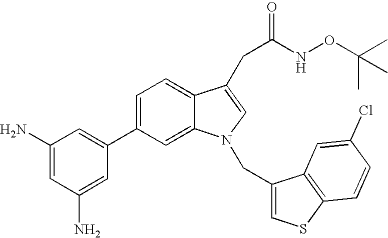 | A |
| 684 | 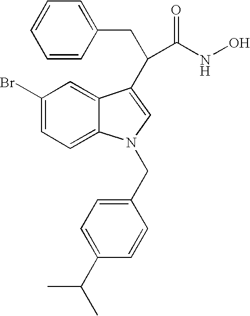 | B |
| 685 | 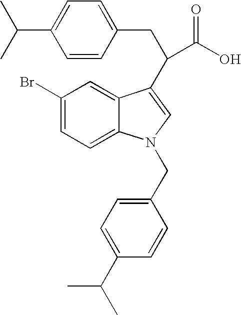 | B |
| 686 | 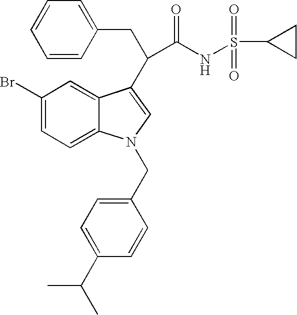 | B |
| 687 | 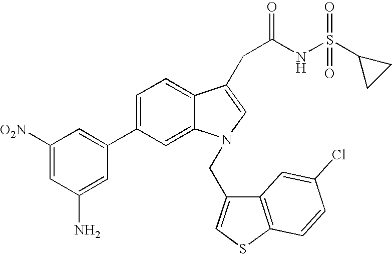 | B |
| 688 | 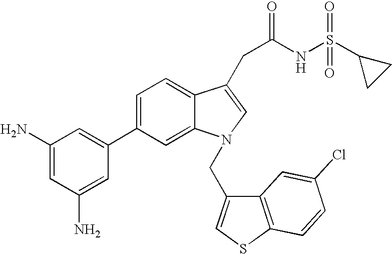 | B |
| 689 | 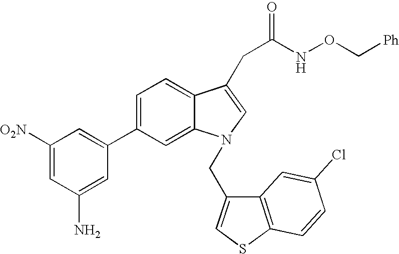 | C |
| 690 | 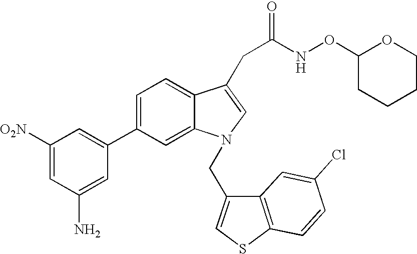 | C |
| 691 | 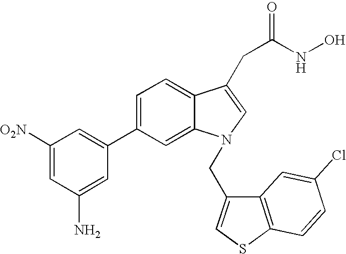 | A |
| 692 | 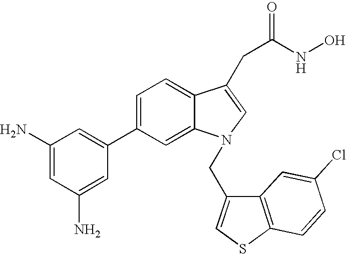 | A |
| 693 | 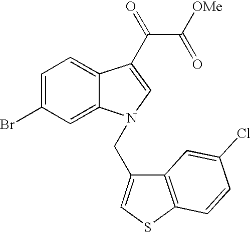 | C |
| 694 | 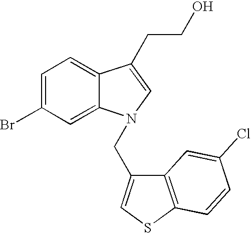 | C |
| 695 | 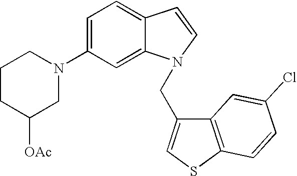 | A |
| 696 | 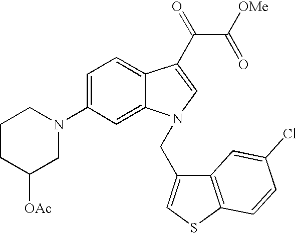 | A |
| 697 | 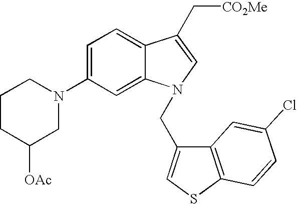 | C |
| 698 | 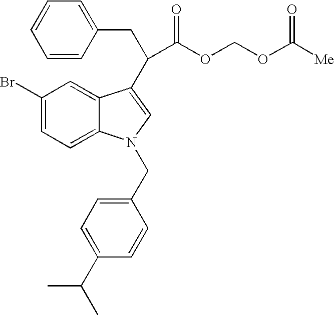 | |
| 699 | 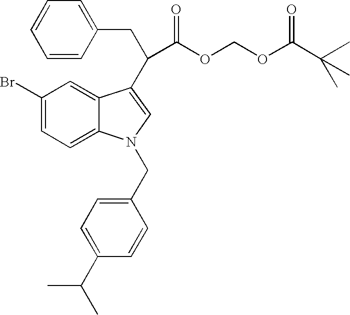 | |
| 700 | 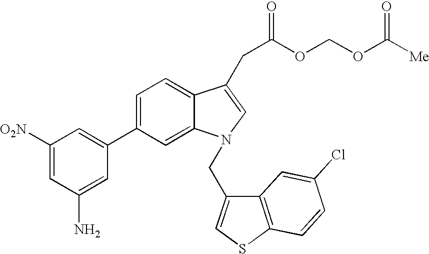 | |
| 701 | 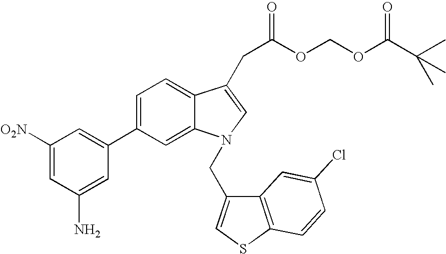 | |

TABLE 35
Examples of compounds made using Scheme 53.

| | | IC50 | Synthetic route to |
|---|---|---|---|
| Example | Structure | Activity | alkyl bromide |
| 734 | 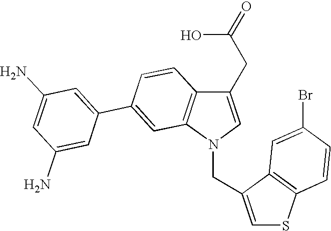 | A | Described inScheme 53 |
| 702 | 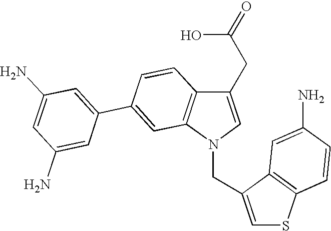 | A | Compound 39 |
| 703 | 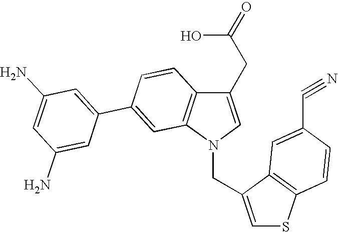 | A | Commercial 5-cyanobenzothio-phene, Scheme 60 |
| 791 | 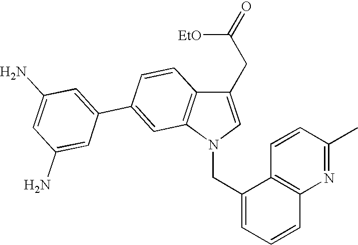 | A | Commercial |
| 704 | 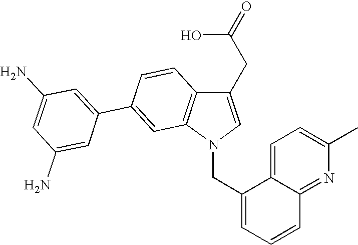 | C | Commercial |
| 792 | 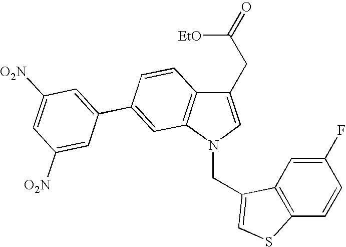 | C | Commercial 3-methylbenzothiophene,Scheme 58 |
| 705 | 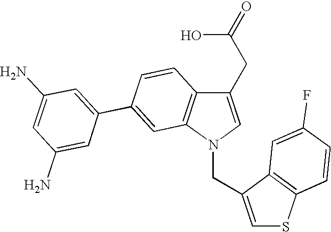 | B | Commercial 3-methylbenzothiophene,Scheme 58 |
| 706 | 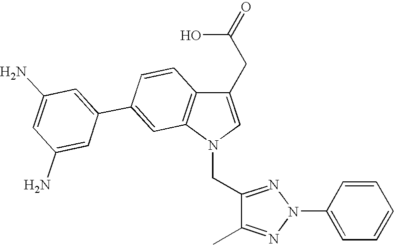 | A | Commercial |
| 793 | 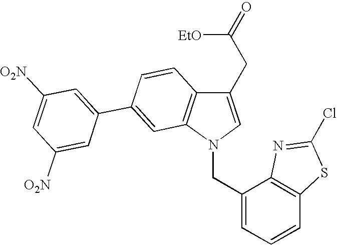 | A | Commercial 2-chlorobenzothiazole |
| 794 | 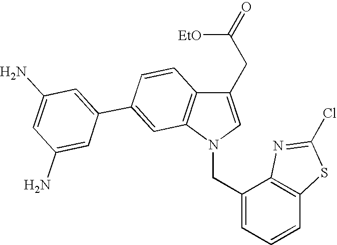 | A | Commercial 2-chlorobenzothiazole |
| 708 | 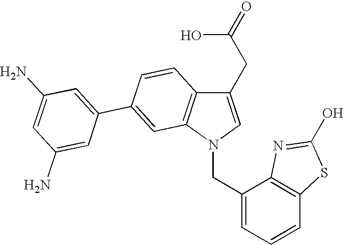 | A | Commercial 2-chlorobenzothiazole |
| 709 | 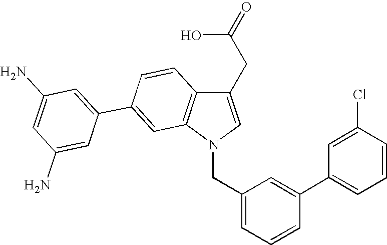 | A | Commercialbromobenzylbromide |
| 710 | 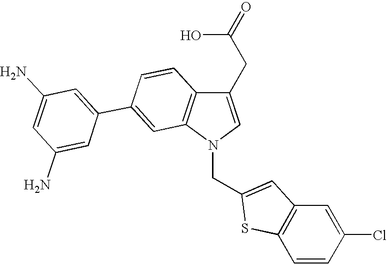 | B | Compound 35,Scheme 58 |

TABLE 36
Examples of compounds made using Scheme 54.

| | | IC50 | Synthetic route to |
|---|---|---|---|
| Example | Structure | Activity | alkyl bromide |
| 735 | 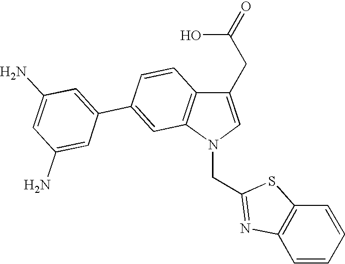 | C | Described inScheme 54 |
| 711 | 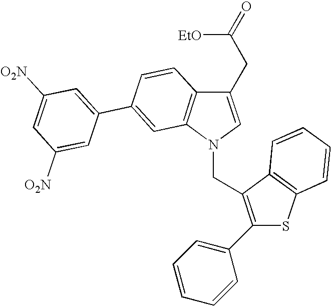 | A | Scheme 59 |
| 712 | 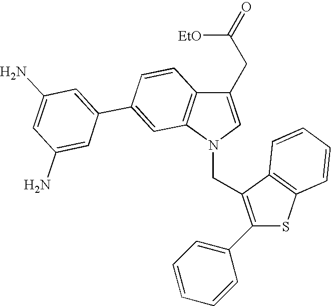 | B | Scheme 59 |
| 713 | 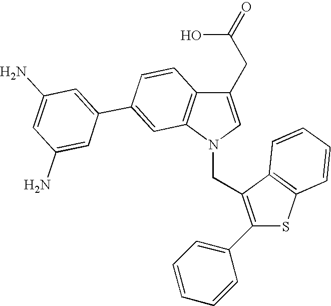 | B | Scheme 59 |
| 714 | 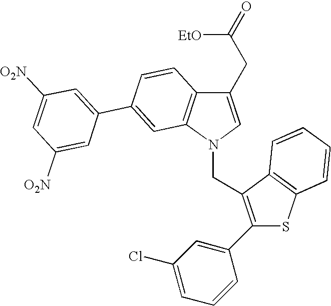 | A | Scheme 59 |
| 715 | 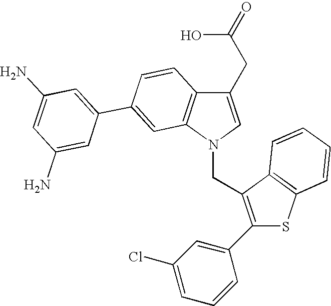 | B | Scheme 59 |
| 775 | 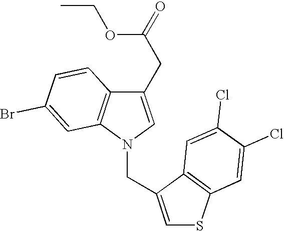 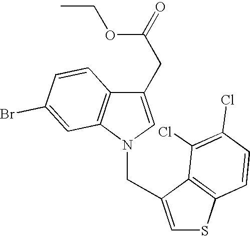 | C | Scheme 58 |
| 716 | 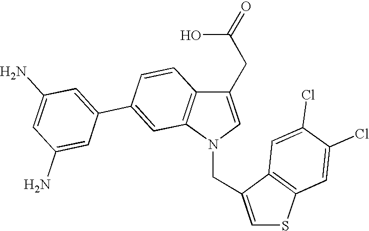 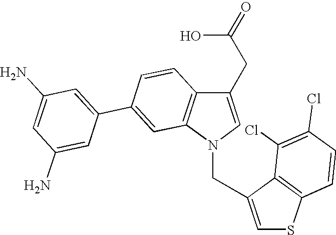 | A | Scheme 58 |
| 776 | 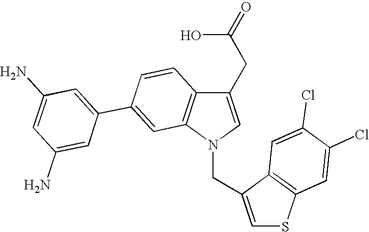 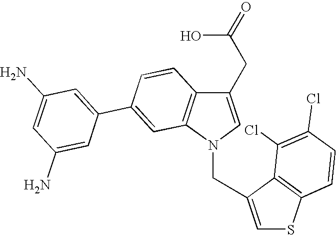 | A | Scheme 58 |
| 717 | 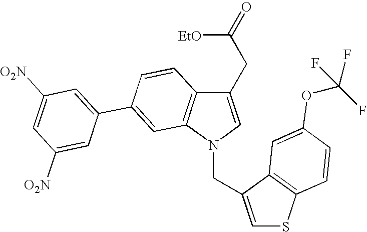 | A | Scheme 58 |
| 718 | 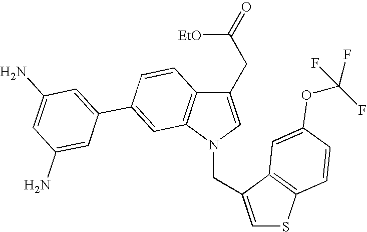 | A | Scheme 58 |
| 719 | 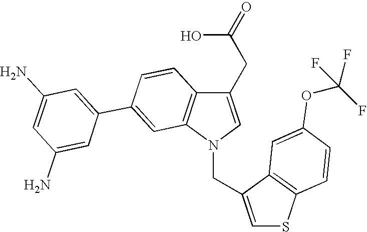 | A | Scheme 58 |
| 777 | 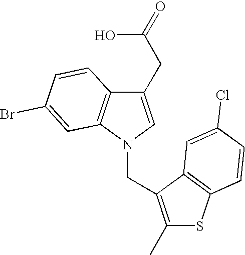 | A | Scheme 60 |
| 778 | 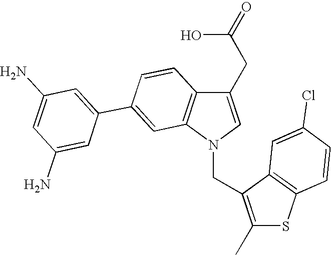 | B | Scheme 60 |
| 720 | 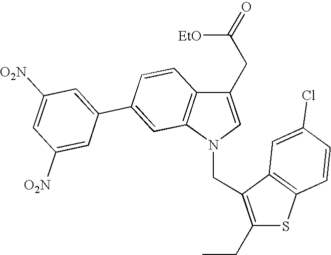 | C | SyntheticCommun., 1998,28, 3479-3490 |
| 721 | 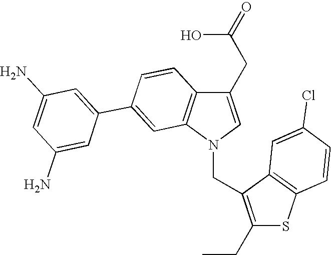 | B | SyntheticCommun., 1998,28, 3479-3490 |
| 722 | 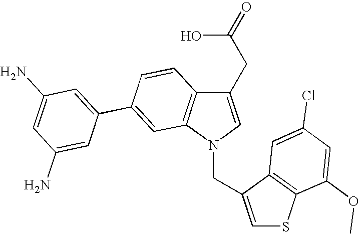 | A | Compound 39 |
| 723 | 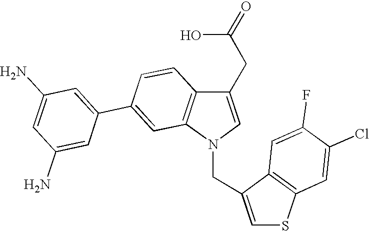 | B | Scheme 58 |
| 779 | 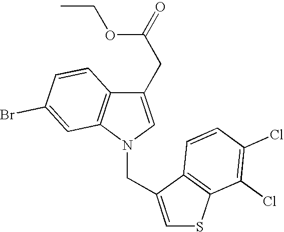 | C | Scheme 58 |
| 780 | 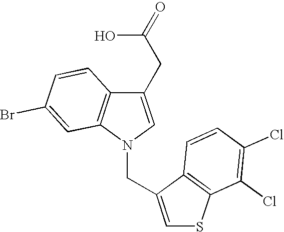 | C | Scheme 58 |
| 724 | 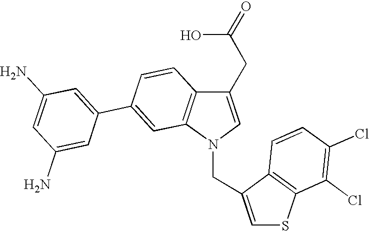 | A | Scheme 58 |
| 782 | 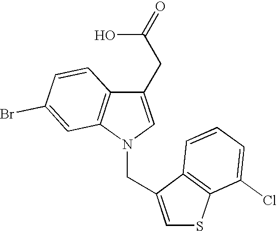 | C | Scheme 58 |
| 725 | 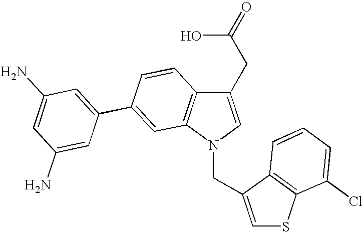 | A | Scheme 58 |
| 783 | 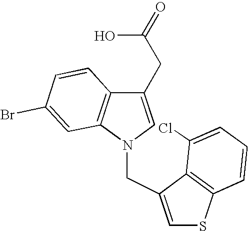 | A | Scheme 58 |
| 726 | 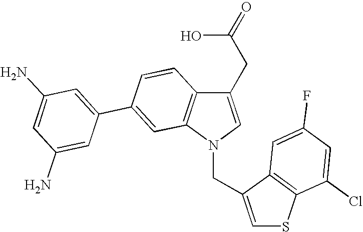 | B | Scheme 58 |
| 784 | 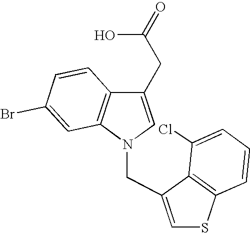 | C | Scheme 58 |
| 727 | 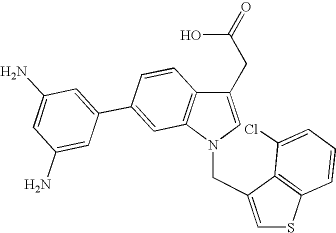 | B | Scheme 58 |
| 785 | 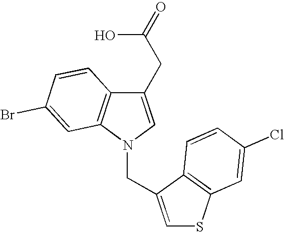 | C | Scheme 58 |
| 728 | 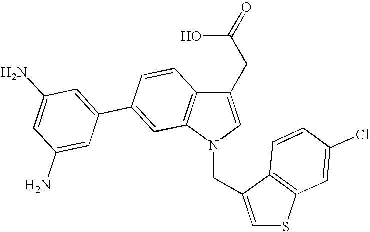 | B | Scheme 58 |
| 786 | 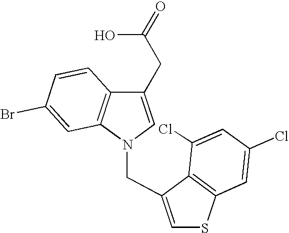 | C | Scheme 58 |
| 787 | 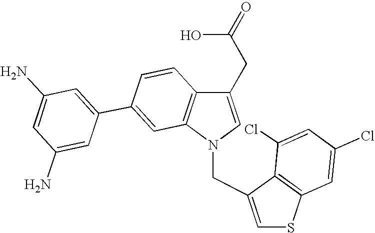 | A | Scheme 58 |
| 788 | 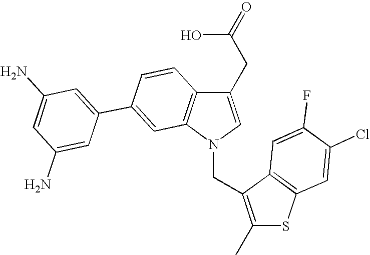 | B | Scheme 60 |
| 789 | 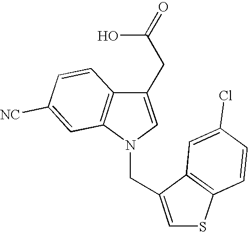 | A | Commercialbenzothiophene.Indole synthesisedviaScheme 53 |
| 790 | 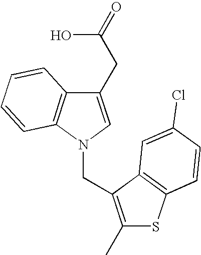 | C | Scheme 60 |

TABLE 37
Examples of compounds made using Scheme 55.

| | | IC50 | Synthetic route to |
|---|---|---|---|
| Example | Structure | Activity | alkyl bromide |
| 745 | 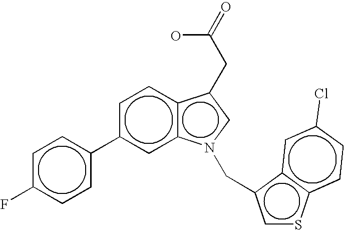 | C | Scheme 55 |
| 746 | 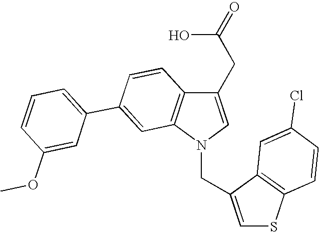 | C | Commercial |
| 747 | 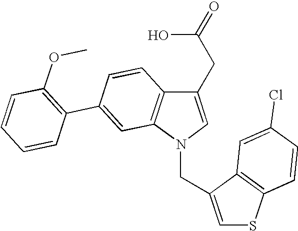 | C | Commercial |
| 748 | 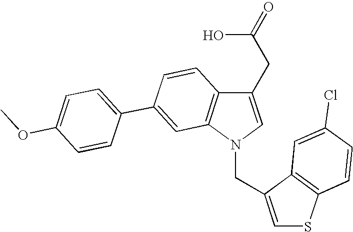 | C | Commercial |
| 749 | 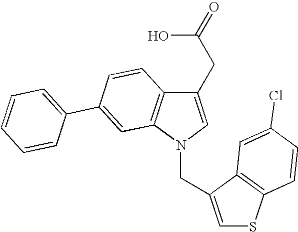 | C | Commercial |
| 750 | 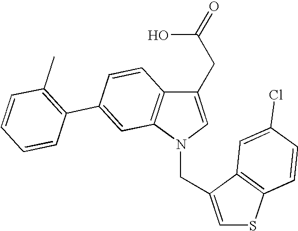 | A | Commercial |
| 751 | 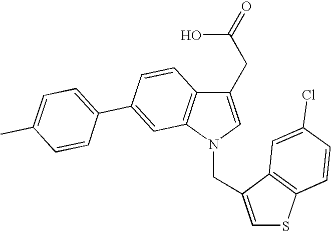 | C | Commercial |
| 752 | 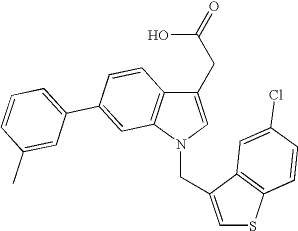 | A | Commercial |
| 753 | 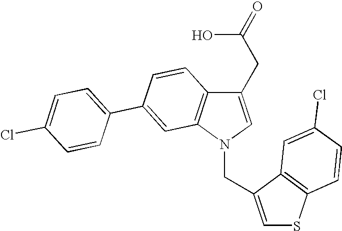 | C | Commercial |
| 754 | 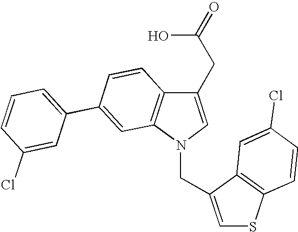 | C | Commercial |
| 755 | 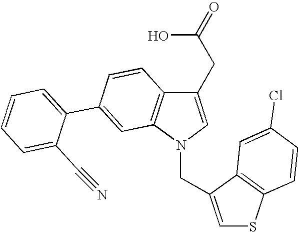 | C | Commercial |
| 756 | 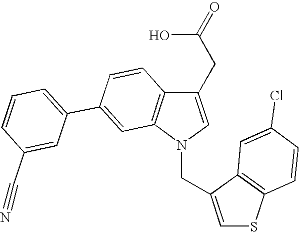 | A | Commercial |
| 757 | 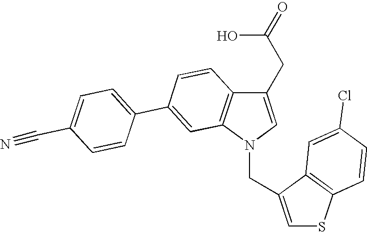 | A | Commercial |
| 758 | 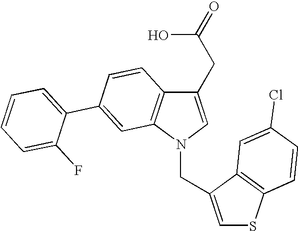 | C | Commercial |
| 759 | 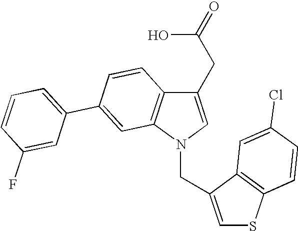 | C | Commercial |
| 760 | 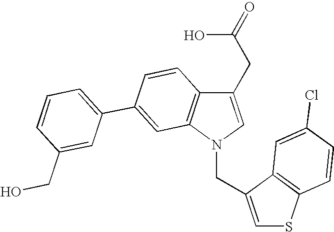 | A | Commercial |
| 761 | 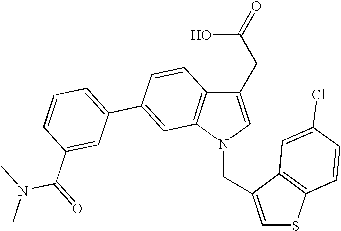 | A | Commercial |
| 762 | 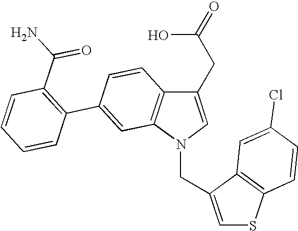 | C | Commercial |
| 763 | 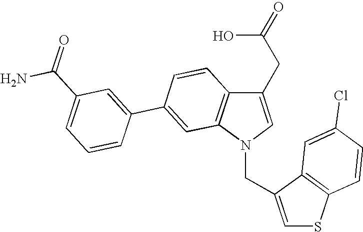 | A | Commercial |
| 764 | 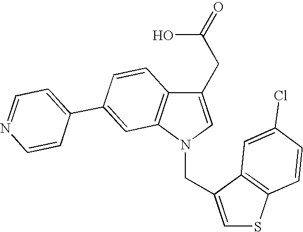 | A | Commercial |
| 765 | 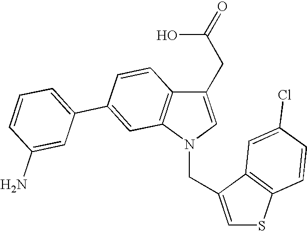 | B | Commercial |
| 766 | 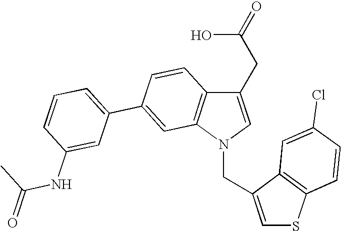 | A | Commercial(3-aminophenylboronic acidused) |
| 767 | 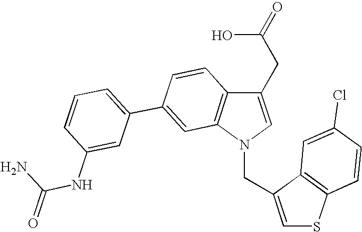 | A | Commercial(3-aminophenylboronic acidused) |
| 768 | 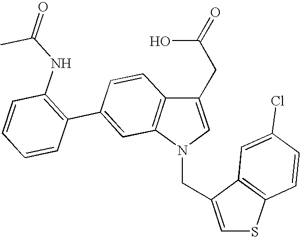 | A | Commercial(2-aminophenylboronic acidused) |
| 769 | 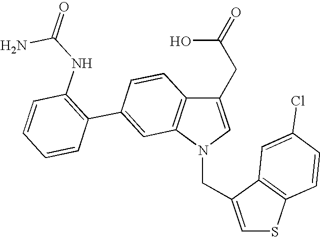 | C | Commercial(2-aminophenylboronic acidused) |
| 770 | 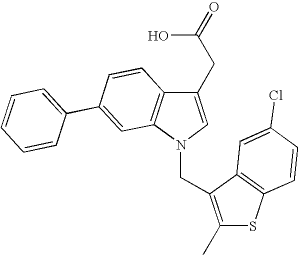 | B | Scheme 60 |

TABLE 38
Examples of compounds made using Scheme 56, Scheme 57 and Scheme 61.

| | | IC50 | Synthetic route to |
|---|---|---|---|
| Example | Structure | Activity | alkyl bromide |
| 736 | 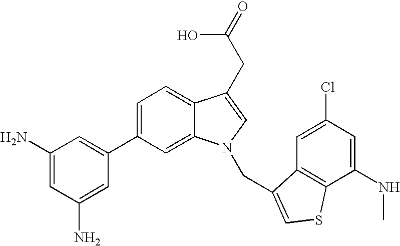 | | Scheme 56 |
| 729 | 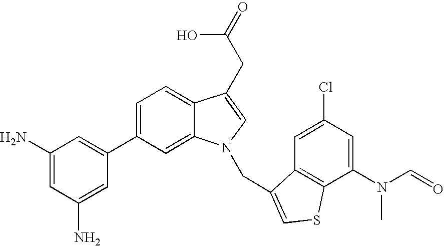 | A | Compound 21hydrogenated andsaponified viaScheme 54. Purifiedby HPLC, high pHmethod. |
| 730 | 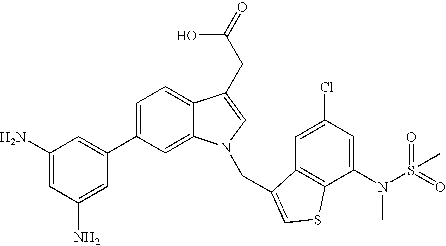 | A | Scheme 56 |
| 771 | 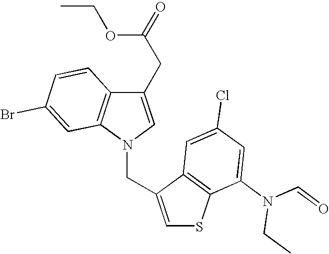 | C | Scheme 56 |
| 772 | 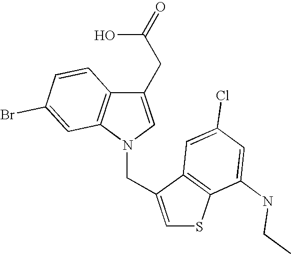 | C | Scheme 56 |
| 731 | 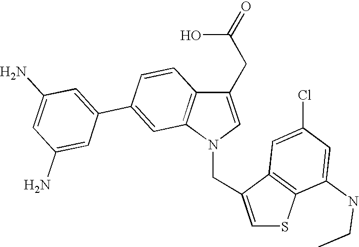 | B | Compound 24 |
| 732 | 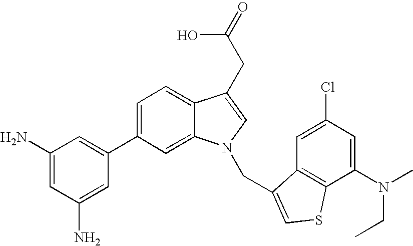 | B | Compound 25 |
| 773 | 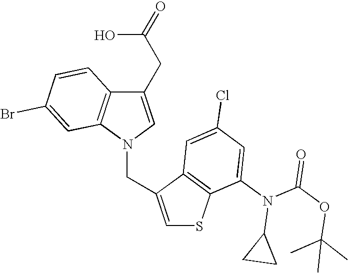 | C | Scheme 57 |
| 774 | 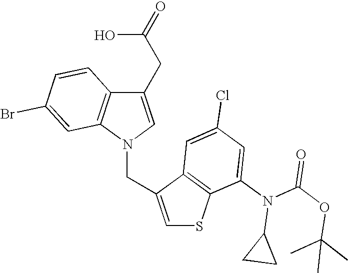 | B | Scheme 57 |
| 795 | 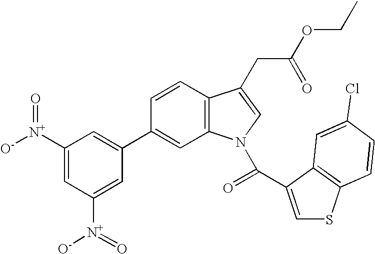 | C | Commercial |

TABLE 39
Examples of compounds made using Scheme 62.

| Example | Structure |
|---|---|
| 737 | 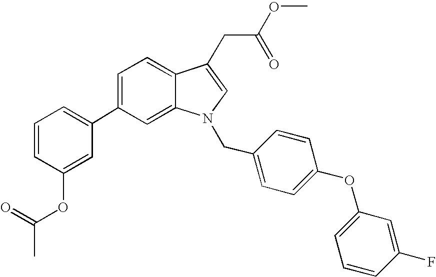 |
| 738 | 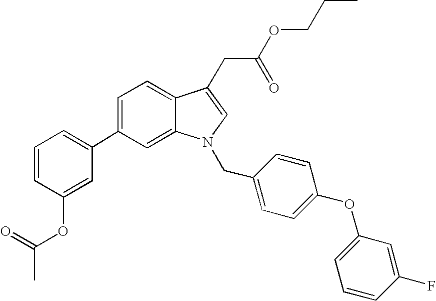 |
| 739 | 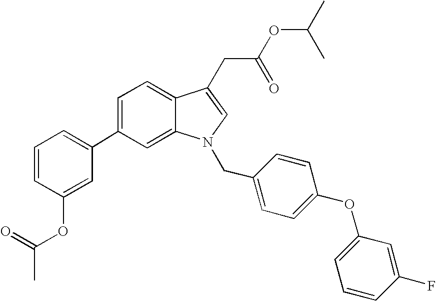 |
| 740 | 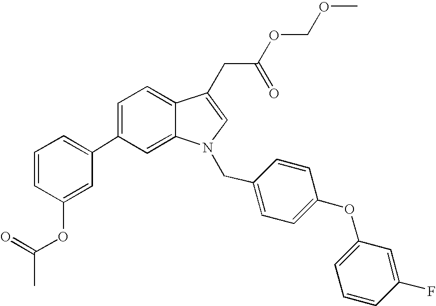 |
| 741 | 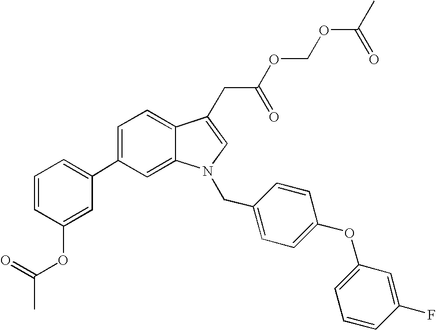 |
| 742 | 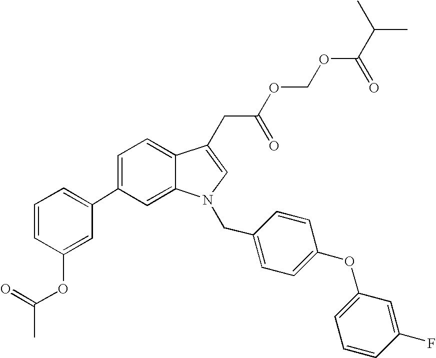 |
| 743 | 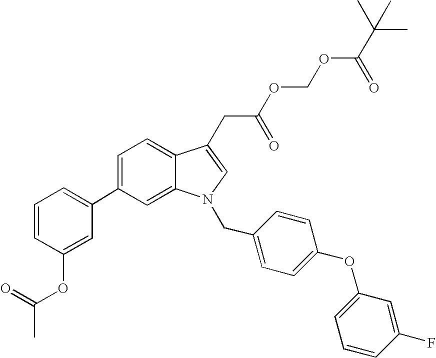 |
| 744 | 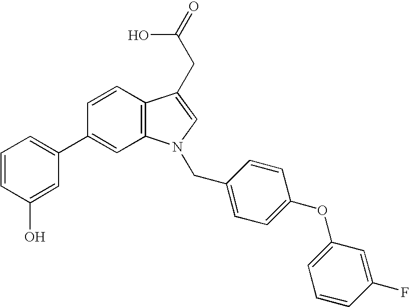 |

### CONCLUSION

Potent small molecule inhibitors of the HCV NS3 helicase have been developed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A compound having the structure of formula I: 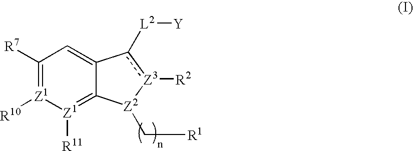
or a pharmaceutically acceptable salt, solvate, polymorph, or prodrug thereof, wherein:
n is an integer from 0 to 3;
R¹ is selected from the group consisting of H, -A¹-L¹-A², and an optionally substituted: alkyl, alkenyl, alkynyl, cycloalkyl, heterocyclyl, aryl, heteroaryl, —C(O)-aryl, —C(O)-aralkyl, —C(O)-heteroaryl, or —C(O)-heterocyclyl-aralkyl; or R¹ is absent and n is 0 when Z² is O or S;
wherein if R¹ is —C(O)-aryl, —C(O)-aralkyl, or —C(O)-heterocyclyl-aralkyl, then n is not 0;
A¹ and A² are independently selected from the group consisting of optionally substituted aryl and optionally substituted heteroaryl;
L¹ is oxy, C₁₋₆ alkoxy, —NR⁵C(O)-alkyl-, —NR⁵C(O)CH₂S—, —NR⁵CH₂—, —NR⁵ or absent;
L² is —CR^{3a}R^{3b}—, —CR^{3a}R^{3b}CR^{3a}R^{3b}—, —CR^{3a}═CR^{3a}—, or absent;
each R^{3a} and each R^{3b} are independently selected from the group consisting of H, halo, hydroxy, NH₃⁺, —NHC(O)NH₂, —NHC(O)OR⁹, —NHC(O)R⁹, —C(O)R⁴ and an optionally substituted: C₁₋₆ alkyl, cycloalkyl-alkyl, heterocyclyl-alkyl, heteroaralkyl, aralkyl, or aryl, or an R^{3a} and R^{3b} together form an oxo;
an R^{3a} together with R² optionally form an optionally substituted cycloalkyl or optionally substituted heterocyclyl;
Y is selected from the group consisting of H, halo, ethynyl, —C(O)H, —CN, —C(O)OR⁴, —C(O)NR⁵R⁶, —C(O)NHSO₂R⁹, —C(O)NHOR⁴, —C(O)OCH₃OC(O)R⁴, —NHC(O)R⁴, —C(O)NHOR⁴, —C(O)OCH₃OR⁴, —PO₃H₂, 1H-tetrazol-5-yl, 1H-1,2,4-triazol-5-yl, 1H-pyrazol-5-yl, 1,2-dihydro-1,2,4-triazol-3-on-5-yl, and 1,2-dihydro-pyrazol-3-on-5-yl,
wherein if Y is H, then:
at least one R^{3a} or R^{3b} is an optionally substituted aryl, or
R¹ is -A¹-L¹-A² or an optionally substituted: aryl, heteroaryl, —C(O)-aryl, —C(O)-aralkyl, or —C(O)-heterocyclyl-aralkyl;
R⁷ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl;
R¹⁰ is selected from the group consisting of H, halo, —CN, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, heterocyclyl, cycloalkylalkoxy, aryl, aralkyl, heteroaryl, heteroaralkyl, or is absent, or R⁷ and R¹⁰ together form an optionally substituted ring or ring system;
R¹¹ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl, or is absent;
each Z¹ are independently C or N;
Z² is CH, N, O, or S;
Z³ is C or N;
R² is selected from the group consisting of H, —C(O)OR⁴, —C(O)NR⁵R⁶, —C(O)-A¹-L¹-A², —CH₂-A¹-L¹-A², —C(O)CH₂-A¹-L¹-A², —C(O)NHCH₂-A¹-L¹-A², and an optionally substituted: alkyl, —C(O)-alkyl, aryl, —C(O)-aryl, aralkyl, —C(O)-aralkyl, or heteroaralkyl,
wherein if
R¹ is not -A¹-L¹-A² or an optionally substituted: aryl, heteroaryl, —C(O)-aryl, —C(O)-aralkyl, or —C(O)-heterocyclyl-aralkyl, then:
R² is selected from the group consisting of —C(O)-A¹-L¹-A², —CH₂-A¹-L¹-A², —C(O)CH₂-A¹-L¹-A², —CH₂-(optionally substituted heteroaryl), and optionally substituted —C(O)-aralkyl,
at least one R^{3a} or R^{3b} is an optionally substituted heteroaralkyl,
Y is —C(O)OH or —C(O)H and at least one Z¹ is N,
Y is —C(O)OH or —C(O)H and R¹⁰ is phenyl, phenyl substituted with one or more amino, or —O-benzyl,
Y is —C(O)OH or —C(O)H and R¹¹ is —O-(optionally substituted phenyl), or
Y is —C(O)OH or —C(O)H, R⁷ is —O-benzyl, and R¹⁰ is —O-methyl;
R⁴ is H or optionally substituted: alkyl, alkenyl, alkynyl, aryl, aralkyl, heteroaryl, heterocyclyl, or heteroaralkyl;
R⁵ and R⁶ are each independently selected from the group consisting of H, CN, and an optionally substituted: C₁₋₆ alkyl, C₃₋₇ cycloalkyl, hetereocyclyl, -hetereocyclyl-C(O)OR⁴, aryl, heteroaryl, aralkyl, heteroaralkyl, or cycloalkyl-alkyl, or R⁵ and R⁶ together form an optionally substituted ring or ring system; and
R⁹ is selected from the group consisting of alkyl, cycloalkyl, and aryl;
with the proviso that:
if R¹ is a pyridine, pyrimidine, or quinoline, or if R¹ is naphthalene and n is not 0, then Y is not CO₂H;
if R¹ is an unsubstituted phenyl, then Y is not —C(O)OMe, —C(O)OEt, —C(O)O-t-Bu, —C(O)OBn, —C(O)NMe₂, —C(O)NEt₂, or —C(O)N(i-Pr)₂;
if n is less than 3 and R¹ is an unsubstituted phenyl or unsubstituted biphenyl and Y is —C(O)OH, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn, Br, or phenyl substituted with one or more amino;
if Y is —C(O)OH and R¹ is phenyl substituted with a single halogen, —SO₂Me, —OCF₃, —OCF₂CF₃, —OCF₂CF₂H, —NC(O)CH₂Br, -Me, —SCH₃, or -t-Bu or R¹ is phenyl fused with a dioxolane ring, then R⁷ is —OBn or Br;
if Y is —C(O)OMe and R¹ is phenyl substituted with a single Cl, then R⁷ is —OBn;
if Y is —C(O)OEt and R¹ is phenyl substituted with a single halogen, —SO₂Me, —NH₂, —OH, —OCH₃, or —NO₂, or two Cl, then R⁷ is —OBn or R¹⁰ is phenyl substituted with one or more nitro;
if Y is —C(O)O-(substituted phenyl) and R¹ is phenyl substituted with two Cl, then R⁷ is —OBn;
if Y is —C(O)O-alkyl-phenyl and R¹ is unsubstituted phenyl or phenyl substituted with a single Br, then R⁷ is —OBn;
if n is 0 and R¹ is unsubstituted phenyl or phenyl substituted by a single methyl, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn;
if R¹ is -A¹-L¹-A², L¹ is methoxy, A¹ is unsubstituted phenyl, A² is phenyl substituted with a single CF₃, and Y is —C(O)OH, then R⁷ is —OBn;
if R¹ is -A¹-L¹-A², L¹ is absent, A¹ is benzofuran, A² is thiazole, and Y is —C(O)OH, then R⁷ is —OBn; and
if R¹ is -A¹-L¹-A², L¹ is methoxy or absent, A¹ is unsubstituted phenyl, A² is unsubstituted phenyl, R² is alkyl, and Y is —C(O)O-alkyl, then R⁷ is —OBn.

2. The compound of claim 1, wherein R¹ is an optionally substituted phenyl.

3. The compound of claim 1, wherein R¹ is an optionally substituted heteroaryl.

4. The compound of claim 1, wherein R¹ is -A¹-L¹-A².

5. The compound of claim 4, wherein A¹ and A² or optionally substituted phenyl.

6. The compound of claim 1, wherein Y is —C(O)OR⁴.

7. The compound of claim 1, wherein Y is —C(O)OH.

8. The compound of claim 1, wherein R⁷ is not H.

9. The compound of claim 1, wherein R⁷ is selected from the group consisting of halo, —CH═CH—C(O)OR⁴, —OR⁴, —SR⁴, —CH₂NHC(O)OR⁴, and —CH₂NHC(O)R⁴.

10. The compound of claim 1, wherein R⁷ is bromine or —O-benzyl.

11. The compound of claim 1, wherein R² is —C(O)NR⁵R⁶.

12. The compound of claim 1, wherein R² is an optionally substituted heteroaralkyl.

13. The compound of claim 1, wherein R² is selected from the group consisting of —C(O)NHCH₂-A¹-L¹-A² and —C(O)-A¹-L¹-A².

14. The compound of claim 13, wherein A¹ and A² are optionally substituted phenyl.

15. The compound of claim 1, wherein R¹⁰ is not H.

16. The compound of claim 1, wherein R¹⁰ is selected from the group consisting of halo, —CH═CH—C(O)OR⁴, —OR⁴, —SR⁴, —CH₂NHC(O)OR⁴, —CH₂NHC(O)R⁴, optionally substituted aryl, and optionally substituted heteroaryl.

17. The compound of claim 1, wherein at least one R^{3a} or R^{3b} is an optionally substituted aralkyl.

18. The compound of claim 1 having a formula selected from the group consisting of the formulas of compounds in Tables 1 through 39 as described in the specification.

19. The compound of claim 1, wherein the compound is a prodrug.

20. A pharmaceutical composition, comprising a compound claim 1 and a pharmaceutically acceptable excipient or carrier.

21. A method of inhibiting NS3/NS4 helicase activity comprising contacting a NS3/NS4 helicase with the compound of claim 1.

22. The method of claim 21 in which the contacting is conducted in vivo.

23. The method of claim 22, further comprising identifying a subject suffering from a hepatitis C infection and administering the compound or composition to the subject in an amount effective to treat the infection.

24. The method of claim 23 in which the contacting is conducted ex vivo.

25. The method of claim 24, wherein a sustained viral response is achieved.

26. The method of claim 24, wherein the method further comprises administering to the individual an effective amount of a nucleoside analog.

27. The method of claim 26, wherein the nucleoside analog is selected from ribavirin, levovirin, viramidine, an L-nucleoside, and isatoribine.

28. The method of claim 23, wherein the method further comprises administering to the individual pirfenidone or a pirfenidone analog administered orally daily in an amount of from about 400 mg to about 3600 mg.

29. The method of claim 23, wherein the method further comprises administering to the individual an effective amount of an NS3 protease inhibitor.

30. The method of claim 23, wherein the method further comprises administering to the individual an effective amount of an NS5B RNA-dependent RNA polymerase inhibitor.

31. The method of claim 23, wherein the method further comprises administering to the individual an effective amount of a tumor necrosis factor antagonist selected from the group consisting of etanercept, infliximab, and adalimumab.

32. The method of claim 23, wherein the method further comprises administering to the individual an effective amount of ritonavir.

33. The method of claim 23, wherein the method further comprises administering to the individual an effective amount of interferon-gamma (IFN-γ).

34. The method of claim 33, wherein the IFN-γ is administered subcutaneously in an amount of from about 10 μg to about 300 μg.

35. The method of claim 23, wherein the method further comprises administering to the individual an effective amount of interferon-alpha (IFN-α).

36. The method of claim 35, wherein the IFN-α is INFERGEN consensus IFN-α.

37. The method of claim 23, further comprising administering an effective amount of an agent selected from 3′-azidothymidine, 2′,3′-dideoxyinosine, 2′,3′-dideoxycytidine, 2-3-didehydro-2′,3′-dideoxythymidine, combivir, abacavir, adefovir dipoxil, cidofovir, ritonavir, and an inosine monophosphate dehydrogenase inhibitor.

38. A compound of claim 1 that is a salt.

39. The pharmaceutical composition of claim 20 wherein the compound is a salt.

40. A compound having the structure of formula I: 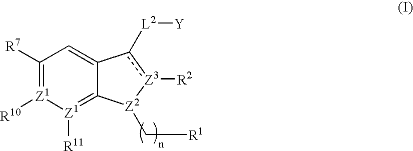
or a pharmaceutically acceptable salt, solvate, polymorph, or prodrug thereof, wherein:
n is an integer from 0 to 3;
R¹ is selected from the group consisting of H, -A¹-L¹-A², and an optionally substituted: alkyl, alkenyl, alkynyl, cycloalkyl, heterocyclyl, aryl, heteroaryl, —C(O)-aryl, —C(O)-aralkyl, or —C(O)-heterocyclyl-aralkyl; or R¹ is absent and n is 0 when Z² is O or S;
wherein if R¹ is —C(O)-aryl, —C(O)-aralkyl, or —C(O)-heterocyclyl-aralkyl, then n is not 0;
A¹ and A² are independently selected from the group consisting of optionally substituted aryl and optionally substituted heteroaryl;
L¹ is oxy, C₁₋₆ alkoxy, —NR⁵C(O)-alkyl-, —NR⁵C(O)CH₂S—, —NR⁵CH₂—, or absent;
L² is —CR^{3a}R^{3b}—, —CR^{3a}R^{3b}CR^{3a}R^{3b}—, —CR^{3a}═CR^{3a}—, or absent;
each R^{3a} and each R^{3b} are independently selected from the group consisting of H, halo, hydroxy, NH₃⁺, —NHC(O)NH₂, —NHC(O)OR⁹, —NHC(O)R⁹, and an optionally substituted: C₁₋₆ alkyl, cycloalkyl-alkyl, heterocyclyl-alkyl, heteroaralkyl, aralkyl, or aryl, or an R^{3a} and R^{3b} together form an oxo;
an R^{3a} together with R² optionally form an optionally substituted cycloalkyl or optionally substituted heterocyclyl;
Y is selected from the group consisting of H, halo, ethynyl, —C(O)H, —CN, —C(O)OR⁴, —C(O)NR⁵R⁶, —C(O)NHSO₂R⁹, —PO₃H₂, 1H-tetrazol-5-yl, 1H-1,2,4-triazol-5-yl, 1H-pyrazol-5-yl, 1,2-dihydro-1,2,4-triazol-3-on-5-yl, and 1,2-dihydro-pyrazol-3-on-5-yl,
wherein if Y is H, then:
at least one R^{3a} or R^{3b} is an optionally substituted aryl, or
R¹ is -A¹-L¹-A² or an optionally substituted: aryl, heteroaryl, —C(O)-aryl, —C(O)-aralkyl, or —C(O)-heterocyclyl-aralkyl;
R⁷ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl;
R¹⁰ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, heteroaralkyl, or is absent, or R⁷ and R¹⁰ together form an optionally substituted ring or ring system;
R¹¹ is selected from the group consisting of H, halo, —CH═CH—C(O)OR⁴, —OR⁴, —SR⁴, —CH₂NHC(O)OR⁴, —CH₂NHSO₂R⁹, —CH₂NHC(O)R⁴, and an optionally substituted: alkyl, alkenyl, alkynyl, alkoxy, cycloalkyl, cycloalkylalkoxy, aryl, aralkyl, heteroaryl, or heteroaralkyl, or is absent;
each Z¹ are independently C or N;
Z² is CH, N, O, or S;
Z³ is C or N;
R² is selected from the group consisting of H, —C(O)OR⁴, —C(O)NR⁵R⁶, —C(O)-A¹-L¹-A², —CH₂-A¹-L¹-A², —C(O)CH₂-A¹-L¹-A², —C(O)NHCH₂-A¹-L¹-A², and an optionally substituted: alkyl, —C(O)-alkyl, aryl, —C(O)-aryl, aralkyl, —C(O)-aralkyl, or heteroaralkyl,
wherein if
R¹ is not -A¹-L¹-A² or an optionally substituted: aryl, heteroaryl, —C(O)-aryl, —C(O)-aralkyl, or —C(O)-heterocyclyl-aralkyl, then:
R² is selected from the group consisting of —C(O)-A¹-L¹-A², —CH₂-A¹-L¹-A², —C(O)CH₂-A¹-L¹-A², —CH₂— (optionally substituted heteroaryl), and optionally substituted —C(O)-aralkyl,
at least one R^{3a} or R^{3b} is an optionally substituted heteroaralkyl,
Y is —C(O)OH or —C(O)H and at least one Z¹ is N,
Y is —C(O)OH or —C(O)H and R¹⁰ is phenyl or —O-benzyl,
Y is —C(O)OH or —C(O)H and R¹¹ is —O-(optionally substituted phenyl), or
Y is —C(O)OH or —C(O)H, R⁷ is —O-benzyl, and R¹⁰ is —O-methyl;
R⁴ is H or optionally substituted: alkyl, alkenyl, alkynyl, aryl, aralkyl, heteroaryl, or heteroaralkyl;
R⁵ and R⁶ are each independently selected from the group consisting of H, CN, and an optionally substituted: C₁₋₆ alkyl, C₃₋₇ cycloalkyl, hetereocyclyl, -hetereocyclyl-C(O)OR⁴, aryl, heteroaryl, aralkyl, heteroaralkyl, or cycloalkyl-alkyl, or R⁵ and R⁶ together form an optionally substituted ring or ring system; and
R⁹ is selected from the group consisting of alkyl, cycloalkyl, and aryl;
with the proviso that:
if R¹ is a pyridine, pyrimidine, or quinoline, or if R¹ is naphthalene and n is not 0, then Y is not CO₂H;
if R¹ is an unsubstituted phenyl, then Y is not —C(O)OMe, —C(O)OEt, —C(O)O-t-Bu, —C(O)OBn, —C(O)NMe₂, —C(O)NEt₂, or —C(O)N(i-Pr)₂;
if n is less than 3 and R¹ is an unsubstituted phenyl or unsubstituted biphenyl and Y is —C(O)OH, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn or Br;
if Y is —C(O)OH and R¹ is phenyl substituted with a single halogen, —SO₂Me, —OCF₃, —OCF₂CF₃, —OCF₂CF₂H, —NC(O)CH₂Br, -Me, —SCH₃, or -t-Bu or R¹ is phenyl fused with a dioxolane ring, then R⁷ is —OBn or Br;
if Y is —C(O)OMe and R¹ is phenyl substituted with a single Cl, then R⁷ is —OBn;
if Y is —C(O)OEt and R¹ is phenyl substituted with a single halogen, —SO₂Me, —NH₂, —OH, —OCH₃, or —NO₂, or two Cl, then R⁷ is —OBn;
if Y is —C(O)O-(substituted phenyl) and R¹ is phenyl substituted with two Cl, then R⁷ is —OBn;
if Y is —C(O)O-alkyl-phenyl and R¹ is unsubstituted phenyl or phenyl substituted with a single Br, then R⁷ is —OBn;
if n is 0 and R¹ is unsubstituted phenyl or phenyl substituted by a single methyl, then R² is selected from the group consisting of —C(O)-A¹-L¹-A², —CH₂-A¹-L¹-A², —C(O)CH₂-A¹-L¹-A², and an optionally substituted: —C(O)-aryl, aralkyl, —C(O)-aralkyl, or heteroaralkyl, or R⁷ is —OBn;
if R¹ is -A¹-L¹-A², L¹ is methoxy, A¹ is unsubstituted phenyl, A² is phenyl substituted with a single CF₃, and Y is —C(O)OH, then R⁷ is —OBn;
if R¹ is -A¹-L¹-A², L¹ is absent, A¹ is benzofuran, A² is thiazole, and Y is —C(O)OH, then R⁷ is —OBn; and
if R¹ is -A¹-L¹-A², L¹ is methoxy or absent, A¹ is unsubstituted phenyl, A² is unsubstituted phenyl, R² is alkyl, and Y is —C(O)O-alkyl, then R⁷ is —OBn.

41. The compound of claim 40, wherein R¹ is an optionally substituted phenyl.

42. The compound of claim 40, wherein R¹ is an optionally substituted heteroaryl.

43. The compound of claim 40, wherein R¹ is -A¹-L¹-A².

44. The compound of claim 43, wherein A¹ and A² or optionally substituted phenyl.

45. The compound of claim 40, wherein Y is —C(O)OR⁴.

46. The compound of claim 40, wherein Y is —C(O)OH.

47. The compound of claim 40, wherein R⁷ is not H.

48. The compound of claim 40, wherein R⁷ is selected from the group consisting of halo, —CH═CH—C(O)OR⁴, —OR⁴, —SR⁴, —CH₂NHC(O)OR⁴, and —CH₂NHC(O)R⁴.

49. The compound of claim 40, wherein R⁷ is bromine or —O-benzyl.

50. The compound of claim 40, wherein R² is —C(O)NR⁵R⁶.

51. The compound of claim 40, wherein R² is an optionally substituted heteroaralkyl.

52. The compound of claim 40, wherein R² is selected from the group consisting of —C(O)NHCH₂-A¹-L¹-A² and —C(O)-A¹-L¹-A².

53. The compound of claim 52, wherein A¹ and A² are optionally substituted phenyl.

54. The compound of claim 40, wherein R¹⁰ is not H.

55. The compound of claim 40, wherein R¹⁰ is selected from the group consisting of halo, —CH═CH—C(O)OR⁴, —OR⁴, —SR⁴, —CH₂NHC(O)OR⁴, —CH₂NHC(O)R⁴, optionally substituted aryl, and optionally substituted heteroaryl.

56. The compound of claim 40, wherein at least one R^{3a} or R^{3b} is an optionally substituted aralkyl.

57. The compound of claim 40 having a formula selected from the group consisting of the formulas of compounds in Tables 1 through 39 as described in the specification.

58. The compound of claim 40, wherein the compound is a prodrug.

59. A pharmaceutical composition, comprising a compound claim 40 and a pharmaceutically acceptable excipient or carrier.

60. A method of inhibiting NS3/NS4 helicase activity comprising contacting a NS3/NS4 helicase with the compound of claim 40.

61. The method of claim 60 in which the contacting is conducted in vivo.

62. The method of claim 61, further comprising identifying a subject suffering from a hepatitis C infection and administering the compound or composition to the subject in an amount effective to treat the infection.

63. The method of claim 62 in which the contacting is conducted ex vivo.

64. The method of claim 63, wherein a sustained viral response is achieved.

65. The method of claim 63, wherein the method further comprises administering to the individual an effective amount of a nucleoside analog.

66. The method of claim 65, wherein the nucleoside analog is selected from ribavirin, levovirin, viramidine, an L-nucleoside, and isatoribine.

67. The method of claim 62, wherein the method further comprises administering to the individual pirfenidone or a pirfenidone analog administered orally daily in an amount of from about 400 mg to about 3600 mg.

68. The method of claim 62, wherein the method further comprises administering to the individual an effective amount of an NS3 protease inhibitor.

69. The method of claim 62, wherein the method further comprises administering to the individual an effective amount of an NS5B RNA-dependent RNA polymerase inhibitor.

70. The method of claim 62, wherein the method further comprises administering to the individual an effective amount of a tumor necrosis factor antagonist selected from the group consisting of etanercept, infliximab, and adalimumab.

71. The method of claim 62, wherein the method further comprises administering to the individual an effective amount of ritonavir.

72. The method of claim 62, wherein the method further comprises administering to the individual an effective amount of interferon-gamma (IFN-γ).

73. The method of claim 72, wherein the IFN-γ is administered subcutaneously in an amount of from about 10 μg to about 300 μg.

74. The method of claim 62, wherein the method further comprises administering to the individual an effective amount of interferon-alpha (IFN-α).

75. The method of claim 74, wherein the IFN-α is INFERGEN consensus IFN-α.

76. The method of claim 62, further comprising administering an effective amount of an agent selected from 3′-azidothymidine, 2′,3′-dideoxyinosine, 2′,3′-dideoxycytidine, 2-,3-didehydro-2′,3′-dideoxythymidine, combivir, abacavir, adefovir dipoxil, cidofovir, ritonavir, and an inosine monophosphate dehydrogenase inhibitor.

77. A compound of claim 40 that is a salt.

78. The pharmaceutical composition of claim 59 wherein the compound is a salt.

79. A method of increasing protease activity of NS3 in solution comprising adding an effective amount of detergent to said solution.

80. The method of claim 79 wherein the detergent is LDAO.

81. The method of claim 80 wherein said LDAO is at a concentration between 0.1 mM and 1.0 mM in said solution.

82. The method of claim 80 further comprising adding a second detergent selected from the group consisting of Tween 20, Triton X100, Pluronic F127, CHAPS, β-octyl glucoside, laurylmaltoside, N-lauroylsarcosine, and hexadecyltrimethylammonium bromide.

83. The method of claim 82 wherein Triton X100 is added.

84. The method of claim 82 wherein Triton X100 is at a concentration between 0.01% and 0.10% in said solution.

85. The method of claim 79 wherein the increased protease activity of NS3 is at least 200% of basal NS3 protease activity.

86. A method of increasing helicase activity of NS3 in solution comprising adding an effective amount of detergent to said solution.

87. The method of claim 86 wherein the detergent is LDAO.

88. The method of claim 87 wherein said LDAO is at a concentration between 0.1 mM and 1.0 mM in said solution.

89. The method of claim 87 further comprising adding a second detergent selected from the group consisting of Tween 20, Triton X100, Pluronic F127, CHAPS, β-octyl glucoside, laurylmaltoside, N-lauroylsarcosine, and hexadecyltrimethylammonium bromide.

90. The method of claim 89 wherein Triton X100 is added.

91. The method of claim 90 wherein Triton X100 is at a concentration between 0.01% and 0.10% in said solution.

92. The method of claim 79 wherein the increased helicase activity of NS3 is at least 200% of basal NS3 helicase activity.

93. A method of increasing protease activity of NS3 in solution comprising adding an effective amount of an amine oxide to said solution.

94. The method of claim 93 wherein said amine oxide is selected from the group consisting of N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide.

95. The method of claim 93 wherein said amine oxide is LDAO.

96. The method of claim 95 wherein said LDAO is at a concentration between 0.1 mM and 1.0 mM in said solution.

97. The method of claim 93 further comprising adding a detergent selected from the group consisting of Tween 20, Triton X100, Pluronic F127, CHAPS, β-octyl glucoside, laurylmaltoside, N-lauroylsarcosine, and hexadecyltrimethylammonium bromide.

98. The method of claim 97 wherein Triton X100 is added.

99. The method of claim 98 wherein Triton X100 is at a concentration between 0.01% and 0.10% in said solution.

100. The method of claim 93 wherein the increased protease activity of NS3 is at least 200% of basal NS3 protease activity.

101. A method of increasing helicase activity of NS3 in solution comprising adding an effective amount of an amine oxide to said solution.

102. The method of claim 101 wherein said amine oxide is selected from the group consisting of N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide.

103. The method of claim 101 wherein said amine oxide is LDAO.

104. The method of claim 103 wherein said LDAO is at a concentration between 0.1 mM and 1.0 mM in said solution.

105. The method of claim 101 further comprising adding a detergent selected from the group consisting of Tween 20, Triton X100, Pluronic F127, CHAPS, β-octyl glucoside, laurylmaltoside, N-lauroylsarcosine, and hexadecyltrimethylammonium bromide.

106. The method of claim 105 wherein Triton X100 is added.

107. The method of claim 106 wherein Triton X100 is at a concentration between 0.01% and 0.10% in said solution.

108. The method of claim 101 wherein the increased helicase activity of NS3 is at least 200% of basal NS3 helicase activity.

109. A method of measuring helicase activity of NS3 in solution, comprising:
adding an effective amount of a detergent to the solution to increase the helicase activity of the NS3;
adding a double stranded oligonucleotide to the solution, wherein said oligonucleotide comprises a detectable marker on one strand and a moiety that quenches signal from said detectable marker on opposite strand;
allowing the NS3 to unwind the oligonucleotide, resulting in the separation of the two strands; and
measuring the signal generated by said detectable marker.

110. The method of claim 109, further comprising adding a capture oligonucleotide complimentary to the strand comprising the detectable marker.

111. The method of claim 109, wherein (+) strand of said oligonucleotide contains the detectable marker and (−) strand contains the quenching moiety.

112. The method of claim 109, wherein said detectable marker is a fluorescent marker.

113. The method of claim 112, wherein said fluorescent marker is a red-shifted dye.

114. The method of claim 113, wherein said red-shifted dye is selected from the group consisting of MR121 and Atto647 and the quenching moiety is three consecutive guanosine residues.

115. The method of claim 114, wherein said quenching moiety further comprises a biotin label.

116. The method of claim 115, wherein streptavidin is added to said solution to bind the biotin label to further quench the signal generated by the detectable marker.

117. The method of claim 109 wherein the detergent is LDAO.

118. The method of claim 117 wherein said LDAO is at a concentration between 0.1 mM and 1.0 mM in said solution.

119. The method of claim 117 further comprising adding a second detergent selected from the group consisting of Tween 20, Triton X100, Pluronic F127, CHAPS, β-octyl glucoside, laurylmaltoside, N-lauroylsarcosine, and hexadecyltrimethylammonium bromide.

120. The method of claim 119 wherein Triton X100 is added.

121. The method of claim 120 wherein Triton X100 is at a concentration between 0.01% and 0.10% in said solution.

122. The method of claim 119 wherein the increased helicase activity of NS3 is at least 200% of basal NS3 helicase activity.

123. A method of measuring a compound's ability to inhibit helicase activity of NS3 in solution, comprising:
adding the compound to the solution;
adding an effective amount of a detergent to the solution to increase the helicase activity of the NS3;
adding a double stranded oligonucleotide to the solution, wherein said oligonucleotide comprises a detectable marker on one strand and a moiety that quenches signal from said detectable marker on opposite strand;
allowing the NS3 to unwind the oligonucleotide, resulting in the separation of the two strands;
measuring the signal generated by said detectable marker; and
comparing the signal to a signal generated in absence of the compound to determine the ability of the compound to inhibit the helicase activity of NS3.

124. The method of claim 123, further comprising adding a capture oligonucleotide complimentary to the strand comprising the detectable marker.

125. The method of claim 123, wherein (+) strand of said oligonucleotide contains the detectable marker and (−) strand contains the quenching moiety.

126. The method of claim 123, wherein said detectable marker is a fluorescent marker.

127. The method of claim 126, wherein said fluorescent marker is a red-shifted dye.

128. The method of claim 127, wherein said red-shifted dye is selected from the group consisting of MR121 and Atto647 and the quenching moiety is three consecutive guanosine residues.

129. The method of claim 128, wherein said quenching moiety further comprises a biotin label.

130. The method of claim 129, wherein streptavidin is added to said solution to bind the biotin label to further quench the signal generated by the detectable marker.

131. The method of claim 123 wherein the detergent is LDAO.

132. The method of claim 131 wherein said LDAO is at a concentration between 0.1 mM and 1.0 mM in said solution.

133. The method of claim 131 further comprising adding a second detergent selected from the group consisting of Tween 20, Triton X100, Pluronic F127, CHAPS, β-octyl glucoside, laurylmaltoside, N-lauroylsarcosine, and hexadecyltrimethylammonium bromide.

134. The method of claim 133 wherein Triton X100 is added.

135. The method of claim 134 wherein Triton X100 is at a concentration between 0.01% and 0.10% in said solution.

136. The method of claim 123 wherein the increased helicase activity of NS3 is at least 200% of basal NS3 helicase activity.

137. A method of measuring ATPase activity of NS3 in solution, comprising:
adding an effective amount of a detergent to the solution to increase the ATPase activity of the NS3;
adding an ATP substrate to the solution;
adding antibody specific for ADP to the solution;
adding ADP linked to a detectable marker to the solution;
incubating the solution to allow NS3 to dephosphorylate the ATP to ADP; and
measuring the amount of detectable marker bound to the antibody, wherein decreased signal from the detectable marker correlates to increased ATPase activity of the NS3.

138. The method of claim 137 wherein a stop solution is added to stop the ATPase activity of NS3 before the signal from the detectable marker is measured.

139. The method of claim 137 wherein the detectable marker is a fluorescent label.

140. The method of claim 137 wherein the detergent is LDAO.

141. The method of claim 140 wherein said LDAO is at a concentration between 0.1 mM and 1.0 mM in said solution.

142. The method of claim 140 further comprising adding a second detergent selected from the group consisting of Tween 20, Triton X100, Pluronic F127, CHAPS, β-octyl glucoside, laurylmaltoside, N-lauroylsarcosine, and hexadecyltrimethylammonium bromide.

143. The method of claim 142 wherein Triton X100 is added.

144. The method of claim 143 wherein Triton X100 is at a concentration between 0.01% and 0.10% in said solution.

145. The method of claim 137 wherein the increased ATPase activity of NS3 is at least 200% of basal NS3 ATPase activity.

146. A method of measuring a compound's ability to inhibit ATPase activity of NS3 in solution, comprising:
adding the compound to the solution:
adding an effective amount of a detergent to the solution to increase the ATPase activity of the NS3;
adding an ATP substrate to the solution;
adding antibody specific for ADP to the solution;
adding ADP linked to a detectable marker to the solution;
incubating the solution to allow NS3 to dephosphorylate the ATP to ADP;
measuring the amount of detectable marker bound to the antibody, wherein decreased signal from the detectable marker correlates to increased ATPase activity of the NS3; and
comparing the signal to a signal generated in absence of the compound to determine the ability of the compound to inhibit the helicase activity of NS3.

147. The method of claim 146 wherein a stop solution is added to stop the ATPase activity of NS3 before the signal from the detectable marker is measured.

148. The method of claim 146 wherein the detectable marker is a fluorescent label.

149. The method of claim 146 wherein the detergent is LDAO.

150. The method of claim 149 wherein said LDAO is at a concentration between 0.1 mM and 1.0 mM in said solution.

151. The method of claim 149 further comprising adding a second detergent selected from the group consisting of Tween 20, Triton X100, Pluronic F127, CHAPS, β-octyl glucoside, laurylmaltoside, N-lauroylsarcosine, and hexadecyltrimethylammonium bromide.

152. The method of claim 151 wherein Triton X100 is added.

153. The method of claim 152 wherein Triton X100 is at a concentration between 0.01% and 0.10% in said solution.

154. The method of claim 146 wherein the increased ATPase activity of NS3 is at least 200% of basal NS3 ATPase activity.

155. A method of measuring helicase activity of NS3 in solution, comprising:
adding an effective amount of an amine oxide to the solution to increase the helicase activity of the NS3;
adding a double stranded oligonucleotide to the solution, wherein said oligonucleotide comprises a detectable marker on one strand and a moiety that quenches the signal from said detectable marker on opposite strand;
allowing the NS3 to unwind the oligonucleotide, resulting in the separation of the two strands; and
measuring the signal generated by said detectable marker.

156. The method of claim 155, further comprising adding a capture oligonucleotide complimentary to the strand comprising the detectable marker.

157. The method of claim 155, wherein (+) strand of said oligonucleotide contains the detectable marker and (−) strand contains the quenching moiety.

158. The method of claim 155, wherein said detectable marker is a fluorescent marker.

159. The method of claim 158, wherein said fluorescent marker is a red-shifted dye.

160. The method of claim 159, wherein said red-shifted dye is selected from the group consisting of MR121 and Atto647 and the quenching moiety is three consecutive guanosine residues.

161. The method of claim 160, wherein said quenching moiety further comprises a biotin label.

162. The method of claim 161, wherein streptavidin is added to said solution to bind the biotin label to further quench the signal generated by the detectable marker.

163. The method of claim 155 wherein said amine oxide is selected from the group consisting of N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide.

164. The method of claim 155 wherein said amine oxide is LDAO.

165. The method of claim 164 wherein said LDAO is at a concentration between 0.1 mM and 1.0 mM in said solution.

166. The method of claim 155 further comprising adding a detergent selected from the group consisting of Tween 20, Triton X100, Pluronic F127, CHAPS, β-octyl glucoside, laurylmaltoside, N-lauroylsarcosine, and hexadecyltrimethylammonium bromide.

167. The method of claim 166 wherein Triton X100 is added.

168. The method of claim 167 wherein Triton X100 is at a concentration between 0.01% and 0.10% in said solution.

169. The method of claim 155 wherein the increased protease activity of NS3 is at least 200% of basal NS3 protease activity.

170. A method of measuring a compound's ability to inhibit helicase activity of NS3 in solution, comprising:
adding the compound to the solution;
adding an effective amount of an amine oxide to the solution to increase the helicase activity of the NS3;
adding a double stranded oligonucleotide to the solution, wherein said oligonucleotide comprises a detectable marker on one strand and a moiety that quenches the signal from said detectable marker on opposite strand;
allowing the NS3 to unwind the oligonucleotide, resulting in the separation of the two strands;
measuring the signal generated by said detectable marker; and
comparing the signal to a signal generated in absence of the compound to determine the ability of the compound to inhibit the helicase activity of NS3.

171. The method of claim 170, further comprising adding a capture oligonucleotide complimentary to the strand comprising the detectable marker.

172. The method of claim 170, wherein (+) strand of said oligonucleotide contains the detectable marker and (−) strand contains the quenching moiety.

173. The method of claim 170, wherein said detectable marker is a fluorescent marker.

174. The method of claim 173, wherein said fluorescent marker is a red-shifted dye.

175. The method of claim 174, wherein said red-shifted dye is selected from the group consisting of MR121 and Atto647 and the quenching moiety is three consecutive guanosine residues.

176. The method of claim 175, wherein said quenching moiety further comprises a biotin label.

177. The method of claim 176, wherein streptavidin is added to said solution to bind the biotin label to further quench the signal generated by the detectable marker.

178. The method of claim 170 wherein said amine oxide is selected from the group consisting of N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide.

179. The method of claim 170 wherein said amine oxide is LDAO.

180. The method of claim 179 wherein said LDAO is at a concentration between 0.1 mM and 1.0 mM in said solution.

181. The method of claim 170 further comprising adding a detergent selected from the group consisting of Tween 20, Triton X100, Pluronic F127, CHAPS, β-octyl glucoside, laurylmaltoside, N-lauroylsarcosine, and hexadecyltrimethylammonium bromide.

182. The method of claim 181 wherein Triton X100 is added.

183. The method of claim 182 wherein Triton X100 is at a concentration between 0.01% and 0.10% in said solution.

184. The method of claim 170 wherein the increased helicase activity of NS3 is at least 200% of basal NS3 helicase activity.

185. A method of measuring ATPase activity of NS3 in solution, comprising:
adding an effective amount of an amine oxide to the solution to increase the ATPase activity of the NS3;
adding an ATP substrate to the solution;
adding antibody specific for ADP to the solution;
adding ADP linked to a detectable marker to the solution;
incubating the solution to allow NS3 to dephosphorylate the ATP to ADP; and
measuring the amount of detectable marker bound to the antibody, wherein decreased signal from the detectable marker correlates to increased ATPase activity of the NS3.

186. The method of claim 185 wherein a stop solution is added to stop the ATPase activity of NS3 before the signal from the detectable marker is measured.

187. The method of claim 185 wherein the detectable marker is a fluorescent label.

188. The method of claim 185 wherein said amine oxide is selected from the group consisting of N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide.

189. The method of claim 185 wherein said amine oxide is LDAO.

190. The method of claim 189 wherein said LDAO is at a concentration between 0.1 mM and 1.0 mM in said solution.

191. The method of claim 185 further comprising adding a detergent selected from the group consisting of Tween 20, Triton X100, Pluronic F127, CHAPS, β-octyl glucoside, laurylmaltoside, N-lauroylsarcosine, and hexadecyltrimethylammonium bromide.

192. The method of claim 191 wherein Triton X100 is added.

193. The method of claim 192 wherein Triton X100 is at a concentration between 0.01% and 0.10% in said solution.

194. The method of claim 185 wherein the increased ATPase activity of NS3 is at least 200% of basal NS3 ATPase activity.

195. A method of measuring the ability of a compound to inhibit ATPase activity of NS3 in solution, comprising:
adding the compound to the solution:
adding an effective amount of an amine oxide to the solution to increase the ATPase activity of the NS3;
adding an ATP substrate to the solution;
adding antibody specific for ADP to the solution;
adding ADP linked to a detectable marker to the solution;
incubating the solution to allow NS3 to dephosphorylate the ATP to ADP;
measuring the amount of detectable marker bound to the antibody, wherein decreased signal from the detectable marker correlates to increased ATPase activity of the NS3; and
comparing the signal to a signal generated in absence of the compound to determine the ability of the compound to inhibit the helicase activity of NS3.

196. The method of claim 195 wherein a stop solution is added to stop the ATPase activity of NS3 before the signal from the detectable marker is measured.

197. The method of claim 195 wherein the detectable marker is a fluorescent label.

198. The method of claim 195 wherein said amine oxide is selected from the group consisting of N,N-Dimethylhexylamine N-oxide, N,N-Dimethyloctylamine N-oxide, N,N-Dimethylnonylamine N-oxide, N,N-Dimethyldecylamine N-oxide, and N,N-Dimethyldodecylamine N-oxide.

199. The method of claim 195 wherein said amine oxide is LDAO.

200. The method of claim 199 wherein said LDAO is at a concentration between 0.1 mM and 1.0 mM in said solution.

201. The method of claim 195 further comprising adding a detergent selected from the group consisting of Tween 20, Triton X100, Pluronic F127, CHAPS, β-octyl glucoside, laurylmaltoside, N-lauroylsarcosine, and hexadecyltrimethylammonium bromide.

202. The method of claim 201 wherein Triton X100 is added.

203. The method of claim 202 wherein Triton X100 is at a concentration between 0.01% and 0.10% in said solution.

204. The method of claim 195 wherein the increased ATPase activity of NS3 is at least 200% of basal NS3 ATPase activity.
